SEQUENCE LISTING (2 sequences)

SEQ ID NO: 1 - DNA, 32 bp, Artificial Sequence
  synthetic primer
cagggcagaggatggatgcaaggataagtgga

SEQ ID NO: 2 - DNA, 32 bp, Artificial Sequence
  synthetic primer
tccacttatccttgcatccatcctctgccctg

(19) United States
(12) Patent Application Publication
(10) Pub. No.: US 2015/0307932 A1
(43) Pub. Date: Oct. 29, 2015

(54) Methods for Analyzing Minute Cellular Nucleic Acids

(71) Applicant: Fluidigm Corporation, South San Francisco, CA (US)

(72) Inventors: Fatih Ozsolak, Boston, MA (US); Patrice M. Milos, Cranston, RI (US)

(21) Appl. No.: 14/158,618

(22) Filed: Jan. 17, 2014

Related U.S. Application Data
(62) Division of application No. 12/904,683, filed on Oct. 14, 2010, now abandoned.
Application No. 12/904,683 is a continuation-in-part of application No. PCT/US10/24547, filed on Feb. 18, 2010.
(60) Provisional application No. 61/251,966, filed on Oct. 15, 2009.
(60) Provisional application No. 61/153,548, filed on Feb. 18, 2009.

(51) Int. Cl. C12Q 1/68

(52) U.S. Cl. CPC C12Q 1/6869

(57) ABSTRACT

The invention generally relates to methods for analyzing cellular nucleic acid. Methods of the invention involve capturing RNA from a lysed cell onto a substrate, producing a cDNA/RNA duplex, removing the RNA from the cDNA/RNA duplex, priming the cDNA to produce a primer/cDNA duplex, exposing the primer/cDNA duplex to at least one detectably labeled nucleotide in the presence of a polymerase capable of catalyzing addition of the nucleotide to the primer/cDNA duplex, detecting incorporation of the nucleotide into the primer portion, and repeating the exposing and detecting steps at least once.

### RELATED APPLICATIONS

The present invention is a continuation-in-part of PCT international patent application number PCT/US10/24547, filed Feb. 18, 2010, which claims the benefit of and priority to U.S. provisional patent application Ser. No. 61/153,548, filed Feb. 18, 2009. The present invention also claims the benefit of and priority to U.S. provisional patent application Ser. No. 61/251,966, filed Oct. 15, 2009. The content of each of these applications is incorporated by reference herein in its entirety.

### FIELD OF THE INVENTION

The invention generally relates to methods for analyzing cellular nucleic.

### BACKGROUND

Sequencing-by-synthesis involves template-dependent addition of nucleotides to a template/primer duplex. Nucleotide addition is mediated by a polymerase enzyme and added nucleotides may be labeled in order to facilitate their detection. Single molecule sequencing has been used to obtain high-throughput sequence information on individual DNA or RNA. See, Braslaysky, Proc. Natl. Acad. Sci. USA 100: 3960-64 (2003). Recently, all four Watson-Crick nucleotides may be added simultaneously, each with a different detectable label or nucleotides may be added one at a time in a step-and-repeat manner for imaging incorporations.

Although in most applications of this technology the amount of template nucleic acid is not limiting, a number of applications start from small quantities of nucleic acid. For example, when bacteria that cannot be cultured (Rappe et al., Annu. Rev. Microbiol. 57:369-394, 2003) or when cDNA libraries from a small number of cells (Schutze et al., Nat. Biotechnol. 16:737-742, 1998) are sequenced, template nucleic acid amounts limit the number of sequences that may be determined.

In order to increase the amount of template nucleic acid in a sample, an amplification reaction, e.g., PCR, typically is conducted. However, due to the stochastic nature of the amplification reaction, a population of molecules that is present in a small amount in the sample often is overlooked. In fact, if rare nucleic acid is not amplified in the first few rounds of amplification, it becomes increasingly unlikely that the rare event will ever be detected. Thus, the resulting biased post-amplification nucleic acid population does not represent the true condition of the sample from which it was obtained.

There is a need for methods that allow for analysis of samples that include only a small quantity of nucleic acid.

### SUMMARY

Methods of the invention allow for amplification-free analysis of very small quantities (e.g., nanogram, picogram, or fentogram amounts) of nucleic acids from a cell by direct sequencing methodologies. In this manner, methods of the invention allow for sequencing of biologically important rare cells without potentially biasing nucleic acid manipulation steps such as amplification. In particular embodiments, methods of the invention analyze nucleic acids obtained from only a single cell, such as a cancer cell.

Methods of the invention involve capturing RNA from a lysed cell onto a substrate, producing a cDNA/RNA duplex, removing the RNA from the cDNA/RNA duplex, priming the cDNA to produce a primer/cDNA duplex, exposing the primer/cDNA duplex to at least one detectably labeled nucleotide in the presence of a polymerase capable of catalyzing addition of the nucleotide to the primer/cDNA duplex, detecting incorporation of the nucleotide into the primer portion, and repeating the exposing and detecting steps at least once. In certain embodiments, methods of the invention further involve, prior to the capturing step, lysing a cell to release RNA from that cell. In certain embodiments, methods of the invention further involve removing a complementary strand of the cDNA, and resequencing the cDNA at least once.

Any surface capture method known in the art may be used to capture the RNA onto a substrate. In certain embodiments, the RNA is captured by adding a poly(A) tail to the RNA, and hybridizing the poly(A) tailed RNA to poly(T) primers that are attached to the substrate.

In certain embodiments, producing the cDNA/RNA duplex includes performing a sequencing reaction on the poly(A) tailed RNA that is hybridized to the poly(T) primer, thereby producing the cDNA/RNA duplex. Any sequencing method known in the art may be used. In particular embodiments, the sequencing reaction is a sequencing-by-synthesis reaction.

Either unique primers or universal primers, i.e., primers that all have the same sequence, may be used with methods of the invention. In certain embodiments, priming includes determining the sequence of the cDNA, synthesizing a primer that corresponds to a portion of the cDNA, and hybridizing the primer to the corresponding portion of the cDNA. In other embodiments, priming involves adding a poly(G) tail to a 3′ end of the cDNA, and hybridizing a poly(C) primer to the poly(G) tail. In certain embodiments, prior to the exposing step, the method further involves adding dCTP to the primer/cDNA duplex to fill remaining unpaired poly(G) nucleotides on the poly(G) tail of the cDNA, thereby ensuring alignment of the poly(C) primer with the poly(G) tail. Methods of the invention may also include exposing the primer/cDNA duplex to locked nucleic acids. Methods of the invention may also include, prior to the exposing step, adding dATP in order to fill poly(T) primers on the surface of the substrate that did not pair with an RNA. In certain embodiments, at least a portion of the primer/cDNA duplexes are individually optically resolvable.

### BRIEF DESCRIPTION OF THE FIGURES

FIG. 1 is a schematic showing steps of certain embodiments of methods of the invention. An RNA template having a poly(A) tail is captured on a surface by hybridization to a poly(T) primer covalently attached to the surface. After capturing the poly(A) tailed RNA, dNTPs and reverse transcriptase are used to obtain a cDNA/RNA hybrid along the length of the original RNA template. The RNA template is removed by passing hot water through the flow channel. cDNA generated during the reverse transcription step remains covalently attached on the surface. Terminal transferase is used to add approximately 25 nt G residues to the 3′ end of the cDNA, followed by 3′ blocking with ddGTP and ddATP. Poly(C) primers (approximately 15 nt long) are hybridized to the generated poly(G) priming sites. A stepwise “fill” with dCTP is performed to ensure that the alignment of the poly(C) primer within the poly(G) tail is suitable for initiation of single molecule sequencing at the 3′ end of the cDNA. After surface washes, another fill step with dATP is performed in order to fill up the poly(dT) primers on the surface that do not contain a template, but still can be G-tailed by terminal transferase. This step prevents undesirable sequencing of G-tailed poly(dT) primers during sequencing, which would result in the abundance of poly-A reads. A “lock” step with virtual terminator-C, G and T (commercially available from Helicos Biosciences Corporation, Cambridge Mass.) is performed, followed by single molecule sequencing.

FIG. 2 is a set of graphs showing throughput, reproducibility and counting power of methods of the invention. Panel A is a graph showing representative usable read yields obtained per channel. The X axis shows the number of cells introduced into a flow cell channel. The Y axis shows the number of reads (in millions) aligned to a mouse transcriptome. Black columns are the 490 cells; grey columnsthe SM25 cells.

Panel B is a graph showing the reproducibility of methods of the invention across independent runs. The Pearson correlation coefficient between two experiments was 0.991. A thousand 490 cells per channel in the AmpliGrid lysis system were used.

Panel C is a graph showing correlation of gene counts obtained with methods of the invention compared to established digital gene expression assays performed using standard approaches (e.g., Lipson et al., Nat Biotechnol 27:652-658, 2009). The Expression profile obtained with methods of the invention of a thousand 490 cells was compared to a standard digital gene expression approach performed with 10 microgram 490 RNA isolated with Trizol from five million cells (Lipson et al., Nat Biotechnol 27:652-658, 2009). Both datasets are in high agreement (r=0.901). In panels B and C, X and Y axes indicate log₁₀ counts obtained per gene, each axes normalized to one million total reads.

Panel D is a graph showing the cumulative read length distribution of raw (diamond), filtered (square) and aligned (triangle) reads in a single channel. The mean aligned read length is 36.5nts.

FIG. 3 is a set of graphs showing differential expression. Panel A is a graph showing profiles obtained from 490 and SM25 cells exhibit gene expression differences (r=0.81). Panel B is a graph showing validation of differentially expressed genes with the QRT-PCR assay. Black columns indicate fold differences observed with the LQ-DGE; gray columns with QRT-PCR. Y-axis indicates relative transcript level in SM25 relative to 490 cells.

FIG. 4 is a set of graphs showing similarity of gene counts obtained using different cell lysis conditions. The lysis buffer and conditions were done using the following commercial kits: buffer 1: Molecular Devices PicoPure RNA Isolation Kit; buffer 2: Advalytix AmpliGrid Cell Extraction Kit; buffer 3: Invitrogen CellsDirect Two-Step qRT-PCR Kit; and buffer 4: Qiagen FastLane Cell cDNA kit. The Pearson correlation coefficients were: buffer 1-2: r=0.946; buffer 2-3: r=0.942; buffer 3-4: r=0.943; buffer 2-4: r=0.937; buffer 1-3: r=0.94; and buffer 1-4: r=0.945. X and Y axes indicate counts obtained per reference sequence gene. Each experiment normalized to one million total read count.

FIG. 5 is a graph showing reproducibility of measurements using two different reverse transcriptases. The Pearson correlation coefficient between the two experiments was 0.993. A thousand SM25 cells per channel in the AmpliGrid lysis system were used. X and Y axes indicate counts obtained per reference sequence gene, each axes normalized to one million total read count.

### DETAILED DESCRIPTION

The invention generally relates to methods for analyzing cellular nucleic acids. Methods of the invention involve capturing RNA from a lysed cell onto a substrate, producing a cDNA/RNA duplex, removing the RNA from the cDNA/RNA duplex, priming the cDNA to produce a primer/cDNA duplex, exposing the primer/cDNA duplex to at least one detectably labeled nucleotide in the presence of a polymerase capable of catalyzing addition of the nucleotide to the primer/cDNA duplex, detecting incorporation of the nucleotide into the primer portion, and repeating the exposing and detecting steps at least once.

Methods of the invention are advantageous because they do not require lossy RNA isolation or poly(A) tailed RNA selection steps. Methods of the invention take advantage of the higher-affinity RNA-DNA hybridization kinetics relative to DNA-DNA hybridization kinetics, allowing a higher fraction of poly(A) tailed templates to be captured on flow cell surfaces. Methods of the invention do not require RNA or cDNA fragmentation or size selection steps, thus minimizing bias particularly against short transcripts. Since each transcript captured on the surface can give rise to only one read, expression levels are independent of transcript length and thus do not require normalization for transcript length or other factors. Due to the lack of biasing, sample manipulation steps such as ligation, restriction digestion or amplification, artifacts are minimized.

### RNA Samples

Ribonucleic acids (RNA) are derived from naturally occurring sources, e.g., cells. In one embodiment, RNA molecules are isolated from a biological sample containing a variety of other components, such as proteins, lipids and non-template nucleic acids. RNA molecules can be obtained from any cellular material, obtained from an animal, plant, bacterium, fungus, or any other cellular organism. In certain embodiments, the RNA templates are obtained from a single cell. Biological samples for use in the present invention include viral particles or preparations. RNA molecules can be obtained directly from an organism or from a biological sample obtained from an organism, e.g., from blood, urine, cerebrospinal fluid, seminal fluid, saliva, sputum, stool and tissue. Any tissue or body fluid specimen may be used as a source for RNA for use in the invention. RNA molecules can also be isolated from cultured cells, such as a primary cell culture or a cell line. The cells or tissues from which RNA molecules are obtained can be infected with a virus or other intracellular pathogen. A sample can also be total RNA extracted from a biological specimen, a cDNA library, viral, or genomic DNA.

RNA obtained from biological samples typically is fragmented to produce suitable fragments for analysis. In one embodiment, RNA from a biological sample is fragmented by sonication. RNA molecules can be obtained as described in U.S. Patent Application Publication Number US2002/0190663 A1, published Oct. 9, 2003. Generally, RNA can be extracted from a biological sample by a variety of techniques such as those described by Maniatis, et al., Molecular Cloning: A Laboratory Manual, Cold Spring Harbor, N.Y., pp. 280-281 (1982). Generally, individual RNA template molecules can be from about 5 bases to about 20 kb. RNA molecules may be single-stranded, double-stranded, or double-stranded with single-stranded regions (for example, stem- and loop-structures).

A biological sample as described herein may be homogenized or fractionated in the presence of a detergent or surfactant. The concentration of the detergent in the buffer may be about 0.05% to about 10.0%. The concentration of the detergent can be up to an amount where the detergent remains soluble in the solution. In a preferred embodiment, the concentration of the detergent is between 0.1% to about 2%. The detergent, particularly a mild one that is nondenaturing, can act to solubilize the sample. Detergents may be ionic or nonionic. Examples of nonionic detergents include triton, such as the Triton® X series (Triton® X-100 t-Oct-C₆H₄—(OCH₂—CH₂)ₓOH, x=9-10, Triton® X-100R, Triton® X-114 x=7-8), octyl glucoside, polyoxyethylene(9)dodecyl ether, digitonin, IGEPAL® CA630 octylphenyl polyethylene glycol, n-octyl-beta-D-glucopyranoside (betaOG), n-dodecyl-beta, Tween® 20 polyethylene glycol sorbitan monolaurate, Tween® 80 polyethylene glycol sorbitan monooleate, polidocanol, n-dodecyl beta-D-maltoside (DDM), NP-40 nonylphenyl polyethylene glycol, C12E8 (octaethylene glycol n-dodecyl monoether), hexaethyleneglycol mono-n-tetradecyl ether (C14EO6), octyl-beta-thioglucopyranoside (octyl thioglucoside, OTG), Emulgen, and polyoxyethylene 10 lauryl ether (C12E10). Examples of ionic detergents (anionic or cationic) include deoxycholate, sodium dodecyl sulfate (SDS), N-lauroylsarcosine, and cetyltrimethylammoniumbromide (CTAB). A zwitterionic reagent may also be used in the purification schemes of the present invention, such as Chaps, zwitterion 3-14, and 3-[(3-cholamidopropyl)dimethylammonio]-1-propanesulf-onate. It is contemplated also that urea may be added with or without another detergent or surfactant.

Lysis or homogenization solutions may further contain other agents, such as reducing agents. Examples of such reducing agents include dithiothreitol (DTT), β-mercaptoethanol, DTE, GSH, cysteine, cysteamine, tricarboxyethyl phosphine (TCEP), or salts of sulfurous acid.

### RNA Capture

The RNA capture step prior to producing a cDNA/RNA duplex may be any suitable hybrid capture method. For example, capture can occur in solution, on beads (polystyrene beads), in a column (such as a chromatography column), in a gel (such as a polyacrylamide gel), or directly on the surface to be used for producing the cDNA/RNA. An array of support-bound capture oligos can be used to hybridize specifically to a target sequence. Additionally, chromatography-based capture techniques are useful. For example, ion exchange chromatography, HPLC, gas chromatography, and gel-based chromatography all are useful. In one embodiment, gel-based capture is used in order to achieve sequence-specific capture. Using this method, multiple different sequences are captured simultaneously using immobilized probes in the gel. The target sequences are isolated by removing portions of the gel containing them and eluting target from the gel portions for sequencing.

In an alternative embodiment, the target RNA molecule either includes, or is modified to include, an adaptor sequence (such as a polyadenylation region) that is complementary to a portion of a capture probe in order to aid in the capture of the RNA. A preferred embodiment involves an immobilized capture probe having a sequence that hybridizes (e.g., is complementary to) with the adaptor sequence. Methods of the invention are conducted by contacting capture probes with a sample including RNA molecules from a lysed cell under conditions suitable for specific hybridization between the target RNA molecule and immobilized capture probe, thereby forming target/capture probe duplexes. A wash step removes debris and unhybridized nucleic acid in the sample. In one embodiment, the cDNA/RNA duplex is produced using the capture probe as a primer. In another embodiment, the target/capture probe duplex is melted to release the target RNA. The resulting purified target population is analyzed as described below.

If target nucleic acid is melted off the capture probe, the targets are either attached to a surface for production of cDNA/RNA duplexes or hybridized to primers that have been attached to the surface. Surface attachment of oligonucleotides for sequencing can be direct or indirect. For example, nucleic acids are attached to an epoxide surface via a direct amine linkage as described below. Alternatively, the surface is prepared with a binding partner, the opposite of which is attached to the RNA. For example, the surface can be streptavidinated and biotinylated nucleic acids can be used to form an attachment at the surface. Other binding pairs (e.g., antibody/antigen, such as digoxigenen/anti-digoxigenen and dinitorphenol/anti-dinitrophenol) can also be used.

Further description of hybrid capture is provided in Lapidus (U.S. patent application number 2007/0048744), the content of which is incorporated by reference herein in its entirety.

### Surfaces and Attachment

In a preferred embodiment, RNA molecules are attached to a substrate (also referred to herein as a surface). In certain embodiments, RNA molecules are attached to the surface such that the molecules are individually optically resolvable. Substrates for use in the invention can be two- or three-dimensional and can comprise a planar surface (e.g., a glass slide) or can be shaped. A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites.

Suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring a nucleic acid. Substrates can include planar arrays or matrices capable of having regions that include populations of template nucleic acids or primers. Examples include nucleoside-derivatized CPG and polystyrene slides; derivatized magnetic slides; polystyrene grafted with polyethylene glycol, and the like.

Substrates are preferably coated to allow optimum optical processing and nucleic acid attachment. Substrates for use in the invention can also be treated to reduce background. Exemplary coatings include epoxides, and derivatized epoxides (e.g., with a binding molecule, such as an oligonucleotide or streptavidin).

Various methods can be used to anchor or immobilize the nucleic acid molecule to the surface of the substrate. The immobilization can be achieved through direct or indirect bonding to the surface. The bonding can be by covalent linkage. See, Joos et al., Analytical Biochemistry 247:96-101, 1997; Oroskar et al., Clin. Chem. 42:1547-1555, 1996; and Khandjian, Mol. Bio. Rep. 11:107-115, 1986. A preferred attachment is direct amine bonding of a terminal nucleotide of the template or the 5′ end of the primer to an epoxide integrated on the surface. The bonding also can be through non-covalent linkage. For example, biotin-streptavidin (Taylor et al., J. Phys. D. Appl. Phys. 24:1443, 1991) and digoxigenin with anti-digoxigenin (Smith et al., Science 253:1122, 1992) are common tools for anchoring nucleic acids to surfaces and parallels. Alternatively, the attachment can be achieved by anchoring a hydrophobic chain into a lipid monolayer or bilayer. Other methods for known in the art for attaching nucleic acid molecules to substrates also can be used.

### RNA Tailing

In certain embodiments, a tail is attached to the RNA molecules. RNA tailing is described for example in Steinman et al. (International patent application number PCT/US09/64001), the content of which is incorporated by reference herein in its entirety. The RNA tails act as a primer binding sites. The primer binding site may be used to hybridize the template RNA molecule to a sequencing primer (e.g., a poly(T) sequence), which may optionally be anchored to a substrate. The primer binding sequence may be a unique sequence including at least 2 bases but likely contains a unique order of all 4 bases and is generally 20-50 bases in length. One example of a specific sequence binding primer is: 5′-CAG GGC AGA GGA TGG ATG CAA GGA TAA GTG GA-3′ (SEQ ID NO: 1). In a particular embodiment, the primer binding sequence is a homopolymer of a single base, e.g. poly(T), generally 20-200 bases in length.

The RNA tail also may include a blocker, e.g., a chain terminating nucleotide, on the 3′-end. The blocker prevents unintended sequence information from being obtained using the 3′-end of the primer binding site inadvertently as a second sequencing primer, particularly when using homopolymeric primer sequences. The blocker may be any moiety that prevents a polymerase from adding bases during incubation with a dNTPs. An exemplary blocker is a nucleotide terminator that lacks a 3′-OH, i.e., a dideoxynucleotide (ddNTP). Common nucleotide terminators are 2′,3′-dideoxynucleotides, 3′-aminonucleotides, 3′-deoxynucleotides, 3′-azidonucleotides, acyclonucleotides, etc. The blocker may have attached a detectable label, e.g. a fluorophore. The label may be attached via a labile linkage, e.g., a disulfide, so that following hybridization of the template RNA to the surface, the locations of the template nucleic acids may be identified by imaging. Generally, the detectable label is removed before commencing with sequencing. Depending upon the linkage, the cleaved product may or may not require further chemical modification to prevent undesirable side reactions, for example following cleavage of a disulfide by TCEP the produced reactive thiol is blocked with iodoacetamide.

Methods of the invention involve attaching the oligonucleotide tail to the template RNA molecules. In certain embodiments, the oligonucleotide tail is attached to the template RNA molecule with an enzyme, such as terminal transferase. The enzyme may be a ligase or a polymerase. The ligase may be any enzyme capable of ligating an oligonucleotide to the template RNA molecule. Suitable ligases include T4 RNA ligase (such ligases are available commercially, from New England Biolabs). Methods for using ligases are well known in the art. The polymerase may be any enzyme capable of adding nucleotides to the 3′ terminus of template RNA molecules. The polymerase may be, for example, yeast poly(A) polymerase, commercially available from USB. The polymerase is used according to the manufacturer's instructions. In a particular embodiment, the enzyme is a terminal transferase, which is commercially from New England Biolabs. The enzyme is used according to the manufacturer's instructions.

The ligation may be blunt ended or via use of complementary over hanging ends. In certain embodiments, the ends of the template RNA are repaired, trimmed (e.g. using an exonuclease), or filled (e.g., using a polymerase and dNTPs), to form blunt ends. Upon generating blunt ends, the ends may be treated with a polymerase and dATP to form a template independent addition to the 3′-end of the template RNA, thus producing a single A overhanging. This single A is used to guide ligation of fragments with a single T overhanging from the 5′-end in a method referred to as T-A cloning.

Alternatively, because the possible combination of overhangs left by the restriction enzymes are known after a restriction digestion, the ends may be left as is, i.e., ragged ends. In certain embodiments double stranded oligonucleotides with complementary over hanging ends are used. In a particular example, the A:T single base over hang method is used (see Steinman et al., International patent application number PCT/US09/64001).

In a particular embodiment, a substrate has anchored a reverse complement to the primer binding sequence of the oligonucleotide, for example 5′-TC CAC TTA TCC TTG CAT CCA TCC TCT GCC CTG (SEQ ID NO: 2) or a poly(T)(50). When homopolymeric sequences are used for the primer, it may be advantageous to perform a procedure known in the art as a “fill and lock”. When poly(A) (20-70) on the template RNA and poly(T)(50) on the surface hybridize there is a high likelihood that there will not be perfect alignment, so the hybrid is filled in by incubating the sample with polymerase and TTP. Following the fill step, the sample is washed and the polymerase is incubated with one or two dNTPs complementary to the base(s) used in the lock sequence. The fill and lock can also be performed in a single step process in which polymerase, TTP and one or two reversible terminators (complements of the lock bases) are mixed together and incubated. The reversible terminators stop addition during this stage and can be made functional again (reversal of inhibitory mechanism) by treatments specific to the analogs used. Some reversible terminators have functional blocks on the 3′-OH which need to be removed while others, for example Helicos BioSciences Virtual Terminators have inhibitors attached to the base via a disulfide which can be removed by treatment with TCEP.

After tailing, the tailed RNA are introduced to primers and RNA template/primer duplexes are formed. A cDNA/RNA duplex is then produced as described below. Further description is provided in Kahvejian (U.S. patent application number 2008/0081330), the content of which is incorporated by reference herein in its entirety.

cDNA/RNA Duplexes

Single molecule sequencing methodologies may be used to produce the cDNA/RNA duplexes, and such methodologies are discussed in further detail below. Single molecule sequencing is shown for example in Lapidus et al. (U.S. Pat. No. 7,169,560), Lapidus et al. (U.S. patent application number 2009/0191565), Quake et al. (U.S. Pat. No. 6,818,395), Harris (U.S. Pat. No. 7,282,337), Quake et al. (U.S. patent application number 2002/0164629), and Braslaysky, et al., PNAS (USA), 100: 3960-3964 (2003), the contents of each of these references is incorporated by reference herein in its entirety.

Briefly, the tailed RNA are attached to a surface of a flow cell. The RNA may be covalently attached to the surface or various attachments other than covalent linking as known to those of ordinary skill in the art may be employed. Moreover, the attachment may be indirect, e.g., via hybridization to a primer already attached to the surface or via a polymerase directly or indirectly attached to the surface. The surface may be planar or otherwise, and/or may be porous or non-porous, or any other type of surface known to those of ordinary skill to be suitable for attachment. A strand of cDNA is then produced by polymerase-mediated addition of nucleotides incorporated into the growing strand surface primer. In certain embodiments, the nucleotides used in the sequencing reaction are not chain terminating nucleotides.

In certain embodiments, it may be beneficial to know the sequence of the cDNA strand, and thus fluorescently-labeled nucleotides are used in the reaction. The cDNA sequence is obtained by imaging the polymerase-mediated addition of fluorescently-labeled nucleotides incorporated into the growing strand surface oligonucleotide, at single molecule resolution. Knowing the sequence of the cDNA allows for design of primers that are capable of hybridizing to the 3′ end of the cDNA molecule for subsequence sequencing reactions.

Alternatively, certain embodiments do not require knowledge of the sequence of the cDNA strand for subsequent sequencing. In these embodiments, the cDNA strand may be produced using unlabeled nucleotides, known as dark filling. The reaction occurs as described above, however, unlabeled nucleotides are used to produce the cDNA strand.

### Removal, Priming, and Filling

Once the cDNA/RNA duplex is produced, the RNA strand is removed and the cDNA strand is primed with a primer. The template can be removed by any suitable means, for example by raising the temperature of the surface of the flow cell such that the duplex is melted, or by changing the buffer conditions to destabilize the duplex, or combination thereof. Methods for melting nucleic acid duplexes are well known in the art and are described, for example, in chapter 10 of Molecular Cloning, a Laboratory Manual, 3.sup.rd Edition, J. Sambrook, and D. W. Russell, Cold Spring Harbor Press (2001), Lander et al. (U.S. patent application number 2009/0305248), Quake (U.S. patent application number 2006/0019267), and Harris (U.S. patent application number 2009/0053705), content of each of which is incorporated herein by reference. In certain embodiments, the RNA strand of the cDNAIRNA duplex is removed by passing hot water over the duplex. Once dissociated, the RNA may then be removed from the surface, for example, by rinsing the surface with a suitable rinsing solution. The complementary cDNA generated during the copy step remains on the surface because it is extended from the covalently attached poly(T) oligonucleotide.

Once the RNA template has been removed, leaving only a cDNA template attached to the solid support, the cDNA template is primed for subsequent sequencing. There are numerous methods for priming the cDNA template. In certain embodiments, a tail, such as a poly(G) tail is added to the 3′ end of the cDNA strand. The added tail acts as a primer binding site on the cDNA molecule for a subsequent sequencing reaction. In certain embodiments, an oligonucleotide of poly(G) is added to the 3′ end of the cDNA. Adding the oligonucleotide tail of poly(G) may be accomplished by methods described herein, such as using a terminal transferase enzyme. The poly(G) tail is then blocked used a ddGTP. Blocking is described herein. A poly(C) primer is then hybridized to the poly(G) tail. Further description is provided in Harris (U.S. patent application number 2009/0053705), Harris (U.S. Pat. No. 7,282,337), and Harris (U.S. patent application number 2009/0197257), the content of each of which is incorporated by reference herein in its entirety.

In certain embodiments, the poly(C) primer will not perfectly align with the poly(G) tail on the cDNA molecules, so dCTP is added to the primer/cDNA duplex in a stepwise manner to fill remaining unpaired poly(G) nucleotides on the poly(G) tail of the cDNA, thereby ensuring alignment of the poly(C) primer. Once primed, a sequencing reaction is performed. The sequencing reaction is conducted as described herein.

In certain embodiments, after priming of the cDNA molecules, dATP is added in order to fill poly(T) primers on the substrate that did not pair with an RNA, but could still have been G-tailed by terminal transferase. This step prevents undesirable sequencing of G-tailed poly(dT) primers during sequencing, which would result in the abundance of poly-A reads.

In other embodiments, the poly(G) tail is not necessary, because generation of the cDNA strand can provide the sequence of the terminal portion of the cDNA, which information may be used to design a primer that will hybridize to the terminal portion of the cDNA for the second sequencing reaction.

Methods of synthesizing primers are known in the art. See, e.g., Sambrook et al. (DNA microarray: A Molecular Cloning Manual, Cold Spring Harbor, N.Y., 2003) or Maniatis, et al. (Molecular Cloning: A Laboratory Manual, Cold Spring Harbor, N.Y., 1982), the contents of each of which are incorporated by reference herein in their entirety. Suitable methods for synthesizing oligonucleotide primers are also described in Caruthers (Science 230:281-285, 1985), the contents of which are incorporated by reference. The primer includes a nucleotide sequence with substantial complementarity to the cDNA strand, so that the primer hybridize with the cDNA strand. Complementarity between the primer and the cDNA strand need only be sufficient to specifically bind the primer to the cDNA sequence.

Primers suitable for use in the present invention include those formed from nucleic acids, nucleic acid analogs, locked nucleic acids, modified nucleic acids, and chimeric primers of a mixed class including a nucleic acid with another organic component such as peptide nucleic acids. Exemplary nucleotide analogs include phosphate esters of deoxyadenosine, deoxycytidine, deoxyguanosine, deoxythymidine, adenosine, cytidine, guanosine, and uridine. Other examples of non-natural nucleotides include a xanthine or hypoxanthine; 5-bromouracil, 2-aminopurine, deoxyinosine, or methylated cytosine, such as 5-methylcytosine, and N4-methoxydeoxycytosine. Also included are bases of polynucleotide mimetics, such as methylated nucleic acids, e.g., 2′-O-methRNA, peptide nucleic acids, modified peptide nucleic acids, and any other structural moiety that can act substantially like a nucleotide or base, for example, by exhibiting base-complementarity with one or more bases that occur in DNA or RNA.

The length of the primer is not critical, as long as the primer is capable of hybridizing to the cDNA. In fact, primers may be of any length. For example, primers may be as few as 5 nucleotides, or as much as 100 nucleotides. Exemplary primers are 5-mers, 10-mers, 15-mers, 20-mers, 25-mers, 50-mers, or 100-mers. Methods for determining an optimal primer length are known in the art. See, e.g., Shuber (U.S. Pat. No. 5,888,778).

### Sequencing

Single molecule sequencing is shown for example in Lapidus et al. (U.S. Pat. No. 7,169,560), Lapidus et al. (U.S. patent application number 2009/0191565), Quake et al. (U.S. Pat. No. 6,818,395), Harris (U.S. Pat. No. 7,282,337), Quake et al. (U.S. patent application number 2002/0164629), Braslaysky, et al., PNAS (USA), 100: 3960-3964 (2003), Harris et al. (Science 320:106-109, 2008), Lipson et al. (Nat Biotechnol 27:652-658, 2009), and Pushkarev et al. (Nat Biotechnol 2009), the contents of each of these references is incorporated by reference herein in its entirety. The following sections discuss general considerations for nucleic acid sequencing, for example, polymerases useful in sequencing-by-synthesis, reaction conditions, signal detection and analysis.

Nucleotides

Nucleotides useful in the invention include any nucleotide or nucleotide analog, whether naturally-occurring or synthetic. For example, preferred nucleotides include phosphate esters of deoxyadenosine, deoxycytidine, deoxyguanosine, deoxythymidine, adenosine, cytidine, guanosine, and uridine. Other nucleotides useful in the invention comprise an adenine, cytosine, guanine, thymine base, a xanthine or hypoxanthine; 5-bromouracil, 2-aminopurine, deoxyinosine, or methylated cytosine, such as 5-methylcytosine, and N4-methoxydeoxycytosine. Also included are bases of polynucleotide mimetics, such as methylated nucleic acids, e.g., 2′-O-methRNA, peptide nucleic acids, modified peptide nucleic acids, locked nucleic acids and any other structural moiety that can act substantially like a nucleotide or base, for example, by exhibiting base-complementarity with one or more bases that occur in DNA or RNA and/or being capable of base-complementary incorporation, and includes chain-terminating analogs. A nucleotide corresponds to a specific nucleotide species if they share base-complementarity with respect to at least one base.

Nucleotides for nucleic acid sequencing according to the invention preferably include a detectable label that is directly or indirectly detectable. Preferred labels include optically-detectable labels, such as fluorescent labels. Examples of fluorescent labels include, but are not limited to, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine 13, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine. Preferred fluorescent labels are cyanine-3 and cyanine-5. Labels other than fluorescent labels are contemplated by the invention, including other optically-detectable labels.

Polymerases

Nucleic acid polymerases generally useful in the invention include DNA polymerases, RNA polymerases, reverse transcriptases, and mutant or altered forms of any of the foregoing. DNA polymerases and their properties are described in detail in, among other places, DNA Replication 2nd edition, Kornberg and Baker, W. H. Freeman, New York, N.Y. (1991). Known conventional DNA polymerases useful in the invention include, but are not limited to, Pyrococcus furiosus (Pfu) DNA polymerase (Lundberg et al., 1991, Gene, 108: 1, Stratagene), Pyrococcus woesei (Pwo) DNA polymerase (Hinnisdaels et al., 1996, Biotechniques, 20:186-8, Boehringer Mannheim), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent™ DNA polymerase, Cariello et al., 1991, Polynucleotides Res, 19: 4193, New England Biolabs), 9.degree.Nm™ DNA polymerase (New England Biolabs), Stoffel fragment, ThermoSequenase® (Amersham Pharmacia Biotech UK), Therminator™ (New England Biolabs), Thermotoga maritima (Tma) DNA polymerase (Diaz and Sabino, 1998 Braz J. Med. Res, 31:1239), Thermus aquaticus (Taq) DNA polymerase (Chien et al., 1976, J. Bacteoriol, 127: 1550), DNA polymerase, Pyrococcus kodakaraensis KOD DNA polymerase (Takagi et al., 1997, Appl. Environ. Microbiol. 63:4504), JDF-3 DNA polymerase (from thermococcus sp. JDF-3, Patent application WO 0132887), Pyrococcus GB-D (PGB-D) DNA polymerase (also referred as Deep Vent™ DNA polymerase, Juncosa-Ginesta et al., 1994, Biotechniques, 16:820, New England Biolabs), UlTma DNA polymerase (from thermophile Thermotoga maritima; Diaz and Sabino, 1998 Braz J. Med. Res, 31:1239; PE Applied Biosystems), Tgo DNA polymerase (from thermococcus gorgonarius, Roche Molecular Biochemicals), E. coli DNA polymerase I (Lecomte and Doubleday, 1983, Polynucleotides Res. 11:7505), T7 DNA polymerase (Nordstrom et al., 1981, J. Biol. Chem. 256:3112), and archaeal DP1I/DP2 DNA polymerase II (Cann et al, 1998, Proc. Natl. Acad. Sci. USA 95:14250).

Both mesophilic polymerases and thermophilic polymerases are contemplated. Thermophilic DNA polymerases include, but are not limited to, ThermoSequenase®, 9.degree.Nm™, Therminator™, Taq, Tne, Tma, Pfu, Tfl, Tth, Tli, Stoffel fragment, Vent™ and Deep Vent™ DNA polymerase, KOD DNA polymerase, Tgo, JDF-3, and mutants, variants and derivatives thereof. A highly-preferred form of any polymerase is a 3′ exonuclease-deficient mutant.

Reverse transcriptases useful in the invention include, but are not limited to, reverse transcriptases from HIV, HTLV-1, HTLV-II, FeLV, FIV, SIV, AMV, MMTV, MoMuLV and other retroviruses (see Levin, Cell 88:5-8 (1997); Verma, Biochim Biophys Acta. 473:1-38 (1977); Wu et al., CRC Crit. Rev Biochem. 3:289-347 (1975)).

Detection

Any detection method can be used that is suitable for the type of label employed. Thus, exemplary detection methods include radioactive detection, optical absorbance detection, e.g., UV-visible absorbance detection, optical emission detection, e.g., fluorescence or chemiluminescence. For example, extended primers can be detected on a substrate by scanning all or portions of each substrate simultaneously or serially, depending on the scanning method used. For fluorescence labeling, selected regions on a substrate may be serially scanned one-by-one or row-by-row using a fluorescence microscope apparatus, such as described in Fodor (U.S. Pat. No. 5,445,934) and Mathies et al. (U.S. Pat. No. 5,091,652). Devices capable of sensing fluorescence from a single molecule include scanning tunneling microscope (siM) and the atomic force microscope (AFM). Hybridization patterns may also be scanned using a CCD camera (e.g., Model TE/CCD512SF, Princeton Instruments, Trenton, N.J.) with suitable optics (Ploem, in Fluorescent and Luminescent Probes for Biological Activity Mason, T. G. Ed., Academic Press, Landon, pp. 1-11 (1993), such as described in Yershov et al., Proc. Natl. Acad. Sci. 93:4913 (1996), or may be imaged by TV monitoring. For radioactive signals, a phosphorimager device can be used (Johnston et al., Electrophoresis, 13:566, 1990; Drmanac et al., Electrophoresis, 13:566, 1992; 1993). Other commercial suppliers of imaging instruments include General Scanning Inc., (Watertown, Mass. on the World Wide Web at genscan.com), Genix Technologies (Waterloo, Ontario, Canada; on the World Wide Web at confocal.com), and Applied Precision Inc. Such detection methods are particularly useful to achieve simultaneous scanning of multiple attached template nucleic acids.

A number of approaches can be used to detect incorporation of fluorescently-labeled nucleotides into a single nucleic acid molecule. Optical setups include near-field scanning microscopy, far-field confocal microscopy, wide-field epi-illumination, light scattering, dark field microscopy, photoconversion, single and/or multiphoton excitation, spectral wavelength discrimination, fluorophor identification, evanescent wave illumination, and total internal reflection fluorescence (TIRF) microscopy. In general, certain methods involve detection of laser-activated fluorescence using a microscope equipped with a camera. Suitable photon detection systems include, but are not limited to, photodiodes and intensified CCD cameras. For example, an intensified charge couple device (ICCD) camera can be used. The use of an ICCD camera to image individual fluorescent dye molecules in a fluid near a surface provides numerous advantages. For example, with an ICCD optical setup, it is possible to acquire a sequence of images (movies) of fluorophores.

Some embodiments of the present invention use TIRF microscopy for imaging. TIRF microscopy uses totally internally reflected excitation light and is well known in the art. See, e.g., the World Wide Web at nikon-instruments.jp/eng/page/products/tirf.aspx. In certain embodiments, detection is carried out using evanescent wave illumination and total internal reflection fluorescence microscopy. An evanescent light field can be set up at the surface, for example, to image fluorescently-labeled nucleic acid molecules. When a laser beam is totally reflected at the interface between a liquid and a solid substrate (e.g., a glass), the excitation light beam penetrates only a short distance into the liquid. The optical field does not end abruptly at the reflective interface, but its intensity falls off exponentially with distance. This surface electromagnetic field, called the “evanescent wave”, can selectively excite fluorescent molecules in the liquid near the interface. The thin evanescent optical field at the interface provides low background and facilitates the detection of single molecules with high signal-to-noise ratio at visible wavelengths.

The evanescent field also can image fluorescently-labeled nucleotides upon their incorporation into the attached template/primer complex in the presence of a polymerase. Total internal reflectance fluorescence microscopy is then used to visualize the attached template/primer duplex and/or the incorporated nucleotides with single molecule resolution.

Some embodiments of the invention use non-optical detection methods such as, for example, detection using nanopores (e.g., protein or solid state) through which molecules are individually passed so as to allow identification of the molecules by noting characteristics or changes in various properties or effects such as capacitance or blockage current flow (see, for example, Stoddart et al, Proc. Nat. Acad. Sci., 106:7702, 2009; Purnell and Schmidt, ACS Nano, 3:2533, 2009; Branton et al, Nature Biotechnology, 26:1146, 2008; Polonsky et al, U.S. Application 2008/0187915; Mitchell & Howorka, Angew. Chem. Int. Ed. 47:5565, 2008; Borsenberger et al, J. Am. Chem. Soc., 131, 7530, 2009); or other suitable non-optical detection methods.

Analysis

Alignment and/or compilation of sequence results obtained from the image stacks produced as generally described above utilizes look-up tables that take into account possible sequences changes (due, e.g., to errors, mutations, etc.). Essentially, sequencing results obtained as described herein are compared to a look-up type table that contains all possible reference sequences plus 1 or 2 base errors.

### Resequencing

In certain embodiments, the cDNA is resequenced by denaturing the extended complementary strand of the cDNA, removing the newly-synthesized complementary strand, annealing a new primer, and then repeating the experiment with fresh reagents to sequentially analyze the sequence of the same cDNA molecule. This approach is very sensitive because only a single copy of the cDNA molecule is needed to obtain sequence information. Further, releasing the extension product from the cDNA template, e.g., by denaturing, and annealing the cDNA template with a different primer, provides the opportunity to re-read the same cDNA molecule with different sets of nucleotides (e.g., different combinations of two types of labeled nucleotides and two types of unlabeled nucleotides).

In some embodiments, nucleotides lacking any labeling moiety are provided for a period of time to allow unlabeled nucleotides to fill in regions, for example regions that are an already known, until the complementary strand extends to reach unknown regions further downstream. At this point, nucleotides bearing a labeling moiety can be added and analysis begun or continued.

Further description of resequencing is provided in Lander et al. (U.S. patent application serial number 2009/0305248) and Quake (U.S. patent application serial number 2006/0019267), the content of each of which is incorporated by reference herein in its entirety.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

### EXAMPLES

Methods of the invention were used to profile a mouse pancreatic ductal cell line (SM25) and a mouse pancreatic adenocarcinoma cell line (490). SM25 was derived from a mouse with the pancreas targeted expression of KRAS G12D, which induces a precursor pancreatic cancer lesion called PanIN (Aguirre et al., Genes Dev 17:3112-3126, 2003). 490 was derived from a pancreatic adenocarcinoma that was developed in a genetically engineered mouse model incorporating activating mutation KRAS G12D and the conditional loss of p53 targeted specifically to the pancreas (Bardeesy et al. Proc Natl Acad Sci USA 103:5947-5952, 2006).

### Example 1

### Sample Loading and Flow Cell Preparation

SM25 and 490 cells in quantities indicated in the figures were lysed in the lysis buffers of PicoPure RNA Isolation (Molecular Devices), AmpliGrid Cell Extraction (Advalytix), CellsDirect Two-Step qRT-PCR (Invitrogen) or FastLane Cell cDNA (Qiagen) kits as instructed by the manufacturers. The lysates were hybridized in 10 μl volume to Helicos poly(dT)-coated sequencing flow cell channels in 1×SSC, 0.05% SDS at 37° C. for 30 minutes. First-strand cDNA was synthesized with the SuperScript III first-strand cDNA synthesis kit (Invitrogen) using manufacturer's recommendations, except no additional primers were added, and the incubation steps were modified as follows: 37° C. 15 minutes, and 55° C. 45 minutes. Subsequent to cDNA synthesis, hot water was passed through the channels to degrade and melt away the RNA strands. Guanine tailing was performed using terminal transferase, adding 500 μl guanine in 20 μl volume in 1×TdT buffer, 2.5 mM CoCl₂ and 20 units terminal transferase per channel. The reaction took place at 37° C. for 30 minutes, followed by 3′ blocking with 100 μM ddGTP and ddATP under the same reaction conditions. The poly-C primers were hybridized at 50 nM in 1×SSC, 0.05% SDS at 55° C. for 30 minutes, followed by step-wise “fill” steps with 500 μM cytosine and adenine nucleotides with 5 units Klenow fragment (NEB) in 1×NEB2 buffer and 20 μl reaction volume per channel. The lock step was then performed using virtual terminator guanine and thymidine nucleotide analogs. Sequencing by synthesis was then initiated as described herein. For the experiments with the HIV RT, the only step that was altered was the cDNA synthesis step, which was performed in the 1×HIV RT buffer, 150 μM dNTPs and 10 units enzyme in 20 μl reaction volume. The reaction took place at 42° C. for 30 minutes, followed by 55° C. for 30 minutes.

### Example 2

### RNA Isolation and QRT-PCR Validation

For RNA extraction, cells were detached from cell culture plates by standard trypsinization and centrifuged into a pellet. The supernatant was removed and cells were flash frozen in liquid nitrogen. Frozen pellets were then thawed and cells were homogenized with the QIAshredder Kit (Qiagen). Total RNA was then extracted with the RNeasy Mini Kit (Qiagen) and treated with on column DNase I (Qiagen) as instructed. Total RNA quantification and quality assessment was done with Nanodrop OD260 and OD260/280 measurements. RNA was then subjected to reverse transcriptase using the Invitrogen Superscript III First-Strand Synthesis System for RT-PCR kit per protocol to make cDNA. Oligo(dT)₂₀ primers from the Invitrogen kit were used for reverse transcription and RNase H was used for RNA removal after cDNA synthesis. The cDNA was then aliquoted for quantitative PCR at 10 ng per reaction. Primers for each gene were obtained from a pre-validated source, PrimerBank (http://pga.mghlarvard.edu/primerbank/). The primers were prepped at a final concentration of 0.4 μM using Power SYBR Green Master Mix per protocol and each condition was done in triplicate. qPCR reactions were run and analyzed using the ABI 7500 Real-Time PCR system (Applied Biosystems). Beta-actin (actb) was used as the endogenous control for each cell line.

### Example 3

### Data Analysis

Read filtering, alignment (using the IndexDP algorithm) and transcript counting were done as described (Lipson et al. Nat Biotechnol 27:652-658, 2009). The mouse reference used was the MM9 assembly downloaded from the UCSC Genome Browser. For the whole genome alignment of reads, the IndexDP alignment threshold used was 4.3 (Lipson et al. Nat Biotechnol 27:652-658, 2009).

### Example 4

### Sequencing

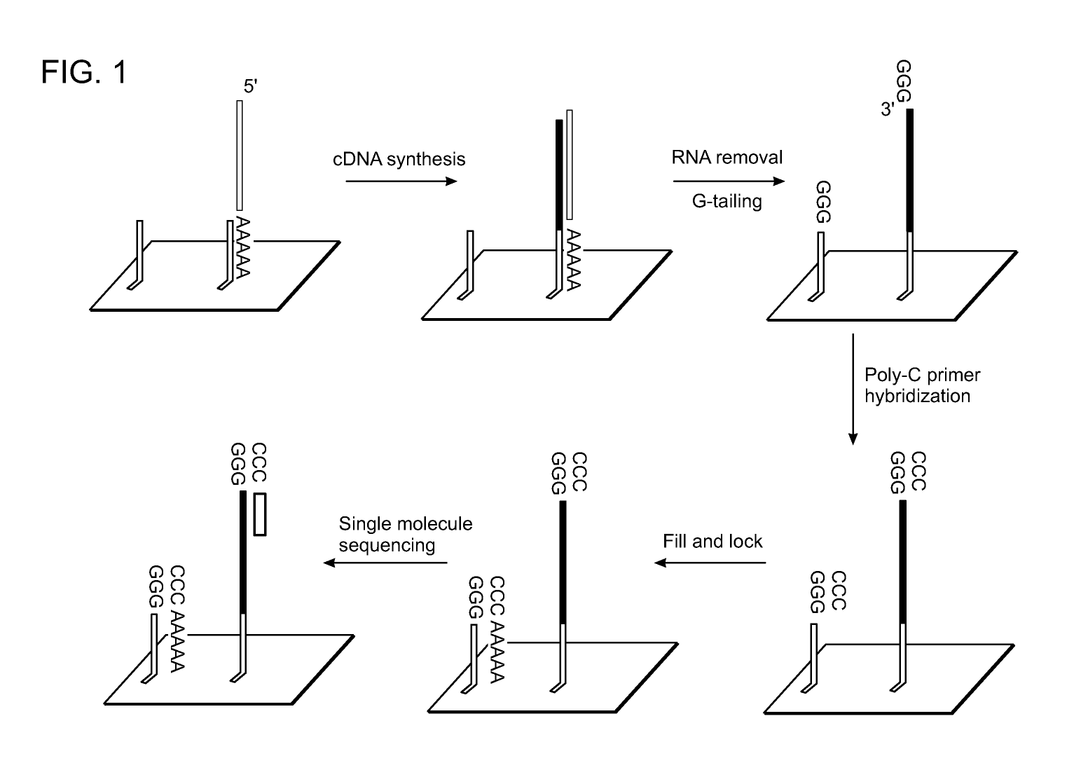

FIG. 1 is a schematic showing the protocol used for sequencing RNA from cells. Poly(A) tailed mRNA from 250 to 16000 lysed SM25 and 490 cells were captured directly on poly(dT)-coated sequencing flow cells without the need for intermediate RNA isolation or poly(A) tailed RNA purification steps. On-surface cDNA synthesis was initiated by the use of SuperScript III (SSIII) reverse transcriptase (RT), followed by terminal transferase (TdT)-mediated on-surface G-tailing of cDNAs covalently attached to surfaces to generate priming sites for sequencing from the 3′ ends of cDNAs. Poly(C) primers were hybridized to the G-tailed templates, followed by a “fill and lock” step in order to ensure that the poly(C) primer alignment within the poly(G) stretch was suitable for the initiation of sequencing at the 3′ end of the generated cDNA. Sequencing was then performed as described above.

### Example 5

### cDNA Priming

Guanine was chosen over other nucleotides for on-surface TdT-mediated tailing for several reasons: guanine tailing with TdT was generally limited to about 25-30 nts whereas TdT-mediated tailing with other nucleotides exhibit variable lengths and was not as controllable as guanine tailing; adenine tailing would have caused the poly(A) tail at the 3′ end of the cDNA molecules to interact with the poly(dT) capture primers on the surface; and thymidine tailing would have required a poly(A) primer to be used for the sequencing initiation purposes rather than a poly(C) primer, which was have been problematic because the poly(A) primers would hybridize not only to the TdT-generated poly(T) tails, but also to the poly(dT) capture primers on the sequencing surfaces.

### Example 6

### Polymerases

SSIII was chosen as the reverse transcriptase for production of the cDNA/RNA duplex because it is a commonly-used reverse transcriptase reported to give satisfactory performance for on-surface applications (Taniguchi et al. Nat Methods 6:503-506, 2009). Profiles that were obtained with this reverse transcriptase were compared to HIV reverse transcriptase to determine any reverse transcriptase specific differences (FIG. 5). High concordance was obtained of transcript counts with both reverse transcriptases (Pearson correlation, r=0.993), indicating the accuracy and reproducibility of the profiles.

### Example 7

### Sequence Reads

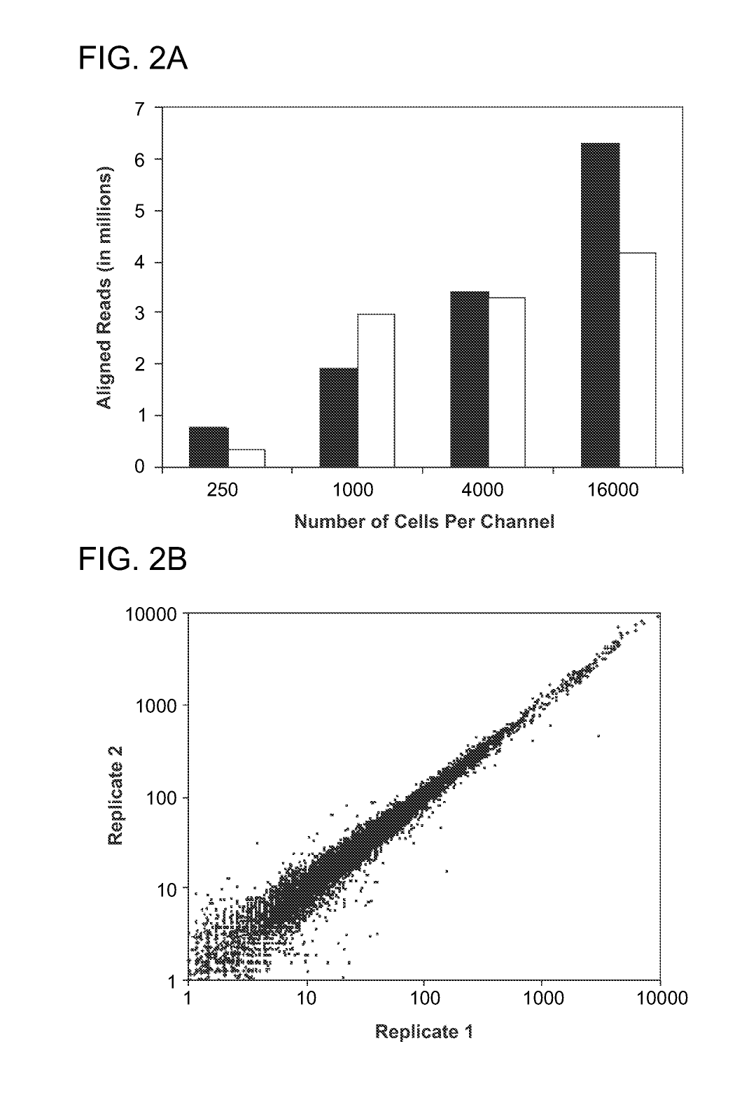

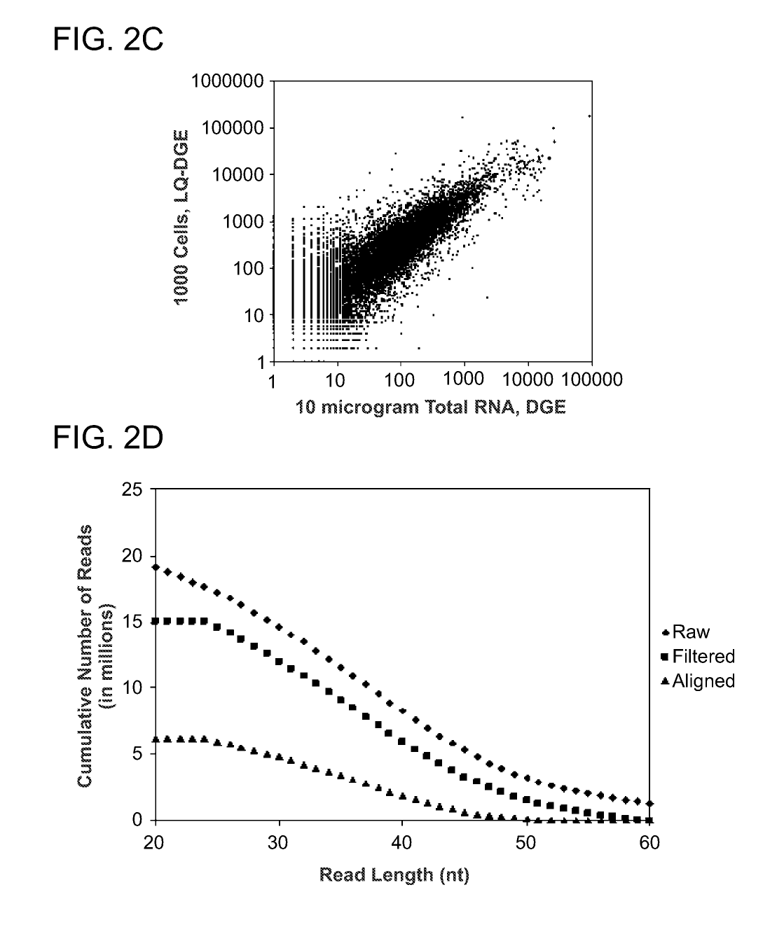

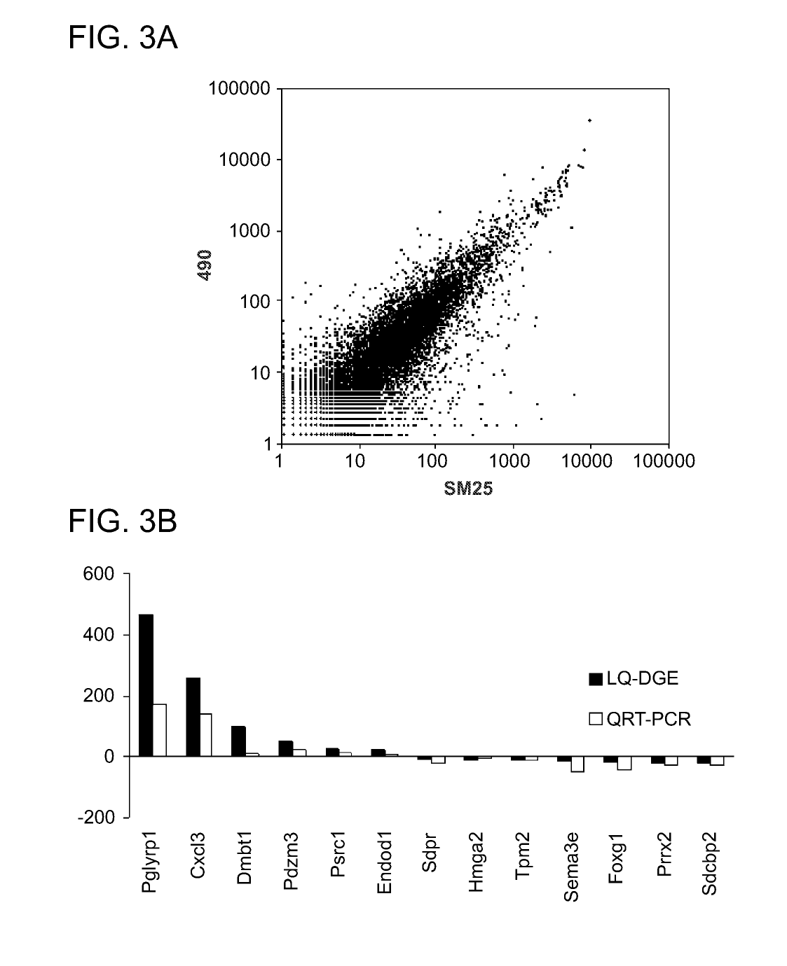

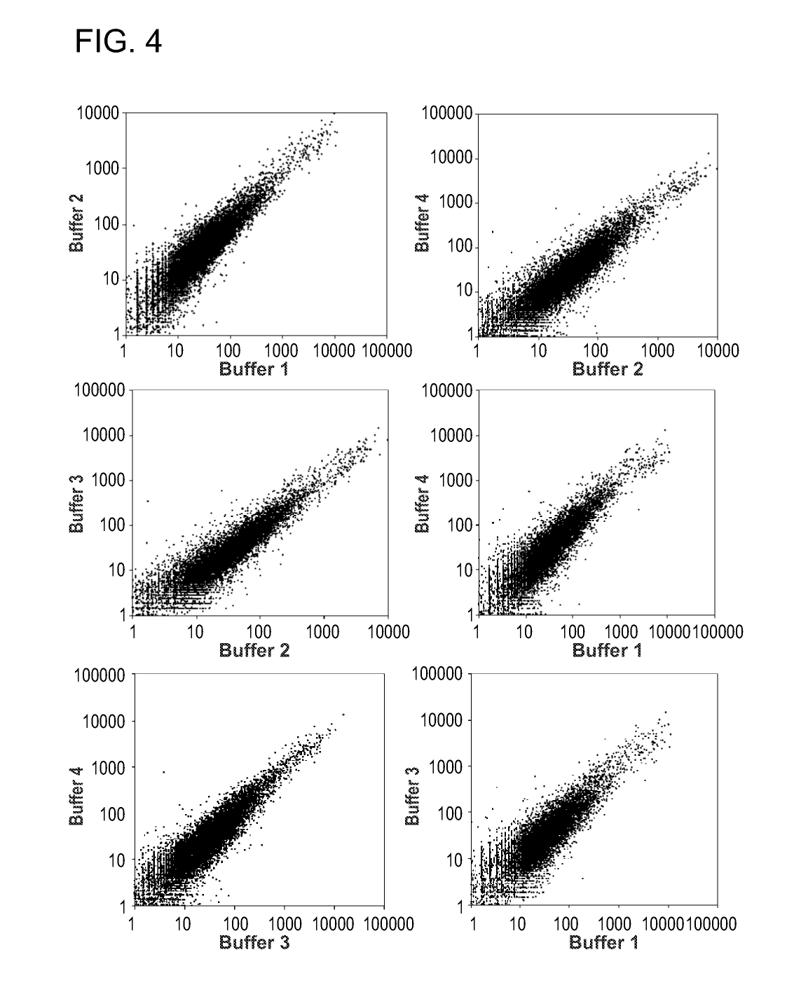

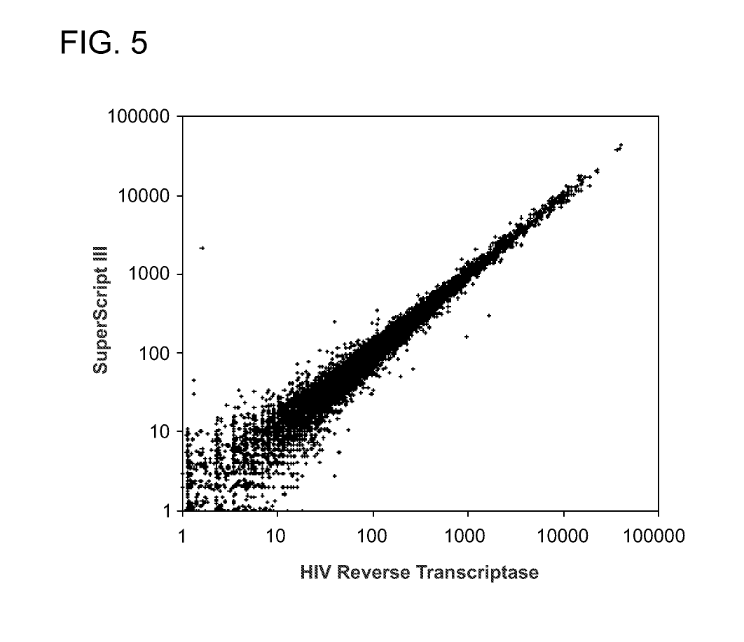

To determine the effect of cell quantity used per channel to the number of usable reads obtained, a titration protocol was performed, results of which are shown in FIG. 2 panel A. Data herein show that as low as 250 cells could generate sufficient usable reads for general gene expression profiling purposes. The measurements were highly reproducible as demonstrated by profiling a thousand 490 cells prepared at separate times in two independent runs (FIG. 2 panel B). Comparison of four different commercial cell lysis conditions revealed high correlation of transcript counts obtained (FIG. 4), suggesting independence of the quantitative power of gene expression profiles obtained with methods of the invention from cell lysis methods.

### Example 8

### Comparison to Other Profiling Methods

To determine the correlation of methods of the invention to standard digital gene expression methods, profiling of 10 micrograms of 490 RNA (from about 4 million cells) was compared to one thousand 490 cells profiled with methods of the invention. High correlation was observed between both datasets (FIG. 2 panel C), demonstrating the quantification ability of methods of the invention and accuracy of the counts. The rRNA sequence level with standard digital gene expression methods profiling was 2-3%, while this proportion was 0.01-0.02% with the methods of the invention. Mitochondrial sequences proportions were similar in both methods (2-3%). Since rRNA species can also be poly-adenylated (Ozsolak et al. Nature, 2009; and Slomovic et al. Nucleic Acids Res, 34:2966-2975, 2006), the presence of rRNA reads from methods of the invention was expected.

### Example 9

### Sequencing Statistics

Over 97% of the reads were 24-60 nucleotides (nts) in length, with a median length of 36 nts (FIG. 2 panel D). This length was 3 nts higher than previously reported performance involving single molecule sequencing (See Lipson, Nat Biotechnol 27:652-658, 2009; and Pushkarev et al., Nat Biotechnol, 2009). While the cause is not known, the slightly longer read lengths might have been caused by sequencing-by-synthesis steps taking place further away from the flow cell surface, thus allowing easier diffusion of enzymes and other components of single molecule sysnthesis chemistry. The average error rates were around 4.7% to 5.1% per base. Examination of the strandedness of the procedure revealed 1.5-1.9% of the sequences mapping to known genes to be aligned opposite to their known transcription direction (Table 1). This percentage is lower than the 4.6% level obtained with the standard digital gene expression approach and may indicate potential antisense transcription events. The primary contributor of these reads is likely to be the reverse transcriptase tendency to generate spurious second-strand cDNA products.

TABLE 1

| | | | Aligned | Percentage |
|---|---|---|---|---|
| | Quality | | Read | of Reads |
| Sample | Filtered | Aligned | Mean | Mapping in the |
| Name | Reads | Reads | Length | Antisense Direction |
| Ampligrid- | 8553680 | 2505107 | 36.5916 | 1.065 |
| 490-SSIII | | | | |
| Qiagen- | 11270742 | 4186003 | 37.0209 | 1.218 |
| 490-SSIII | | | | |
| Ampligrid- | 15093695 | 6298058 | 35.6237 | 1.619 |
| SM25-SSIII | | | | |
| Qiagen- | 11557689 | 4970093 | 35.4661 | 1.212 |
| SM25-SSIII | | | | |
| Ampligrid- | 14099553 | 6974285 | 34.9793 | 1.998 |
| SM25-HIV RT | | | | |
| Standard | 25506692 | 14318515 | 32.8482 | 4.678 |
| DGE-490 | | | | |

Table 1 provides data showing filtered and aligned read yields, read lengths and percentage of reads aligning in the antisense direction of known gene transcription directions are shown. Each row indicates data from single channels of a 50-channel HELISCOPE system (single molecule sequencing by synthesis system, Helicos Biosciences Corporation, Cambridge Mass.). Sample names contain the cell lysis method, cell line, and the reverse transcriptase enzyme used. The last row indicate the yields from 10 microgram 490 RNA profiled with an established digital gene expression method.

### Example 10

### Expression Profiling

To further investigate the quantification ability of methods of the invention and their use in identifying differentially expressed genes, profiles of the 490 and SM25 cells produced by methods of the invention were compared (FIG. 3 panel A). Methods of the invention were used to identify 2088 genes exhibiting greater than 2-fold differences and with 10 t.p.m (transcripts per million) minimum expression levels in at least one of the cell lines (See Table 2 below). Thirteen genes exhibiting a wide-range of expression level differences were validated with the quantitative real-time polymerase chain reaction approach (FIG. 3, panel C). Data herein demonstrate that methods of the invention may be used to clearly distinguish expression profiles between cell lines derived from pancreatic pre-malignant tissue and fully developed adenocarcinoma. The differentiation of gene expression of similar cell types is a critical element for comprehensive analyses of rare cell populations such as circulating tumor cells and stem cells.

TABLE 2

| | | | | | | Normalized | Normalized | Normalized | Normalized | |
|---|---|---|---|---|---|---|---|---|---|---|
| | Ampligrid 490 | Qiagen 490 | Ampligrid SM25 | Qiagen SM25 | Ampligrid SM25 HIV-RT | Ampligrid 490 | Qiagen 490 | Ampligrid SM25 | Qiagen SM25 | SM25/490 |
| Sox17 | 16.9119 | 18 | 400 | 284.82 | 478.212 | 56.43866 | 36.18424 | 455.3551 | 435.3444 | 9.616408 |
| Atp6v1h | 53.4408 | 57.0929 | 329.491 | 215.416 | 359.225 | 178.3435 | 114.7702 | 375.0885 | 329.2611 | 2.402992 |
| Pcmtd1 | 27 | 8 | 125 | 76.7745 | 154 | 90.10482 | 16.08188 | 142.2985 | 117.349 | 2.445198 |
| Rdh10 | 419 | 553 | 504 | 312 | 564 | 1398.293 | 1111.66 | 573.7474 | 476.8888 | 0.418588 |
| Mcm3 | 25.8173 | 130.163 | 67.9502 | 48.5 | 95.7074 | 86.1579 | 261.6583 | 77.35367 | 74.13175 | 0.435533 |
| Paqr8 | 15.4556 | 29.8943 | 12.1218 | 8.35167 | 13.1911 | 51.57867 | 60.09458 | 13.79931 | 12.76544 | 0.237879 |
| Tmem14a | 17 | 10 | 70 | 50 | 83 | 56.73267 | 20.10235 | 79.68713 | 76.42448 | 2.031777 |
| Gsta3 | 6 | 1.00877 | 1286.95 | 953 | 1438.94 | 20.02329 | 2.027865 | 1465.048 | 1456.651 | 132.4964 |
| Ogfrl1 | 9.11415 | 10.0373 | 56.1174 | 29.8335 | 44.5281 | 30.41588 | 20.17733 | 63.88335 | 45.6002 | 2.163997 |
| Lmbrd1 | 44.4604 | 45.5996 | 240.765 | 196.089 | 299.54 | 148.3739 | 91.66593 | 274.0839 | 299.72 | 2.390453 |
| Dst | 349.874 | 142 | 274.582 | 154.498 | 355.309 | 1167.605 | 285.4534 | 312.5808 | 236.1486 | 0.377638 |
| Neurl3 | 9 | 13.1085 | 63.5425 | 33.3162 | 86.194 | 30.03494 | 26.35117 | 72.336 | 50.92347 | 2.18599 |
| uc007arr.1 | 16 | 11 | 158 | 76 | 161 | 53.39545 | 22.11259 | 179.8652 | 116.1652 | 3.920516 |
| Tbc1d8 | 5.0365 | 7.01833 | 54.7068 | 29.2605 | 64.8978 | 16.80789 | 14.10849 | 62.27754 | 44.72437 | 3.46101 |
| Il1r1 | 40 | 28.0117 | 189 | 135.343 | 245.663 | 133.4886 | 56.31011 | 215.1553 | 206.8704 | 2.223543 |
| Fhl2 | 140 | 219 | 125 | 107 | 153 | 467.2102 | 440.2415 | 142.2985 | 163.5484 | 0.337039 |
| Ercc5 | 10 | 8 | 107.429 | 53.3985 | 100 | 33.37216 | 16.08188 | 122.2958 | 81.61906 | 4.123321 |
| Slc39a10 | 63.9387 | 196.179 | 76.2128 | 33.6053 | 76.7081 | 213.3772 | 394.366 | 86.75971 | 51.36535 | 0.227275 |
| Sdpr | 267 | 391 | 107 | 55 | 96 | 891.0366 | 786.002 | 121.8075 | 84.06693 | 0.122761 |
| Myo1b | 57.5365 | 27.0456 | 286.379 | 134.037 | 264.333 | 192.0117 | 54.36802 | 326.0103 | 204.8742 | 2.154741 |
| Stat1 | 7 | 8.65211 | 181.392 | 112.425 | 244.381 | 23.36051 | 17.39278 | 206.4944 | 171.8405 | 9.283542 |
| Nab1 | 24 | 28 | 127 | 92 | 158 | 80.09318 | 56.28659 | 144.5752 | 140.621 | 2.091192 |
| Inpp1 | 6 | 7 | 52 | 37 | 51 | 20.02329 | 14.07165 | 59.19616 | 56.55412 | 3.39494 |
| Sgol2 | 20 | 59 | 41 | 16 | 48 | 66.74431 | 118.6039 | 46.67389 | 24.45583 | 0.383763 |
| Aox1 | 13 | 9 | 79 | 31 | 62 | 43.3838 | 18.09212 | 89.93262 | 47.38318 | 2.233652 |
| Stradb | 8 | 7 | 73 | 35 | 92 | 26.69773 | 14.07165 | 83.1023 | 53.49714 | 3.350541 |
| Wdr12 | 62 | 247.996 | 154 | 99.2847 | 178 | 206.9074 | 498.5303 | 175.3117 | 151.7556 | 0.463637 |
| Plekhm3 | 5 | 5 | 62.2369 | 20 | 72.2286 | 16.68608 | 10.05118 | 70.84972 | 30.56979 | 3.793191 |
| Idh1 | 163 | 86.0919 | 757.696 | 511.456 | 873.997 | 543.9661 | 173.065 | 862.5518 | 781.7552 | 2.293216 |
| Rpe | 46.9429 | 67.8772 | 350.069 | 205.774 | 424.887 | 156.6586 | 136.4491 | 398.5142 | 314.5234 | 2.432681 |
| 1110028C15Rik | 6.05714 | 5.12281 | 81.931 | 64.6213 | 96.478 | 20.21398 | 10.29805 | 93.26924 | 98.77299 | 6.293983 |
| Acadl | 126 | 187 | 1252 | 1013 | 1702 | 420.4892 | 375.914 | 1425.261 | 1548.36 | 3.733814 |
| Vil1 | 48 | 44 | 9 | 2 | 5 | 160.1864 | 88.45035 | 10.24549 | 3.056979 | 0.053502 |
| Wnt10a | 14 | 21 | 138 | 59 | 146 | 46.72102 | 42.21494 | 157.0975 | 90.18089 | 2.780409 |
| Atg9a | 21.0328 | 8.10163 | 131.008 | 67.9516 | 183.338 | 70.19099 | 16.28618 | 149.1379 | 103.8633 | 2.925642 |
| Ptprn | 140 | 132 | 144 | 78 | 199 | 467.2102 | 265.3511 | 163.9278 | 119.2222 | 0.386521 |
| Obsl1 | 62 | 27 | 39 | 15 | 55 | 206.9074 | 54.27635 | 44.39712 | 22.92735 | 0.257767 |
| Stk11ip | 20 | 3.00047 | 86.2729 | 51.4554 | 130.471 | 66.74431 | 6.031651 | 98.212 | 78.64905 | 2.430212 |
| Ap1s3 | 5.00964 | 11.0468 | 62 | 29.1106 | 76.3601 | 16.71825 | 22.20667 | 70.58003 | 44.49525 | 2.95634 |
| Serpine2 | 56 | 50 | 610.657 | 415.671 | 699.972 | 186.8841 | 100.5118 | 695.1644 | 635.3488 | 4.629549 |
| Irs1 | 44.5426 | 31.101 | 24 | 20 | 40.2439 | 148.6483 | 62.52033 | 27.3213 | 30.56979 | 0.274146 |
| Ccl20 | 16 | 21.0027 | 2083 | 2158 | 2449 | 53.39545 | 42.22037 | 2371.261 | 3298.481 | 59.29711 |
| Cab39 | 18 | 27 | 25.4433 | 17.0055 | 32 | 60.06988 | 54.27635 | 28.96434 | 25.99273 | 0.48062 |
| Ugt1a10 | 12 | 21.4039 | 287.053 | 113.399 | 310.719 | 40.04659 | 43.02688 | 326.7776 | 173.3292 | 6.020055 |
| Ugt1a9 | 12 | 4.14219 | 239.372 | 103.651 | 310.719 | 40.04659 | 8.326777 | 272.4981 | 158.4295 | 8.908366 |
| Ugt1a7c | 12 | 59.1445 | 375.69 | 442.122 | 310.719 | 40.04659 | 118.8944 | 427.6809 | 675.7789 | 6.942577 |
| Ugt1a6a | 12 | 41.8828 | 328.295 | 264.572 | 413.685 | 40.04659 | 84.19428 | 373.727 | 404.3956 | 6.263016 |
| Ugt1a5 | 12 | 4.14219 | 258.846 | 74.2183 | 310.719 | 40.04659 | 8.326777 | 294.6671 | 113.4419 | 8.436647 |
| Ugt1a2 | 12 | 4.14219 | 239.372 | 74.2183 | 310.719 | 40.04659 | 8.326777 | 272.4981 | 113.4419 | 7.978358 |
| Ugt1a1 | 12 | 4.14219 | 239.372 | 89.3992 | 310.719 | 40.04659 | 8.326777 | 272.4981 | 136.6458 | 8.458041 |
| Glrp1 | 67 | 36 | 2.02218 | 1 | 1 | 223.5934 | 72.36847 | 2.302025 | 1.52849 | 0.012943 |
| Cxcr7 | 171 | 334.997 | 128 | 118 | 127 | 570.6639 | 673.4228 | 145.7136 | 180.3618 | 0.2621 |
| Hes6 | 48 | 57 | 412 | 262 | 533 | 160.1864 | 114.5834 | 469.0157 | 400.4643 | 3.164395 |
| Per2 | 17 | 11 | 95 | 43 | 105.317 | 56.73267 | 22.11259 | 108.1468 | 65.72506 | 2.20523 |
| 2310007B03Rik | 43 | 38 | 214 | 149 | 261 | 143.5003 | 76.38894 | 243.615 | 227.745 | 2.143625 |
| Mterfd2 | 28.0834 | 22.0125 | 142 | 95 | 208 | 93.72036 | 44.2503 | 161.651 | 145.2065 | 2.224078 |
| Fam174a | 29 | 31 | 218 | 164 | 213 | 96.77925 | 62.31729 | 248.1685 | 250.6723 | 3.13546 |
| St8sia4 | 8 | 1 | 95 | 65 | 109.12 | 26.69773 | 2.010235 | 108.1468 | 99.35183 | 7.227914 |
| Tcfcp2l1 | 13.0117 | 3.02065 | 232.619 | 79.6743 | 256.472 | 43.42285 | 6.072217 | 264.8106 | 121.7813 | 7.810717 |
| Ralb | 63 | 36 | 441.784 | 160.638 | 524.376 | 210.2446 | 72.36847 | 502.9214 | 245.5335 | 2.648338 |
| Tmem185b | 39 | 48.9796 | 239.693 | 147.98 | 293 | 130.1514 | 98.46052 | 272.8635 | 226.1859 | 2.182955 |
| Epb4.1l5 | 5 | 3 | 67 | 51 | 78 | 16.68608 | 6.030706 | 76.27197 | 77.95297 | 6.789031 |
| Tmem37 | 15 | 34 | 116 | 88 | 102 | 50.05823 | 68.348 | 132.053 | 134.5071 | 2.251233 |
| En1 | 94 | 90 | 0 | 1 | 1.00901 | 313.6983 | 180.9212 | 0 | 1.52849 | 0.00309 |
| Slc35f5 | 29 | 30 | 172 | 113 | 211 | 96.77925 | 60.30706 | 195.8027 | 172.7193 | 2.345984 |
| uc007clk.1 | 7.18701 | 14 | 23.1889 | 57.1369 | 20.593 | 23.9846 | 28.14329 | 26.39796 | 87.33316 | 2.181771 |
| Cd55 | 12 | 17 | 105.309 | 71.7306 | 116.837 | 40.04659 | 34.174 | 119.8825 | 109.6395 | 3.09243 |
| C4bp | 2 | 3 | 377 | 271 | 426 | 6.674431 | 6.030706 | 429.1721 | 414.2207 | 66.38203 |
| Pfkfb2 | 2 | 5 | 87 | 61 | 93 | 6.674431 | 10.05118 | 99.03972 | 93.23787 | 11.496 |
| AA986860 | 38 | 31 | 590 | 300 | 713.913 | 126.8142 | 62.31729 | 671.6487 | 458.5469 | 5.975714 |
| Mapkapk2 | 190 | 175 | 1042 | 574 | 1285 | 634.071 | 351.7912 | 1186.2 | 877.3531 | 2.093146 |
| Dyrk3 | 23 | 27 | 158 | 92 | 153 | 76.75596 | 54.27635 | 179.8652 | 140.621 | 2.445857 |
| Ikbke | 20 | 14 | 325 | 147 | 416 | 66.74431 | 28.14329 | 369.976 | 224.688 | 6.267035 |
| Rab7l1 | 5 | 6 | 58 | 44 | 61 | 16.68608 | 12.06141 | 66.02648 | 67.25355 | 4.636232 |
| Mfsd4 | 28.3982 | 16.6159 | 27.0052 | 18.9926 | 43.4285 | 94.77092 | 33.40187 | 30.74239 | 29.02999 | 0.466342 |
| Klhdc8a | 10 | 6 | 61 | 38 | 76 | 33.37216 | 12.06141 | 69.44165 | 58.08261 | 2.806829 |
| Nuak2 | 17 | 14 | 205 | 89.2409 | 209.067 | 56.73267 | 28.14329 | 233.3695 | 136.4038 | 4.356631 |
| uc007cpj.1 | 65 | 26 | 327.797 | 121.35 | 356.989 | 216.919 | 52.26612 | 373.1601 | 185.4822 | 2.075309 |
| Btg2 | 96.4528 | 26 | 681 | 424.186 | 763.878 | 321.8838 | 52.26612 | 775.242 | 648.3639 | 3.804908 |
| Lgr6 | 21 | 22 | 0 | 0 | 0 | 70.08153 | 44.22518 | 0 | 0 | 0 |
| Phlda3 | 82 | 144 | 725 | 435 | 928.722 | 273.6517 | 289.4739 | 825.331 | 664.893 | 2.646344 |
| Lad1 | 213 | 228.071 | 195 | 98 | 243.149 | 710.8269 | 458.4764 | 221.9856 | 149.792 | 0.317948 |
| Tnnt2 | 112 | 158 | 58 | 47 | 69 | 373.7682 | 317.6172 | 66.02648 | 71.83901 | 0.199405 |
| Pkp1 | 20 | 6 | 108 | 34 | 120.403 | 66.74431 | 12.06141 | 122.9459 | 51.96865 | 2.219566 |
| Kif21b | 47 | 6 | 37 | 23 | 54.7318 | 156.8491 | 12.06141 | 42.12034 | 35.15526 | 0.457494 |
| Cfh | 27 | 2 | 766.403 | 543.985 | 866.291 | 90.10482 | 4.020471 | 872.4637 | 831.4755 | 18.10288 |
| Rgs2 | 28 | 17 | 181 | 131 | 213.182 | 93.44204 | 34.174 | 206.0482 | 200.2321 | 3.183615 |
| Pla2g4a | 156 | 109 | 130 | 110 | 142 | 520.6056 | 219.1156 | 147.9904 | 168.1339 | 0.427356 |
| Edem3 | 37 | 40.111 | 203 | 117.278 | 246.186 | 123.477 | 80.63255 | 231.0927 | 179.2582 | 2.010445 |
| Rgl1 | 5 | 1 | 130 | 70 | 137 | 16.68608 | 2.010235 | 147.9904 | 106.9943 | 13.63823 |
| uc007czl.1 | 6.03746 | 0 | 148.418 | 108.603 | 286.026 | 20.14831 | 0 | 168.9572 | 165.9986 | 16.62451 |
| Xpr1 | 27.1126 | 16.3574 | 157.861 | 67 | 155.363 | 90.48059 | 32.88222 | 179.707 | 102.4088 | 2.286879 |
| Tor3a | 9 | 11 | 127 | 79 | 107 | 30.03494 | 22.11259 | 144.5752 | 120.7507 | 5.087986 |
| Ralgps2 | 65.2726 | 60 | 87.04 | 30.007 | 71.8396 | 217.8287 | 120.6141 | 99.08526 | 45.86539 | 0.428287 |
| 2810025M15Rik | 93 | 132.01 | 98 | 62.0052 | 127 | 310.3611 | 265.3712 | 111.562 | 94.77431 | 0.358389 |
| Gas5 | 546 | 1052 | 2619.89 | 3307.99 | 2803 | 1822.12 | 2114.768 | 2982.45 | 5056.229 | 2.041887 |
| Pigc | 9 | 16 | 75 | 51 | 104 | 30.03494 | 32.16377 | 85.37907 | 77.95297 | 2.625972 |
| Slc19a2 | 11 | 22 | 219 | 131 | 233 | 36.70937 | 44.22518 | 249.3069 | 200.2321 | 5.554353 |
| Blzf1 | 15 | 15.1184 | 73 | 52 | 86.0244 | 50.05823 | 30.39154 | 83.1023 | 79.48146 | 2.020935 |
| Nme7 | 103.276 | 112.529 | 3006.8 | 1869.91 | 3436.8 | 344.6543 | 226.2098 | 3422.904 | 2858.138 | 11.00269 |
| Atp1b1 | 18.3324 | 44.7812 | 1107.64 | 872.341 | 1180.36 | 61.17917 | 90.02075 | 1260.924 | 1333.364 | 17.158 |
| Creg1 | 27 | 25.0193 | 213.34 | 169.379 | 222 | 90.10482 | 50.29468 | 242.8636 | 258.8941 | 3.573785 |
| Tada1l | 14 | 27.2227 | 121.572 | 59.7121 | 133.45 | 46.72102 | 54.72403 | 138.3961 | 91.26933 | 2.263939 |
| Uck2 | 76 | 189 | 139 | 95.4847 | 170 | 253.6284 | 379.9345 | 158.2359 | 145.9474 | 0.480115 |
| Mgst3 | 157 | 578 | 327 | 251.954 | 345 | 523.9429 | 1161.916 | 372.2528 | 385.1091 | 0.449244 |
| Uap1 | 28 | 44.1981 | 260 | 161 | 249 | 93.44204 | 88.84858 | 295.9808 | 246.0868 | 2.973645 |
| Pcp4l1 | 3 | 0 | 86 | 62 | 80 | 10.01165 | 0 | 97.90134 | 94.76636 | 19.24436 |
| Nit1 | 44 | 34.8173 | 261.637 | 214.991 | 304 | 146.8375 | 69.99097 | 297.8443 | 328.6115 | 2.889177 |
| F11r | 60 | 137 | 406 | 417 | 680.759 | 200.2329 | 275.4022 | 462.1854 | 637.3802 | 2.311784 |
| Ifi204 | 48.7752 | 54.7817 | 34.0787 | 24.9412 | 29.0717 | 162.7734 | 110.1241 | 38.79477 | 38.12237 | 0.281854 |
| Ifi203 | 113.838 | 45.3499 | 66.4755 | 33.9486 | 74.1698 | 379.902 | 91.16397 | 75.67489 | 51.89008 | 0.270801 |
| Ifi202b | 66 | 44 | 34 | 28.9395 | 48 | 220.2562 | 88.45035 | 38.70518 | 44.23373 | 0.268666 |
| Akt3 | 62 | 18 | 25 | 22 | 32 | 206.9074 | 36.18424 | 28.45969 | 33.62677 | 0.255404 |
| Cabc1 | 4.05884 | 0 | 75.2603 | 21.2822 | 75.7858 | 13.54522 | 0 | 85.6754 | 32.52962 | 8.726693 |
| Ephx1 | 20 | 12 | 1097 | 531 | 1235 | 66.74431 | 24.12282 | 1248.811 | 811.628 | 22.6753 |
| Nvl | 49.9516 | 45.6626 | 56.1507 | 42.2582 | 37.0595 | 166.6993 | 91.79257 | 63.92126 | 64.59122 | 0.497163 |
| Cnih4 | 248 | 577 | 391 | 357 | 485 | 827.6295 | 1159.906 | 445.1096 | 545.6708 | 0.498497 |
| uc007dxm.1 | 9.90001 | 0.936348 | 54.3468 | 28.7726 | 127.29 | 33.03847 | 1.88228 | 61.86773 | 43.97862 | 3.031045 |
| Capn2 | 389 | 332 | 379.808 | 214.656 | 399 | 1298.177 | 667.3981 | 432.3687 | 328.0995 | 0.386894 |
| Dusp10 | 53 | 57.1 | 29.0256 | 17.1239 | 32.0624 | 176.8724 | 114.7844 | 33.04238 | 26.1737 | 0.203033 |
| C130074G19Rik | 68 | 40 | 461 | 183 | 436.672 | 226.9307 | 80.40941 | 524.7967 | 279.7136 | 2.617655 |
| Lyplal1 | 15 | 19 | 92 | 63 | 86 | 50.05823 | 38.19447 | 104.7317 | 96.29485 | 2.277851 |
| Cenpf | 37.8947 | 775.576 | 111.976 | 186.187 | 182.196 | 126.4628 | 1559.09 | 127.4721 | 284.5849 | 0.244464 |
| Ptpn14 | 89.8962 | 41 | 545.361 | 216.997 | 560.266 | 300.003 | 82.41965 | 620.8322 | 331.6777 | 2.490726 |
| Vash2 | 38 | 8 | 25 | 11 | 22 | 126.8142 | 16.08188 | 28.45969 | 16.81339 | 0.316825 |
| Dtl | 32 | 52.0315 | 51.5482 | 29.0249 | 60.4304 | 106.7909 | 104.5956 | 58.68183 | 44.36426 | 0.487477 |
| Slc30a1 | 5 | 17 | 104 | 41.3829 | 94.0545 | 16.68608 | 34.174 | 118.3923 | 63.25334 | 3.571478 |
| Sertad4 | 9 | 10 | 91 | 36.0599 | 75 | 30.03494 | 20.10235 | 103.5933 | 55.11718 | 3.165517 |
| Lamb3 | 69 | 50 | 361.435 | 208 | 462.154 | 230.2679 | 100.5118 | 411.4531 | 317.9259 | 2.20503 |
| uc007eer.1 | 3 | 3 | 71 | 33 | 61 | 10.01165 | 6.030706 | 80.82552 | 50.44016 | 8.182446 |
| Fbxo5 | 13.65 | 125.165 | 30.5008 | 28.5916 | 61.1214 | 45.55299 | 251.6111 | 34.72173 | 43.70197 | 0.263907 |
| Akap12 | 19 | 6.02859 | 170.438 | 64.793 | 178.27 | 63.4071 | 12.11888 | 194.0245 | 99.03543 | 3.880253 |
| Lrp11 | 7 | 6 | 69 | 43 | 64 | 23.36051 | 12.06141 | 78.54875 | 65.72506 | 4.073009 |
| BC013529 | 39 | 36 | 287 | 210.525 | 295 | 130.1514 | 72.36847 | 326.7173 | 321.7853 | 3.202167 |
| Stxbp5 | 13 | 14 | 78 | 37 | 84.186 | 43.3838 | 28.14329 | 88.79424 | 56.55412 | 2.032074 |
| Rab32 | 3 | 5 | 247 | 213 | 356 | 10.01165 | 10.05118 | 281.1817 | 325.5683 | 30.24251 |
| Fuca2 | 9 | 12 | 157.405 | 107.87 | 189.146 | 30.03494 | 24.12282 | 179.1879 | 164.8782 | 6.353033 |
| Hivep2 | 31.4717 | 13.3049 | 150.175 | 88.5893 | 152.321 | 105.0278 | 26.74598 | 170.9574 | 135.4078 | 2.324932 |
| Pex7 | 32 | 40 | 172 | 130 | 193 | 106.7909 | 80.40941 | 195.8027 | 198.7037 | 2.107402 |
| uc007epl.1 | 40.1643 | 35.8705 | 12.2703 | 7 | 21.2068 | 134.0369 | 72.10815 | 13.96836 | 10.69943 | 0.119662 |
| Vnn1 | 40 | 31 | 292 | 202 | 335 | 133.4886 | 62.31729 | 332.4092 | 308.7549 | 3.274488 |
| Enpp1 | 29 | 31 | 11 | 10 | 22 | 96.77925 | 62.31729 | 12.52226 | 15.2849 | 0.174782 |
| C030003D03Rik | 49 | 25 | 11 | 7 | 4 | 163.5236 | 50.25588 | 12.52226 | 10.69943 | 0.108625 |
| Ptprk | 82 | 77.2849 | 458.75 | 233 | 585.933 | 273.6517 | 155.3608 | 522.2353 | 356.1381 | 2.047431 |
| uc007etc.1 | 80.0389 | 366.287 | 160.893 | 141.092 | 168.32 | 267.1071 | 736.3231 | 183.1586 | 215.6577 | 0.397453 |
| 2610036L11Rik | 44.1301 | 138.425 | 24.0252 | 28.388 | 43.1469 | 147.2717 | 278.2668 | 27.34999 | 43.39076 | 0.166238 |
| Hint3 | 10 | 11 | 58 | 54 | 97 | 33.37216 | 22.11259 | 66.02648 | 82.53844 | 2.677581 |
| Tpd52l1 | 89 | 67 | 26 | 27 | 26 | 297.0122 | 134.6858 | 29.59808 | 41.26922 | 0.164159 |
| Zufsp | 6 | 12 | 57 | 32 | 54 | 20.02329 | 24.12282 | 64.8881 | 48.91167 | 2.577798 |
| Dse | 31 | 35 | 37.3484 | 18 | 46.2837 | 103.4537 | 70.35824 | 42.51696 | 27.51281 | 0.402905 |
| Frk | 11 | 23 | 70 | 66 | 93 | 36.70937 | 46.23541 | 79.68713 | 100.8803 | 2.17696 |
| Lama4 | 2.00121 | 0 | 111.146 | 50.9126 | 114 | 6.678469 | 0 | 126.5272 | 77.81938 | 30.59782 |
| Fyn | 52 | 20 | 35 | 25 | 45 | 173.5352 | 40.20471 | 39.84357 | 38.21224 | 0.365191 |
| Rev3l | 14 | 8 | 65 | 35 | 93 | 46.72102 | 16.08188 | 73.9952 | 53.49714 | 2.030039 |
| Gtf3c6 | 27 | 86 | 39 | 55 | 48 | 90.10482 | 172.8802 | 44.39712 | 84.06693 | 0.488484 |
| uc007eyk.1 | 17.0147 | 2 | 218.2 | 65 | 252.446 | 56.78172 | 4.020471 | 248.3962 | 99.35183 | 5.719333 |
| Ostm1 | 32 | 17.3479 | 208.611 | 121.438 | 202.468 | 106.7909 | 34.87336 | 237.4802 | 185.6167 | 2.986617 |
| Cd24a | 471 | 813 | 3772.94 | 2881 | 4755.92 | 1571.829 | 1634.321 | 4295.068 | 4403.579 | 2.713113 |
| Gopc | 31 | 50 | 255 | 168 | 265 | 103.4537 | 100.5118 | 290.2888 | 256.7863 | 2.682195 |
| Man1a | 15 | 6 | 106 | 42.3445 | 90.2083 | 50.05823 | 12.06141 | 120.6691 | 64.72313 | 2.984438 |
| Gja1 | 101.058 | 176.977 | 1007.6 | 481.405 | 1079.86 | 337.2523 | 355.7654 | 1147.039 | 735.8226 | 2.716903 |
| Smpdl3a | 10 | 4 | 60 | 50 | 69 | 33.37216 | 8.040941 | 68.30326 | 76.42448 | 3.494734 |
| Ddit4 | 159 | 73 | 2118 | 1755.97 | 2548.93 | 530.6173 | 146.7472 | 2411.105 | 2683.982 | 7.521928 |
| Ascc1 | 21 | 20 | 105 | 68 | 132 | 70.08153 | 40.20471 | 119.5307 | 103.9373 | 2.026255 |
| Ppa1 | 196 | 611 | 420 | 258 | 447 | 654.0943 | 1228.254 | 478.1228 | 394.3503 | 0.463503 |
| Slc25a16 | 18.0555 | 18.3155 | 155.118 | 156.273 | 185.56 | 60.2551 | 36.81846 | 176.5844 | 238.8617 | 4.279704 |
| Cdc2a | 133 | 902 | 378 | 389 | 434 | 443.8497 | 1813.232 | 430.3105 | 594.5825 | 0.454079 |
| uc007foe.1 | 7 | 4 | 91 | 52 | 76 | 23.36051 | 8.040941 | 103.5933 | 79.48146 | 5.830136 |
| Chchd10 | 38 | 34 | 164.662 | 142 | 149.62 | 126.8142 | 68.348 | 187.4492 | 217.0455 | 2.072608 |
| Shc2 | 19.9041 | 21.9778 | 11.7695 | 7.74069 | 22.8828 | 66.42427 | 44.18055 | 13.39825 | 11.83156 | 0.228108 |
| Wdr18 | 122.641 | 235.633 | 198.611 | 127.317 | 239.295 | 409.2795 | 473.6778 | 226.0963 | 194.6027 | 0.476466 |
| Cnn2 | 144 | 330 | 144 | 137 | 225 | 480.5591 | 663.3777 | 163.9278 | 209.4031 | 0.326356 |
| Hmha1 | 21 | 27 | 3 | 2 | 7 | 70.08153 | 54.27635 | 3.415163 | 3.056979 | 0.052044 |
| Polr2e | 273 | 730 | 472 | 347 | 438 | 911.0599 | 1467.472 | 537.319 | 530.3859 | 0.448892 |
| Cirbp | 21 | 22 | 261.951 | 173.72 | 266.903 | 70.08153 | 44.22518 | 298.2018 | 265.5292 | 4.93174 |
| Gamt | 20 | 57 | 25 | 21 | 16 | 66.74431 | 114.5834 | 28.45969 | 32.09828 | 0.33397 |
| Pcsk4 | 21 | 29 | 25 | 13 | 25 | 70.08153 | 58.29682 | 28.45969 | 19.87037 | 0.376466 |
| Adamtsl5 | 84 | 66 | 31 | 15 | 32 | 280.3261 | 132.6755 | 35.29002 | 22.92735 | 0.140962 |
| Mobkl2a | 62 | 28.129 | 54 | 24.1297 | 95 | 206.9074 | 56.54591 | 61.47293 | 36.882 | 0.37333 |
| Lsm7 | 166 | 807.985 | 374.871 | 401.635 | 323.349 | 553.9778 | 1624.24 | 426.7485 | 613.8949 | 0.47775 |
| 3110056O03Rik | 13 | 12.0147 | 102.214 | 40.3653 | 147 | 43.3838 | 24.15237 | 116.3592 | 61.69794 | 2.63647 |
| Tjp3 | 16 | 3 | 186 | 98 | 211 | 53.39545 | 6.030706 | 211.7401 | 149.792 | 6.08372 |
| uc007gje.1 | 96.4132 | 1524.51 | 69.2571 | 429.858 | 69.1607 | 321.7516 | 3064.624 | 78.84143 | 657.0335 | 0.217305 |
| uc007gjh.1 | 96.4132 | 1503.47 | 69.2571 | 429.858 | 51.9782 | 321.7516 | 3022.328 | 78.84143 | 657.0335 | 0.220053 |
| Chst11 | 20 | 18 | 163 | 62 | 205 | 66.74431 | 36.18424 | 185.5572 | 94.76636 | 2.723477 |
| A230046K03Rik | 98.4948 | 508.781 | 202.064 | 228.272 | 216.789 | 328.6984 | 1022.77 | 230.0272 | 348.9114 | 0.428378 |
| Tcp11l2 | 15 | 2 | 196.438 | 135 | 194 | 50.05823 | 4.020471 | 223.6226 | 206.3461 | 7.950795 |
| Syn3 | 4.76915 | 5.02074 | 800.463 | 342.623 | 948.066 | 15.91568 | 10.09287 | 911.2372 | 523.6957 | 55.17158 |
| Timp3 | 29.2309 | 9.97926 | 1613.39 | 838.067 | 1652.71 | 97.54982 | 20.06066 | 1836.663 | 1280.977 | 26.50818 |
| Nt5dc3 | 94.701 | 51.541 | 64.1128 | 39.0716 | 79.4481 | 316.0377 | 103.6095 | 72.98522 | 59.72054 | 0.316232 |
| Nup37 | 51 | 173 | 77 | 64 | 76 | 170.198 | 347.7707 | 87.65585 | 97.82334 | 0.35809 |
| 1200002N14Rik | 3 | 0 | 1467 | 755 | 1458 | 10.01165 | 0 | 1670.015 | 1154.01 | 282.0739 |
| Gnptab | 61 | 46.8172 | 420 | 235.679 | 482.53 | 203.5702 | 94.11359 | 478.1228 | 360.2329 | 2.816263 |
| Chpt1 | 61.6282 | 1802.04 | 238.46 | 597.091 | 235.503 | 205.6666 | 3622.524 | 271.4599 | 912.6474 | 0.309313 |
| Gas2l3 | 58.9601 | 336.119 | 85.2132 | 56.2223 | 89.0132 | 196.7626 | 675.6783 | 97.00565 | 85.9352 | 0.209689 |
| Anks1b | 15 | 44.6174 | 1.0118 | 2 | 1 | 50.05823 | 89.69147 | 1.151821 | 3.056979 | 0.030117 |
| Tmcc3 | 77.4518 | 61 | 75 | 46.3654 | 89.2189 | 258.4734 | 122.6244 | 85.37907 | 70.86904 | 0.409995 |
| Ccdc41 | 43.4344 | 118.766 | 74.5208 | 48.748 | 80.3856 | 144.95 | 238.7476 | 84.83356 | 74.51081 | 0.415286 |
| 4930430F08Rik | 18.3334 | 11.8182 | 89.9818 | 57.8 | 99.755 | 61.18251 | 23.75736 | 102.4342 | 88.3467 | 2.24607 |
| Rassf9 | 2 | 3 | 66 | 63 | 77 | 6.674431 | 6.030706 | 75.13358 | 96.29485 | 13.49284 |
| Tmtc2 | 2 | 3 | 125.2 | 63 | 185 | 6.674431 | 6.030706 | 142.5261 | 96.29485 | 18.7972 |
| Pawr | 9 | 13 | 132 | 99 | 56 | 30.03494 | 26.13306 | 150.2672 | 151.3205 | 5.369386 |
| Glipr1 | 62.5465 | 89.9248 | 741.766 | 657.018 | 982.969 | 208.7312 | 180.77 | 844.4172 | 1004.245 | 4.746231 |
| Tmem19 | 30 | 16 | 150 | 69 | 165 | 100.1165 | 32.16377 | 170.7581 | 105.4658 | 2.088172 |
| Lgr5 | 4.00241 | 0 | 61.0312 | 42.2393 | 85.1836 | 13.35691 | 0 | 69.47716 | 64.56233 | 10.03522 |
| Mdm2 | 55 | 49 | 587.907 | 378 | 706.772 | 183.5469 | 98.50153 | 669.2661 | 577.7691 | 4.421352 |
| Tmbim4 | 104 | 141 | 599 | 513 | 637 | 347.0704 | 283.4432 | 681.8942 | 784.1152 | 2.325104 |
| Hmga2 | 1092.48 | 949.8 | 277.426 | 112.899 | 362.923 | 3645.841 | 1909.322 | 315.8183 | 172.565 | 0.087915 |
| Msrb3 | 14 | 9 | 274.028 | 162.354 | 351.978 | 46.72102 | 18.09212 | 311.9501 | 248.1564 | 8.641867 |
| Gns | 43.6506 | 54 | 392.71 | 136.577 | 460.131 | 145.6715 | 108.5527 | 447.0562 | 208.7565 | 2.579663 |
| Tbk1 | 35 | 39 | 296 | 174 | 292 | 116.8025 | 78.39918 | 336.9627 | 265.9572 | 3.088702 |
| Os9 | 53 | 43 | 368 | 160 | 467 | 176.8724 | 86.44012 | 418.9267 | 244.5583 | 2.519762 |
| B4galnt1 | 11 | 6 | 127.541 | 72.6041 | 141.434 | 36.70937 | 12.06141 | 145.1911 | 110.9746 | 5.252442 |
| uc007hih.1 | 11 | 6 | 70.4593 | 27.3959 | 78.5664 | 36.70937 | 12.06141 | 80.21 | 41.87435 | 2.503227 |
| Dtx3 | 67 | 13 | 301 | 147 | 312 | 223.5934 | 26.13306 | 342.6547 | 224.688 | 2.271856 |
| Gls2 | 46 | 91 | 34 | 22 | 39 | 153.5119 | 182.9314 | 38.70518 | 33.62677 | 0.21499 |
| Timeless | 25 | 35.1171 | 37 | 15.9375 | 60 | 83.43039 | 70.59363 | 42.12034 | 24.3603 | 0.431625 |
| Stat2 | 16 | 8 | 171 | 73 | 177 | 53.39545 | 16.08188 | 194.6643 | 111.5797 | 4.407827 |
| Coq10a | 13 | 13 | 95.0678 | 48 | 86 | 43.3838 | 26.13306 | 108.224 | 73.3675 | 2.612194 |
| Smarcc2 | 28 | 30 | 206 | 67.9853 | 164 | 93.44204 | 60.30706 | 234.5079 | 103.9148 | 2.201136 |
| Suox | 36.1011 | 74.2464 | 41 | 19 | 39 | 120.4772 | 149.2527 | 46.67389 | 29.0413 | 0.280707 |
| Dgka | 2.66674 | 2 | 59.5 | 34.3636 | 94.1379 | 8.899486 | 4.020471 | 67.73406 | 52.52441 | 9.307962 |
| Pik3ip1 | 35 | 11 | 176 | 111 | 202 | 116.8025 | 22.11259 | 200.3562 | 169.6624 | 2.66363 |
| Selm | 45 | 44 | 416 | 306 | 289 | 150.1747 | 88.45035 | 473.5693 | 467.7178 | 3.944628 |
| Morc2a | 91 | 55 | 95 | 41 | 124 | 303.6866 | 110.5629 | 108.1468 | 62.66808 | 0.412348 |
| Lif | 34 | 41 | 212.408 | 103 | 153 | 113.4653 | 82.41965 | 241.8026 | 157.4344 | 2.03812 |
| Ascc2 | 68 | 26 | 56 | 30 | 58 | 226.9307 | 52.26612 | 63.74971 | 45.85469 | 0.39257 |
| Kremen1 | 18.1081 | 10.0099 | 137.062 | 57.6782 | 107.649 | 60.43063 | 20.12225 | 156.0297 | 88.16053 | 3.031427 |
| Znrf3 | 34.9766 | 62.0721 | 237.907 | 202.255 | 209.447 | 116.7245 | 124.7795 | 270.8304 | 309.1447 | 2.401513 |
| Xbp1 | 129 | 67 | 2352 | 1122 | 2427 | 430.5008 | 134.6858 | 2677.488 | 1714.965 | 7.771687 |
| Ccdc117 | 19 | 21 | 153.267 | 86 | 175 | 63.4071 | 42.21494 | 174.4773 | 131.4501 | 2.896435 |
| Pold2 | 85 | 179 | 144 | 77 | 166 | 283.6633 | 359.8321 | 163.9278 | 117.6937 | 0.437643 |
| uc007hxv.1 | 19.7223 | 14.6774 | 2132.97 | 833.932 | 3070.15 | 65.81757 | 29.50503 | 2428.147 | 1274.656 | 38.84497 |
| Ogdh | 328.702 | 3101.46 | 853.912 | 1330.7 | 1139.94 | 1096.949 | 6234.664 | 972.0829 | 2033.961 | 0.410011 |
| Igfbp3 | 653 | 799.832 | 226 | 140.843 | 208 | 2179.202 | 1607.851 | 257.2756 | 215.2771 | 0.124781 |
| Ddc | 17 | 24 | 0 | 0 | 1 | 56.73267 | 48.24565 | 0 | 0 | 0 |
| Wdr92 | 784.943 | 364.935 | 7423.81 | 3900.47 | 8140.56 | 2619.524 | 733.6052 | 8451.174 | 5961.828 | 4.298373 |
| Sertad2 | 116 | 93.9807 | 152.065 | 71.04 | 158.735 | 387.117 | 188.9233 | 173.1089 | 108.5839 | 0.489016 |
| uc007iew.1 | 221.683 | 6628.35 | 762.286 | 2314.82 | 1120.38 | 739.804 | 13324.54 | 867.777 | 3538.178 | 0.313271 |
| Fancl | 7.33105 | 24.05 | 96.2738 | 72.68 | 96.663 | 24.46529 | 48.34616 | 109.5969 | 111.0906 | 3.030945 |
| Ccdc104 | 22 | 30 | 182 | 141 | 247 | 73.41874 | 60.30706 | 207.1866 | 215.517 | 3.160973 |
| Chac2 | 25 | 38 | 230 | 200 | 265 | 83.43039 | 76.38894 | 261.8292 | 305.6979 | 3.551054 |
| Stc2 | 2 | 3 | 76 | 53.0842 | 98 | 6.674431 | 6.030706 | 86.51746 | 81.13865 | 13.19593 |
| Snrnp25 | 40 | 153 | 76 | 61 | 64 | 133.4886 | 307.566 | 86.51746 | 93.23787 | 0.407558 |
| Ranbp17 | 7.23847 | 12.0699 | 38.2208 | 45.3777 | 45.1137 | 24.15634 | 24.26334 | 43.51009 | 69.35935 | 2.331065 |
| Gabrp | 2 | 0 | 599 | 304 | 884 | 6.674431 | 0 | 681.8942 | 464.6609 | 171.7832 |
| Wwc1 | 9 | 18 | 46 | 63.9844 | 22 | 30.03494 | 36.18424 | 52.36583 | 97.79949 | 2.267702 |
| Ccng1 | 86 | 63.2862 | 1613.39 | 974.037 | 1819.21 | 287.0005 | 127.2202 | 1836.663 | 1488.805 | 8.028253 |
| Pttg1 | 62 | 358 | 191 | 122 | 258 | 206.9074 | 719.6642 | 217.432 | 186.4757 | 0.435916 |
| Nhp2 | 274.955 | 960.922 | 482.664 | 397.424 | 546.372 | 917.5841 | 1931.679 | 549.4587 | 607.4585 | 0.406041 |
| Phf15 | 45 | 26 | 270.991 | 76 | 250 | 150.1747 | 52.26612 | 308.4928 | 116.1652 | 2.09769 |
| Zcchc10 | 58 | 118 | 74.1056 | 60.9676 | 87.4701 | 193.5585 | 237.2078 | 84.3609 | 93.18835 | 0.412171 |
| Shroom1 | 10.6487 | 8.0485 | 111.416 | 52.2948 | 140.688 | 35.53701 | 16.17938 | 126.8346 | 79.93206 | 3.998088 |
| Irf1 | 55 | 31.0753 | 293.252 | 162.256 | 382.643 | 183.5469 | 62.46867 | 333.8345 | 248.0066 | 2.365058 |
| Slc22a5 | 12 | 15.7143 | 121.856 | 52.9811 | 103 | 40.04659 | 31.58944 | 138.7194 | 80.98106 | 3.066899 |
| Pdlim4 | 29 | 10 | 356.281 | 181 | 430 | 96.77925 | 20.10235 | 405.5859 | 276.6566 | 5.837039 |
| Cdc42se2 | 161 | 247 | 288 | 123 | 279 | 537.2917 | 496.5281 | 327.8556 | 188.0042 | 0.498984 |
| Hint1 | 878 | 3140 | 1459 | 1589 | 1870 | 2930.075 | 6312.139 | 1660.908 | 2428.77 | 0.4425 |
| Gpx3 | 4 | 14 | 335.094 | 205.235 | 452 | 13.34886 | 28.14329 | 381.4669 | 313.6996 | 16.75417 |
| Slc36a1 | 16 | 8.18428 | 81.8678 | 34.0399 | 89.8198 | 53.39545 | 16.45233 | 93.19729 | 52.02964 | 2.079192 |
| Sparc | 175.911 | 151 | 146 | 72 | 186.244 | 587.0529 | 303.5455 | 166.2046 | 110.0513 | 0.310191 |
| Larp1 | 18.6487 | 20.1241 | 21.0247 | 11.1667 | 29.1524 | 62.23473 | 40.45418 | 23.93426 | 17.06819 | 0.399288 |
| Mrpl22 | 130 | 368 | 215 | 211 | 259 | 433.838 | 739.7666 | 244.7533 | 322.5113 | 0.483352 |
| uc007jat.1 | 7 | 2.5 | 82.299 | 59.5787 | 72.029 | 23.36051 | 5.025588 | 93.68816 | 91.06543 | 6.508594 |
| Irgm2 | 7 | 2.5 | 82.299 | 59.5787 | 72.029 | 23.36051 | 5.025588 | 93.68816 | 91.06543 | 6.508594 |
| Zfp672 | 9 | 15 | 80 | 54 | 71 | 30.03494 | 30.15353 | 91.07101 | 82.53844 | 2.88443 |
| Hist3h2a | 9.49633 | 6.44009 | 62.6059 | 58.4924 | 61.7379 | 31.6913 | 12.9461 | 71.26978 | 89.40503 | 3.599556 |
| Shmt1 | 54 | 51.0433 | 31.0218 | 15.0016 | 35.0607 | 180.2096 | 102.609 | 35.31483 | 22.92979 | 0.205943 |
| Aldh3a1 | 18 | 26 | 427 | 272 | 541 | 60.06988 | 52.26612 | 486.0915 | 415.7492 | 8.028065 |
| B9d1 | 8 | 64 | 124 | 126 | 135 | 26.69773 | 128.6551 | 141.1601 | 192.5897 | 2.148335 |
| Cenpv | 96.9588 | 149 | 121 | 69.971 | 134.985 | 323.5724 | 299.5251 | 137.7449 | 106.95 | 0.392707 |
| uc007jjk.1 | 1802.16 | 731.954 | 1327.8 | 1328.2 | 1290.16 | 6014.197 | 1471.4 | 1511.551 | 2030.14 | 0.473134 |
| uc007jjl.1 | 703.218 | 825.886 | 365.874 | 790.135 | 345.662 | 2346.79 | 1660.225 | 416.5064 | 1207.713 | 0.405344 |
| AU040829 | 36.1278 | 5 | 320.286 | 112.406 | 334.776 | 120.5663 | 10.05118 | 364.6096 | 171.8114 | 4.10681 |
| Pik3r5 | 5.00318 | 1.00332 | 88 | 46 | 70.0815 | 16.69669 | 2.016909 | 100.1781 | 70.31052 | 9.110414 |
| uc007jou.1 | 40.6408 | 35.9459 | 66.601 | 17.5826 | 83.5063 | 135.6271 | 72.25972 | 75.81776 | 26.87482 | 0.493983 |
| Aurkb | 41 | 183 | 95 | 73 | 147.652 | 136.8258 | 367.8731 | 108.1468 | 111.5797 | 0.435362 |
| Vamp2 | 21 | 29 | 184.565 | 87 | 171 | 70.08153 | 58.29682 | 210.1065 | 132.9786 | 2.672453 |
| Per1 | 21 | 8 | 151 | 48 | 203 | 70.08153 | 16.08188 | 171.8965 | 73.3675 | 2.846499 |
| Trp53 | 13 | 22 | 217 | 133 | 217 | 43.3838 | 44.22518 | 247.0301 | 203.2891 | 5.140104 |
| Cldn7 | 4 | 19.0283 | 185 | 129.205 | 257.991 | 13.34886 | 38.25136 | 210.6017 | 197.4885 | 7.908691 |
| Acadvl | 32.5557 | 54.5351 | 248.536 | 114.887 | 325.014 | 108.6454 | 109.6284 | 282.9303 | 175.6036 | 2.100728 |
| Cxcl16 | 66 | 32.0543 | 978.657 | 514.997 | 1048.99 | 220.2562 | 64.43669 | 1114.091 | 787.1676 | 6.678279 |
| Eno3 | 90 | 123 | 19.0078 | 12.0081 | 22 | 300.3494 | 247.2589 | 21.63824 | 18.35426 | 0.073031 |
| 6720460F02Rik | 36 | 280 | 101 | 73 | 85 | 120.1398 | 562.8659 | 114.9772 | 111.5797 | 0.331706 |
| Mybbp1a | 363 | 385 | 441 | 249 | 529 | 1211.409 | 773.9406 | 502.0289 | 380.5939 | 0.444568 |
| uc007jyz.1 | 22.0697 | 7 | 666.885 | 341.439 | 728.72 | 73.65135 | 14.07165 | 759.1736 | 521.886 | 14.60346 |
| uc007jza.1 | 1.93029 | 7 | 67.115 | 17.5612 | 97.2714 | 6.441794 | 14.07165 | 76.40289 | 26.84211 | 5.033041 |
| Ankfy1 | 263.253 | 540.139 | 383.403 | 258.638 | 419.507 | 878.532 | 1085.806 | 436.4612 | 395.3255 | 0.423444 |
| Gsg2 | 13 | 32 | 21 | 12 | 27 | 43.3838 | 64.32753 | 23.90614 | 18.34188 | 0.392234 |
| Ctns | 13.1434 | 4.2126 | 73.246 | 27.38 | 64.2669 | 43.86236 | 8.468317 | 83.38234 | 41.85005 | 2.393097 |
| Aspa | 19 | 8 | 1709.88 | 1105 | 1707 | 63.4071 | 16.08188 | 1946.506 | 1688.981 | 45.73574 |
| Mett10d | 45 | 96 | 86 | 47 | 106 | 150.1747 | 192.9826 | 97.90134 | 71.83901 | 0.494643 |
| Tsr1 | 94.9722 | 146.188 | 134.416 | 86.6878 | 172.105 | 316.9427 | 293.8723 | 153.0175 | 132.5014 | 0.467439 |
| uc007kdv.1 | 19 | 36.3689 | 219 | 158.825 | 253.811 | 63.4071 | 73.11005 | 249.3069 | 242.7624 | 3.60445 |
| Slc43a2 | 3 | 11.0661 | 63 | 33.0683 | 63.1415 | 10.01165 | 22.24547 | 71.71842 | 50.54456 | 3.790264 |
| Glod4 | 40.2263 | 26.3314 | 163.182 | 124.293 | 178.533 | 134.2438 | 52.93231 | 185.7644 | 189.9806 | 2.00744 |
| Fam57a | 15.1141 | 38.3857 | 30.659 | 12.9104 | 27.0181 | 50.43901 | 77.16429 | 34.90183 | 19.73341 | 0.428165 |
| Timm22 | 373.891 | 1709.54 | 779.189 | 765.481 | 1162.9 | 1247.755 | 3436.578 | 887.0191 | 1170.03 | 0.439134 |
| Cpd | 103 | 74.1186 | 1498 | 704 | 1549.99 | 343.7332 | 148.9958 | 1705.305 | 1076.057 | 5.644809 |
| Blmh | 35 | 24 | 182 | 108 | 265 | 116.8025 | 48.24565 | 207.1866 | 165.0769 | 2.255483 |
| Ccdc55 | 49.108 | 168.273 | 121.44 | 72.5736 | 160.463 | 163.884 | 338.2683 | 138.2458 | 110.928 | 0.496212 |
| Ankrd13b | 122 | 46 | 103 | 37 | 136 | 407.1403 | 92.47082 | 117.2539 | 56.55412 | 0.347887 |
| Spag5 | 27 | 92 | 72 | 35 | 76 | 90.10482 | 184.9416 | 81.96391 | 53.49714 | 0.492502 |
| Aldoc | 66 | 9.01084 | 248 | 151 | 286.067 | 220.2562 | 18.11391 | 282.3201 | 230.8019 | 2.152627 |
| Myo1d | 65 | 15 | 436.268 | 210 | 467 | 216.919 | 30.15353 | 496.6421 | 320.9828 | 3.30925 |
| Ccl2 | 1 | 5 | 7095.9 | 6372.99 | 7436 | 3.337216 | 10.05118 | 8077.885 | 9741.049 | 1330.924 |
| Ccl7 | 0 | 1 | 772.098 | 782 | 782 | 0 | 2.010235 | 878.9468 | 1195.279 | 1031.832 |
| Slfn2 | 6.87212 | 4.08333 | 353.755 | 186.285 | 396.369 | 22.93375 | 8.208454 | 402.7103 | 284.7347 | 22.07439 |
| Hnf1b | 48 | 49 | 307 | 117 | 333 | 160.1864 | 98.50153 | 349.485 | 178.8333 | 2.0423 |
| Ap1gbp1 | 41.345 | 1637.13 | 42.8039 | 56.568 | 35.2987 | 137.9772 | 3291.017 | 48.72743 | 86.4636 | 0.039426 |
| Aatf | 62 | 79 | 62 | 41 | 104.732 | 206.9074 | 158.8086 | 70.58003 | 62.66808 | 0.364349 |
| Ppm1d | 10 | 18 | 97 | 62 | 86.0835 | 33.37216 | 36.18424 | 110.4236 | 94.76636 | 2.94998 |
| Brip1 | 27.4441 | 135.342 | 22.801 | 42.658 | 29.5474 | 91.58688 | 272.0693 | 25.95638 | 65.20231 | 0.250673 |
| uc007ksg.1 | 97.7617 | 313.196 | 193.167 | 124.859 | 222.094 | 326.2519 | 629.5977 | 219.8989 | 190.8457 | 0.429717 |
| Ypel2 | 17 | 3 | 92 | 31.0654 | 87 | 56.73267 | 6.030706 | 104.7317 | 47.48314 | 2.425217 |
| Prr11 | 10.4409 | 58.9288 | 16.4062 | 13.884 | 24.3496 | 34.84353 | 118.4608 | 18.67662 | 21.22155 | 0.260255 |
| 1110001A07Rik | 66 | 159.975 | 130.139 | 77.8152 | 111.074 | 220.2562 | 321.5874 | 148.1486 | 118.9397 | 0.492925 |
| Rnf43 | 9 | 7 | 131 | 55 | 170 | 30.03494 | 14.07165 | 149.1288 | 84.06693 | 5.287095 |
| Mmd | 248 | 347 | 167 | 136.403 | 173 | 827.6295 | 697.5517 | 190.1107 | 208.4906 | 0.261347 |
| Nme2 | 1291.85 | 5791.84 | 2986.84 | 2766.97 | 3257.81 | 4311.182 | 11642.96 | 3400.182 | 4229.285 | 0.478212 |
| Nme1 | 572.149 | 2471.16 | 1294.16 | 1163.03 | 1360.19 | 1909.385 | 4967.613 | 1473.256 | 1777.679 | 0.472726 |
| Tob1 | 108.263 | 77 | 761.988 | 509.998 | 837.208 | 361.297 | 154.7881 | 867.4377 | 779.5267 | 3.191265 |
| Abcc3 | 46 | 27 | 322 | 217 | 350 | 153.5119 | 54.27635 | 366.5608 | 331.6823 | 3.360358 |
| Epn3 | 10 | 6 | 79 | 37 | 114 | 33.37216 | 12.06141 | 89.93262 | 56.55412 | 3.224196 |
| Eme1 | 17.2213 | 28.4585 | 28.1719 | 9.7711 | 24.3696 | 57.47119 | 57.20828 | 32.07054 | 14.93503 | 0.409887 |
| Samd14 | 26 | 15 | 21 | 7 | 32 | 86.76761 | 30.15353 | 23.90614 | 10.69943 | 0.295974 |
| Igf2bp1 | 48.1345 | 48.0514 | 5.01911 | 3.60651 | 6.05161 | 160.6352 | 96.59462 | 5.713693 | 5.512513 | 0.043643 |
| Atp5g1 | 408.819 | 2422.37 | 869.815 | 1259.27 | 1046.78 | 1364.317 | 4869.534 | 990.1866 | 1924.781 | 0.467603 |
| Cbx1 | 9.36317 | 38.66 | 22.1157 | 14.3232 | 24.4225 | 31.24692 | 77.7157 | 25.17624 | 21.89286 | 0.431975 |
| Atad4 | 10 | 3 | 391 | 271 | 481 | 33.37216 | 6.030706 | 445.1096 | 414.2207 | 21.80883 |
| Pnpo | 26 | 25 | 157 | 71 | 144 | 86.76761 | 50.25588 | 178.7269 | 108.5228 | 2.096353 |
| Ppp1r1b | 2 | 1 | 120 | 76 | 129 | 6.674431 | 2.010235 | 136.6065 | 116.1652 | 29.10552 |
| Csf3 | 1 | 1 | 471 | 340 | 580.732 | 3.337216 | 2.010235 | 536.1806 | 519.6865 | 197.4524 |
| Nr1d1 | 17.8605 | 28.3636 | 31.6981 | 5 | 23.6794 | 59.60434 | 57.01751 | 36.08473 | 7.642448 | 0.374948 |
| Cdc6 | 12 | 55 | 34 | 23 | 34.1562 | 40.04659 | 110.5629 | 38.70518 | 35.15526 | 0.49041 |
| Igfbp4 | 1755.99 | 1881.96 | 384 | 177 | 414.816 | 5860.117 | 3783.182 | 437.1409 | 270.5427 | 0.073386 |
| Tns4 | 106.556 | 70.4691 | 9.04045 | 5.07277 | 9.10402 | 355.6003 | 141.6595 | 10.29154 | 7.753677 | 0.036289 |
| Krt23 | 90.0378 | 51 | 20.0023 | 4 | 8.00073 | 300.4756 | 102.522 | 22.77037 | 6.113959 | 0.071674 |
| Hap1 | 12 | 4 | 217 | 106 | 224 | 40.04659 | 8.040941 | 247.0301 | 162.0199 | 8.506364 |
| 1110036O03Rik | 99.6839 | 276.384 | 171 | 161.87 | 246.258 | 332.6667 | 555.5969 | 194.6643 | 247.4166 | 0.497691 |
| Fkbp10 | 60 | 79 | 36 | 15 | 50 | 200.2329 | 158.8086 | 40.98195 | 22.92735 | 0.178 |
| Stat3 | 74.3394 | 47 | 668 | 377.599 | 724.84 | 248.0866 | 94.48106 | 760.4429 | 577.1562 | 3.904627 |
| Vps25 | 71 | 103 | 447 | 288 | 521.715 | 236.9423 | 207.0542 | 508.8593 | 440.205 | 2.137549 |
| Ifi35 | 2.38791 | 5.0566 | 16.4157 | 78.704 | 13.1695 | 7.968971 | 10.16496 | 18.68743 | 120.2983 | 7.664401 |
| Brca1 | 20 | 28 | 27 | 14 | 32 | 66.74431 | 56.28659 | 30.73647 | 21.39886 | 0.423758 |
| Rdm1 | 18 | 30 | 23 | 11 | 44 | 60.06988 | 60.30706 | 26.18292 | 16.81339 | 0.357181 |
| Etv4 | 116.398 | 94.2686 | 54.5871 | 29.533 | 77.5544 | 388.4452 | 189.5021 | 62.14128 | 45.14089 | 0.185626 |
| Hdac5 | 38 | 25 | 211 | 97 | 253 | 126.8142 | 50.25588 | 240.1998 | 148.2635 | 2.193839 |
| Gjc1 | 33 | 34.0775 | 2.00315 | 1.02872 | 0 | 110.1281 | 68.50379 | 2.280361 | 1.572388 | 0.021568 |
| Plcd3 | 56.0831 | 45.9508 | 75.1939 | 34.0509 | 109.228 | 187.1614 | 92.37192 | 85.59981 | 52.04645 | 0.492414 |
| Fmnl1 | 39 | 39.5512 | 4 | 0 | 4 | 130.1514 | 79.50722 | 4.553551 | 0 | 0.021719 |
| Arhgap27 | 87 | 74 | 86 | 41 | 94 | 290.3378 | 148.7574 | 97.90134 | 62.66808 | 0.365682 |
| Cyb561 | 17 | 11 | 208 | 121 | 221 | 56.73267 | 22.11259 | 236.7846 | 184.9473 | 5.348856 |
| Ace | 5 | 1 | 92 | 53 | 106 | 16.68608 | 2.010235 | 104.7317 | 81.00995 | 9.934665 |
| 1810010H24Rik | 4 | 2 | 265 | 198.98 | 279 | 13.34886 | 4.020471 | 301.6727 | 304.1389 | 34.87823 |
| Cog1 | 6.97762 | 12.9717 | 51.6172 | 30.6659 | 43.3813 | 23.28582 | 26.07617 | 58.76038 | 46.87251 | 2.139964 |
| C630004H02Rik | 25 | 13 | 176 | 73 | 220 | 83.43039 | 26.13306 | 200.3562 | 111.5797 | 2.847081 |
| Llgl2 | 36 | 22 | 233 | 91 | 226 | 120.1398 | 44.22518 | 265.2443 | 139.0926 | 2.459995 |
| Unc13d | 14.4058 | 76.4643 | 15.7554 | 19.283 | 20.6029 | 48.07526 | 153.7112 | 17.93575 | 29.47387 | 0.234949 |
| Foxj1 | 8.625 | 9.47716 | 170.305 | 98.7624 | 180.737 | 28.78348 | 19.05132 | 193.8731 | 150.9573 | 7.208776 |
| Sphk1 | 6 | 10 | 265.993 | 155.987 | 316 | 20.02329 | 20.10235 | 302.8031 | 238.4245 | 13.48832 |
| Rhbdf2 | 47.979 | 17 | 263.25 | 106 | 220 | 160.1163 | 34.174 | 299.6805 | 162.0199 | 2.376344 |
| 1810032O08Rik | 42 | 70 | 47 | 52 | 64 | 140.1631 | 140.7165 | 53.50422 | 79.48146 | 0.473462 |
| Afmid | 25.1953 | 23.356 | 32.1075 | 14.2109 | 22.1434 | 84.08215 | 46.95106 | 36.55078 | 21.72121 | 0.444712 |
| Tk1 | 103.805 | 730 | 237 | 171.924 | 247 | 346.4197 | 1467.472 | 269.7979 | 262.7841 | 0.293613 |
| Birc5 | 55.0614 | 599.881 | 162.308 | 147.43 | 209.743 | 183.7518 | 1205.902 | 184.7694 | 225.3452 | 0.29512 |
| Socs3 | 75 | 36 | 350 | 244.946 | 433 | 250.2912 | 72.36847 | 398.4357 | 374.3974 | 2.395196 |
| Rnf213 | 68.7479 | 33.5988 | 511.494 | 230.132 | 511.224 | 229.4266 | 67.54149 | 582.2784 | 351.7544 | 3.14523 |
| 1810049H13Rik | 18 | 17.3421 | 92.4817 | 79.6172 | 85 | 60.06988 | 34.8617 | 105.28 | 121.6941 | 2.390923 |
| Ccdc137 | 62.61 | 84.0389 | 99.5183 | 49.3828 | 91.4185 | 208.9431 | 168.938 | 113.2904 | 75.4811 | 0.499553 |
| Anapc11 | 209 | 420.877 | 271.146 | 270.646 | 299 | 697.4781 | 846.0618 | 308.6693 | 413.6796 | 0.467982 |
| Pycr1 | 101.99 | 13 | 70.2091 | 55.9483 | 81.4028 | 340.3626 | 26.13306 | 79.92517 | 85.5164 | 0.451415 |
| Dcxr | 7 | 6 | 224.462 | 171.523 | 246.683 | 23.36051 | 12.06141 | 255.5248 | 262.1711 | 14.61513 |
| Tbcd | 39.743 | 29.9997 | 220.681 | 97.9745 | 235.384 | 132.631 | 60.30646 | 251.2205 | 149.753 | 2.078257 |
| Atad2b | 12 | 12.0018 | 82 | 30 | 93 | 40.04659 | 24.12644 | 93.34779 | 45.85469 | 2.169174 |
| Mycn | 20 | 8 | 84 | 53 | 94 | 66.74431 | 16.08188 | 95.62456 | 81.00995 | 2.132592 |
| Fam84a | 97 | 80 | 23 | 9 | 23 | 323.7099 | 160.8188 | 26.18292 | 13.75641 | 0.082429 |
| Trib2 | 23.039 | 5.06016 | 196.551 | 77.2325 | 205.159 | 76.88611 | 10.17211 | 223.7512 | 118.0491 | 3.926112 |
| Pqlc3 | 7 | 14 | 117 | 67 | 142 | 23.36051 | 28.14329 | 133.1914 | 102.4088 | 4.574423 |
| Ddef2 | 56.6094 | 54.3291 | 23.4079 | 23.5754 | 17.8626 | 188.9178 | 109.2143 | 26.64726 | 36.03476 | 0.210249 |
| Adam17 | 69.9928 | 44.8968 | 436.82 | 254.691 | 488.886 | 233.5811 | 90.25313 | 497.2705 | 389.2926 | 2.737707 |
| uc007neg.1 | 11.7334 | 2.97607 | 61.068 | 40.4029 | 142.677 | 39.15689 | 5.982601 | 69.51906 | 61.75542 | 2.908196 |
| Klf11 | 10 | 7 | 55 | 37 | 49 | 33.37216 | 14.07165 | 62.61132 | 56.55412 | 2.511718 |
| Mboat2 | 16 | 25 | 7 | 2 | 14.004 | 53.39545 | 50.25588 | 7.968713 | 3.056979 | 0.106373 |
| Kidins220 | 37 | 23 | 199 | 117 | 239 | 123.477 | 46.23541 | 226.5391 | 178.8333 | 2.388585 |
| Rsad2 | 2 | 1 | 52.0298 | 36.4761 | 43.0353 | 6.674431 | 2.010235 | 59.23008 | 55.75334 | 13.23982 |
| Cmpk2 | 0 | 1 | 58 | 32 | 65 | 0 | 2.010235 | 66.02648 | 48.91167 | 57.17647 |
| Fam110c | 108 | 66 | 66 | 27 | 55 | 360.4193 | 132.6755 | 75.13358 | 41.26922 | 0.236066 |
| Bcap29 | 33 | 35 | 169 | 115 | 203 | 110.1281 | 70.35824 | 192.3875 | 175.7763 | 2.039843 |
| Atxn7l1 | 10 | 20.1592 | 83 | 67.0104 | 100 | 33.37216 | 40.52474 | 94.48617 | 102.4247 | 2.664671 |
| Twist1 | 135 | 135 | 6 | 9 | 5 | 450.5241 | 271.3818 | 6.830326 | 13.75641 | 0.028517 |
| Ahr | 63.9719 | 42 | 382 | 210.533 | 445 | 213.488 | 84.42988 | 434.8641 | 321.7975 | 2.539833 |
| Tspan13 | 43 | 33.0316 | 43 | 22 | 31.0154 | 143.5003 | 66.40129 | 48.95067 | 33.62677 | 0.39341 |
| Dnajb9 | 61 | 27 | 340 | 258.977 | 344 | 203.5702 | 54.27635 | 387.0518 | 395.8437 | 3.036285 |
| Stxbp6 | 27 | 35 | 261 | 176.907 | 285.458 | 90.10482 | 70.35824 | 297.1192 | 270.4005 | 3.536762 |
| Foxg1 | 252.013 | 430.275 | 32.8125 | 39.4357 | 43.6495 | 841.0217 | 864.954 | 37.35334 | 60.27706 | 0.057228 |
| uc007nmh.1 | 72.1437 | 220.492 | 2.1875 | 0 | 0 | 240.7591 | 443.2408 | 2.490223 | 0 | 0.003641 |
| uc007nmi.1 | 30.8431 | 0 | 0 | 0 | 0 | 102.9301 | 0 | 0 | 0 | 0 |
| Ap4s1 | 5 | 35 | 77 | 61 | 93 | 16.68608 | 70.35824 | 87.65585 | 93.23787 | 2.07818 |
| Heatr5a | 87.1442 | 117.205 | 82.1237 | 33.1683 | 105.191 | 290.819 | 235.6096 | 93.4886 | 50.6974 | 0.273895 |
| Egln3 | 13 | 5 | 903 | 560 | 1077.53 | 43.3838 | 10.05118 | 1027.964 | 855.9542 | 35.25627 |
| 1110002B05Rik | 82 | 169 | 594 | 516 | 540 | 273.6517 | 339.7298 | 676.2023 | 788.7007 | 2.388241 |
| Srp54a | 80.6629 | 91.774 | 388.475 | 309.119 | 394.177 | 269.1895 | 184.4873 | 442.2351 | 472.4852 | 2.016238 |
| Nfkbia | 8 | 22 | 191 | 197 | 225 | 26.69773 | 44.22518 | 217.432 | 301.1125 | 7.311383 |
| Pax9 | 3 | 0 | 91 | 67 | 88 | 10.01165 | 0 | 103.5933 | 102.4088 | 20.57624 |
| C79407 | 14 | 39 | 34 | 11 | 39 | 46.72102 | 78.39918 | 38.70518 | 16.81339 | 0.443722 |
| Ppil5 | 8 | 53 | 21 | 16 | 22 | 26.69773 | 106.5425 | 23.90614 | 24.45583 | 0.362968 |
| Dhrs7 | 22 | 20 | 131 | 118 | 156 | 73.41874 | 40.20471 | 149.1288 | 180.3618 | 2.899846 |
| Slc38a6 | 18.0685 | 26.5598 | 25.5761 | 8.26537 | 25.5428 | 60.29848 | 53.39145 | 29.11552 | 12.63353 | 0.367219 |
| Hif1a | 75 | 80.2535 | 628.036 | 347.997 | 650.994 | 250.2912 | 161.3284 | 714.9484 | 531.9098 | 3.029152 |
| uc007nxj.1 | 2.42631 | 75.8537 | 4.87921 | 11.3384 | 2.25518 | 8.09712 | 152.4838 | 5.554432 | 17.33063 | 0.142514 |
| Sgpp1 | 10 | 24 | 107.034 | 62.7779 | 139.683 | 33.37216 | 48.24565 | 121.8462 | 95.95537 | 2.668554 |
| Churc1 | 17.595 | 128.631 | 133.51 | 344.334 | 204.485 | 58.71831 | 258.5786 | 151.9861 | 526.311 | 2.137736 |
| Rab15 | 3 | 0 | 53 | 30 | 68 | 10.01165 | 0 | 60.33454 | 45.85469 | 10.60657 |
| Fut8 | 31 | 21 | 209 | 135 | 216 | 103.4537 | 42.21494 | 237.923 | 206.3461 | 3.049861 |
| Gphn | 9 | 16 | 89 | 55 | 87 | 30.03494 | 32.16377 | 101.3165 | 84.06693 | 2.980503 |
| Atp6v1d | 78 | 52 | 388 | 247 | 432 | 260.3028 | 104.5322 | 441.6944 | 377.5369 | 2.245484 |
| Pigh | 13.295 | 23.8467 | 101.38 | 60.3452 | 162.994 | 44.36828 | 47.93748 | 115.4097 | 92.23701 | 2.249554 |
| Rad51l1 | 31 | 30 | 27 | 19 | 32 | 103.4537 | 60.30706 | 30.73647 | 29.0413 | 0.365031 |
| Wdr22 | 31 | 21 | 135 | 94 | 181 | 103.4537 | 42.21494 | 153.6823 | 143.678 | 2.041348 |
| 4933426M11Rik | 32.9125 | 27 | 1229.86 | 736.999 | 1440 | 109.8361 | 54.27635 | 1400.057 | 1126.495 | 15.39525 |
| Acot1 | 13.4643 | 5 | 157.415 | 100.872 | 182.522 | 44.93327 | 10.05118 | 179.1993 | 154.1818 | 6.063189 |
| Entpd5 | 9 | 10 | 120 | 69 | 141.103 | 30.03494 | 20.10235 | 136.6065 | 105.4658 | 4.828188 |
| Ltbp2 | 3.01357 | 3.02415 | 598.736 | 207.411 | 840.75 | 10.05693 | 6.079253 | 681.5937 | 317.0256 | 61.88694 |
| Pgf | 35 | 31 | 5 | 1 | 2 | 116.8025 | 62.31729 | 5.691938 | 1.52849 | 0.040311 |
| Tmed10 | 156 | 261.15 | 1181.14 | 691.721 | 1662.21 | 520.6056 | 524.973 | 1344.595 | 1057.288 | 2.297181 |
| Fos | 149 | 51 | 3287 | 2424 | 3429 | 497.2451 | 102.522 | 3741.88 | 3705.059 | 12.41638 |
| Ttll5 | 17 | 12.0147 | 93 | 55.3045 | 131.514 | 56.73267 | 24.15237 | 105.8701 | 84.53236 | 2.353988 |
| Tgfb3 | 27 | 6 | 926 | 400.856 | 951 | 90.10482 | 12.06141 | 1054.147 | 612.7043 | 16.31509 |
| Pomt2 | 9 | 8.8539 | 82.3796 | 39.0444 | 103 | 30.03494 | 17.79842 | 93.77992 | 59.67896 | 3.208198 |
| uc007ojl.1 | 3.75854 | 213.484 | 31.2128 | 96.255 | 30.1905 | 12.54306 | 429.1531 | 35.53227 | 147.1248 | 0.413536 |
| Adck1 | 8 | 14 | 61 | 58 | 83 | 26.69773 | 28.14329 | 69.44165 | 88.6524 | 2.88277 |
| 9030617O03Rik | 7 | 7 | 106 | 57 | 127.17 | 23.36051 | 14.07165 | 120.6691 | 87.12391 | 5.55119 |
| Btbd7 | 11.589 | 136.955 | 40.9494 | 62.3365 | 53.9041 | 38.67499 | 275.3118 | 46.61629 | 95.2807 | 0.45192 |
| Ifi27l1 | 32 | 13.9272 | 1037.5 | 710.744 | 1279.83 | 106.7909 | 27.99695 | 1181.077 | 1086.365 | 16.8223 |
| Ifi27l2b | 9 | 51.9794 | 11 | 17 | 26 | 30.03494 | 104.4908 | 12.52226 | 25.98432 | 0.28624 |
| Serpina3h | 0 | 1 | 811.794 | 374.65 | 863.838 | 0 | 2.010235 | 924.1363 | 572.6487 | 744.5819 |
| Clmn | 16.2609 | 7.60525 | 129.589 | 36.432 | 127.339 | 54.26613 | 15.28834 | 147.5225 | 55.68594 | 2.921573 |
| Eml1 | 46.1879 | 44.3882 | 49.6928 | 26.294 | 57.4066 | 154.139 | 89.23073 | 56.56967 | 40.19011 | 0.397583 |
| uc007par.1 | 32.5772 | 13.2264 | 1.42082 | 1.53643 | 6.29627 | 108.7171 | 26.58818 | 1.617444 | 2.348417 | 0.02931 |
| uc007pat.1 | 42.0315 | 2.13145 | 0.421698 | 2.4743 | 6.29627 | 140.2682 | 4.284716 | 0.480056 | 3.781942 | 0.029484 |
| uc007pav.1 | 42.1938 | 0.490696 | 20.1575 | 0 | 6.29627 | 140.8098 | 0.986414 | 22.94705 | 0 | 0.161831 |
| uc007pbc.1 | 132.164 | 62 | 27.0303 | 18 | 37.0903 | 441.0598 | 124.6346 | 30.77096 | 27.51281 | 0.103031 |
| Tnfaip2 | 230 | 123 | 2099 | 1005 | 2110 | 767.5596 | 247.2589 | 2389.476 | 1536.132 | 3.868285 |
| BC022687 | 8 | 16 | 57 | 37 | 71 | 26.69773 | 32.16377 | 64.8881 | 56.55412 | 2.063186 |
| Nudt14 | 34 | 93 | 270 | 257 | 260 | 113.4653 | 186.9519 | 307.3647 | 392.8218 | 2.330714 |
| Tmem121 | 16 | 31 | 24 | 14 | 38 | 53.39545 | 62.31729 | 27.3213 | 21.39886 | 0.421044 |
| Akr1c13 | 253.271 | 305.473 | 43.9535 | 38.8235 | 46 | 845.2199 | 614.0726 | 50.03612 | 59.34132 | 0.074952 |
| Akr1c12 | 148.729 | 146.527 | 1.04651 | 1.17647 | 2 | 496.3407 | 294.5537 | 1.191334 | 1.798222 | 0.00378 |
| Pitrm1 | 207.561 | 274.404 | 226.746 | 133.091 | 225.313 | 692.6758 | 551.6166 | 258.1248 | 203.4282 | 0.370936 |
| Adarb2 | 121.497 | 144.306 | 6.10992 | 0 | 1.59335 | 405.4617 | 290.089 | 6.955457 | 0 | 0.01 |
| Lgals8 | 36.3871 | 39.4333 | 239.34 | 185.701 | 275.422 | 121.4316 | 79.27021 | 272.4617 | 283.8421 | 2.771792 |
| Prl2c3 | 30.507 | 212.476 | 3 | 1 | 4.5 | 101.8084 | 427.1268 | 3.415163 | 1.52849 | 0.009346 |
| Prl2c2 | 13.493 | 723.524 | 3 | 1 | 4.5 | 45.02905 | 1454.453 | 3.415163 | 1.52849 | 0.003297 |
| Nid1 | 129 | 183 | 64.0247 | 48.0961 | 82 | 430.5008 | 367.8731 | 72.88493 | 73.51439 | 0.183372 |
| AW209491 | 37 | 21 | 168 | 98.9703 | 151.195 | 123.477 | 42.21494 | 191.2491 | 151.2751 | 2.067235 |
| Zkscan3 | 30.184 | 38.0275 | 171.975 | 105.608 | 173.2 | 100.7305 | 76.44422 | 195.7742 | 161.4207 | 2.01606 |
| Zfp192 | 22 | 10.0712 | 161.97 | 63 | 140.365 | 73.41874 | 20.24548 | 184.3846 | 96.29485 | 2.996656 |
| Zfp322a | 7 | 10 | 60 | 29 | 65 | 23.36051 | 20.10235 | 68.30326 | 44.3262 | 2.591395 |
| Hist1h2bc | 47.7692 | 126.504 | 403.453 | 412.813 | 492.695 | 159.4161 | 254.3028 | 459.2859 | 630.9804 | 2.635283 |
| Hist1h1c | 13 | 20 | 306.984 | 188 | 337 | 43.3838 | 40.20471 | 349.4668 | 287.3561 | 7.618545 |
| Mboat1 | 153.993 | 203 | 1226.81 | 838.719 | 1350.94 | 513.9078 | 408.0778 | 1396.585 | 1281.973 | 2.905207 |
| Dusp22 | 23 | 27.0299 | 21.1045 | 11 | 26 | 76.75596 | 54.33646 | 24.0251 | 16.81339 | 0.311524 |
| Foxf2 | 54 | 67.151 | 356.994 | 239.469 | 365.77 | 180.2096 | 134.9893 | 406.3976 | 366.0259 | 2.45059 |
| Foxc1 | 6.01226 | 2 | 59 | 34.1193 | 61.1045 | 20.06421 | 4.020471 | 67.16487 | 52.151 | 4.954015 |
| Gmds | 17 | 16 | 202 | 123 | 195 | 56.73267 | 32.16377 | 229.9543 | 188.0042 | 4.701635 |
| Serpinb1a | 8.88889 | 5 | 64.9143 | 54.5638 | 68.9855 | 29.66414 | 10.05118 | 73.89764 | 83.4002 | 3.960634 |
| Serpinb9 | 40 | 48 | 391.164 | 200.112 | 432.098 | 133.4886 | 96.4913 | 445.2963 | 305.8691 | 3.266222 |
| Serpinb9b | 23 | 28 | 17.2927 | 20.1348 | 23 | 76.75596 | 56.28659 | 19.6858 | 30.77583 | 0.379289 |
| uc007qaj.1 | 0.666667 | 0.729307 | 62.2962 | 29.1244 | 54.0345 | 2.224812 | 1.466079 | 70.91722 | 44.51634 | 31.27526 |
| Nqo2 | 33 | 100 | 60 | 53 | 64 | 110.1281 | 201.0235 | 68.30326 | 81.00995 | 0.479873 |
| Bphl | 48 | 35 | 261 | 198 | 278 | 160.1864 | 70.35824 | 297.1192 | 302.641 | 2.601493 |
| Tubb2a | 20.1198 | 47.5945 | 324.551 | 212.201 | 346.01 | 67.14411 | 95.67614 | 369.4648 | 324.347 | 4.261214 |
| 1300014I06Rik | 84 | 93 | 77 | 51 | 69.1829 | 280.3261 | 186.9519 | 87.65585 | 77.95297 | 0.354412 |
| uc007qca.1 | 6.67144 | 6.06459 | 162.968 | 63.8419 | 210.786 | 22.26403 | 12.19125 | 185.5208 | 97.58168 | 8.216517 |
| Nrn1 | 188.117 | 403.901 | 2.01103 | 1 | 2 | 627.787 | 811.9361 | 2.289332 | 1.52849 | 0.002652 |
| Txndc5 | 179 | 173 | 1158 | 678 | 1229 | 597.3616 | 347.7707 | 1318.253 | 1036.316 | 2.491258 |
| Muted | 15 | 15 | 77 | 50 | 77 | 50.05823 | 30.15353 | 87.65585 | 76.42448 | 2.045589 |
| Slc35b3 | 28 | 22 | 127.3 | 86.1583 | 155 | 93.44204 | 44.22518 | 144.9167 | 131.6921 | 2.009257 |
| Gcnt2 | 27.2967 | 16.2319 | 448.576 | 251.932 | 450.838 | 91.09497 | 32.62994 | 510.6534 | 385.0755 | 7.23968 |
| Tmem14c | 118 | 366 | 998 | 1090 | 1353 | 393.7914 | 735.7461 | 1136.111 | 1666.054 | 2.480807 |
| Hivep1 | 43 | 18 | 202.472 | 111 | 230 | 143.5003 | 36.18424 | 230.4916 | 169.6624 | 2.226981 |
| Edn1 | 15 | 1 | 158.69 | 143.654 | 137.765 | 50.05823 | 2.010235 | 180.6507 | 219.5737 | 7.686502 |
| Dtnbp1 | 42 | 48 | 239 | 145 | 226 | 140.1631 | 96.4913 | 272.0746 | 221.631 | 2.086189 |
| Tpmt | 16 | 52 | 21.0324 | 18 | 24 | 53.39545 | 104.5322 | 23.94302 | 27.51281 | 0.325819 |
| Phf2 | 28 | 15 | 132 | 70 | 151.987 | 93.44204 | 30.15353 | 150.2672 | 106.9943 | 2.081478 |
| Ninj1 | 87 | 104 | 531 | 335 | 562 | 290.3378 | 209.0645 | 604.4838 | 512.044 | 2.235729 |
| Omd | 1.73128 | 1350.09 | 8.27492 | 13.8834 | 12.7941 | 5.777655 | 2713.999 | 9.420067 | 21.22063 | 0.011266 |
| Cks2 | 224 | 1299 | 526 | 594.876 | 535 | 747.5363 | 2611.296 | 598.7919 | 909.2618 | 0.448982 |
| Sema4d | 11 | 17.0608 | 98 | 54 | 86.2892 | 36.70937 | 34.29622 | 111.562 | 82.53844 | 2.733593 |
| Auh | 38.3073 | 32.2481 | 188.301 | 144.847 | 240.914 | 127.8397 | 64.82627 | 214.3595 | 221.3971 | 2.261721 |
| Nfil3 | 45 | 25 | 225 | 171 | 292 | 150.1747 | 50.25588 | 256.1372 | 261.3717 | 2.581986 |
| Pcdh24 | 23 | 41 | 0 | 0 | 0 | 76.75596 | 82.41965 | 0 | 0 | 0 |
| Dbn1 | 79 | 74 | 59 | 38 | 70 | 263.64 | 148.7574 | 67.16487 | 58.08261 | 0.303706 |
| 5133401N09Rik | 13.3867 | 29.0907 | 115.681 | 89.3808 | 139.657 | 44.6743 | 58.47915 | 131.6898 | 136.6176 | 2.601051 |
| Gkap1 | 19 | 17.7526 | 109.823 | 90.3203 | 130.609 | 63.4071 | 35.6869 | 125.0211 | 138.0536 | 2.6548 |
| Isca1 | 53 | 88 | 310 | 241 | 352 | 176.8724 | 176.9007 | 352.9002 | 368.366 | 2.038782 |
| Gas1 | 27 | 19.9048 | 30 | 19 | 30 | 90.10482 | 40.01333 | 34.15163 | 29.0413 | 0.485658 |
| 4930486L24Rik | 30 | 55 | 10 | 6 | 7 | 100.1165 | 110.5629 | 11.38388 | 9.170938 | 0.097564 |
| uc007qwr.1 | 9 | 29 | 341 | 377.979 | 419 | 30.03494 | 58.29682 | 388.1902 | 577.737 | 10.93522 |
| Gm7036 | 25.5977 | 18.4717 | 28.5498 | 17.1151 | 38.7577 | 85.42504 | 37.13246 | 32.50074 | 26.16025 | 0.478641 |
| 2010111I01Rik | 12.4615 | 26.0312 | 286.303 | 237.941 | 315.831 | 41.58671 | 52.32884 | 325.9238 | 363.6904 | 7.342918 |
| uc007qxo.1 | 14.5385 | 22.9688 | 382.697 | 182.994 | 391.36 | 48.51811 | 46.17269 | 435.6575 | 279.7044 | 7.554714 |
| 0610007P08Rik | 24 | 32.9689 | 134 | 94 | 133.522 | 80.09318 | 66.27525 | 152.5439 | 143.678 | 2.023811 |
| Habp4 | 11 | 6 | 53 | 44.0238 | 83.051 | 36.70937 | 12.06141 | 60.33454 | 67.28992 | 2.616822 |
| Hiatl1 | 32 | 30 | 203 | 106 | 197 | 106.7909 | 60.30706 | 231.0927 | 162.0199 | 2.352588 |
| Srd5a1 | 24 | 52 | 34 | 25 | 61 | 80.09318 | 104.5322 | 38.70518 | 38.21224 | 0.416613 |
| Trip13 | 29 | 91.6647 | 51 | 23 | 43.0549 | 96.77925 | 184.2676 | 58.05777 | 35.15526 | 0.331664 |
| Erap1 | 10 | 13 | 106.082 | 76 | 133 | 33.37216 | 26.13306 | 120.7624 | 116.1652 | 3.981628 |
| Glrx | 17 | 35 | 512 | 312 | 531 | 56.73267 | 70.35824 | 582.8545 | 476.8888 | 8.338467 |
| Rhobtb3 | 111 | 71 | 118.116 | 75.1678 | 169.518 | 370.4309 | 142.7267 | 134.4618 | 114.8932 | 0.485923 |
| Nr2f1 | 61 | 36 | 0 | 0 | 2 | 203.5702 | 72.36847 | 0 | 0 | 0 |
| Tmem161b | 18 | 20 | 95 | 66 | 76 | 60.06988 | 40.20471 | 108.1468 | 100.8803 | 2.084548 |
| Ssbp2 | 23.8261 | 7 | 149.051 | 107 | 134.344 | 79.51283 | 14.07165 | 169.6778 | 163.5484 | 3.560699 |
| Dhfr | 59.1058 | 192.774 | 104.132 | 86.2251 | 121.806 | 197.2488 | 387.5211 | 118.5426 | 131.7942 | 0.428094 |
| Serinc5 | 29 | 8.72727 | 154 | 72 | 190.02 | 96.77925 | 17.54387 | 175.3117 | 110.0513 | 2.496109 |
| Arsb | 14.1687 | 22 | 115.12 | 72 | 125.274 | 47.28401 | 44.22518 | 131.0512 | 110.0513 | 2.634735 |
| Wdr41 | 4 | 15 | 73 | 40 | 79 | 13.34886 | 30.15353 | 83.1023 | 61.13959 | 3.315723 |
| F2r | 409.818 | 623.646 | 224 | 105 | 214.091 | 1367.651 | 1253.675 | 254.9988 | 160.4914 | 0.158504 |
| Iqgap2 | 19 | 37 | 312 | 186 | 350.831 | 63.4071 | 74.37871 | 355.1769 | 284.2991 | 4.641088 |
| Rgnef | 92 | 41 | 62 | 38 | 95 | 307.0238 | 82.41965 | 70.58003 | 58.08261 | 0.330376 |
| Tmem171 | 66 | 122 | 94 | 66 | 90 | 220.2562 | 245.2487 | 107.0084 | 100.8803 | 0.446588 |
| Ocln | 7.00778 | 0 | 190.199 | 103 | 157.925 | 23.38647 | 0 | 216.5202 | 157.4344 | 15.99021 |
| Marveld2 | 6 | 6 | 95 | 47 | 116 | 20.02329 | 12.06141 | 108.1468 | 71.83901 | 5.609708 |
| Ccdc125 | 26 | 19.0309 | 119.621 | 87.055 | 110.027 | 86.76761 | 38.25659 | 136.1751 | 133.0627 | 2.153485 |
| Cenph | 22 | 50 | 31 | 27 | 48 | 73.41874 | 100.5118 | 35.29002 | 41.26922 | 0.440171 |
| Ccnb1 | 107 | 571 | 261 | 202 | 282 | 357.0821 | 1147.844 | 297.1192 | 308.7549 | 0.402594 |
| Pik3r1 | 17 | 32.2059 | 234.042 | 128.239 | 301.776 | 56.73267 | 64.74144 | 266.4305 | 196.012 | 3.806923 |
| Mast4 | 91.9968 | 68.5706 | 100.733 | 48.3283 | 105.996 | 307.0132 | 137.843 | 114.6732 | 73.86931 | 0.423828 |
| Kif2a | 72.0769 | 68.113 | 79.7579 | 35.0912 | 93.4573 | 240.5362 | 136.9232 | 90.79541 | 53.63654 | 0.382642 |
| Il6st | 16 | 14 | 199 | 65.1801 | 226.303 | 53.39545 | 28.14329 | 226.5391 | 99.62711 | 4.000138 |
| Gpx8 | 7 | 18 | 109 | 114 | 122 | 23.36051 | 36.18424 | 124.0843 | 174.2478 | 5.010217 |
| Mocs2 | 62 | 76 | 352 | 330 | 446 | 206.9074 | 152.7779 | 400.7124 | 504.4016 | 2.516406 |
| Parp8 | 35.1577 | 24 | 280 | 149.262 | 263.513 | 117.3288 | 48.24565 | 318.7485 | 228.1454 | 3.303009 |
| Emb | 0 | 2 | 157 | 106 | 173 | 0 | 4.020471 | 178.7269 | 162.0199 | 84.75295 |
| 544988 | 39.8812 | 43.4819 | 61.85 | 23.4677 | 54.8993 | 133.0922 | 87.40885 | 70.40928 | 35.87014 | 0.481991 |
| Pdhb | 87 | 109 | 544 | 435 | 649 | 290.3378 | 219.1156 | 619.2829 | 664.893 | 2.520693 |
| Kctd6 | 13 | 18 | 99 | 91 | 129 | 43.3838 | 36.18424 | 112.7004 | 139.0926 | 3.164498 |
| Acox2 | 9 | 0 | 95 | 60 | 102 | 30.03494 | 0 | 108.1468 | 91.70938 | 6.654124 |
| Fam107a | 1.00254 | 1 | 166.562 | 106 | 217 | 3.345692 | 2.010235 | 189.6121 | 162.0199 | 65.65287 |
| 3830406C13Rik | 22 | 21 | 117.517 | 73 | 121 | 73.41874 | 42.21494 | 133.7799 | 111.5797 | 2.12187 |
| Ngly1 | 25.2164 | 15 | 148.322 | 93.7122 | 172.186 | 84.15256 | 30.15353 | 168.8479 | 143.2381 | 2.730266 |
| 1810063B07Rik | 272.449 | 4068.31 | 152.93 | 482.745 | 198.331 | 909.2211 | 8178.26 | 174.0936 | 737.8707 | 0.100354 |
| Fam149b | 17.021 | 20 | 96.069 | 58.9252 | 89.0087 | 56.80275 | 40.20471 | 109.3638 | 90.06656 | 2.055825 |
| Chchd1 | 192.957 | 633.925 | 312.886 | 376.501 | 350.752 | 643.9391 | 1274.338 | 356.1856 | 575.4779 | 0.485677 |
| Zfp503 | 2 | 0 | 73.0817 | 47.0978 | 66 | 6.674431 | 0 | 83.1953 | 71.9885 | 23.25049 |
| Fam116a | 25.0469 | 26.6645 | 141.891 | 90.9264 | 145.905 | 83.58691 | 53.60192 | 161.527 | 138.9801 | 2.190463 |
| Arhgef3 | 13 | 17 | 204.433 | 108 | 232.416 | 43.3838 | 34.174 | 232.724 | 165.0769 | 5.129089 |
| Tkt | 245.633 | 294 | 1719 | 1114 | 2922 | 819.7303 | 591.0092 | 1956.888 | 1702.737 | 2.594119 |
| Nt5dc2 | 63.2034 | 129.237 | 96.5602 | 62.1507 | 118.434 | 210.9234 | 259.7968 | 109.9229 | 94.9967 | 0.435332 |
| Sh3bp5 | 35 | 43.0129 | 27 | 21 | 21 | 116.8025 | 86.46605 | 30.73647 | 32.09828 | 0.309122 |
| Btd | 16 | 16 | 91 | 67 | 107 | 53.39545 | 32.16377 | 103.5933 | 102.4088 | 2.407714 |
| Ankrd28 | 117 | 108 | 109.983 | 60 | 118 | 390.4542 | 217.1054 | 125.2033 | 91.70938 | 0.357023 |
| Arhgap22 | 35 | 42 | 17 | 14 | 32 | 116.8025 | 84.42988 | 19.35259 | 21.39886 | 0.202509 |
| Anxa8 | 147 | 293 | 2600 | 1477 | 2520 | 490.5707 | 588.9989 | 2959.808 | 2257.579 | 4.83284 |
| Bmpr1a | 74.2914 | 77 | 423.028 | 247.972 | 451.603 | 247.9264 | 154.7881 | 481.5698 | 379.0226 | 2.136979 |
| Pcdh21 | 0 | 1 | 197 | 169 | 223 | 0 | 2.010235 | 224.2624 | 258.3148 | 240.06 |
| Tspan14 | 45 | 65 | 457 | 267 | 516.987 | 150.1747 | 130.6653 | 520.2432 | 408.1067 | 3.305618 |
| 5730469M10Rik | 147.77 | 138.96 | 2181.85 | 1971.22 | 2648.19 | 493.1404 | 279.3423 | 2483.791 | 3012.989 | 7.115733 |
| Txndc16 | 5.28198 | 1.05157 | 64.3806 | 35.7615 | 47.6099 | 17.62711 | 2.113903 | 73.29008 | 54.66108 | 6.48149 |
| Fermt2 | 68 | 55.0127 | 323 | 224 | 447.709 | 226.9307 | 110.5885 | 367.6992 | 342.3817 | 2.103824 |
| uc007thb.1 | 1039.99 | 302.633 | 4167.51 | 2988.41 | 4696.33 | 3470.671 | 608.3635 | 4744.242 | 4567.754 | 2.282892 |
| Bmp4 | 17.9998 | 6 | 207.396 | 249.975 | 316.984 | 60.06921 | 12.06141 | 236.097 | 382.0842 | 8.570302 |
| Gch1 | 75 | 39 | 324 | 275 | 403 | 250.2912 | 78.39918 | 368.8376 | 420.3347 | 2.40096 |
| Wdhd1 | 11.901 | 57.8814 | 34.0343 | 25.6717 | 30.42 | 39.7162 | 116.3552 | 38.74423 | 39.23893 | 0.499663 |
| Lgals3 | 759 | 3119 | 1637 | 1388 | 2020 | 2532.947 | 6269.924 | 1863.541 | 2121.544 | 0.452703 |
| Dlgap5 | 9.07556 | 38.92 | 16.2345 | 18.6008 | 17.2963 | 30.2871 | 78.23836 | 18.48115 | 28.43113 | 0.43227 |
| Fbxo34 | 18 | 19 | 88.0068 | 79 | 95 | 60.06988 | 38.19447 | 100.1859 | 120.7507 | 2.248389 |
| 6720456H20Rik | 19 | 8.00968 | 107.19 | 50 | 97.1177 | 63.4071 | 16.10134 | 122.0238 | 76.42448 | 2.49594 |
| Rnase4 | 15 | 10 | 111.726 | 111.714 | 108 | 50.05823 | 20.10235 | 127.1875 | 170.7537 | 4.246561 |
| Ndrg2 | 45.3482 | 27.2269 | 26.1396 | 21.1209 | 33.1131 | 151.3367 | 54.73248 | 29.757 | 32.28308 | 0.301064 |
| Zfp219 | 9.96547 | 6.85288 | 42.5051 | 38.1055 | 49.811 | 33.25692 | 13.7759 | 48.38728 | 58.24386 | 2.267164 |
| Rab2b | 10.0158 | 7 | 77.1164 | 47.126 | 96.2403 | 33.42488 | 14.07165 | 87.78836 | 72.0316 | 3.364876 |
| Abhd4 | 52 | 20 | 400 | 195 | 414 | 173.5352 | 40.20471 | 455.3551 | 298.0555 | 3.524894 |
| Lrp10 | 88 | 78 | 612.065 | 257 | 703 | 293.675 | 156.7984 | 696.7672 | 392.8218 | 2.418765 |
| Homez | 6.12529 | 4.00859 | 62.8253 | 34.2947 | 61.4866 | 20.44141 | 8.058209 | 71.51954 | 52.41909 | 4.348782 |
| Slc22a17 | 10.3365 | 0 | 146.433 | 90.5198 | 157.594 | 34.49513 | 0 | 166.6975 | 138.3586 | 8.843454 |
| Ap1g2 | 18.5714 | 15.6 | 77.2846 | 72.8875 | 96.5625 | 61.97677 | 31.35967 | 87.97983 | 111.4078 | 2.136225 |
| Psme2 | 204 | 320 | 1329 | 1110 | 1629 | 680.792 | 643.2753 | 1512.917 | 1696.624 | 2.424001 |
| Rnf31 | 12 | 9 | 84 | 36 | 101 | 40.04659 | 18.09212 | 95.62456 | 55.02563 | 2.59122 |
| Irf9 | 20.8842 | 27.0849 | 183.06 | 101.69 | 244.051 | 69.69508 | 54.44702 | 208.3932 | 155.4321 | 2.930717 |
| Dhrs1 | 51 | 50 | 272 | 178 | 373 | 170.198 | 100.5118 | 309.6414 | 272.0712 | 2.148842 |
| Ripk3 | 13 | 9 | 136 | 85 | 149 | 43.3838 | 18.09212 | 154.8207 | 129.9216 | 4.63177 |
| uc007uch.1 | 28.6971 | 24.469 | 24.4045 | 22.9763 | 68.5772 | 95.76841 | 49.18845 | 27.78178 | 35.11904 | 0.433928 |
| uc007uci.1 | 3.80897 | 3.04955 | 68.4293 | 25.5957 | 42.1836 | 12.71135 | 6.130313 | 77.89907 | 39.12276 | 6.2108 |
| Cryl1 | 10 | 9 | 56 | 55 | 73 | 33.37216 | 18.09212 | 63.74971 | 84.06693 | 2.872218 |
| Ift88 | 12.1463 | 6 | 68 | 29 | 76 | 40.53482 | 12.06141 | 77.41036 | 44.3262 | 2.314549 |
| F630043A04Rik | 6.09391 | 41.928 | 20.5994 | 12.5515 | 27.4498 | 20.33669 | 84.28515 | 23.4501 | 19.18484 | 0.407515 |
| D14Ertd668e | 2 | 2 | 100.455 | 85.3889 | 110.323 | 6.674431 | 4.020471 | 114.3567 | 130.5161 | 22.89622 |
| Cdadc1 | 13 | 29 | 114.149 | 53.2602 | 118 | 43.3838 | 58.29682 | 129.9458 | 81.40767 | 2.078601 |
| Gata4 | 30 | 23 | 2 | 0 | 2 | 100.1165 | 46.23541 | 2.276775 | 0 | 0.015557 |
| Msra | 6 | 22 | 85 | 37.0128 | 74.5674 | 20.02329 | 44.22518 | 96.762955 | 6.57368 | 2.386619 |
| Scara5 | 1 | 0 | 635 | 360.798 | 677 | 3.337216 | 0 | 722.8762 | 551.476 | 381.8609 |
| Scara3 | 138 | 92 | 905 | 316 | 1012 | 460.5358 | 184.9416 | 1030.241 | 483.0027 | 2.344379 |
| Stc1 | 19 | 15.0464 | 102.336 | 71.1123 | 100.441 | 63.4071 | 30.2468 | 116.498 | 108.6944 | 2.404518 |
| 9930012K11Rik | 20 | 28 | 14 | 6 | 22 | 66.74431 | 56.28659 | 15.93743 | 9.170938 | 0.204082 |
| Pdlim2 | 47 | 53.0101 | 31.5425 | 11 | 38.0111 | 156.8491 | 106.5628 | 35.90759 | 16.81339 | 0.200147 |
| Ppp3cc | 6 | 7 | 64 | 41.8605 | 53 | 20.02329 | 14.07165 | 72.85681 | 63.98334 | 4.013503 |
| Nudt18 | 21 | 11 | 103.842 | 45 | 121.134 | 70.08153 | 22.11259 | 118.2124 | 68.78204 | 2.028269 |
| P2ry5 | 32 | 28 | 284 | 153 | 303 | 106.7909 | 56.28659 | 323.3021 | 233.8589 | 3.416542 |
| Lrch1 | 8.77294 | 8.03259 | 162.077 | 84.3144 | 188.02 | 29.27719 | 16.1474 | 184.5065 | 128.8737 | 6.89891 |
| Cog3 | 29 | 26 | 155 | 91 | 181 | 96.77925 | 52.26612 | 176.4501 | 139.0926 | 2.117091 |
| Tsc22d1 | 1568 | 2118.98 | 1900 | 1379.62 | 2045.76 | 5232.754 | 4259.648 | 2162.937 | 2108.735 | 0.45001 |
| Serp2 | 7 | 44 | 12 | 5 | 8 | 23.36051 | 88.45035 | 13.66065 | 7.642448 | 0.190528 |
| 9030625A04Rik | 13 | 14.0265 | 77 | 39 | 76.0182 | 43.3838 | 28.19657 | 87.65585 | 59.6111 | 2.057365 |
| Ccdc122 | 8 | 11 | 53 | 41.317 | 59 | 26.69773 | 22.11259 | 60.33454 | 63.15261 | 2.52994 |
| Dnajc15 | 27 | 68 | 197 | 186 | 210 | 90.10482 | 136.696 | 224.2624 | 284.2991 | 2.242326 |
| Elf1 | 30.1347 | 48.6861 | 238 | 204.064 | 292.573 | 100.566 | 97.87052 | 270.9363 | 311.9097 | 2.937191 |
| Klhl1 | 87 | 28 | 1 | 1 | 0 | 290.3378 | 56.28659 | 1.138388 | 1.52849 | 0.007694 |
| 6720463M24Rik | 19 | 20 | 22 | 16 | 25 | 63.4071 | 40.20471 | 25.04453 | 24.45583 | 0.477748 |
| Dis3 | 83 | 99 | 111 | 67 | 118 | 276.9889 | 199.0133 | 126.361 | 102.4088 | 0.480607 |
| Kctd12 | 1 | 0 | 52.1562 | 44 | 63.0956 | 3.337216 | 0 | 59.37397 | 67.25355 | 37.94406 |
| Irg1 | 2 | 0 | 335 | 261.944 | 419 | 6.674431 | 0 | 381.3599 | 400.3787 | 117.1244 |
| Fbxl3 | 71.1383 | 65.0626 | 351.112 | 226.228 | 468.065 | 237.4038 | 130.7911 | 399.7016 | 345.7872 | 2.024712 |
| Slain1 | 2 | 2 | 92 | 58 | 90 | 6.674431 | 4.020471 | 104.7317 | 88.6524 | 18.08189 |
| Dnajc3 | 18 | 24 | 140 | 75 | 176 | 60.06988 | 48.24565 | 159.3743 | 114.6367 | 2.529748 |
| Dock9 | 43 | 30 | 348 | 117 | 421 | 143.5003 | 60.30706 | 396.1589 | 178.8333 | 2.821254 |
| Clybl | 10 | 3 | 81 | 57 | 105 | 33.37216 | 6.030706 | 92.2094 | 87.12391 | 4.551276 |
| A2ld1 | 19 | 20 | 121.984 | 64 | 84 | 63.4071 | 40.20471 | 138.8651 | 97.82334 | 2.284377 |
| Ghr | 137.419 | 57 | 20.0412 | 8 | 20 | 458.5968 | 114.5834 | 22.81465 | 12.22792 | 0.061137 |
| Osmr | 21 | 1.01601 | 310.842 | 183.275 | 318.442 | 70.08153 | 2.042419 | 353.8587 | 280.1339 | 8.790321 |
| Nup155 | 64 | 76 | 80 | 44 | 78 | 213.5818 | 152.7779 | 91.07101 | 67.25355 | 0.432156 |
| Skp2 | 41 | 48 | 59 | 28 | 65 | 136.8258 | 96.4913 | 67.16487 | 42.79771 | 0.471301 |
| Rad1 | 40.3318 | 62.7434 | 45.6578 | 51.2485 | 59.3175 | 134.5959 | 126.129 | 51.97628 | 78.3328 | 0.499795 |
| Zfr | 16 | 24.1856 | 21 | 14 | 29 | 53.39545 | 48.61875 | 23.90614 | 21.39886 | 0.444105 |
| uc007vie.1 | 12.0358 | 22.0957 | 114.257 | 50.8622 | 192.523 | 40.16606 | 44.41756 | 130.0688 | 77.74235 | 2.456872 |
| Fam134b | 22 | 21 | 198.202 | 116.446 | 240.838 | 73.41874 | 42.21494 | 225.6307 | 177.9865 | 3.490481 |
| Pop1 | 48 | 69 | 33 | 16 | 36.028 | 160.1864 | 138.7062 | 37.56679 | 24.45583 | 0.207508 |
| Zfp706 | 632.637 | 1705.91 | 1121.51 | 912.162 | 1338.79 | 2111.246 | 3429.281 | 1276.713 | 1394.23 | 0.482074 |
| Fzd6 | 33.4517 | 19 | 203.467 | 113 | 205 | 111.6355 | 38.19447 | 231.6243 | 172.7193 | 2.698683 |
| Oxr1 | 224 | 144 | 194.913 | 111 | 203 | 747.5363 | 289.4739 | 221.8865 | 169.6624 | 0.377575 |
| Ext1 | 149.836 | 111.175 | 1316.82 | 693.397 | 1593.98 | 500.035 | 223.4879 | 1499.052 | 1059.85 | 3.536725 |
| Mal2 | 22 | 20 | 504 | 163 | 541 | 73.41874 | 40.20471 | 573.7474 | 249.1438 | 7.242265 |
| Enpp2 | 6 | 4.00404 | 178.08 | 91.335 | 166.472 | 20.02329 | 8.049063 | 202.7241 | 139.6046 | 12.19451 |
| Mrpl13 | 568.261 | 1731.36 | 842.866 | 878.154 | 832.82 | 1896.409 | 3480.441 | 959.5082 | 1342.249 | 0.428087 |
| Wdr67 | 47 | 52 | 47 | 27.0175 | 50 | 156.8491 | 104.5322 | 53.50422 | 41.29597 | 0.362689 |
| Zhx1 | 13 | 12 | 133 | 69 | 138.473 | 43.3838 | 24.12282 | 151.4056 | 105.4658 | 3.805128 |
| Myc | 636.876 | 370 | 611.821 | 418 | 595.954 | 2125.393 | 743.7871 | 696.4895 | 638.9087 | 0.465429 |
| Adcy8 | 1.01276 | 2 | 92 | 35 | 84.3206 | 3.379798 | 4.020471 | 104.7317 | 53.49714 | 21.38149 |
| Wisp1 | 1 | 1 | 65 | 44.0957 | 99.3056 | 3.337216 | 2.010235 | 73.9952 | 67.39982 | 26.44157 |
| Ndrg1 | 174.074 | 37.3915 | 176.258 | 80.3242 | 174.831 | 580.9225 | 75.16571 | 200.6499 | 122.7747 | 0.492959 |
| Khdrbs3 | 59 | 94.05 | 46 | 20.0128 | 36.0426 | 196.8957 | 189.0626 | 52.36583 | 30.58936 | 0.214933 |
| uc007wbo.1 | 10 | 17 | 100 | 42.3495 | 107.843 | 33.37216 | 34.174 | 113.8388 | 64.73077 | 2.643667 |
| Ptk2 | 354.369 | 3750.34 | 354.118 | 835.77 | 391.895 | 1182.606 | 7539.066 | 403.1236 | 1277.466 | 0.192691 |
| Lynx1 | 7 | 3 | 63 | 28 | 92 | 23.36051 | 6.030706 | 71.71842 | 42.79771 | 3.896271 |
| Ly6e | 87 | 134 | 10623 | 5458 | 12525 | 290.3378 | 269.3715 | 12093.09 | 8342.497 | 36.51108 |
| Ly6a | 102.168 | 125.092 | 7118.52 | 6770.01 | 7094.99 | 340.9566 | 251.4644 | 8103.635 | 10347.89 | 31.14597 |
| Ly6c1 | 26.832 | 9.90826 | 135.466 | 101.944 | 131.981 | 89.54417 | 19.91793 | 154.2128 | 155.8204 | 2.832333 |
| Naprt1 | 9 | 9 | 67 | 31 | 68 | 30.03494 | 18.09212 | 76.27197 | 47.38318 | 2.569348 |
| Nrbp2 | 36 | 8 | 201 | 100.188 | 240.307 | 120.1398 | 16.08188 | 228.8159 | 153.1363 | 2.803903 |
| Plec1 | 602 | 258.031 | 810.249 | 174.079 | 983.706 | 2009.004 | 518.703 | 922.3774 | 266.078 | 0.470171 |
| uc007wke.1 | 57.4795 | 0.686859 | 54.6049 | 4.64562 | 85.9294 | 191.8215 | 1.380748 | 62.16154 | 7.100782 | 0.358497 |
| Scx | 69 | 57 | 421 | 283 | 472 | 230.2679 | 114.5834 | 479.2612 | 432.5626 | 2.644107 |
| Slc39a4 | 52 | 34 | 1604 | 803 | 1808 | 173.5352 | 68.348 | 1825.974 | 1227.377 | 12.62324 |
| Gpt | 22 | 32 | 34 | 11 | 35 | 73.41874 | 64.32753 | 38.70518 | 16.81339 | 0.403049 |
| Txn2 | 399 | 558.803 | 545.051 | 349.332 | 630.56 | 1331.549 | 1123.326 | 620.4793 | 533.9504 | 0.47026 |
| Kctd17 | 28 | 10 | 135 | 64 | 191 | 93.44204 | 20.10235 | 153.6823 | 97.82334 | 2.215043 |
| C1qtnf6 | 7 | 2 | 63 | 30 | 65 | 23.36051 | 4.020471 | 71.71842 | 45.85469 | 4.29397 |
| Lgals2 | 120.677 | 1007.9 | 96.9113 | 111.789 | 132.028 | 402.7252 | 2026.116 | 110.3226 | 170.8683 | 0.115772 |
| Lgals1 | 2451 | 13329 | 2222 | 2137 | 2173 | 8179.516 | 26794.43 | 2529.497 | 3266.382 | 0.16572 |
| Triobp | 116 | 100 | 56 | 35 | 72.2003 | 387.117 | 201.0235 | 63.74971 | 53.49714 | 0.199352 |
| H1f0 | 122 | 207 | 1298 | 857 | 1585.99 | 407.1403 | 416.1187 | 1477.627 | 1309.916 | 3.385985 |
| Gcat | 85 | 103 | 93 | 52 | 106 | 283.6633 | 207.0542 | 105.8701 | 79.48146 | 0.377715 |
| Polr2f | 291 | 826 | 528 | 420.971 | 612 | 971.1297 | 1660.454 | 601.0687 | 643.4498 | 0.472916 |
| Mgat3 | 76 | 70 | 85 | 47 | 112 | 253.6284 | 140.7165 | 96.76295 | 71.83901 | 0.42755 |
| Rps19bp1 | 95 | 341 | 142 | 157 | 181 | 317.0355 | 685.4902 | 161.651 | 239.9729 | 0.400612 |
| Sgsm3 | 34.076 | 40 | 219.27 | 149 | 329.068 | 113.719 | 80.40941 | 249.6143 | 227.745 | 2.458987 |
| Nhp2l1 | 692 | 1803.98 | 1019 | 805 | 1088 | 2309.353 | 3626.424 | 1160.017 | 1230.434 | 0.402719 |
| Parvb | 67 | 72 | 11.0216 | 8 | 7.00358 | 223.5934 | 144.7369 | 12.54685 | 12.22792 | 0.067262 |
| Prr5 | 38 | 55 | 29 | 16 | 24 | 126.8142 | 110.5629 | 33.01324 | 24.45583 | 0.2421 |
| 5031439G07Rik | 68 | 53 | 86 | 28 | 111 | 226.9307 | 106.5425 | 97.90134 | 42.79771 | 0.42192 |
| Fbln1 | 33 | 33 | 5 | 2 | 8 | 110.1281 | 66.33777 | 5.691938 | 3.056979 | 0.049579 |
| Wnt7b | 55 | 27 | 998.799 | 358 | 1309.74 | 183.5469 | 54.27635 | 1137.02 | 547.1993 | 7.081814 |
| Celsr1 | 42 | 16.4497 | 294.772 | 110.049 | 319.669 | 140.1631 | 33.06777 | 335.5648 | 168.2088 | 2.908106 |
| Pim3 | 459 | 306 | 486 | 279 | 657 | 1531.782 | 615.132 | 553.2564 | 426.4486 | 0.456332 |
| Mapk12 | 28 | 32.0097 | 23 | 10.0525 | 44 | 93.44204 | 64.34703 | 26.18292 | 15.36514 | 0.263314 |
| Lmf2 | 104 | 184 | 184 | 66 | 207 | 347.0704 | 369.8833 | 209.4633 | 100.8803 | 0.432864 |
| Cpne8 | 59.9374 | 56.8402 | 287.122 | 197.424 | 320.862 | 200.024 | 114.2622 | 326.8561 | 301.7605 | 2.000141 |
| Slc48a1 | 56 | 48 | 375.612 | 176.303 | 474.854 | 186.8841 | 96.4913 | 427.5921 | 269.4773 | 2.45988 |
| Ccnt1 | 8.16908 | 88.6433 | 10.4999 | 41.0169 | 13.4682 | 27.26198 | 178.1939 | 11.95296 | 62.69391 | 0.363323 |
| Fkbp11 | 79 | 198 | 96 | 99 | 132 | 263.64 | 398.0266 | 109.2852 | 151.3205 | 0.393863 |
| Rhebl1 | 44 | 42 | 21 | 15 | 28 | 146.8375 | 84.42988 | 23.90614 | 22.92735 | 0.202508 |
| Dhh | 23 | 52 | 3 | 3 | 8 | 76.75596 | 104.5322 | 3.415163 | 4.585469 | 0.044132 |
| Tuba1b | 1251.73 | 6117.14 | 2929.39 | 2388.67 | 4029.28 | 4177.293 | 12296.89 | 3334.781 | 3651.057 | 0.424048 |
| Tuba1a | 387.807 | 761.297 | 475.043 | 286.035 | 482.145 | 1294.196 | 1530.386 | 540.7831 | 437.2015 | 0.346241 |
| Troap | 13 | 88 | 38 | 19 | 39.0262 | 43.3838 | 176.9007 | 43.25873 | 29.0413 | 0.328212 |
| Dnajc22 | 50 | 154 | 69 | 55 | 80 | 166.8608 | 309.5762 | 78.54875 | 84.06693 | 0.341316 |
| Fmnl3 | 121 | 93 | 90 | 36 | 77 | 403.8031 | 186.9519 | 102.4549 | 55.02563 | 0.266575 |
| Racgap1 | 56 | 284.19 | 166 | 79 | 159 | 186.8841 | 571.2888 | 188.9723 | 120.7507 | 0.408512 |
| Gpd1 | 19 | 7 | 289 | 170 | 396 | 63.4071 | 14.07165 | 328.994 | 259.8432 | 7.599985 |
| Mettl7a1 | 25 | 90 | 229 | 229 | 306.416 | 83.43039 | 180.9212 | 260.6908 | 350.0241 | 2.310237 |
| BC004728 | 15.9375 | 22 | 219.978 | 169 | 245 | 53.18687 | 44.22518 | 250.4202 | 258.3148 | 5.222506 |
| cela1 | 17 | 38 | 23 | 22 | 22 | 56.73267 | 76.38894 | 26.18292 | 33.62677 | 0.449286 |
| Krt8 | 2801 | 6140.34 | 4615 | 2406 | 5108.89 | 9347.541 | 12343.53 | 5253.659 | 3677.546 | 0.411746 |
| Tenc1 | 35.2 | 28.7887 | 32.3622 | 23.7778 | 54.8881 | 117.47 | 57.87206 | 36.84073 | 36.34412 | 0.417383 |
| Espl1 | 27 | 81 | 39 | 15 | 36 | 90.10482 | 162.8291 | 44.39712 | 22.92735 | 0.266174 |
| Tcfap4 | 58.7342 | 66.4078 | 55.7639 | 28.014 | 47 | 196.0087 | 133.4953 | 63.48093 | 42.81911 | 0.322606 |
| Vasn | 30 | 16 | 263 | 178 | 300 | 100.1165 | 32.16377 | 299.3959 | 272.0712 | 4.320125 |
| Nudt16l1 | 22 | 25.8257 | 129.875 | 97.9238 | 143.959 | 73.41874 | 51.91573 | 147.8481 | 149.6755 | 2.373837 |
| uc007ybi.1 | 5 | 4.1 | 111 | 78 | 110.106 | 16.68608 | 8.241965 | 126.361 | 119.2222 | 9.851685 |
| Rogdi | 17 | 15.0452 | 100 | 55 | 109.438 | 56.73267 | 30.24439 | 113.8388 | 84.06693 | 2.275378 |
| Ubn1 | 8.08769 | 13.0639 | 82.1978 | 36.0428 | 132.66 | 26.99037 | 26.26151 | 93.57296 | 55.09105 | 2.791714 |
| Ppl | 62.1076 | 69.7274 | 455.579 | 166.885 | 520.271 | 207.2665 | 140.1685 | 518.6255 | 255.082 | 2.226913 |
| uc007ydb.1 | 13.0034 | 14.3361 | 117 | 48 | 117 | 43.39515 | 28.81893 | 133.1914 | 73.3675 | 2.860368 |
| Litaf | 546 | 379 | 2518 | 1544.96 | 2633 | 1822.12 | 761.8792 | 2866.46 | 2361.455 | 2.023188 |
| Snn | 24.8538 | 37.0523 | 155.581 | 95.2488 | 198.481 | 82.94249 | 74.48384 | 177.1115 | 145.5868 | 2.049837 |
| Txndc11 | 27.4007 | 36.1031 | 189.562 | 127.62 | 212.26 | 91.44204 | 72.57573 | 215.795 | 195.0659 | 2.504978 |
| uc007yfd.1 | 19 | 39 | 154 | 82 | 203 | 63.4071 | 78.39918 | 175.3117 | 125.3362 | 2.120131 |
| Cpped1 | 27 | 28 | 206.519 | 98.1078 | 310 | 90.10482 | 56.28659 | 235.0987 | 149.9568 | 2.630314 |
| uc007ygf.1 | 14 | 13.57 | 62.8421 | 84.0773 | 107.058 | 46.72102 | 27.27889 | 71.53867 | 128.5113 | 2.703381 |
| Mpv17l | 126.792 | 2653.05 | 217.099 | 661.065 | 292.686 | 423.1322 | 5333.255 | 247.1428 | 1010.431 | 0.218466 |
| Snai2 | 29 | 24 | 7.01589 | 7.00691 | 15.0127 | 96.77925 | 48.24565 | 7.986802 | 10.70999 | 0.128921 |
| Cebpd | 102.942 | 35.5359 | 1557.91 | 1170.93 | 1640.65 | 343.5397 | 71.43552 | 1773.505 | 1789.754 | 8.586682 |
| 2310008H04Rik | 6.06769 | 11.4704 | 61.7798 | 45.2409 | 47.9142 | 20.24919 | 23.0582 | 70.32936 | 69.15025 | 3.220688 |
| Top3b | 45.5136 | 40.1547 | 361.186 | 159.584 | 368.346 | 151.8887 | 80.7204 | 411.1697 | 243.9225 | 2.816279 |
| 2610318N02Rik | 16 | 54 | 18 | 5 | 11 | 53.39545 | 108.5527 | 20.49098 | 7.642448 | 0.173719 |
| Slc7a4 | 7 | 4 | 61 | 21 | 76 | 23.36051 | 8.040941 | 69.44165 | 32.09828 | 3.233606 |
| Scarf2 | 93 | 44 | 35 | 9 | 33 | 310.3611 | 88.45035 | 39.84357 | 13.75641 | 0.134399 |
| Arvcf | 65.8638 | 38.0924 | 316.641 | 215.62 | 391.943 | 219.8017 | 76.57469 | 360.4602 | 329.5729 | 2.328232 |
| Klhl24 | 51 | 13 | 314.858 | 193.452 | 330.267 | 170.198 | 26.13306 | 358.4305 | 295.6894 | 3.331719 |
| Clcn2 | 47.3021 | 28.3183 | 33.3129 | 33.0427 | 56.5329 | 157.8573 | 56.92645 | 37.92299 | 50.50543 | 0.411709 |
| Masp1 | 61 | 35 | 6 | 1 | 8 | 203.5702 | 70.35824 | 6.830326 | 1.52849 | 0.030515 |
| Rtp4 | 2 | 2 | 351 | 247 | 385 | 6.674431 | 4.020471 | 399.5741 | 377.5369 | 72.66182 |
| Bcl6 | 28 | 17.0375 | 169.188 | 104 | 218 | 93.44204 | 34.24938 | 192.6015 | 158.9629 | 2.753235 |
| uc007yvq.1 | 105.164 | 105 | 643 | 401 | 703 | 350.9549 | 211.0747 | 731.9833 | 612.9244 | 2.392948 |
| 1500031L02Rik | 16 | 26 | 89 | 110.466 | 88 | 53.39545 | 52.26612 | 101.3165 | 168.8461 | 2.556868 |
| Tctex1d2 | 7 | 22 | 65 | 70 | 73 | 23.36051 | 44.22518 | 73.9952 | 106.9943 | 2.677926 |
| Heg1 | 3 | 1.00055 | 57.859 | 31.0128 | 61.0119 | 10.01165 | 2.011341 | 65.86597 | 47.40274 | 9.421012 |
| Umps | 156 | 156 | 174 | 139 | 183.057 | 520.6056 | 313.5967 | 198.0794 | 212.4601 | 0.492134 |
| Kalrn | 7.3384 | 6.02127 | 76.2148 | 40.8652 | 106.364 | 24.48982 | 12.10417 | 86.76199 | 62.46204 | 4.077828 |
| Sec22a | 38 | 33 | 177 | 144 | 185 | 126.8142 | 66.33777 | 201.4946 | 220.1025 | 2.182722 |
| Dirc2 | 33.3777 | 13 | 348.817 | 252.91 | 440.723 | 111.3886 | 26.13306 | 397.089 | 386.5703 | 5.698443 |
| Parp14 | 6.03965 | 3.10109 | 256.6 | 139.331 | 281.922 | 20.15561 | 6.233921 | 292.1103 | 212.966 | 19.13926 |
| Dtx3l | 6.05581 | 4.01018 | 161.374 | 80.6768 | 225.392 | 20.20954 | 8.061405 | 183.7062 | 123.3137 | 10.8599 |
| Parp9 | 11 | 7 | 211 | 122 | 250 | 36.70937 | 14.07165 | 240.1998 | 186.4757 | 8.402264 |
| Fstl1 | 10 | 5 | 1210.75 | 431.573 | 1333 | 33.37216 | 10.05118 | 1378.303 | 659.6549 | 46.93232 |
| Slc35a5 | 14 | 13 | 115 | 73.9798 | 111 | 46.72102 | 26.13306 | 130.9146 | 113.0774 | 3.34905 |
| C330027C09Rik | 56.4177 | 199.041 | 125.775 | 72.3724 | 118.946 | 188.278 | 400.1192 | 143.1807 | 110.6205 | 0.431343 |
| Ift57 | 41 | 30 | 205 | 133 | 210 | 136.8258 | 60.30706 | 233.3695 | 203.2891 | 2.215047 |
| Cd47 | 349.839 | 275.907 | 1766.62 | 1077.1 | 2225.56 | 1167.488 | 554.638 | 2011.098 | 1646.336 | 2.12379 |
| Nfkbiz | 151 | 99 | 885 | 534 | 998 | 503.9196 | 199.0133 | 1007.473 | 816.2135 | 2.594397 |
| Tmem45a | 3 | 0 | 1164 | 846 | 1185 | 10.01165 | 0 | 1325.083 | 1293.102 | 261.514 |
| Tmem30c | 11.1774 | 124.461 | 3.27157 | 4.82826 | 4.04056 | 37.30139 | 250.1959 | 3.724315 | 7.379946 | 0.038624 |
| uc007znj.1 | 72 | 54 | 448 | 236 | 459 | 240.2795 | 108.5527 | 509.9977 | 360.7236 | 2.496103 |
| uc007zns.1 | 30 | 61 | 5 | 3 | 5 | 100.1165 | 122.6244 | 5.691938 | 4.585469 | 0.046141 |
| St3gal6 | 215 | 197 | 17 | 15 | 31 | 717.5014 | 396.0164 | 19.35259 | 22.92735 | 0.03797 |
| Arl6 | 25 | 37 | 162 | 110 | 196 | 83.43039 | 74.37871 | 184.4188 | 168.1339 | 2.234045 |
| Arl13b | 20 | 14 | 85.1437 | 68.1032 | 96 | 66.74431 | 28.14329 | 96.92654 | 104.095 | 2.118523 |
| Pros1 | 13.0328 | 9 | 117.231 | 60.1119 | 131 | 43.49326 | 18.09212 | 133.4543 | 91.88042 | 3.6589 |
| Gbe1 | 4 | 9 | 52 | 51 | 81.0088 | 13.34886 | 18.09212 | 59.19616 | 77.95297 | 4.362114 |
| Usp25 | 39 | 43 | 264 | 155 | 295 | 130.1514 | 86.44012 | 300.5343 | 236.9159 | 2.4814 |
| App | 714.993 | 308 | 4303 | 2395.99 | 5541.89 | 2386.086 | 619.1525 | 4898.482 | 3662.246 | 2.848602 |
| Adamts1 | 40 | 5 | 123 | 98.5891 | 120 | 133.4886 | 10.05118 | 140.0217 | 150.6924 | 2.02532 |
| 4931408A02Rik | 8.18642 | 14.1254 | 129.724 | 64.1876 | 136.741 | 27.31985 | 28.39538 | 147.6762 | 98.11008 | 4.411474 |
| Synj1 | 22 | 17 | 118.095 | 63.0496 | 120 | 73.41874 | 34.174 | 134.4379 | 96.37066 | 2.145206 |
| Ifnar2 | 35 | 31 | 292 | 212 | 298 | 116.8025 | 62.31729 | 332.4092 | 324.0398 | 3.664859 |
| Il10rb | 66 | 79 | 507 | 476.994 | 659 | 220.2562 | 158.8086 | 577.1625 | 729.0804 | 3.445962 |
| Ifnar1 | 27.3435 | 17.0128 | 214.39 | 118.647 | 262.621 | 91.25116 | 34.19973 | 244.0589 | 181.3507 | 3.391045 |
| Ifngr2 | 55.1054 | 40 | 579.075 | 434.532 | 787.081 | 183.8986 | 80.40941 | 659.2118 | 664.1777 | 5.006997 |
| Tmem50b | 9 | 14 | 252.843 | 158 | 307 | 30.03494 | 28.14329 | 287.8333 | 241.5014 | 9.098501 |
| Rcan1 | 35 | 27 | 186 | 113 | 207 | 116.8025 | 54.27635 | 211.7401 | 172.7193 | 2.247264 |
| Clic6 | 32 | 32 | 19 | 15.4499 | 29.3666 | 106.7909 | 64.32753 | 21.62937 | 23.61501 | 0.264404 |
| Kcnj15 | 0 | 1 | 57.2441 | 24 | 39.1787 | 0 | 2.010235 | 65.16598 | 36.68375 | 50.66557 |
| Igsf5 | 20 | 26 | 142 | 97 | 189 | 66.74431 | 52.26612 | 161.651 | 148.2635 | 2.604096 |
| Mx2 | 1 | 0 | 66 | 22 | 68 | 3.337216 | 0 | 75.13358 | 33.62677 | 32.59015 |
| Tmprss2 | 6 | 4 | 340 | 144 | 415.276 | 20.02329 | 8.040941 | 387.0518 | 220.1025 | 21.63445 |
| Tiam2 | 47.4146 | 53.8235 | 31.1055 | 11.5693 | 21 | 158.2327 | 108.1979 | 35.41012 | 17.68356 | 0.199278 |
| Arid1b | 48.713 | 27.0622 | 265.423 | 131.475 | 287.901 | 162.5658 | 54.40139 | 302.1543 | 200.9582 | 2.318841 |
| Tulp4 | 70.5139 | 139.211 | 596.388 | 306.2 | 709.584 | 235.3201 | 279.8469 | 678.9207 | 468.0235 | 2.226354 |
| Rshl2b | 93.7386 | 66.2365 | 1136.57 | 456.867 | 1132.18 | 312.8259 | 133.151 | 1293.857 | 698.3165 | 4.466989 |
| Rnaset2b | 2.56546 | 40.0076 | 137.344 | 104.591 | 116.64 | 8.561493 | 80.42469 | 156.3507 | 159.8663 | 3.553551 |
| uc008aiy.1 | 194.418 | 189.972 | 867.81 | 774.3 | 995.866 | 648.8148 | 381.8884 | 987.9042 | 1183.51 | 2.10673 |
| Brp44l | 53.9623 | 81 | 424 | 316 | 460 | 180.0838 | 162.8291 | 482.6764 | 483.0027 | 2.816106 |
| uc008aka.1 | 36 | 24.0426 | 31 | 16 | 45 | 120.1398 | 48.33128 | 35.29002 | 24.45583 | 0.354636 |
| Qk | 106.908 | 66.9396 | 75.9584 | 37.3951 | 61.0961 | 356.775 | 134.5643 | 86.4701 | 57.15802 | 0.29232 |
| Park2 | 2.98349 | 123.392 | 1.34678 | 1.96487 | 4.00002 | 9.956549 | 248.047 | 1.533158 | 3.003284 | 0.017583 |
| Igf2r | 86.409 | 21 | 415.062 | 182.421 | 486.091 | 288.3655 | 42.21494 | 472.5014 | 278.8286 | 2.27276 |
| Acat3 | 2.8765 | 2.08893 | 130.623 | 34.8802 | 100.118 | 9.599501 | 4.199241 | 148.6996 | 53.31403 | 14.64 |
| Acat2 | 113.713 | 50.3641 | 555.27 | 240.871 | 597.852 | 379.4848 | 101.2437 | 632.1125 | 368.1688 | 2.080761 |
| Wtap | 41 | 71.7944 | 294.101 | 210 | 332.172 | 136.8258 | 144.3236 | 334.8009 | 320.9828 | 2.332509 |
| Sod2 | 158.98 | 203.941 | 1379.33 | 1021.57 | 1528.42 | 530.5505 | 409.9694 | 1570.212 | 1561.459 | 3.329724 |
| uc008anr.1 | 5.72164 | 2.14557 | 56.2988 | 43.9945 | 70.7489 | 19.09435 | 4.313101 | 64.08986 | 67.24514 | 5.610821 |
| uc008aod.1 | 5.72164 | 2.14557 | 56.2988 | 43.9945 | 70.7489 | 19.09435 | 4.313101 | 64.08986 | 67.24514 | 5.610821 |
| Dll1 | 2 | 2 | 63 | 41 | 54 | 6.674431 | 4.020471 | 71.71842 | 62.66808 | 12.56547 |
| 4930546H06Rik | 9 | 11 | 75 | 23 | 54 | 30.03494 | 22.11259 | 85.37907 | 35.15526 | 2.31141 |
| 4930432O21Rik | 12 | 11 | 55.6954 | 47.6395 | 54.6053 | 40.04659 | 22.11259 | 63.40295 | 72.81648 | 2.191461 |
| Abca3 | 72.7732 | 22.2829 | 438.841 | 163.504 | 549.548 | 242.8599 | 44.79387 | 499.5712 | 249.9142 | 2.605512 |
| uc008awt.1 | 14 | 106 | 63 | 35 | 53 | 46.72102 | 213.0849 | 71.71842 | 53.49714 | 0.481958 |
| Nthl1 | 19 | 23.501 | 9 | 23 | 24 | 63.4071 | 47.24254 | 10.24549 | 35.15526 | 0.410311 |
| Noxo1 | 1 | 2 | 126 | 115 | 150 | 3.337216 | 4.020471 | 143.4368 | 175.7763 | 43.38499 |
| Nme3 | 17.5147 | 61.9471 | 153.505 | 145.165 | 167.667 | 58.45033 | 124.5282 | 174.7482 | 221.8832 | 2.167638 |
| Gnptg | 13 | 29.6215 | 175.85 | 127.454 | 181.744 | 43.3838 | 59.54619 | 200.1855 | 194.8121 | 3.837537 |
| Haghl | 2.78788 | 17.9524 | 49.7463 | 55.6471 | 28.9381 | 9.303757 | 36.08855 | 56.63057 | 85.05602 | 3.121379 |
| 9530058B02Rik | 44 | 118 | 57 | 75 | 72 | 146.8375 | 237.2078 | 64.8881 | 114.6367 | 0.467457 |
| Pigq | 29.883 | 48.8011 | 231.027 | 98.8826 | 284.274 | 99.72601 | 98.10169 | 262.9983 | 151.141 | 2.093434 |
| Arhgdig | 19 | 26 | 3 | 3 | 13 | 63.4071 | 52.26612 | 3.415163 | 4.585469 | 0.069166 |
| EG240055 | 4.07435 | 6.82171 | 97.1659 | 58.31 | 109.253 | 13.59698 | 13.71324 | 110.6125 | 89.12623 | 7.313696 |
| Dusp1 | 270 | 115 | 1167 | 805 | 1126 | 901.0482 | 231.1771 | 1328.498 | 1230.434 | 2.260091 |
| Bnip1 | 13 | 11 | 67.278 | 40 | 79 | 43.3838 | 22.11259 | 76.58844 | 61.13959 | 2.102834 |
| Hmga1 | 1449 | 1758 | 695 | 361 | 854 | 4835.625 | 3533.994 | 791.1794 | 551.7848 | 0.160457 |
| Zfp523 | 8 | 11 | 71 | 28 | 85 | 26.69773 | 22.11259 | 80.82552 | 42.79771 | 2.532728 |
| Ppard | 97 | 46.2912 | 123.28 | 44 | 136.701 | 323.7099 | 93.0562 | 140.3404 | 67.25355 | 0.498107 |
| Tead3 | 16 | 20.3333 | 100 | 60.8451 | 92.9027 | 53.39545 | 40.87472 | 113.8388 | 93.00111 | 2.194118 |
| Fkbp5 | 22 | 43 | 461 | 230 | 671 | 73.41874 | 86.44012 | 524.7967 | 351.5526 | 5.482019 |
| Srpk1 | 118.64 | 78 | 144 | 59 | 132 | 395.9273 | 156.7984 | 163.9278 | 90.18089 | 0.459738 |
| Mapk14 | 39 | 54 | 258 | 140 | 295 | 130.1514 | 108.5527 | 293.704 | 213.9886 | 2.12687 |
| Mapk13 | 5 | 9 | 92 | 62 | 128.062 | 16.68608 | 18.09212 | 104.7317 | 94.76636 | 5.736296 |
| Cdkn1a | 85.4745 | 47 | 789.148 | 348.237 | 907.577 | 285.2468 | 94.48106 | 898.3563 | 532.2767 | 3.767521 |
| Ppil1 | 75.4386 | 335 | 158 | 129 | 194 | 251.7549 | 673.4288 | 179.8652 | 197.1752 | 0.40753 |
| BC004004 | 24 | 37 | 141 | 108.063 | 217 | 80.09318 | 74.37871 | 160.5127 | 165.1732 | 2.108383 |
| Glo1 | 149.61 | 224.736 | 884.047 | 649.208 | 941.827 | 499.2808 | 451.7722 | 1006.388 | 992.3077 | 2.101561 |
| Tff2 | 67 | 26 | 21 | 27 | 27 | 223.5934 | 52.26612 | 23.90614 | 41.26922 | 0.236263 |
| Tff1 | 27 | 227 | 9 | 9.375 | 7.35 | 90.10482 | 456.3234 | 10.24549 | 14.32959 | 0.044974 |
| Slc37a1 | 5 | 2 | 128.063 | 73.2366 | 142 | 16.68608 | 4.020471 | 145.7853 | 111.9414 | 12.44663 |
| Sik1 | 38.364 | 15.4814 | 247.935 | 139.499 | 216.152 | 128.0289 | 31.12126 | 282.2461 | 213.2228 | 3.113216 |
| Wiz | 190.111 | 110.694 | 213.962 | 80.8825 | 231.267 | 634.4414 | 222.521 | 243.5717 | 123.6281 | 0.42849 |
| Zfp870 | 31.4062 | 1342.55 | 54.3193 | 395.734 | 67.0832 | 104.8093 | 2698.841 | 61.83642 | 604.8753 | 0.237801 |
| Cyp4f13 | 23.3966 | 9 | 115.829 | 59.5641 | 155.156 | 78.0795 | 18.09212 | 131.8583 | 91.04311 | 2.317746 |
| 2-Mar | 19.3505 | 37.3847 | 124.082 | 91.8678 | 113.702 | 64.57679 | 75.15204 | 141.2534 | 140.419 | 2.01585 |
| Angptl4 | 128 | 328 | 90 | 64 | 126 | 427.1636 | 659.3572 | 102.4549 | 97.82334 | 0.18433 |
| Zbtb22 | 16 | 18 | 110.956 | 36.798 | 134 | 53.39545 | 36.18424 | 126.3109 | 56.24536 | 2.037921 |
| Tapbp | 47 | 23.0863 | 792.276 | 319.431 | 971.375 | 156.8491 | 46.4089 | 901.9172 | 488.247 | 6.839406 |
| Wdr46 | 51 | 121 | 85 | 71 | 150 | 170.198 | 243.2385 | 96.76295 | 108.5228 | 0.496535 |
| H2-K1 | 152.851 | 63.1994 | 2427.99 | 1536.42 | 2510.91 | 510.0967 | 127.0457 | 2763.994 | 2348.402 | 8.023945 |
| Ring1 | 4 | 8 | 52 | 37 | 48 | 13.34886 | 16.08188 | 59.19616 | 56.55412 | 3.932971 |
| C4b | 0.5 | 0 | 53.5 | 37.6664 | 40.8645 | 1.668608 | 0 | 60.90374 | 57.5727 | 71.00317 |
| Cfb | 30 | 47 | 1909.91 | 997.929 | 2477.92 | 100.1165 | 94.48106 | 2174.218 | 1525.324 | 19.01125 |
| Slc44a4 | 1 | 0 | 51 | 28 | 57 | 3.337216 | 0 | 58.05777 | 42.79771 | 30.22145 |
| uc008cek.1 | 728.613 | 1019.56 | 713.089 | 671.758 | 748.241 | 2431.539 | 2049.556 | 811.7717 | 1026.775 | 0.41029 |
| Hspa1b | 21.5253 | 46.75 | 237.171 | 133.594 | 231.572 | 71.83457 | 93.9785 | 269.9925 | 204.197 | 2.859784 |
| Hspa1a | 11.7044 | 4.25 | 68.8221 | 37.4062 | 51.4605 | 39.06011 | 8.5435 | 78.34623 | 57.17499 | 2.846869 |
| Lsm2 | 70 | 277 | 165 | 135 | 209 | 233.6051 | 556.8352 | 187.834 | 206.3461 | 0.498684 |
| uc008cev.1 | 7.11716 | 81.1304 | 676 | 1097.63 | 651.556 | 23.7515 | 163.0912 | 769.55 | 1677.716 | 13.098 |
| Tnf | 3 | 0 | 100 | 85.9884 | 125 | 10.01165 | 0 | 113.8388 | 131.4324 | 24.49858 |
| H2-D1 | 143.739 | 212.674 | 2794.63 | 2434.2 | 3562.15 | 479.688 | 427.5248 | 3181.372 | 3720.65 | 7.607941 |
| uc008chm.1 | 2.4219 | 0 | 42.743 | 47.6941 | 36.5509 | 8.082403 | 0 | 48.6581 | 72.89994 | 15.03984 |
| 2310014H01Rik | 95.5 | 57 | 66.1209 | 29.5 | 83.2503 | 318.7041 | 114.5834 | 75.27122 | 45.09045 | 0.277787 |
| uc008civ.1 | 95.5 | 57 | 46.8791 | 29.5 | 83.2503 | 318.7041 | 114.5834 | 53.36659 | 45.09045 | 0.227233 |
| uc008cke.1 | 0 | 1.10718 | 64.9167 | 33.432 | 81.0039 | 0 | 2.225692 | 73.90037 | 51.10047 | 56.16268 |
| H2-T10 | 0.125951 | 0 | 56.4503 | 28.6531 | 48.0429 | 0.420326 | 0 | 64.26232 | 43.79597 | 257.0823 |
| Crisp1 | 335.906 | 282.747 | 2 | 5.71429 | 7.875 | 1120.991 | 568.388 | 2.276775 | 8.734233 | 0.006518 |
| 9130008F23Rik | 33 | 34 | 30 | 30 | 33 | 110.1281 | 68.348 | 34.15163 | 45.85469 | 0.448275 |
| Cenpq | 40.3173 | 72 | 43 | 59 | 78 | 134.5475 | 144.7369 | 48.95067 | 90.18089 | 0.498171 |
| Pla2g7 | 15 | 32 | 10 | 14 | 16 | 50.05823 | 64.32753 | 11.38388 | 21.39886 | 0.286598 |
| Cyp39a1 | 6.00962 | 7 | 69.492 | 58 | 142.242 | 20.0554 | 14.07165 | 79.10883 | 88.6524 | 4.915786 |
| Enpp5 | 17 | 9 | 138 | 104 | 148 | 56.73267 | 18.09212 | 157.0975 | 158.9629 | 4.224007 |
| Enpp4 | 28 | 29 | 394.394 | 154.322 | 385.791 | 93.44204 | 58.29682 | 448.9733 | 235.8796 | 4.513365 |
| Nfkbie | 24 | 22 | 168 | 115.084 | 210 | 80.09318 | 44.22518 | 191.2491 | 175.9047 | 2.953336 |
| BC048355 | 26.0831 | 140.335 | 64.1663 | 40.0375 | 69.6671 | 87.04493 | 282.1064 | 73.04612 | 61.19691 | 0.363653 |
| Mocs1 | 15 | 15.0687 | 133.832 | 76.2104 | 164.643 | 50.05823 | 30.29163 | 152.3527 | 116.4868 | 3.345861 |
| Plcl2 | 23.1997 | 17.111 | 1 | 1 | 1 | 77.4224 | 34.39714 | 1.138388 | 1.52849 | 0.02385 |
| Kat2b | 20.3171 | 16 | 228.598 | 157.651 | 261.981 | 67.80254 | 32.16377 | 260.2331 | 240.9679 | 5.0137 |
| Sgol1 | 12 | 48 | 33.9706 | 17 | 28.0121 | 40.04659 | 96.4913 | 38.67171 | 25.98432 | 0.473539 |
| Stap2 | 33 | 24 | 327 | 240 | 382 | 110.1281 | 48.24565 | 372.2528 | 366.8375 | 4.666747 |
| Ticam1 | 11 | 3 | 66.0534 | 24 | 63 | 36.70937 | 6.030706 | 75.19437 | 36.68375 | 2.61764 |
| AI662250 | 7 | 3 | 75 | 55 | 116 | 23.36051 | 6.030706 | 85.37907 | 84.06693 | 5.765192 |
| C3 | 11 | 0 | 6891 | 3071 | 8509 | 36.70937 | 0 | 7844.629 | 4693.992 | 341.5646 |
| Gpr108 | 36 | 57 | 256 | 133 | 297 | 120.1398 | 114.5834 | 291.4272 | 203.2891 | 2.107659 |
| Twsg1 | 72 | 114.472 | 457.144 | 318.308 | 450.691 | 240.2795 | 230.1157 | 520.4071 | 486.5305 | 2.14062 |
| Ddx11 | 32.2684 | 36.6868 | 37.2174 | 27.2123 | 32.2472 | 107.6866 | 73.7491 | 42.36783 | 41.59372 | 0.462762 |
| Myl12b | 292.457 | 562.134 | 2332.17 | 2273.76 | 2689.33 | 975.9921 | 1130.022 | 2654.913 | 3475.419 | 2.91087 |
| Ndc80 | 22 | 83.8257 | 42.9 | 34.7336 | 51.8293 | 73.41874 | 168.5094 | 48.83683 | 53.08995 | 0.42131 |
| Lbh | 76 | 82.5716 | 30 | 24 | 48 | 253.6284 | 165.9883 | 34.15163 | 36.68375 | 0.16881 |
| Xdh | 1 | 4.74609 | 964 | 594.625 | 1219.65 | 3.337216 | 9.540758 | 1097.406 | 908.8782 | 155.7919 |
| Ttc27 | 47 | 57 | 69 | 32 | 72 | 156.8491 | 114.5834 | 78.54875 | 48.91167 | 0.469584 |
| Ltbp1 | 112.842 | 81.1378 | 15.0129 | 13.0361 | 24.0193 | 376.5781 | 163.1061 | 17.0905 | 19.92554 | 0.068588 |
| Fez2 | 203 | 226 | 155 | 94 | 162 | 677.4548 | 454.3132 | 176.4501 | 143.678 | 0.282857 |
| Eif2ak2 | 20.368 | 23.1556 | 132.806 | 64.7496 | 167.882 | 67.97241 | 46.5482 | 151.1847 | 98.96909 | 2.184356 |
| Cdc42ep3 | 8 | 3 | 51 | 38 | 35 | 26.69773 | 6.030706 | 58.05777 | 58.08261 | 3.548608 |
| Cyp1b1 | 16 | 15 | 921 | 431.997 | 1032 | 53.39545 | 30.15353 | 1048.455 | 660.303 | 20.45217 |
| Morn2 | 0 | 21.392 | 55.4357 | 51.6474 | 62.9931 | 0 | 43.00295 | 63.10732 | 78.94252 | 3.303258 |
| Map4k3 | 65 | 26 | 380 | 239 | 406 | 216.919 | 52.26612 | 432.5873 | 365.309 | 2.964117 |
| Pkdcc | 11 | 12 | 204.214 | 140 | 218 | 36.70937 | 24.12282 | 232.4747 | 213.9886 | 7.339259 |
| Dync2li1 | 34 | 17 | 165 | 110 | 173 | 113.4653 | 34.174 | 187.834 | 168.1339 | 2.411064 |
| Mcfd2 | 96 | 105 | 546.85 | 405 | 622 | 320.3727 | 211.0747 | 622.5273 | 619.0383 | 2.336197 |
| Ttc7 | 13 | 4 | 141.682 | 33 | 199.692 | 43.3838 | 8.040941 | 161.289 | 50.44016 | 4.117263 |
| Epcam | 6.10714 | 7 | 856.323 | 531.428 | 832.969 | 20.38084 | 14.07165 | 974.8275 | 812.2822 | 51.87171 |
| Klraq1 | 25 | 21 | 137 | 67 | 120 | 83.43039 | 42.21494 | 155.9591 | 102.4088 | 2.056327 |
| Map3k8 | 3 | 0 | 57 | 36 | 50 | 10.01165 | 0 | 64.8881 | 55.02563 | 11.97742 |
| Svil | 46.3459 | 27 | 301 | 131.307 | 313.444 | 154.6663 | 54.27635 | 342.6547 | 200.7014 | 2.600504 |
| Kctd1 | 7 | 0 | 48 | 31 | 69 | 23.36051 | 0 | 54.64261 | 47.38318 | 4.367447 |
| Lims2 | 5 | 5 | 166 | 95 | 192 | 16.68608 | 10.05118 | 188.9723 | 145.2065 | 12.49862 |
| Epb4.1l4a | 1 | 0 | 208.165 | 111 | 250 | 3.337216 | 0 | 236.9725 | 169.6624 | 121.8485 |
| Kif20a | 13.615 | 221.588 | 64.2507 | 39.9452 | 42.6718 | 45.43619 | 445.444 | 73.1422 | 61.05583 | 0.273382 |
| Sil1 | 28 | 30 | 203 | 120 | 186 | 93.44204 | 60.30706 | 231.0927 | 183.4188 | 2.696025 |
| 5133400G04Rik | 11 | 8 | 49 | 36 | 52 | 36.70937 | 16.08188 | 55.78099 | 55.02563 | 2.098958 |
| Ecscr | 69 | 62 | 417 | 267.814 | 445 | 230.2679 | 124.6346 | 474.7076 | 409.3509 | 2.49099 |
| Tmem173 | 28 | 26 | 306 | 169 | 334 | 93.44204 | 52.26612 | 348.3466 | 258.3148 | 4.163538 |
| Apbb3 | 28 | 15 | 175 | 84.7568 | 197.108 | 93.44204 | 30.15353 | 199.2178 | 129.5499 | 2.660028 |
| Cd14 | 154 | 147 | 5401 | 3287.96 | 6041 | 513.9312 | 295.5046 | 6148.432 | 5025.613 | 13.80473 |
| Arap3 | 40 | 21.0007 | 7 | 3 | 18 | 133.4886 | 42.21635 | 7.968713 | 4.585469 | 0.07145 |
| Spry4 | 37.5489 | 17.0204 | 47.0741 | 14.0056 | 38.0975 | 125.3088 | 34.21501 | 53.58857 | 21.40741 | 0.470124 |
| Ppp2r2b | 4 | 1 | 74.1436 | 38.122 | 74 | 13.34886 | 2.010235 | 84.40416 | 58.26908 | 9.289168 |
| uc008euf.1 | 106.301 | 69.893 | 16.0203 | 5.0401 | 23.1475 | 354.7494 | 140.5014 | 18.23731 | 7.703741 | 0.05238 |
| uc008evj.1 | 17.04 | 20.0495 | 100.285 | 53.1953 | 136.452 | 56.86615 | 40.30421 | 114.1632 | 81.30847 | 2.011639 |
| Commd10 | 14 | 38.1447 | 132.655 | 110.52 | 164.783 | 46.72102 | 76.67982 | 151.0128 | 168.9287 | 2.592701 |
| Srfbp1 | 13 | 33 | 19 | 19 | 22 | 43.3838 | 66.33777 | 21.62937 | 29.0413 | 0.461811 |
| Ppic | 58 | 53 | 547.017 | 385 | 709.76 | 193.5585 | 106.5425 | 622.7174 | 588.4685 | 4.035928 |
| Gramd3 | 19 | 5 | 80.5841 | 42.2523 | 90.5624 | 63.4071 | 10.05118 | 91.73594 | 64.5822 | 2.127986 |
| 2010002N04Rik | 46 | 71 | 26 | 23 | 33 | 153.5119 | 142.7267 | 29.59808 | 35.15526 | 0.218585 |
| Synpo | 22 | 2 | 151.966 | 41.5 | 180.746 | 73.41874 | 4.020471 | 172.9962 | 63.43232 | 3.053085 |
| uc008faq.1 | 22 | 2 | 96.0338 | 41.5 | 141.254 | 73.41874 | 4.020471 | 109.3237 | 63.43232 | 2.23086 |
| Tcof1 | 53 | 66.7476 | 78.487 | 34.2464 | 81.0121 | 176.8724 | 134.1784 | 89.34863 | 52.34527 | 0.455533 |
| Pcyox1l | 18 | 19 | 103 | 61 | 50.2633 | 60.06988 | 38.19447 | 117.2539 | 93.23787 | 2.142097 |
| Fech | 30 | 28 | 262.054 | 228.276 | 304.987 | 100.1165 | 56.28659 | 298.319 | 348.9175 | 4.13826 |
| Atp8b1 | 3 | 1.01012 | 113 | 69.0664 | 101.217 | 10.01165 | 2.030579 | 128.6378 | 105.5673 | 19.44865 |
| Nedd4l | 45 | 22.1058 | 261.566 | 139.696 | 257.085 | 150.1747 | 44.43786 | 297.7635 | 213.5239 | 2.627207 |
| Malt1 | 9.08126 | 14.9543 | 68.9011 | 30.4002 | 54.51 | 30.30612 | 30.06166 | 78.43616 | 46.46639 | 2.069027 |
| Sec11c | 23 | 71 | 247 | 283 | 347 | 76.75596 | 142.7267 | 281.1817 | 432.5626 | 3.251939 |
| Ccbe1 | 62 | 55.1851 | 22 | 10 | 33 | 206.9074 | 110.935 | 25.04453 | 15.2849 | 0.126885 |
| Pmaip1 | 26 | 14.0022 | 343.506 | 205 | 353.431 | 86.76761 | 28.14772 | 391.043 | 313.3404 | 6.129586 |
| Chmp1b | 25 | 39 | 140 | 109 | 137 | 83.43039 | 78.39918 | 159.3743 | 166.6054 | 2.014339 |
| Spire1 | 61 | 42.1021 | 50 | 29 | 43 | 203.5702 | 84.63513 | 56.91938 | 44.3262 | 0.351297 |
| Ccdc68 | 43 | 35 | 44 | 28 | 47 | 143.5003 | 70.35824 | 50.08906 | 42.79771 | 0.434337 |
| Rab27b | 44.0786 | 88.8058 | 69.6095 | 45.9332 | 82.6236 | 147.0998 | 178.5206 | 79.24259 | 70.20842 | 0.458973 |
| uc008foo.1 | 17.5632 | 41.2385 | 6.13343 | 28.4575 | 5.13133 | 58.61219 | 82.89909 | 6.982221 | 43.49699 | 0.356715 |
| Me2 | 30 | 137 | 75 | 51 | 87 | 100.1165 | 275.4022 | 85.37907 | 77.95297 | 0.43495 |
| 2810433K01Rik | 4.0036 | 74.3 | 20.0585 | 21 | 40 | 13.36088 | 149.3605 | 22.83435 | 32.09828 | 0.337587 |
| Slc14a1 | 23 | 20 | 1.00085 | 0 | 0 | 76.75596 | 40.20471 | 1.139355 | 0 | 0.009741 |
| Atp9b | 24 | 14.7674 | 147 | 51 | 176 | 80.09318 | 29.68595 | 167.343 | 77.95297 | 2.23445 |
| uc008ftp.1 | 33.4947 | 12.8792 | 26.0265 | 21.4505 | 39.0767 | 111.779 | 25.89022 | 29.62825 | 32.78687 | 0.45337 |
| Tshz1 | 31.0062 | 25.8257 | 229.333 | 113.255 | 249.109 | 103.4744 | 51.91573 | 261.0699 | 173.1091 | 2.794122 |
| Zadh2 | 49 | 35 | 254 | 134 | 272 | 163.5236 | 70.35824 | 289.1505 | 204.8176 | 2.112042 |
| Cndp2 | 26 | 52 | 260 | 77 | 231 | 86.76761 | 104.5322 | 295.9808 | 117.6937 | 2.16244 |
| Rttn | 27.2322 | 26.658 | 42.7374 | 13.3508 | 47.9391 | 90.87972 | 53.58885 | 48.65173 | 20.40656 | 0.478016 |
| Chka | 338.83 | 192.317 | 327.553 | 155.937 | 455.283 | 1130.749 | 386.6024 | 372.8823 | 238.3481 | 0.402827 |
| Tcirg1 | 35 | 36 | 284 | 109 | 315.957 | 116.8025 | 72.36847 | 323.3021 | 166.6054 | 2.58976 |
| Acy3 | 25 | 37 | 36 | 24 | 39 | 83.43039 | 74.37871 | 40.98195 | 36.68375 | 0.49215 |
| Gstp2 | 106.385 | 5.90855 | 37.4971 | 4.87209 | 117.987 | 355.0297 | 11.87758 | 42.68624 | 7.446939 | 0.136637 |
| Rad9 | 128.113 | 127.572 | 154.903 | 56.5323 | 250.027 | 427.5407 | 256.4497 | 176.3397 | 86.40904 | 0.384141 |
| Pold4 | 278 | 436 | 256 | 200 | 251 | 927.7459 | 876.4626 | 291.4272 | 305.6979 | 0.330962 |
| B3gnt1 | 40 | 46.1955 | 225.981 | 139.86 | 207.19 | 133.4886 | 92.86383 | 257.254 | 213.7746 | 2.080952 |
| Drap1 | 245 | 682 | 435 | 380 | 505 | 817.6178 | 1370.98 | 495.1986 | 580.8261 | 0.49165 |
| Ccdc85b | 144.912 | 333.084 | 76.1504 | 95.6877 | 124.193 | 483.6026 | 669.5772 | 86.68867 | 146.2577 | 0.202003 |
| Ehbp1l1 | 66 | 56 | 56 | 21 | 58 | 220.2562 | 112.5732 | 63.74971 | 32.09828 | 0.287979 |
| uc008gfj.1 | 44.3601 | 37.3201 | 223.914 | 155.681 | 229.696 | 148.0392 | 75.02218 | 254.9009 | 237.9568 | 2.209516 |
| uc008gfk.1 | 60 | 108 | 4994.99 | 1813.95 | 5622 | 200.2329 | 217.1054 | 5686.235 | 2772.604 | 20.26854 |
| Slc25a45 | 3 | 11 | 49 | 30 | 54.0793 | 10.01165 | 22.11259 | 55.78099 | 45.85469 | 3.163832 |
| Cdc42ep2 | 5 | 26 | 101 | 58 | 137 | 16.68608 | 52.26612 | 114.9772 | 88.6524 | 2.953199 |
| Pola2 | 29.0214 | 184.153 | 73.3983 | 62.8861 | 116.934 | 96.85067 | 370.1909 | 83.55572 | 96.12075 | 0.384712 |
| Cdca5 | 23 | 71 | 45 | 21 | 40 | 76.75596 | 142.7267 | 51.22744 | 32.09828 | 0.379646 |
| Cdc42bpg | 28 | 24 | 237 | 146 | 328 | 93.44204 | 48.24565 | 269.7979 | 223.1595 | 3.479183 |
| Rcor2 | 23 | 23 | 9 | 13 | 24 | 76.75596 | 46.23541 | 10.24549 | 19.87037 | 0.244862 |
| uc008gmj.1 | 329.995 | 437.242 | 273.559 | 267.482 | 254.427 | 1101.264 | 878.9593 | 311.4162 | 408.8435 | 0.363726 |
| uc008gmk.1 | 27.9727 | 288.88 | 88.1985 | 145.324 | 34.5795 | 93.35093 | 580.7168 | 100.4041 | 222.1262 | 0.478484 |
| uc008gml.1 | 204.563 | 338.35 | 246.998 | 176.44 | 339.026 | 682.6708 | 680.1631 | 281.1795 | 269.6867 | 0.404206 |
| Bscl2 | 15 | 23 | 268.638 | 138 | 335 | 50.05823 | 46.23541 | 305.8142 | 210.9316 | 5.366354 |
| Asrgl1 | 12 | 6 | 63 | 29 | 45 | 40.04659 | 12.06141 | 71.71842 | 44.3262 | 2.227002 |
| Fth1 | 1443 | 2396 | 19685 | 15391 | 21235 | 4815.602 | 4816.524 | 22409.16 | 23524.98 | 4.768848 |
| Cybasc3 | 114.313 | 57.6628 | 103.855 | 37.6118 | 119 | 381.4871 | 115.9158 | 118.2272 | 57.48925 | 0.353268 |
| Stx3 | 12.2155 | 16.1696 | 104.878 | 82.0338 | 140.605 | 40.76576 | 32.5047 | 119.3918 | 125.3878 | 3.340768 |
| Ostf1 | 50 | 94 | 284 | 261 | 341 | 166.8608 | 188.9621 | 323.3021 | 398.9358 | 2.029768 |
| Aldh1a1 | 2 | 7 | 82 | 40.7143 | 77.9167 | 6.674431 | 14.07165 | 93.34779 | 62.23139 | 7.499209 |
| Gda | 29.3409 | 7 | 201 | 84.2003 | 214.159 | 97.91691 | 14.07165 | 228.8159 | 128.6993 | 3.192426 |
| Fam108b | 39.5172 | 41.0653 | 175.555 | 155 | 217.563 | 131.8774 | 82.55092 | 199.8496 | 236.9159 | 2.036884 |
| Tmem2 | 12.0275 | 17.0132 | 172.498 | 94.9125 | 174.7 | 40.13836 | 34.20054 | 196.3696 | 145.0728 | 4.593051 |
| Pip5k1b | 28 | 31 | 26 | 15 | 35 | 93.44204 | 62.31729 | 29.59808 | 22.92735 | 0.337222 |
| Kank1 | 12 | 11 | 87 | 40 | 80 | 40.04659 | 22.11259 | 99.03972 | 61.13959 | 2.576921 |
| Smarca2 | 15 | 19 | 154.103 | 59.8333 | 152.993 | 50.05823 | 38.19447 | 175.429 | 91.45458 | 3.024083 |
| Ppapdc2 | 12 | 13 | 74 | 56 | 96 | 40.04659 | 26.13306 | 84.24069 | 85.59542 | 2.566289 |
| Ak3 | 78 | 34 | 421.744 | 190.575 | 517.765 | 260.3028 | 68.348 | 480.1082 | 291.2919 | 2.347172 |
| 5033414D02Rik | 35 | 75 | 219 | 208 | 218 | 116.8025 | 150.7676 | 249.3069 | 317.9259 | 2.11994 |
| Ranbp6 | 9 | 6 | 49 | 34 | 50 | 30.03494 | 12.06141 | 55.78099 | 51.96865 | 2.559596 |
| Sgms1 | 29 | 42 | 336.882 | 368.909 | 390.949 | 96.77925 | 84.42988 | 383.5023 | 563.8736 | 5.22808 |
| Pten | 139.712 | 86 | 1259.99 | 427.836 | 1336.62 | 466.2491 | 172.8802 | 1434.357 | 653.9429 | 3.267414 |
| Fas | 3 | 2 | 124 | 90 | 141 | 10.01165 | 4.020471 | 141.1601 | 137.5641 | 19.8633 |
| Ch25h | 0 | 1 | 523.93 | 487.879 | 607.67 | 0 | 2.010235 | 596.4354 | 745.718 | 667.6599 |
| Lipa | 36 | 26 | 234 | 121 | 257.992 | 120.1398 | 52.26612 | 266.3827 | 184.9473 | 2.617834 |
| Ifit3 | 0 | 3 | 84.3176 | 49.1915 | 82.6875 | 0 | 6.030706 | 95.98611 | 75.1887 | 28.38388 |
| Ifit1 | 1 | 0 | 81 | 41.0477 | 116.218 | 3.337216 | 0 | 92.2094 | 62.74099 | 46.43104 |
| Hhex | 95 | 71 | 1249 | 875 | 1338 | 317.0355 | 142.7267 | 1421.846 | 1337.428 | 6.001526 |
| Exoc6 | 23 | 22 | 107 | 93 | 110 | 76.75596 | 44.22518 | 121.8075 | 142.1495 | 2.181803 |
| Rbp4 | 378 | 598 | 338 | 267 | 381 | 1261.468 | 1202.121 | 384.775 | 408.1067 | 0.32184 |
| Plce1 | 50.1295 | 34 | 4 | 0 | 5 | 167.293 | 68.348 | 4.553551 | 0 | 0.019324 |
| Noc3l | 27 | 27 | 29 | 15 | 21 | 90.10482 | 54.27635 | 33.01324 | 22.92735 | 0.387451 |
| Marveld1 | 30 | 26 | 185 | 82 | 251 | 100.1165 | 52.26612 | 210.6017 | 125.3362 | 2.204569 |
| 5330431N19Rik | 22.0864 | 45.7258 | 165.864 | 106.869 | 205.508 | 73.70708 | 91.91962 | 188.8175 | 163.3482 | 2.126262 |
| Npm3 | 51.4142 | 326.791 | 114.134 | 113.414 | 155.348 | 171.5803 | 656.9268 | 129.9287 | 173.3521 | 0.366057 |
| Pprc1 | 68.4174 | 61.4443 | 73.6494 | 48.1719 | 131.655 | 228.3236 | 123.5175 | 83.84157 | 73.63025 | 0.447565 |
| uc008hss.1 | 5 | 12 | 64 | 26.2016 | 42 | 16.68608 | 24.12282 | 72.85681 | 40.04887 | 2.766693 |
| D19Wsu162e | 70 | 54.0112 | 495.637 | 239 | 559.703 | 233.6051 | 108.5752 | 564.227 | 365.309 | 2.716509 |
| As3mt | 7 | 5 | 52 | 32 | 44 | 23.36051 | 10.05118 | 59.19616 | 48.91167 | 3.235629 |
| Obfc1 | 111 | 105 | 123 | 89 | 140 | 370.4309 | 211.0747 | 140.0217 | 136.0356 | 0.474728 |
| Slk | 83 | 57 | 84 | 41 | 103 | 276.9889 | 114.5834 | 95.62456 | 62.66808 | 0.404249 |
| Gsto1 | 304 | 532 | 3493 | 2518 | 4060 | 1014.514 | 1069.445 | 3976.388 | 3848.737 | 3.754933 |
| Add3 | 39 | 24 | 179 | 114 | 229 | 130.1514 | 48.24565 | 203.7714 | 174.2478 | 2.118977 |
| Pdcd4 | 17.0488 | 15.6391 | 109.645 | 90.883 | 140.385 | 56.89552 | 31.43827 | 124.8185 | 138.9137 | 2.985632 |
| Casp7 | 4 | 4 | 92.1619 | 52.1543 | 89.79 | 13.34886 | 8.040941 | 104.916 | 79.71731 | 8.631836 |
| 9930023K05Rik | 1 | 0 | 211 | 113 | 266 | 3.337216 | 0 | 240.1998 | 172.7193 | 123.7316 |
| Nhlrc2 | 90 | 59 | 88 | 58 | 89 | 300.3494 | 118.6039 | 100.1781 | 88.6524 | 0.45072 |
| 4930506M07Rik | 24 | 18.0731 | 190.695 | 86 | 243.528 | 80.09318 | 36.33118 | 217.0848 | 131.4501 | 2.99366 |
| Rab11fip2 | 15 | 15 | 75 | 56.129 | 81 | 50.05823 | 30.15353 | 85.37907 | 85.7926 | 2.133997 |
| Csf2ra | 13 | 4 | 58 | 42 | 104 | 43.3838 | 8.040941 | 66.02648 | 64.19657 | 2.532303 |
| Fam171a1 | 57 | 50 | 42 | 21 | 44 | 190.2213 | 100.5118 | 47.81228 | 32.09828 | 0.274859 |
| Nmt2 | 50 | 44 | 8.02873 | 11.0055 | 15 | 166.8608 | 88.45035 | 9.139807 | 16.82179 | 0.101686 |
| Suv39h2 | 17 | 39 | 31 | 14 | 36 | 56.73267 | 78.39918 | 35.29002 | 21.39886 | 0.419508 |
| Frmd4a | 58.1526 | 60.6402 | 47.6676 | 25.0816 | 56.6493 | 194.0678 | 121.9011 | 54.26421 | 38.33697 | 0.293071 |
| Phyh | 25 | 16 | 219 | 177 | 241 | 83.43039 | 32.16377 | 249.3069 | 270.5427 | 4.497196 |
| Ccdc3 | 0 | 1.00458 | 235.453 | 123.196 | 312.276 | 0 | 2.019442 | 268.0368 | 188.3038 | 225.9736 |
| Camk1d | 23 | 18.1452 | 34.0322 | 11 | 33.2867 | 76.75596 | 36.47612 | 38.74184 | 16.81339 | 0.490631 |
| Itih2 | 54 | 24 | 26 | 15 | 23 | 180.2096 | 48.24565 | 29.59808 | 22.92735 | 0.229916 |
| Itih5 | 23.2034 | 12.7486 | 13.0361 | 11.2218 | 12.033 | 77.43475 | 25.62769 | 14.84014 | 17.15241 | 0.310419 |
| Il15ra | 2 | 4 | 50.05 | 54.3897 | 56.8205 | 6.674431 | 8.040941 | 56.9763 | 83.13409 | 9.521362 |
| 2310047O13Rik | 48 | 61 | 352 | 236 | 391 | 160.1864 | 122.6244 | 400.7124 | 360.7236 | 2.692388 |
| Cubn | 28 | 39 | 2 | 0 | 0 | 93.44204 | 78.39918 | 2.276775 | 0 | 0.013249 |
| Vim | 2646 | 5650 | 2849 | 1819 | 3274 | 8830.273 | 11357.83 | 3243.266 | 2780.323 | 0.298373 |
| Ptpla | 90 | 173 | 91 | 80 | 96 | 300.3494 | 347.7707 | 103.5933 | 122.2792 | 0.348504 |
| Mrc1 | 45 | 75 | 0 | 1 | 0 | 150.1747 | 150.7676 | 0 | 1.52849 | 0.005079 |
| Commd3 | 68 | 132 | 665 | 540.938 | 716 | 226.9307 | 265.3511 | 757.0278 | 826.8181 | 3.217357 |
| Pdss1 | 12.1886 | 31.1273 | 13.9851 | 11.2303 | 14.5065 | 40.67599 | 62.5732 | 15.92046 | 17.1654 | 0.320447 |
| Zmynd19 | 68 | 127 | 83 | 58 | 98 | 226.9307 | 255.2999 | 94.48617 | 88.6524 | 0.379774 |
| Nrarp | 86 | 117 | 36 | 38 | 50 | 287.0005 | 235.1975 | 40.98195 | 58.08261 | 0.189707 |
| C430004E15Rik | 21 | 30 | 167 | 60 | 153 | 70.08153 | 60.30706 | 190.1107 | 91.70938 | 2.161386 |
| 2010317E24Rik | 13 | 73.3208 | 30.0111 | 14.0089 | 27.0176 | 43.3838 | 147.3921 | 34.16427 | 21.41246 | 0.291319 |
| Entpd2 | 9 | 3 | 72 | 32 | 75 | 30.03494 | 6.030706 | 81.96391 | 48.91167 | 3.628816 |
| Bmyc | 43 | 92 | 366 | 283 | 447 | 143.5003 | 184.9416 | 416.6499 | 432.5626 | 2.585579 |
| Qsox2 | 15 | 20 | 111 | 57 | 105 | 50.05823 | 40.20471 | 126.361 | 87.12391 | 2.365145 |
| Gpsm1 | 28.7222 | 16.691 | 34.0592 | 13.7375 | 38.6218 | 95.85217 | 33.55284 | 38.77257 | 20.99763 | 0.461885 |
| Fam69b | 28.5696 | 23.9141 | 35.4062 | 14.5775 | 22.4624 | 95.34292 | 48.07297 | 40.30598 | 22.28156 | 0.436406 |
| uc008ixj.1 | 8 | 0 | 40 | 49 | 78 | 26.69773 | 0 | 45.53551 | 74.89599 | 4.510927 |
| Gtf3c4 | 31 | 39 | 28 | 19 | 33 | 103.4537 | 78.39918 | 31.87485 | 29.0413 | 0.334975 |
| Ddx31 | 26 | 40 | 40 | 14 | 42 | 86.76761 | 80.40941 | 45.53551 | 21.39886 | 0.40038 |
| Trub2 | 55 | 159.466 | 110.286 | 61.2766 | 132.683 | 183.5469 | 320.5642 | 125.5482 | 93.66065 | 0.434842 |
| Set | 543.539 | 1059.19 | 641.329 | 387.604 | 610.579 | 1813.907 | 2129.221 | 730.081 | 592.4487 | 0.335401 |
| Pkn3 | 42.2745 | 60.6436 | 33.32 | 1.93222 | 37.9591 | 141.0791 | 121.9079 | 37.93108 | 2.953378 | 0.155462 |
| Phyhd1 | 126.886 | 26.1368 | 74.4503 | 41.0992 | 80.9616 | 423.4459 | 52.54112 | 84.7533 | 62.8197 | 0.310036 |
| Prrx2 | 99 | 179 | 11 | 14 | 18 | 330.3843 | 359.8321 | 12.52226 | 21.39886 | 0.049146 |
| Ptges | 71.2204 | 52.7876 | 9.04292 | 2.05605 | 3.01855 | 237.6778 | 106.1155 | 10.29435 | 3.142651 | 0.039085 |
| Freq | 118 | 78 | 72 | 25.1667 | 77.0376 | 393.7914 | 156.7984 | 81.96391 | 38.46704 | 0.218731 |
| Ass1 | 6 | 2 | 117 | 77.9994 | 138 | 20.02329 | 4.020471 | 133.1914 | 119.2213 | 10.49805 |
| Exosc2 | 44.6145 | 434.673 | 80.3406 | 125.654 | 114.505 | 148.8882 | 873.795 | 91.45875 | 192.0608 | 0.277231 |
| Aif1l | 73 | 65 | 12 | 11 | 11 | 243.6167 | 130.6653 | 13.66065 | 16.81339 | 0.08142 |
| 1110008P14Rik | 36 | 139 | 726 | 639 | 749 | 120.1398 | 279.4227 | 826.4694 | 976.7049 | 4.512872 |
| Lcn2 | 8 | 1 | 32326 | 22908 | 37983 | 26.69773 | 2.010235 | 36799.52 | 35014.64 | 2501.542 |
| St6galnac4 | 38.5234 | 35.3051 | 21.6665 | 14.1047 | 28.5447 | 128.5609 | 70.97156 | 24.66488 | 21.55889 | 0.23166 |
| St6galnac6 | 35.0847 | 33.0646 | 43.35 | 14.1111 | 71.282 | 117.0852 | 66.46763 | 49.3491 | 21.56867 | 0.386362 |
| Ak1 | 32.4189 | 18 | 175.29 | 77.958 | 191 | 108.1889 | 36.18424 | 199.548 | 119.158 | 2.207516 |
| Sh2d3c | 27 | 15 | 22 | 16 | 35 | 90.10482 | 30.15353 | 25.04453 | 24.45583 | 0.411617 |
| Ptrh1 | 38 | 108.138 | 59.6735 | 53.36 | 69.6604 | 126.8142 | 217.3828 | 67.93157 | 81.56021 | 0.43432 |
| uc008jhl.1 | 8 | 2 | 57 | 43 | 58.0762 | 26.69773 | 4.020471 | 64.8881 | 65.72506 | 4.25198 |
| Angptl2 | 74 | 33 | 10 | 8 | 17 | 246.954 | 66.33777 | 11.38388 | 12.22792 | 0.075367 |
| Rabepk | 80 | 110 | 84.0371 | 69.1534 | 120.7 | 266.9773 | 221.1259 | 95.6668 | 105.7003 | 0.41255 |
| Ggta1 | 55 | 72 | 75.9868 | 25 | 77 | 183.5469 | 144.7369 | 86.50243 | 38.21224 | 0.379899 |
| Ptgs1 | 20.2469 | 18.3183 | 227.419 | 95.081 | 308.907 | 67.56827 | 36.82409 | 258.891 | 145.3303 | 3.872135 |
| Zeb2 | 45.5533 | 17.0951 | 24.5903 | 16.7996 | 32.4331 | 152.0212 | 34.36517 | 27.99329 | 25.67802 | 0.287957 |
| Kif5c | 26 | 23 | 20.0065 | 7 | 22 | 86.76761 | 46.23541 | 22.77515 | 10.69943 | 0.251683 |
| Rbm43 | 10 | 6 | 71.106 | 39 | 76 | 33.37216 | 12.06141 | 80.94619 | 59.6111 | 3.093688 |
| Nmi | 3 | 10 | 83 | 68 | 107 | 10.01165 | 20.10235 | 94.48617 | 103.9373 | 6.589077 |
| Cd302 | 14 | 4 | 152 | 151 | 155 | 46.72102 | 8.040941 | 173.0349 | 230.8019 | 7.374405 |
| Itgb6 | 4 | 11 | 125.969 | 52.0784 | 115.94 | 13.34886 | 22.11259 | 143.4016 | 79.6013 | 6.288599 |
| Ifih1 | 2 | 2 | 84 | 42.0027 | 86.1515 | 6.674431 | 4.020471 | 95.62456 | 64.20069 | 14.94406 |
| Gca | 25 | 37 | 197.172 | 131.274 | 203.641 | 83.43039 | 74.37871 | 224.4582 | 200.651 | 2.693819 |
| Stk39 | 11 | 11 | 95 | 55 | 117 | 36.70937 | 22.11259 | 108.1468 | 84.06693 | 3.267721 |
| Sp5 | 3 | 0 | 202 | 178 | 244 | 10.01165 | 0 | 229.9543 | 272.0712 | 50.14414 |
| Itga6 | 27.7326 | 42.2197 | 14.9038 | 15.2696 | 7.36667 | 92.54967 | 84.87153 | 16.9663 | 23.33943 | 0.227175 |
| uc008kbg.1 | 415 | 458.825 | 163.411 | 81 | 211 | 1384.944 | 922.3462 | 186.0251 | 123.8077 | 0.134284 |
| Cdca7 | 136 | 169 | 113 | 53 | 118 | 453.8613 | 339.7298 | 128.6378 | 81.00995 | 0.264176 |
| Wipf1 | 64 | 89 | 6 | 5.83333 | 5 | 213.5818 | 178.9109 | 6.830326 | 8.916185 | 0.040119 |
| Chrna1 | 7.38008 | 65.4306 | 1.61807 | 0 | 0 | 24.62892 | 131.5309 | 1.841991 | 0 | 0.011796 |
| Osbpl6 | 36 | 25 | 5 | 2 | 6 | 120.1398 | 50.25588 | 5.691938 | 3.056979 | 0.051345 |
| Dnajc10 | 147 | 211 | 1531 | 880 | 1712 | 490.5707 | 424.1597 | 1742.871 | 1345.071 | 3.375795 |
| Itgav | 238.867 | 182 | 1406.73 | 859.864 | 1316.88 | 797.1507 | 365.8628 | 1601.404 | 1314.293 | 2.507019 |
| Timm10 | 138 | 289 | 161 | 171 | 179 | 460.5358 | 580.958 | 183.2804 | 261.3717 | 0.426937 |
| Slc43a3 | 303.342 | 277.969 | 118.543 | 85.4984 | 146.887 | 1012.318 | 558.7831 | 134.9479 | 130.6834 | 0.169073 |
| Ptprj | 42.9311 | 27 | 369.568 | 172.69 | 392.99 | 143.2703 | 54.27635 | 420.7116 | 263.9549 | 3.465847 |
| Ckap5 | 115 | 205 | 191 | 100 | 213 | 383.7798 | 412.0982 | 217.432 | 152.849 | 0.465248 |
| Harbi1 | 39.5022 | 309.341 | 75.3599 | 139.792 | 90.4503 | 131.8274 | 621.8482 | 85.78878 | 213.6706 | 0.397332 |
| Creb3l1 | 23 | 7 | 186 | 83 | 208 | 76.75596 | 14.07165 | 211.7401 | 126.8646 | 3.727994 |
| Slc35c1 | 17 | 21 | 156 | 78 | 204 | 56.73267 | 42.21494 | 177.5885 | 119.2222 | 2.999675 |
| Syt13 | 9 | 35 | 2 | 3.0113 | 4 | 30.03494 | 70.35824 | 2.276775 | 4.602741 | 0.068526 |
| Pamr1 | 17 | 7 | 469 | 249 | 531 | 56.73267 | 14.07165 | 533.9038 | 380.5939 | 12.91585 |
| uc008lid.1 | 9 | 3 | 94.8215 | 46.2706 | 111 | 30.03494 | 6.030706 | 107.9436 | 70.72413 | 4.95396 |
| Cd44 | 707.408 | 679.359 | 4029.27 | 2288.31 | 4518.87 | 2360.773 | 1365.671 | 4586.871 | 3497.658 | 2.169502 |
| Ehf | 11 | 5.01778 | 766.98 | 315.755 | 807.401 | 36.70937 | 10.08692 | 873.1206 | 482.6283 | 28.97129 |
| Cd59b | 99.7252 | 161.782 | 7.3479 | 24.6156 | 25.8127 | 332.8045 | 325.2199 | 8.364759 | 37.62469 | 0.06989 |
| Cd59a | 39.1924 | 50.2263 | 34.0745 | 28.6651 | 44.894 | 130.7935 | 100.9667 | 38.78999 | 43.81431 | 0.356421 |
| Tcp11l1 | 12.2795 | 47.6442 | 12.1178 | 6.02672 | 21.0309 | 40.97934 | 95.77605 | 13.79475 | 9.211779 | 0.168231 |
| Depdc7 | 26 | 45 | 21 | 9 | 20 | 86.76761 | 90.46059 | 23.90614 | 13.75641 | 0.212509 |
| Rcn1 | 6.07859 | 6 | 147 | 96 | 168.155 | 20.28557 | 12.06141 | 167.343 | 146.735 | 9.709655 |
| Kif18a | 31.0319 | 73.3339 | 38.0491 | 26.1428 | 40.1466 | 103.5601 | 147.4184 | 43.31463 | 39.959 | 0.331796 |
| Bdnf | 89 | 85 | 11.0092 | 12.0845 | 22.0779 | 297.0122 | 170.87 | 12.53274 | 18.47103 | 0.066264 |
| Fmn1 | 31.2422 | 22 | 28 | 8 | 30 | 104.262 | 44.22518 | 31.87485 | 12.22792 | 0.297014 |
| Arhgap11a | 44 | 130 | 88 | 63 | 96 | 146.8375 | 261.3306 | 100.1781 | 96.29485 | 0.481353 |
| Meis2 | 19.1895 | 39.4268 | 169.93 | 109.389 | 199.837 | 64.0395 | 79.25715 | 193.4462 | 167.2 | 2.51678 |
| Bmf | 21 | 2 | 228 | 100 | 256 | 70.08153 | 4.020471 | 259.5524 | 152.849 | 5.56532 |
| Bub1b | 59 | 140 | 80 | 40 | 101 | 196.8957 | 281.4329 | 91.07101 | 61.13959 | 0.318213 |
| D2Ertd750e | 36 | 237 | 62 | 54 | 94 | 120.1398 | 476.4258 | 70.58003 | 82.53844 | 0.256667 |
| Rad51 | 23 | 129 | 78 | 45 | 61.9217 | 76.75596 | 259.3204 | 88.79424 | 68.78204 | 0.468871 |
| Gchfr | 77 | 199 | 56 | 55 | 59 | 256.9656 | 400.0368 | 63.74971 | 84.06693 | 0.224986 |
| Dnajc17 | 22 | 52 | 37 | 18 | 24 | 73.41874 | 104.5322 | 42.12034 | 27.51281 | 0.391305 |
| Gm14137 | 29.0383 | 53 | 17.5854 | 7 | 9 | 96.90707 | 106.5425 | 20.019 | 10.69943 | 0.150988 |
| Spint1 | 5 | 1 | 176 | 113 | 205 | 16.68608 | 2.010235 | 200.3562 | 172.7193 | 19.9545 |
| Oip5 | 11.8203 | 82.9174 | 15.2271 | 21.4019 | 39.4911 | 39.44689 | 166.6835 | 17.33434 | 32.71258 | 0.242793 |
| Nusap1 | 64.691 | 276.385 | 98 | 88.6602 | 113 | 215.8878 | 555.5989 | 111.562 | 135.5162 | 0.320262 |
| Trp53bp1 | 90.6673 | 32.5658 | 102.482 | 28.1815 | 103.064 | 302.5763 | 65.46492 | 116.6642 | 43.07513 | 0.434026 |
| 2310003F16Rik | 333.365 | 1122.98 | 547.183 | 579.263 | 556 | 1112.511 | 2257.454 | 622.9064 | 885.3975 | 0.447573 |
| Casc4 | 34.353 | 16.0741 | 154.304 | 91.0721 | 134.297 | 114.6434 | 32.31272 | 175.6578 | 139.2028 | 2.142548 |
| B2m | 720.928 | 1228.53 | 4274.45 | 4215.94 | 4755.83 | 2405.892 | 2469.634 | 4865.981 | 6444.021 | 2.31975 |
| Sqrdl | 92 | 64 | 435.806 | 265 | 427 | 307.0238 | 128.6551 | 496.1162 | 405.0498 | 2.068418 |
| Sema6d | 27.2427 | 22 | 29 | 11 | 18.0051 | 90.91476 | 44.22518 | 33.01324 | 16.81339 | 0.368704 |
| Dut | 199 | 667.412 | 310.098 | 363.55 | 372.548 | 664.1059 | 1341.655 | 353.0117 | 555.6824 | 0.453042 |
| Galk2 | 16 | 30 | 156 | 102 | 168 | 53.39545 | 60.30706 | 177.5885 | 155.9059 | 2.933044 |
| Fgf7 | 12 | 11.1929 | 45 | 62.046 | 54.1542 | 40.04659 | 22.50036 | 51.22744 | 94.83667 | 2.335272 |
| Mal | 2 | 29 | 142 | 163.075 | 240 | 6.674431 | 58.29682 | 161.651 | 249.2585 | 6.324481 |
| Mall | 17 | 22 | 9 | 6 | 18.0379 | 56.73267 | 44.22518 | 10.24549 | 9.170938 | 0.192322 |
| uc008mgc.1 | 146 | 329 | 213 | 211 | 188 | 487.2335 | 661.3674 | 242.4766 | 322.5113 | 0.491892 |
| Bub1 | 30 | 147 | 91 | 29 | 73 | 100.1165 | 295.5046 | 103.5933 | 44.3262 | 0.373892 |
| Bcl2l11 | 75 | 47.3762 | 86.8242 | 41.1772 | 92.4868 | 250.2912 | 95.23731 | 98.8396 | 62.93892 | 0.468206 |
| uc008mgo.1 | 52.6617 | 135.636 | 27.1152 | 27.7664 | 22.5017 | 175.7434 | 272.6603 | 30.86761 | 42.44066 | 0.163487 |
| uc008mgp.1 | 15.3927 | 28.5574 | 1.5406 | 0 | 0 | 51.36876 | 57.40709 | 1.7538 | 0 | 0.016123 |
| Zc3h8 | 13 | 28.8229 | 17 | 7 | 31 | 43.3838 | 57.94081 | 19.35259 | 10.69943 | 0.296591 |
| Slc20a1 | 99 | 126 | 99 | 41 | 155 | 330.3843 | 253.2896 | 112.7004 | 62.66808 | 0.300456 |
| Sirpa | 1 | 1.00654 | 166 | 83 | 211 | 3.337216 | 2.023382 | 188.9723 | 126.8646 | 58.91824 |
| Snrpb | 478 | 2041.99 | 1078.84 | 955.999 | 1026 | 1595.189 | 4104.88 | 1228.138 | 1461.235 | 0.471814 |
| Nop56 | 348 | 752 | 527 | 339 | 689 | 1161.351 | 1511.697 | 599.9303 | 518.158 | 0.418282 |
| Fastkd5 | 30.625 | 48.7059 | 46.9293 | 22.5735 | 42.5316 | 102.2022 | 97.91032 | 53.42373 | 34.50336 | 0.439388 |
| Slc4a11 | 12 | 9 | 205 | 90 | 205 | 40.04659 | 18.09212 | 233.3695 | 137.5641 | 6.380148 |
| Cdc25b | 22 | 54 | 34 | 9 | 42 | 73.41874 | 108.5527 | 38.70518 | 13.75641 | 0.288296 |
| Mcm8 | 13.1393 | 30.5807 | 18.1409 | 10.3588 | 28.3935 | 43.84868 | 61.4744 | 20.65138 | 15.83332 | 0.346407 |
| Jag1 | 184.289 | 146.685 | 188.142 | 87.1344 | 267.979 | 615.0121 | 294.8714 | 214.1785 | 133.184 | 0.381766 |
| Btbd3 | 107.308 | 138 | 126.138 | 63.0719 | 116.115 | 358.1099 | 277.4125 | 143.5939 | 96.40475 | 0.37764 |
| Esf1 | 156.509 | 87 | 172 | 93 | 225 | 522.3043 | 174.8905 | 195.8027 | 142.1495 | 0.484731 |
| 8430406I07Rik | 13 | 40 | 30 | 14 | 30 | 43.3838 | 80.40941 | 34.15163 | 21.39886 | 0.448736 |
| Dtd1 | 22 | 29 | 114 | 130 | 168 | 73.41874 | 58.29682 | 129.7762 | 198.7037 | 2.493857 |
| Ncrna00153 | 13 | 6 | 51 | 38 | 87 | 43.3838 | 12.06141 | 58.05777 | 58.08261 | 2.094687 |
| Thbd | 147 | 36 | 4 | 7.04666 | 3 | 490.5707 | 72.36847 | 4.553551 | 10.77075 | 0.027222 |
| Gins1 | 35 | 91 | 62 | 34 | 67 | 116.8025 | 182.9314 | 70.58003 | 51.96865 | 0.408858 |
| Sdcbp2 | 358 | 382 | 41 | 26 | 42 | 1194.723 | 767.9099 | 46.67389 | 39.74073 | 0.04403 |
| Mcts2 | 6 | 14 | 95.3862 | 116.229 | 121 | 20.02329 | 28.14329 | 108.5865 | 177.6548 | 5.942736 |
| Tpx2 | 60 | 199 | 99 | 61.4959 | 115 | 200.2329 | 400.0368 | 112.7004 | 93.99585 | 0.344339 |
| uc008nhr.1 | 30.6194 | 200.178 | 7.32555 | 29.4008 | 15.731 | 102.1835 | 402.4049 | 8.339316 | 44.93882 | 0.105587 |
| Ahcy | 407 | 755.357 | 573.864 | 363 | 663 | 1358.247 | 1518.445 | 653.2797 | 554.8418 | 0.419969 |
| Trp53inp2 | 47 | 9 | 259 | 93 | 291 | 156.8491 | 18.09212 | 294.8424 | 142.1495 | 2.497935 |
| Procr | 159 | 309 | 84 | 68 | 146 | 530.6173 | 621.1627 | 95.62456 | 103.9373 | 0.173264 |
| uc008nna.1 | 39.8338 | 375.442 | 3.64084 | 12.8086 | 7.23881 | 132.934 | 754.7268 | 4.144687 | 19.57781 | 0.026725 |
| Tgif2 | 38.831 | 32 | 50.619 | 13.6857 | 51.7796 | 129.5874 | 64.32753 | 57.62404 | 20.91845 | 0.405036 |
| Dsn1 | 17 | 29 | 20 | 14 | 27 | 56.73267 | 58.29682 | 22.76775 | 21.39886 | 0.383959 |
| Samhd1 | 337.104 | 3284.47 | 485.642 | 911.917 | 689.206 | 1124.989 | 6602.558 | 552.8488 | 1393.856 | 0.251918 |
| Tgm2 | 153 | 39 | 760 | 299 | 817.113 | 510.594 | 78.39918 | 865.1746 | 457.0184 | 2.244836 |
| Lbp | 92.8059 | 16.0633 | 3467.12 | 1989.58 | 3794.89 | 309.7133 | 32.29101 | 3946.927 | 3041.052 | 20.43243 |
| uc008nqb.1 | 41.5 | 52.8819 | 28.5977 | 34.4253 | 57 | 138.4944 | 106.3051 | 32.55527 | 52.61872 | 0.347934 |
| Snhg11 | 33.9376 | 64.2977 | 13.8105 | 16.3472 | 32.2234 | 113.2571 | 129.2535 | 15.7217 | 24.98653 | 0.167862 |
| Fam83d | 7 | 80 | 31 | 17 | 48 | 23.36051 | 160.8188 | 35.29002 | 25.98432 | 0.332688 |
| Dhx35 | 14 | 8 | 73 | 30 | 77 | 46.72102 | 16.08188 | 83.1023 | 45.85469 | 2.05336 |
| Mybl2 | 21 | 55 | 36 | 15 | 46 | 70.08153 | 110.5629 | 40.98195 | 22.92735 | 0.353785 |
| Serinc3 | 437.693 | 281 | 1975.52 | 1220.85 | 2234.82 | 1460.676 | 564.8761 | 2248.908 | 1866.057 | 2.031527 |
| Ube2c | 179.831 | 1981.71 | 221.392 | 220.081 | 268.296 | 600.1348 | 3983.703 | 252.0299 | 336.3915 | 0.128369 |
| Snx21 | 30.0579 | 24.0518 | 29 | 12 | 33.0242 | 100.3097 | 48.34978 | 33.01324 | 18.34188 | 0.345455 |
| 2810408M09Rik | 24.7123 | 36.8292 | 26.9136 | 26.2536 | 43.2774 | 82.47027 | 74.03536 | 30.63811 | 40.12836 | 0.452166 |
| Eya2 | 21 | 32 | 25 | 10 | 49 | 70.08153 | 64.32753 | 28.45969 | 15.2849 | 0.325459 |
| Zmynd8 | 459.383 | 4187.1 | 198.288 | 155.835 | 285.692 | 1533.06 | 8417.056 | 225.7286 | 238.1922 | 0.046625 |
| Ncoa3 | 26 | 22 | 30 | 16 | 52 | 86.76761 | 44.22518 | 34.15163 | 24.45583 | 0.44741 |
| Sulf2 | 8 | 6 | 811 | 397 | 898 | 26.69773 | 12.06141 | 923.2324 | 606.8104 | 39.47567 |
| Znfx1 | 22 | 16.0325 | 214.157 | 91.2507 | 213.608 | 73.41874 | 32.2291 | 243.7937 | 139.4758 | 3.627802 |
| B4galt5 | 72 | 33.2554 | 53 | 19.0741 | 68.0364 | 240.2795 | 66.85118 | 60.33454 | 29.15456 | 0.291371 |
| Cebpb | 205.867 | 120.714 | 1098.71 | 946.529 | 932.568 | 687.0226 | 242.6635 | 1250.758 | 1446.76 | 2.901536 |
| Atp9a | 10 | 6 | 365 | 153 | 546 | 33.37216 | 12.06141 | 415.5115 | 233.8589 | 14.29275 |
| Zfp64 | 69.7438 | 74.027 | 102.934 | 44.8549 | 146.614 | 232.7501 | 148.8117 | 117.1788 | 68.56025 | 0.486786 |
| Aurka | 40 | 277 | 82 | 62 | 84 | 133.4886 | 556.8352 | 93.34779 | 94.76636 | 0.272501 |
| Rbm38 | 68 | 98 | 95 | 57 | 131 | 226.9307 | 197.0031 | 108.1468 | 87.12391 | 0.460616 |
| Zbp1 | 1 | 0 | 61 | 54.1649 | 84 | 3.337216 | 0 | 69.44165 | 82.79049 | 45.61651 |
| Etohi1 | 54.3304 | 45.3482 | 260.733 | 179.285 | 343.07 | 181.3123 | 91.16055 | 296.8152 | 274.0353 | 2.095073 |
| uc008ofy.1 | 2.13955 | 4.62975 | 35.6377 | 71.1744 | 2.74678 | 7.14014 | 9.306887 | 40.56952 | 108.7893 | 9.081207 |
| uc008ogs.1 | 0.958944 | 12.2912 | 20.7951 | 52.68 | 39.7607 | 3.200203 | 24.7082 | 23.67288 | 80.52084 | 3.733417 |
| uc008ogw.1 | 2.13955 | 4.62975 | 35.6377 | 71.1744 | 2.74678 | 7.14014 | 9.306887 | 40.56952 | 108.7893 | 9.081207 |
| uc008ogz.1 | 16.2534 | 6.0981 | 133 | 100.971 | 191.966 | 54.2411 | 12.25862 | 151.4056 | 154.3331 | 4.597594 |
| Gm14403 | 5.78233 | 0 | 57.567 | 34.5671 | 79.2341 | 19.29688 | 0 | 65.53356 | 52.83546 | 6.134101 |
| uc008ohf.1 | 8.45405 | 0 | 50.7649 | 56.6427 | 39.4928 | 28.21299 | 0 | 57.79013 | 86.57778 | 5.117073 |
| uc008ohi.1 | 46.608 | 51.9855 | 39.0601 | 27.4448 | 17.9533 | 155.5409 | 104.5031 | 44.46553 | 41.94909 | 0.332308 |
| Gata5 | 31.1883 | 55.1276 | 0 | 1.00137 | 0 | 104.0821 | 110.8194 | 0 | 1.530584 | 0.007122 |
| Slco4a1 | 24 | 18 | 14 | 7 | 27 | 80.09318 | 36.18424 | 15.93743 | 10.69943 | 0.22908 |
| 2700038C09Rik | 137 | 422 | 232 | 220 | 314 | 457.1985 | 848.3193 | 264.1059 | 336.2677 | 0.459874 |
| BC006779 | 30 | 10 | 174 | 77.7491 | 185 | 100.1165 | 20.10235 | 198.0794 | 118.8387 | 2.636177 |
| Rtel1 | 43.0247 | 87 | 82 | 40 | 92 | 143.5827 | 174.8905 | 93.34779 | 61.13959 | 0.485088 |
| Fam164a | 61.7531 | 37 | 322 | 199 | 317.264 | 206.0834 | 74.37871 | 366.5608 | 304.1694 | 2.391518 |
| Fabp5 | 178 | 1225 | 374 | 431 | 421 | 594.0244 | 2462.538 | 425.757 | 658.779 | 0.354822 |
| Zfand1 | 40 | 34 | 193 | 135 | 181 | 133.4886 | 68.348 | 219.7088 | 206.3461 | 2.11089 |
| Car2 | 26 | 76 | 866 | 773 | 1010 | 86.76761 | 152.7779 | 985.8437 | 1181.523 | 9.047827 |
| Pde7a | 12 | 14 | 120 | 52 | 116 | 40.04659 | 28.14329 | 136.6065 | 79.48146 | 3.168916 |
| Cp | 48.1468 | 4 | 6759.18 | 3049.44 | 8379.84 | 160.6763 | 8.040941 | 7694.567 | 4661.038 | 73.23263 |
| Hps3 | 21.8718 | 6.19737 | 102.077 | 50.7458 | 107.214 | 72.99091 | 12.45817 | 116.2032 | 77.56443 | 2.267638 |
| Gyg | 34 | 36 | 241 | 114 | 217 | 113.4653 | 72.36847 | 274.3514 | 174.2478 | 2.413981 |
| Aadacl1 | 10 | 12 | 90.8108 | 42 | 88 | 33.37216 | 24.12282 | 103.3779 | 64.19657 | 2.914593 |
| Fndc3b | 156.794 | 89 | 1124.22 | 700.893 | 1202.05 | 523.2554 | 178.9109 | 1279.798 | 1071.308 | 3.34836 |
| Pld1 | 31 | 8 | 222 | 127 | 248 | 103.4537 | 16.08188 | 252.7221 | 194.1182 | 3.738136 |
| Zmat3 | 25.1886 | 19.7325 | 652.359 | 239.428 | 653.871 | 84.05979 | 39.66697 | 742.6374 | 365.9632 | 8.960072 |
| Mfn1 | 20.0714 | 15.3091 | 147.645 | 54.0536 | 151.914 | 66.98259 | 30.77489 | 168.0772 | 82.62037 | 2.564485 |
| Gnb4 | 24 | 34 | 2.01089 | 1 | 6.00178 | 80.09318 | 68.348 | 2.289172 | 1.52849 | 0.025718 |
| 4932438A13Rik | 18.0348 | 15.1 | 115.991 | 59.6612 | 111.183 | 60.18602 | 30.35455 | 132.0427 | 91.19153 | 2.465572 |
| Spry1 | 33 | 28 | 186 | 152 | 211 | 110.1281 | 56.28659 | 211.7401 | 232.3304 | 2.668457 |
| Plk4 | 59.1088 | 1190.05 | 100.53 | 142.011 | 118.4 | 197.2588 | 2392.281 | 114.4421 | 217.0623 | 0.128017 |
| Phf17 | 10.0123 | 24 | 77.2648 | 53 | 91.4859 | 33.4132 | 48.24565 | 87.95729 | 81.00995 | 2.069185 |
| Slc7a11 | 113.195 | 12.4955 | 1327.55 | 613.511 | 1335.49 | 377.7561 | 25.1189 | 1511.267 | 937.7452 | 6.078837 |
| Ccrn4l | 10.3763 | 35.9868 | 336.98 | 449.262 | 346.321 | 34.62795 | 72.34194 | 383.6139 | 686.6923 | 10.00568 |
| Rab33b | 179.006 | 5463.12 | 235.513 | 1070.79 | 212.623 | 597.3816 | 10982.16 | 268.1051 | 1636.691 | 0.164497 |
| Mgst2 | 78 | 335 | 56 | 47 | 44 | 260.3028 | 673.4288 | 63.74971 | 71.83901 | 0.145212 |
| Exosc8 | 96.1481 | 276.845 | 152.406 | 154.515 | 183.512 | 320.8669 | 556.5236 | 173.4971 | 236.1746 | 0.466921 |
| Dclk1 | 3.01948 | 2 | 49.4713 | 36.1311 | 64.2068 | 10.07666 | 4.020471 | 56.31752 | 55.22601 | 7.912501 |
| uc008pgt.1 | 9.5 | 10 | 54.2811 | 49 | 54.6418 | 31.70355 | 20.10235 | 61.79293 | 74.89599 | 2.638482 |
| uc008pgu.1 | 9.5 | 10 | 73.7189 | 49 | 91.3582 | 31.70355 | 20.10235 | 83.92068 | 74.89599 | 3.06561 |
| Tm4sf1 | 21 | 50 | 421 | 385 | 485 | 70.08153 | 100.5118 | 479.2612 | 588.4685 | 6.25892 |
| Tm4sf4 | 970 | 1671 | 556.161 | 321 | 536 | 3237.099 | 3359.103 | 633.1268 | 490.6452 | 0.170367 |
| Pfn2 | 3 | 5 | 70 | 21 | 41 | 10.01165 | 10.05118 | 79.68713 | 32.09828 | 5.571769 |
| 4631416L12Rik | 8 | 7 | 169 | 106 | 162 | 26.69773 | 14.07165 | 192.3875 | 162.0199 | 8.692982 |
| Slc33a1 | 36 | 45.1621 | 296.7 | 174 | 358 | 120.1398 | 90.78645 | 337.7596 | 265.9572 | 2.862218 |
| Mfsd1 | 74 | 67 | 410 | 242 | 443.001 | 246.954 | 134.6858 | 466.7389 | 369.8945 | 2.192207 |
| Schip1 | 56.7362 | 32 | 252.563 | 163.157 | 230.382 | 189.3409 | 64.32753 | 287.5146 | 249.3838 | 2.116536 |
| Trim59 | 114 | 292 | 233 | 128 | 223 | 380.4426 | 586.9887 | 265.2443 | 195.6467 | 0.476407 |
| 1110032A04Rik | 30 | 16 | 7 | 6 | 4 | 100.1165 | 32.16377 | 7.968713 | 9.170938 | 0.129571 |
| Serpini1 | 8 | 8 | 50 | 33 | 49.0534 | 26.69773 | 16.08188 | 56.91938 | 50.44016 | 2.509596 |
| Pdgfc | 31 | 22 | 202 | 107.134 | 202.636 | 103.4537 | 44.22518 | 229.9543 | 163.7532 | 2.665971 |
| Ctso | 10.0396 | 5.02395 | 146.295 | 97 | 169 | 33.50431 | 10.09932 | 166.5404 | 148.2635 | 7.219672 |
| Tlr2 | 6 | 4 | 127 | 66 | 124 | 20.02329 | 8.040941 | 144.5752 | 100.8803 | 8.746205 |
| Mnd1 | 16 | 67 | 31 | 34 | 49 | 53.39545 | 134.6858 | 35.29002 | 51.96865 | 0.463941 |
| Fhdc1 | 57 | 40 | 17 | 7.0367 | 16.2489 | 190.2213 | 80.40941 | 19.35259 | 10.75552 | 0.111252 |
| Lrba | 11 | 12.05 | 169 | 97.874 | 205 | 36.70937 | 24.22334 | 192.3875 | 149.5994 | 5.612534 |
| Cd1d1 | 2.00596 | 2.99971 | 49.9592 | 45.6 | 73.9583 | 6.694321 | 6.030123 | 56.87294 | 69.69913 | 9.947159 |
| uc008psp.1 | 58.1601 | 15.4755 | 51.1797 | 17.5278 | 92.2948 | 194.0928 | 31.1094 | 58.26234 | 26.79106 | 0.377676 |
| Pear1 | 98 | 84 | 111 | 50 | 109 | 327.0471 | 168.8598 | 126.361 | 76.42448 | 0.408919 |
| Hdgf | 379.556 | 668.614 | 675 | 311.487 | 796.628 | 1266.66 | 1344.071 | 768.4117 | 476.1047 | 0.476693 |
| Nes | 840 | 275.042 | 20.0149 | 7 | 27.0484 | 2803.261 | 552.8991 | 22.78471 | 10.69943 | 0.009977 |
| Pmf1 | 31 | 166 | 79 | 58 | 88 | 103.4537 | 333.6991 | 89.93262 | 88.6524 | 0.408519 |
| Mex3a | 100 | 68.7955 | 113 | 47 | 132.295 | 333.7216 | 138.2951 | 128.6378 | 71.83901 | 0.424724 |
| Arhgef2 | 41.1642 | 384.288 | 25.8969 | 51.6193 | 21.087 | 137.3738 | 772.5093 | 29.48071 | 78.89957 | 0.119115 |
| Rit1 | 21 | 27 | 126 | 96 | 138 | 70.08153 | 54.27635 | 143.4368 | 146.735 | 2.333361 |
| Syt11 | 11 | 11.7059 | 58.9945 | 40.1705 | 81.2371 | 36.70937 | 23.53161 | 67.15861 | 61.40019 | 2.134075 |
| Thbs3 | 20 | 41 | 33 | 11 | 32 | 66.74431 | 82.41965 | 37.56679 | 16.81339 | 0.364566 |
| Muc1 | 9 | 55 | 213 | 94 | 252 | 30.03494 | 110.5629 | 242.4766 | 143.678 | 2.746518 |
| Cks1b | 371.078 | 1317.68 | 626.622 | 580.86 | 708.841 | 1238.367 | 2648.847 | 713.3387 | 887.8385 | 0.411909 |
| 1700094D03Rik | 25.6041 | 27.8581 | 35.8304 | 17.7114 | 39.0637 | 85.4464 | 56.00134 | 40.78888 | 27.07169 | 0.479757 |
| Ilf2 | 95.4809 | 371.974 | 197.545 | 195.014 | 253.822 | 318.6404 | 747.7553 | 224.8828 | 298.0769 | 0.490399 |
| S100a1 | 159 | 336 | 247 | 203 | 285 | 530.6173 | 675.4391 | 281.1817 | 310.2834 | 0.490413 |
| S100a13 | 183 | 1289 | 143 | 174 | 164 | 610.7105 | 2591.193 | 162.7894 | 265.9572 | 0.133904 |
| S100a14 | 6 | 4 | 43 | 36 | 63 | 20.02329 | 8.040941 | 48.95067 | 55.02563 | 3.70494 |
| S100a3 | 15 | 41 | 9 | 4 | 9 | 50.05823 | 82.41965 | 10.24549 | 6.113959 | 0.123488 |
| S100a6 | 13885 | 78068 | 19065 | 26912 | 20084 | 46337.24 | 156935.1 | 21703.36 | 41134.71 | 0.309133 |
| S100a7a | 64 | 280 | 0 | 1 | 0 | 213.5818 | 562.8659 | 0 | 1.52849 | 0.001969 |
| Sprr2a | 25.6064 | 32.5828 | 6403.95 | 4488.48 | 6085.15 | 85.45408 | 65.4991 | 7290.178 | 6860.595 | 93.7428 |
| uc008qdq.1 | 2.52706 | 67.5148 | 688.64 | 1056.38 | 663.847 | 8.433344 | 135.7206 | 783.9393 | 1614.666 | 16.63919 |
| Sprr1a | 655 | 2296 | 294 | 164 | 363 | 2185.876 | 4615.5 | 334.686 | 250.6723 | 0.086065 |
| Rorc | 16 | 2 | 116.011 | 58.0382 | 99.008 | 53.39545 | 4.020471 | 132.0655 | 88.71079 | 3.84521 |
| Tdrkh | 41.8365 | 42.9395 | 31.7936 | 35.0116 | 46.7998 | 139.6174 | 86.3185 | 36.19344 | 53.51487 | 0.397052 |
| Cgn | 24.0577 | 11.0485 | 205.883 | 63.498 | 216.045 | 80.28573 | 22.21008 | 234.3747 | 97.05604 | 3.233602 |
| Selenbp1 | 16.9999 | 15.9996 | 480 | 329 | 483.997 | 56.73233 | 32.16296 | 546.4261 | 502.8731 | 11.80377 |
| Adamtsl4 | 13 | 9 | 85 | 45 | 115 | 43.3838 | 18.09212 | 96.76295 | 68.78204 | 2.692843 |
| Plekho1 | 33 | 48 | 18 | 6 | 31 | 110.1281 | 96.4913 | 20.49098 | 9.170938 | 0.143558 |
| Txnip | 145 | 62 | 1814 | 1140.91 | 1774.98 | 483.8963 | 124.6346 | 2065.035 | 1743.869 | 6.25918 |
| uc008qnj.1 | 928.85 | 6310.56 | 1803.57 | 2719.06 | 2491.08 | 3099.773 | 12685.71 | 2053.162 | 4156.055 | 0.39335 |
| Pde4dip | 113.934 | 68 | 136.556 | 62 | 192 | 380.2223 | 136.696 | 155.4537 | 94.76636 | 0.484061 |
| Notch2 | 45.6522 | 28.0155 | 280.221 | 141.038 | 349.644 | 152.3512 | 56.31775 | 319.0001 | 215.5751 | 2.561834 |
| Atp1a1 | 352 | 433 | 3075 | 1983 | 3715 | 1174.7 | 870.4319 | 3500.542 | 3030.995 | 3.1937 |
| BC037703 | 0 | 5 | 80.8519 | 42.7088 | 70 | 0 | 10.05118 | 92.0408 | 65.27996 | 15.65197 |
| Ngf | 17 | 64 | 248 | 238 | 313 | 56.73267 | 128.6551 | 282.3201 | 363.7805 | 3.485132 |
| Tspan2 | 83 | 65 | 40 | 40 | 54 | 276.9889 | 130.6653 | 45.53551 | 61.13959 | 0.26168 |
| Nr1h5 | 71 | 76.2655 | 4.02019 | 0 | 7.04389 | 236.9423 | 153.3116 | 4.576535 | 0 | 0.011727 |
| Bcl2l15 | 53.3927 | 90 | 7 | 9 | 10.0301 | 178.183 | 180.9212 | 7.968713 | 13.75641 | 0.060498 |
| Ptpn22 | 72 | 59 | 5 | 2 | 2 | 240.2795 | 118.6039 | 5.691938 | 3.056979 | 0.024378 |
| Phtf1 | 22 | 22 | 132.948 | 67 | 170.994 | 73.41874 | 44.22518 | 151.3464 | 102.4088 | 2.156976 |
| Csf1 | 17 | 15 | 1190 | 558.605 | 1268.99 | 56.73267 | 30.15353 | 1354.681 | 853.822 | 25.41834 |
| Gstm5 | 74.9022 | 73.849 | 839.838 | 700.91 | 912.917 | 249.9648 | 148.4539 | 956.0612 | 1071.334 | 5.088604 |
| Gstm1 | 117.983 | 81.75 | 591.803 | 329.285 | 679.768 | 393.7347 | 164.3367 | 673.7012 | 503.3087 | 2.109067 |
| Sort1 | 13 | 5 | 256 | 110 | 308 | 43.3838 | 10.05118 | 291.4272 | 168.1339 | 8.600379 |
| Psrc1 | 13 | 24 | 634 | 343 | 722 | 43.3838 | 48.24565 | 721.7378 | 524.272 | 13.59835 |
| Celsr2 | 5.02267 | 6.26808 | 69.5811 | 20.6894 | 70.4064 | 16.76173 | 12.60032 | 79.21026 | 31.62353 | 3.77473 |
| Dph5 | 22.2557 | 69.7649 | 38.0955 | 31.4108 | 52.1535 | 74.27207 | 140.2439 | 43.36745 | 48.01108 | 0.425975 |
| Vcam1 | 35 | 20 | 432 | 249 | 474.913 | 116.8025 | 40.20471 | 491.7835 | 380.5939 | 5.556287 |
| Slc35a3 | 8 | 11 | 60 | 37.9487 | 71 | 26.69773 | 22.11259 | 68.30326 | 58.0042 | 2.587721 |
| Palmd | 24 | 11 | 279 | 194 | 350 | 80.09318 | 22.11259 | 317.6102 | 296.527 | 6.008831 |
| Snx7 | 28 | 65 | 314 | 251 | 315 | 93.44204 | 130.6653 | 357.4537 | 383.6509 | 3.306918 |
| Slc44a3 | 1 | 2 | 89.3196 | 44 | 117 | 3.337216 | 4.020471 | 101.6803 | 67.25355 | 22.96019 |
| F3 | 199 | 573 | 164 | 111 | 172 | 664.1059 | 1151.865 | 186.6956 | 169.6624 | 0.196235 |
| Dnttip2 | 157 | 181 | 190.984 | 144 | 228 | 523.9429 | 363.8526 | 217.4138 | 220.1025 | 0.492812 |
| Bcar3 | 110 | 126 | 51 | 40 | 48 | 367.0937 | 253.2896 | 58.05777 | 61.13959 | 0.192135 |
| uc008rfn.1 | 34.4075 | 138.127 | 69.8914 | 73.7906 | 51.7131 | 114.8252 | 277.6678 | 79.56351 | 112.7882 | 0.490077 |
| Tifa | 13 | 7 | 170 | 91 | 173 | 43.3838 | 14.07165 | 193.5259 | 139.0926 | 5.789154 |
| Cfi | 7 | 3 | 140 | 75 | 143 | 23.36051 | 6.030706 | 159.3743 | 114.6367 | 9.322888 |
| Hadh | 541.728 | 5050.43 | 838.093 | 1445.32 | 1040.19 | 1807.863 | 10152.55 | 954.0747 | 2209.157 | 0.264475 |
| Sgms2 | 26 | 15 | 14 | 12 | 17 | 86.76761 | 30.15353 | 15.93743 | 18.34188 | 0.293183 |
| Cenpe | 29 | 79 | 67 | 20 | 77 | 96.77925 | 158.8086 | 76.27197 | 30.56979 | 0.418024 |
| Nfkb1 | 65 | 74 | 440 | 265 | 483 | 216.919 | 148.7574 | 500.8906 | 405.0498 | 2.477437 |
| Ddit4l | 7.01219 | 9.02003 | 85.0438 | 42.0511 | 104.032 | 23.40119 | 18.13238 | 96.81281 | 64.27467 | 3.878488 |
| Adh7 | 6 | 8 | 42 | 37 | 42 | 20.02329 | 16.08188 | 47.81228 | 56.55412 | 2.890621 |
| Gbp6 | 1 | 8.01946 | 54.7394 | 39.4574 | 64.1055 | 3.337216 | 16.121 | 62.31466 | 60.31023 | 6.301959 |
| Gbp2 | 0 | 1 | 48 | 32 | 56 | 0 | 2.010235 | 54.64261 | 48.91167 | 51.51351 |
| Sh3glb1 | 490.917 | 550.939 | 579.86 | 344.848 | 614.368 | 1638.296 | 1107.517 | 660.1055 | 527.0966 | 0.432368 |
| Clca1 | 176.52 | 86.8315 | 4 | 1 | 1 | 589.0853 | 174.5517 | 4.553551 | 1.52849 | 0.007965 |
| Znhit6 | 64.083 | 67.9852 | 75.1484 | 49 | 114.21 | 213.8588 | 136.6662 | 85.54801 | 74.89599 | 0.457725 |
| Lphn2 | 11 | 4 | 360.514 | 188 | 380.224 | 36.70937 | 8.040941 | 410.4047 | 287.3561 | 15.59231 |
| Ifi44 | 0 | 1 | 114.387 | 93 | 155.71 | 0 | 2.010235 | 130.2167 | 142.1495 | 135.4898 |
| Gipc2 | 21 | 43 | 16 | 11 | 17 | 70.08153 | 86.44012 | 18.2142 | 16.81339 | 0.223788 |
| Fam73a | 80.9607 | 22.9375 | 1802.7 | 484.439 | 2117.86 | 270.1833 | 46.10977 | 2052.171 | 740.46 | 8.829252 |
| Ak5 | 62 | 21 | 4 | 7.03057 | 4 | 206.9074 | 42.21494 | 4.553551 | 10.74615 | 0.061414 |
| Cth | 115 | 30 | 72 | 56 | 75 | 383.7798 | 60.30706 | 81.96391 | 85.59542 | 0.377312 |
| Gpr177 | 68.8193 | 60 | 788.438 | 451 | 804.584 | 229.6648 | 120.6141 | 897.5481 | 689.3488 | 4.53038 |
| Tmem68 | 18 | 17 | 120 | 76 | 104 | 60.06988 | 34.174 | 136.6065 | 116.1652 | 2.682102 |
| Lyn | 14 | 11 | 163 | 79 | 185 | 46.72102 | 22.11259 | 185.5572 | 120.7507 | 4.449976 |
| uc008rwv.1 | 2.48652 | 103.463 | 21.8943 | 36.7461 | 8.97623 | 8.298053 | 207.985 | 24.9242 | 56.16603 | 0.374926 |
| Ubxn2b | 4 | 11.1652 | 51.1962 | 38.1915 | 58.0963 | 13.34886 | 22.44468 | 58.28112 | 58.37531 | 3.259148 |
| 2610301B20Rik | 17 | 13 | 88 | 56 | 103 | 56.73267 | 26.13306 | 100.1781 | 85.59542 | 2.241862 |
| Trp53inp1 | 52.4569 | 15 | 801.831 | 359 | 770.714 | 175.06 | 30.15353 | 912.7945 | 548.7278 | 7.121959 |
| Esrp1 | 0 | 2 | 107 | 44 | 125 | 0 | 4.020471 | 121.8075 | 67.25355 | 47.0246 |
| Cdh17 | 180 | 364 | 25 | 9 | 17 | 600.6988 | 731.7257 | 28.45969 | 13.75641 | 0.031684 |
| Ppm2c | 32.084 | 24 | 42 | 13.0061 | 38 | 107.0712 | 48.24565 | 47.81228 | 19.87969 | 0.435831 |
| Ripk2 | 6 | 4 | 59 | 38 | 63 | 20.02329 | 8.040941 | 67.16487 | 58.08261 | 4.462886 |
| Wwp1 | 18 | 14 | 102 | 59 | 114 | 60.06988 | 28.14329 | 116.1155 | 90.18089 | 2.338612 |
| Ggh | 60 | 49 | 333 | 306 | 322 | 200.2329 | 98.50153 | 379.0831 | 467.7178 | 2.834627 |
| uc008sdg.1 | 58.2262 | 44.3131 | 341.708 | 167.278 | 288.275 | 194.3134 | 89.07976 | 388.9962 | 255.6827 | 2.274857 |
| Coq3 | 9.05536 | 19.0583 | 59 | 51.0519 | 95.3181 | 30.21969 | 38.31167 | 67.16487 | 78.0323 | 2.118697 |
| 1810074P20Rik | 15.0811 | 7 | 155.449 | 74 | 158.69 | 50.32888 | 14.07165 | 176.9612 | 113.1082 | 4.504147 |
| Lyrm2 | 21.625 | 62.4753 | 158.128 | 164.019 | 123.41 | 72.16729 | 125.5901 | 180.011 | 250.7013 | 2.177984 |
| Pnrc1 | 110 | 55 | 779 | 497 | 932 | 367.0937 | 110.5629 | 886.804 | 759.6594 | 3.44696 |
| Ddx58 | 10.0915 | 4.20161 | 139.219 | 69.5416 | 181.761 | 33.67751 | 8.446225 | 158.4852 | 106.2936 | 6.285739 |
| uc008shj.1 | 21.3304 | 70.8682 | 189.845 | 199.464 | 183.133 | 71.18414 | 142.4618 | 216.1172 | 304.8787 | 2.438595 |
| B4galt1 | 155 | 78 | 1277.99 | 465.011 | 1648.82 | 517.2684 | 156.7984 | 1454.848 | 710.7645 | 3.212757 |
| Spink4 | 7 | 5 | 1047 | 1081 | 1079 | 23.36051 | 10.05118 | 1191.892 | 1652.297 | 85.12558 |
| Bag1 | 133 | 156 | 1448 | 713 | 1805 | 443.8497 | 313.5967 | 1648.385 | 1089.813 | 3.615039 |
| Unc13b | 27 | 20 | 146 | 83.095 | 190.078 | 90.10482 | 40.20471 | 166.2046 | 127.0098 | 2.250138 |
| Car9 | 9.00811 | 2.00157 | 61.027 | 34 | 69.5396 | 30.06201 | 4.023627 | 69.47238 | 51.96865 | 3.562822 |
| Tpm2 | 134 | 298 | 35 | 23 | 36 | 447.1869 | 599.0501 | 39.84357 | 35.15526 | 0.071684 |
| Hint2 | 47 | 86 | 287 | 279 | 275 | 156.8491 | 172.8802 | 326.7173 | 426.4486 | 2.284194 |
| Glipr2 | 20 | 1 | 79 | 37.0185 | 107 | 66.74431 | 2.010235 | 89.93262 | 56.58239 | 2.130987 |
| Tomm5 | 392 | 1357 | 618 | 567 | 552 | 1308.189 | 2727.889 | 703.5236 | 866.6536 | 0.389035 |
| Wdr32 | 16.3689 | 19.4013 | 98.3376 | 49.5072 | 124.124 | 54.62655 | 39.00118 | 111.9463 | 75.67124 | 2.003867 |
| 1300002K09Rik | 0 | 1 | 103 | 26 | 116 | 0 | 2.010235 | 117.2539 | 39.74073 | 78.09765 |
| Galnt12 | 13 | 5 | 52 | 43 | 92 | 43.3838 | 10.05118 | 59.19616 | 65.72506 | 2.337817 |
| Tgfbr1 | 49 | 46 | 727 | 327 | 784 | 163.5236 | 92.47082 | 827.6078 | 499.8161 | 5.185363 |
| Nipsnap3a | 34 | 62 | 233 | 143 | 226 | 113.4653 | 124.6346 | 265.2443 | 218.574 | 2.031997 |
| Tmem38b | 6 | 6 | 124 | 107 | 134 | 20.02329 | 12.06141 | 141.1601 | 163.5484 | 9.497 |
| D730040F13Rik | 360.052 | 1232 | 442.47 | 502.264 | 489.498 | 1201.571 | 2476.61 | 503.7024 | 767.7053 | 0.345662 |
| Akap2 | 121.581 | 216.812 | 49.704 | 38.5332 | 50.201 | 405.742 | 435.8431 | 56.58242 | 58.8976 | 0.137217 |
| Slc31a2 | 4 | 5 | 56.1583 | 51 | 64.3841 | 13.34886 | 10.05118 | 63.92991 | 77.95297 | 6.063361 |
| uc008teh.1 | 13.8125 | 4.5699 | 101.047 | 47.4198 | 95.5264 | 46.09529 | 9.186574 | 115.0307 | 72.48067 | 3.391914 |
| Slc31a1 | 55 | 66 | 360 | 214 | 443 | 183.5469 | 132.6755 | 409.8195 | 327.0968 | 2.330374 |
| Pole3 | 22 | 126 | 38 | 78 | 51 | 73.41874 | 253.2896 | 43.25873 | 119.2222 | 0.497327 |
| Ambp | 24 | 43 | 545 | 358 | 643 | 80.09318 | 86.44012 | 620.4213 | 547.1993 | 7.011334 |
| Kif12 | 0 | 1 | 85 | 49 | 103 | 0 | 2.010235 | 96.76295 | 74.89599 | 85.39246 |
| Col27a1 | 16.2541 | 11.0806 | 240 | 63.3096 | 241.313 | 54.24344 | 22.27461 | 273.213 | 96.76807 | 4.835213 |
| Orm3 | 0 | 1 | 143.459 | 119.148 | 121.021 | 0 | 2.010235 | 163.312 | 182.1165 | 171.8348 |
| Tnc | 23 | 17.0027 | 178 | 79 | 224 | 76.75596 | 34.17943 | 202.633 | 120.7507 | 2.915063 |
| Tlr4 | 9 | 8.00166 | 76 | 60.602 | 101.022 | 30.03494 | 16.08522 | 86.51746 | 92.62953 | 3.884353 |
| Tle1 | 30 | 22.1729 | 190.443 | 92.4196 | 184.014 | 100.1165 | 44.57275 | 216.798 | 141.2624 | 2.474686 |
| Kdm4c | 11 | 7 | 84.0062 | 53 | 54 | 36.70937 | 14.07165 | 95.63162 | 81.00995 | 3.478496 |
| D4Bwg0951e | 132 | 266 | 1627.18 | 1013 | 1900 | 440.5125 | 534.7226 | 1852.362 | 1548.36 | 3.487079 |
| uc008tjz.1 | 167.717 | 91.8756 | 1583.77 | 661.449 | 1640.96 | 559.7078 | 184.6916 | 1802.944 | 1011.018 | 3.780178 |
| Haus6 | 27 | 42 | 40.0602 | 16 | 64.1208 | 90.10482 | 84.42988 | 45.60404 | 24.45583 | 0.401409 |
| Acer2 | 1.022 | 2.07813 | 54.1506 | 40.7925 | 74.0058 | 3.410634 | 4.17753 | 61.64437 | 62.35091 | 16.34062 |
| uc008toe.1 | 74 | 268 | 24 | 22 | 27 | 246.954 | 538.7431 | 27.3213 | 33.62677 | 0.077572 |
| Cdkn2a | 642 | 1271.97 | 385 | 428.953 | 522.54 | 2142.492 | 2556.959 | 438.2792 | 655.6502 | 0.232778 |
| Cdkn2b | 395 | 357 | 234 | 181 | 251 | 1318.2 | 717.654 | 266.3827 | 276.6566 | 0.266738 |
| Gm12657 | 9.39502 | 15.301 | 96.291 | 43.9324 | 158.017 | 31.35321 | 30.75861 | 109.6165 | 67.15022 | 2.845943 |
| Jun | 186 | 116 | 1042.77 | 738.999 | 1086.72 | 620.7221 | 233.1873 | 1187.076 | 1129.552 | 2.712968 |
| Pde4b | 8.00509 | 4.02721 | 616 | 492.901 | 627.375 | 26.71471 | 8.09564 | 701.2468 | 753.3941 | 41.7876 |
| Pcsk9 | 90.6698 | 37 | 84 | 44 | 93 | 302.5847 | 74.37871 | 95.62456 | 67.25355 | 0.432079 |
| Tmem48 | 35 | 66 | 52 | 28 | 51 | 116.8025 | 132.6755 | 59.19616 | 42.79771 | 0.408829 |
| Scp2 | 54 | 149 | 448 | 369 | 586 | 180.2096 | 299.5251 | 509.9977 | 564.0127 | 2.238759 |
| Orc1l | 16 | 42.9301 | 19 | 12 | 34 | 53.39545 | 86.2996 | 21.62937 | 18.34188 | 0.286132 |
| Rab3b | 57.8262 | 41.3858 | 22.4784 | 35.5368 | 13.0058 | 192.9785 | 83.1952 | 25.58913 | 54.31763 | 0.289335 |
| Cdkn2c | 10 | 23 | 119 | 73.4362 | 105 | 33.37216 | 46.23541 | 135.4681 | 112.2465 | 3.111697 |
| Spata6 | 34.074 | 16.7533 | 156.376 | 99.4328 | 154.432 | 113.7123 | 33.67808 | 178.0165 | 151.982 | 2.238942 |
| Pdzk1ip1 | 32 | 7 | 463 | 422.721 | 446 | 106.7909 | 14.07165 | 527.0735 | 646.1247 | 9.706879 |
| Cyp4b1 | 9 | 2 | 65 | 49 | 87 | 30.03494 | 4.020471 | 73.9952 | 74.89599 | 4.372027 |
| Nsun4 | 18 | 19 | 99 | 61 | 115 | 60.06988 | 38.19447 | 112.7004 | 93.23787 | 2.095757 |
| Pik3r3 | 0 | 2 | 61 | 25.0459 | 66 | 0 | 4.020471 | 69.44165 | 38.2824 | 26.79389 |
| Hectd3 | 18 | 6 | 109 | 48.1294 | 99 | 60.06988 | 12.06141 | 124.0843 | 73.56529 | 2.740136 |
| Kif2c | 21 | 95 | 57 | 31 | 71 | 70.08153 | 190.9724 | 64.8881 | 47.38318 | 0.430069 |
| Tmem53 | 8 | 1 | 85 | 52 | 96 | 26.69773 | 2.010235 | 96.76295 | 79.48146 | 6.139217 |
| Slc6a9 | 29.2895 | 3.50003 | 130.677 | 58.2251 | 85.3554 | 97.74538 | 7.035884 | 148.7611 | 88.99646 | 2.269085 |
| B4galt2 | 40 | 14 | 37 | 22 | 53 | 133.4886 | 28.14329 | 42.12034 | 33.62677 | 0.46864 |
| Ipo13 | 34 | 26 | 197 | 85 | 153 | 113.4653 | 52.26612 | 224.2624 | 129.9216 | 2.137096 |
| St3gal3 | 14 | 9 | 62 | 40 | 64 | 46.72102 | 18.09212 | 70.58003 | 61.13959 | 2.032298 |
| Cdc20 | 92 | 465 | 217 | 134.501 | 241.576 | 307.0238 | 934.7594 | 247.0301 | 205.5834 | 0.364487 |
| Ebna1bp2 | 171 | 422.559 | 274.673 | 241.298 | 294.173 | 570.6639 | 849.443 | 312.6843 | 368.8215 | 0.479898 |
| Ppih | 69.3401 | 219.458 | 151.526 | 102.17 | 194.82 | 231.4029 | 441.1622 | 172.4953 | 156.1658 | 0.488668 |
| Scmh1 | 14 | 17.9444 | 117 | 53 | 134 | 46.72102 | 36.07247 | 133.1914 | 81.00995 | 2.587176 |
| Ctps | 121 | 286 | 893 | 626 | 952.853 | 403.8031 | 574.9273 | 1016.58 | 956.8345 | 2.016301 |
| Smap2 | 26 | 24 | 154 | 72 | 161 | 86.76761 | 48.24565 | 175.3117 | 110.0513 | 2.113592 |
| Bmp8b | 37.8249 | 34.8889 | 5.19428 | 3.08108 | 6.16092 | 126.2298 | 70.1349 | 5.913104 | 4.709399 | 0.054096 |
| Ndufs5 | 14.2269 | 32.4214 | 9.83114 | 15.0568 | 7.34558 | 47.47823 | 65.17464 | 11.19165 | 23.01416 | 0.303639 |
| Cdca8 | 40 | 209.418 | 119 | 80.0956 | 134.047 | 133.4886 | 420.9795 | 135.4681 | 122.4253 | 0.465119 |
| Zc3h12a | 4.64155 | 20.7762 | 245.795 | 226.155 | 323.783 | 15.48985 | 41.76505 | 279.81 | 345.6756 | 10.92458 |
| OTTMUSG00000009332 | 4 | 6 | 49 | 36 | 56 | 13.34886 | 12.06141 | 55.78099 | 55.02563 | 4.360702 |
| Gjb3 | 42 | 128 | 82 | 54 | 102 | 140.1631 | 257.3101 | 93.34779 | 82.53844 | 0.442511 |
| Ccdc28b | 22.2124 | 1543.6 | 51.3637 | 195.888 | 134.861 | 74.12757 | 3102.999 | 58.4718 | 299.4128 | 0.112644 |
| Col16a1 | 17 | 7.00525 | 90 | 47 | 103.113 | 56.73267 | 14.0822 | 102.4549 | 71.83901 | 2.461261 |
| Serinc2 | 50 | 18 | 580 | 364 | 791 | 166.8608 | 36.18424 | 660.2648 | 556.3702 | 5.991947 |
| uc008vaz.1 | 4 | 9 | 153.5 | 140 | 168 | 13.34886 | 18.09212 | 174.7425 | 213.9886 | 12.36383 |
| Smpdl3b | 19 | 35 | 12 | 3 | 10 | 63.4071 | 70.35824 | 13.66065 | 4.585469 | 0.136404 |
| Tmem222 | 12 | 27 | 137 | 86 | 165 | 40.04659 | 54.27635 | 155.9591 | 131.4501 | 3.047077 |
| Fam46b | 9 | 4 | 168 | 94 | 190 | 30.03494 | 8.040941 | 191.2491 | 143.678 | 8.796307 |
| 1810019J16Rik | 1 | 0 | 54 | 36 | 58 | 3.337216 | 0 | 61.47293 | 55.02563 | 34.90891 |
| Sfn | 1 | 1 | 93 | 106 | 155 | 3.337216 | 2.010235 | 105.8701 | 162.0199 | 50.09676 |
| Dhdds | 27 | 24.2812 | 167 | 73.9725 | 182.373 | 90.10482 | 48.81093 | 190.1107 | 113.0662 | 2.182452 |
| Ccdc21 | 13.5599 | 13.8225 | 167.935 | 85.0873 | 171.798 | 45.25231 | 27.78648 | 191.1751 | 130.0551 | 4.398077 |
| Zfp593 | 47.9686 | 154.636 | 73.9512 | 64.6404 | 91.9991 | 160.0816 | 310.8547 | 84.18513 | 98.80218 | 0.388561 |
| Pdik1l | 11 | 15.0093 | 71.5849 | 39.0597 | 84.5806 | 36.70937 | 30.17222 | 81.49137 | 59.70235 | 2.1111 |
| Pafah2 | 14 | 14 | 123 | 55 | 156 | 46.72102 | 28.14329 | 140.0217 | 84.06693 | 2.993263 |
| 2610002D18Rik | 6 | 44 | 6 | 12 | 12 | 20.02329 | 88.45035 | 6.830326 | 18.34188 | 0.232058 |
| Fuca1 | 33 | 18.0333 | 299.09 | 183.12 | 339.321 | 110.1281 | 36.25118 | 340.4804 | 279.897 | 4.23815 |
| Eif4g3 | 18 | 17 | 169.178 | 77.474 | 291.164 | 60.06988 | 34.174 | 192.5901 | 118.4182 | 3.300038 |
| Pink1 | 23 | 10 | 227 | 90 | 214 | 76.75596 | 20.10235 | 258.414 | 137.5641 | 4.08822 |
| Pqlc2 | 11 | 11.0091 | 80 | 37 | 97 | 36.70937 | 22.13088 | 91.07101 | 56.55412 | 2.508914 |
| Aldh4a1 | 13 | 12 | 191 | 79 | 211 | 43.3838 | 24.12282 | 217.432 | 120.7507 | 5.009623 |
| Padi2 | 27.2356 | 31.6592 | 7.29174 | 2.32332 | 6.1679 | 90.89107 | 63.64244 | 8.300827 | 3.551171 | 0.076695 |
| Epha2 | 364 | 336 | 448 | 250 | 384 | 1214.746 | 675.4391 | 509.9977 | 382.1224 | 0.471975 |
| Fblim1 | 271.465 | 144.284 | 256.474 | 152.942 | 309.94 | 905.9372 | 290.0448 | 291.9668 | 233.7703 | 0.439586 |
| Tmem51 | 13 | 16 | 81 | 41 | 107 | 43.3838 | 32.16377 | 92.2094 | 62.66808 | 2.050066 |
| 9030409G11Rik | 6.0411 | 2.02105 | 62.4129 | 40 | 83.3796 | 20.16045 | 4.062786 | 71.05007 | 61.13959 | 5.457142 |
| 4732496O08Rik | 11 | 6 | 62 | 29 | 77.9598 | 36.70937 | 12.06141 | 70.58003 | 44.3262 | 2.356046 |
| Pramel4 | 5.54671 | 60.5526 | 0 | 2.05455 | 0.796408 | 18.51057 | 121.725 | 0 | 3.140358 | 0.022393 |
| Gm13102 | 5.54671 | 60.5526 | 0 | 2.05455 | 0.829868 | 18.51057 | 121.725 | 0 | 3.140358 | 0.022393 |
| Dhrs3 | 24.2005 | 12 | 438.366 | 161.472 | 486.197 | 80.76229 | 24.12282 | 499.0304 | 246.8083 | 7.111007 |
| Tnfrsf1b | 17 | 8.01039 | 188.13 | 69 | 213.07 | 56.73267 | 16.10277 | 214.1649 | 105.4658 | 4.388395 |
| uc008vst.1 | 12.6505 | 4.58566 | 85.5174 | 71.3263 | 31.0615 | 42.21745 | 9.218256 | 97.35195 | 109.0215 | 4.012261 |
| Plod1 | 20 | 16.3637 | 158.28 | 66 | 208.319 | 66.74431 | 32.89489 | 180.184 | 100.8803 | 2.820821 |
| Agtrap | 17.0116 | 11.0305 | 138.639 | 86.7823 | 125.486 | 56.77138 | 22.1739 | 157.8249 | 132.6458 | 3.679394 |
| Pgd | 83 | 168 | 818 | 348 | 1055 | 276.9889 | 337.7195 | 931.2011 | 531.9144 | 2.380178 |
| Kif1b | 45.9506 | 49.7214 | 337.95 | 115.09 | 389.078 | 153.3471 | 99.95171 | 384.7181 | 175.9139 | 2.213323 |
| Nmnat1 | 174.696 | 2987.51 | 159.408 | 690.928 | 284.649 | 582.9982 | 6005.598 | 181.4681 | 1056.076 | 0.187831 |
| Ctnnbip1 | 15 | 33 | 161 | 87 | 190 | 50.05823 | 66.33777 | 183.2804 | 132.9786 | 2.717095 |
| Slc25a33 | 55 | 52 | 54 | 45 | 57 | 183.5469 | 104.5322 | 61.47293 | 68.78204 | 0.45215 |
| H6pd | 60 | 35 | 1665 | 511 | 1592 | 200.2329 | 70.35824 | 1895.415 | 781.0582 | 9.891208 |
| uc008wbb.1 | 10.1899 | 0.12213 | 37.9197 | 76.4038 | 90.9047 | 34.00589 | 0.24551 | 43.16732 | 116.7824 | 4.669874 |
| uc008wbc.1 | 20.4012 | 13.7205 | 200.572 | 128.828 | 107.134 | 68.0832 | 27.58143 | 228.3287 | 196.9123 | 4.445122 |
| uc008wbd.1 | 20.4012 | 32.1348 | 142.292 | 104.592 | 177.54 | 68.0832 | 64.59851 | 161.9835 | 159.8678 | 2.425739 |
| Prkcz | 10 | 9.02648 | 82.2385 | 44.1664 | 86.1977 | 33.37216 | 18.14535 | 93.61929 | 67.50789 | 3.12762 |
| B930041F14Rik | 3 | 8 | 146 | 75 | 154 | 10.01165 | 16.08188 | 166.2046 | 114.6367 | 10.76287 |
| Mxra8 | 45 | 20 | 16.0369 | 12.2438 | 19 | 150.1747 | 40.20471 | 18.25621 | 18.71452 | 0.194195 |
| Agrn | 344 | 249 | 2457 | 1084 | 3124 | 1148.002 | 500.5486 | 2797.018 | 1656.883 | 2.701707 |
| Isg15 | 0 | 2.00083 | 99.4757 | 247.082 | 122.842 | 0 | 4.022139 | 113.2419 | 377.6623 | 122.0505 |
| Pex1 | 10 | 12 | 57.23 | 33 | 72.0914 | 33.37216 | 24.12282 | 65.14992 | 50.44016 | 2.010438 |
| Fzd1 | 23 | 42 | 168 | 103 | 182 | 76.75596 | 84.42988 | 191.2491 | 157.4344 | 2.163239 |
| Steap1 | 111 | 147 | 135 | 100 | 174 | 370.4309 | 295.5046 | 153.6823 | 152.849 | 0.460302 |
| Steap4 | 0 | 2 | 1415 | 1211 | 1487 | 0 | 4.020471 | 1610.819 | 1851.001 | 861.0483 |
| Abcb1a | 163.401 | 104.92 | 14.0591 | 7.27766 | 14.9554 | 545.3044 | 210.9139 | 16.00471 | 11.12383 | 0.035874 |
| Abcb1b | 56.5353 | 87.2369 | 52.1532 | 44.3645 | 47.6593 | 188.6705 | 175.3667 | 59.37056 | 67.81068 | 0.349363 |
| Crot | 45.609 | 26.5384 | 211.832 | 112.466 | 211.372 | 152.2071 | 53.34843 | 241.1469 | 171.9031 | 2.009433 |
| Sema3a | 47.1481 | 52.0754 | 2 | 2.00491 | 1.00351 | 157.3434 | 104.6838 | 2.276775 | 3.064484 | 0.020384 |
| Sema3e | 112.709 | 177.128 | 19.068 | 16.205 | 24.1943 | 376.1342 | 356.069 | 21.70678 | 24.76918 | 0.063474 |
| Gnai1 | 13 | 11 | 97 | 39 | 137 | 43.3838 | 22.11259 | 110.4236 | 59.6111 | 2.596093 |
| Pion | 30.7126 | 8.07351 | 316.824 | 230.641 | 264.953 | 102.4946 | 16.22965 | 360.6685 | 352.5324 | 6.007206 |
| uc008wor.1 | 542.451 | 5439.53 | 1.4007 | 2816.11 | 2152.78 | 1810.276 | 10934.74 | 1.59454 | 4304.395 | 0.337857 |
| BC050254 | 1.00001 | 4.66667 | 57.9831 | 56.9455 | 72 | 3.337249 | 9.381105 | 66.00724 | 87.04061 | 12.03362 |
| uc008wql.1 | 0.606964 | 0 | 31.323 | 50.6853 | 73.2954 | 2.02557 | 0 | 35.65772 | 77.47196 | 55.85079 |
| uc008wqt.1 | 5.91874 | 57.8798 | 4.60534 | 7.4999 | 4.79062 | 19.75211 | 116.352 | 5.242662 | 11.46352 | 0.122746 |
| Galnt11 | 16 | 13 | 148 | 113 | 155 | 53.39545 | 26.13306 | 168.4814 | 172.7193 | 4.290294 |
| Lmbr1 | 29.1872 | 30.0773 | 199.705 | 102.393 | 238.065 | 97.40398 | 60.46245 | 227.3417 | 156.5066 | 2.431475 |
| Hadhb | 45 | 74 | 313 | 195 | 296 | 150.1747 | 148.7574 | 356.3153 | 298.0555 | 2.189028 |
| Cenpa | 81 | 560 | 160 | 113.267 | 169 | 270.3145 | 1125.732 | 182.142 | 173.1274 | 0.254483 |
| Mapre3 | 9 | 0 | 56 | 26 | 54 | 30.03494 | 0 | 63.74971 | 39.74073 | 3.445668 |
| Cgref1 | 12 | 33 | 10 | 9 | 14 | 40.04659 | 66.33777 | 11.38388 | 13.75641 | 0.236316 |
| 0610007C21Rik | 24 | 55 | 373 | 276 | 456 | 80.09318 | 110.5629 | 424.6186 | 421.8631 | 4.439835 |
| Cad | 140 | 252 | 234 | 123 | 314 | 467.2102 | 506.5793 | 266.3827 | 188.0042 | 0.466617 |
| Krtcap3 | 22 | 43 | 226 | 173 | 215 | 73.41874 | 86.44012 | 257.2756 | 264.4287 | 3.263531 |
| Zfp512 | 14 | 16 | 86 | 46 | 104 | 46.72102 | 32.16377 | 97.90134 | 70.31052 | 2.132374 |
| Fosl2 | 61.5865 | 30.0274 | 538.246 | 206.186 | 553.655 | 205.5274 | 60.36214 | 612.7326 | 315.1532 | 3.489741 |
| Spon2 | 24 | 25 | 28 | 16 | 22 | 80.09318 | 50.25588 | 31.87485 | 24.45583 | 0.432153 |
| uc008xbx.1 | 72 | 12 | 544 | 235 | 583 | 240.2795 | 24.12282 | 619.2829 | 359.1951 | 3.700716 |
| Mxd4 | 7 | 10 | 54 | 28 | 58 | 23.36051 | 20.10235 | 61.47293 | 42.79771 | 2.399074 |
| BC037112 | 42.6323 | 159.188 | 104.716 | 59.8836 | 108.132 | 142.2732 | 320.0053 | 119.2074 | 91.53146 | 0.45587 |
| Lrpap1 | 37 | 41 | 419 | 282 | 457 | 123.477 | 82.41965 | 476.9844 | 431.0341 | 4.41007 |
| Mrfap1 | 416.786 | 471.989 | 2461 | 1398.22 | 3121.61 | 1390.905 | 948.809 | 2801.572 | 2137.165 | 2.11083 |
| Man2b2 | 13 | 8 | 87 | 45 | 80 | 43.3838 | 16.08188 | 99.03972 | 68.78204 | 2.822161 |
| Msx1 | 2.0071 | 0 | 40.0839 | 43.0598 | 43.2189 | 6.698125 | 0 | 45.63102 | 65.81646 | 16.63861 |
| Stx18 | 22 | 17 | 101 | 71 | 133 | 73.41874 | 34.174 | 114.9772 | 108.5228 | 2.077277 |
| Hs3st1 | 25 | 34 | 355 | 225 | 419.919 | 83.43039 | 68.348 | 404.1276 | 343.9102 | 4.928487 |
| Bst1 | 25 | 12 | 3226.89 | 2588 | 3418 | 83.43039 | 24.12282 | 3673.452 | 3955.731 | 70.93403 |
| Cd38 | 18.7753 | 6.05292 | 129.45 | 70.7154 | 137.464 | 62.65722 | 12.16779 | 147.3643 | 108.0878 | 3.413992 |
| Fgfbp1 | 36 | 16 | 148 | 113 | 132 | 120.1398 | 32.16377 | 168.4814 | 172.7193 | 2.240268 |
| Prom1 | 4 | 0 | 636 | 281 | 732 | 13.34886 | 0 | 724.0145 | 429.5056 | 86.41337 |
| Tapt1 | 22 | 36 | 165 | 79 | 138 | 73.41874 | 72.36847 | 187.834 | 120.7507 | 2.116678 |
| uc008xjf.1 | 3.69231 | 54.0967 | 26.9836 | 6.27322 | 20.6769 | 12.32203 | 108.7471 | 30.7178 | 9.588552 | 0.33292 |
| Sod3 | 1 | 2 | 781 | 458 | 952.766 | 3.337216 | 4.020471 | 889.0807 | 700.0483 | 215.9822 |
| Rbpj | 248.601 | 148.162 | 232.948 | 108 | 223.061 | 829.6351 | 297.8405 | 265.1851 | 165.0769 | 0.381615 |
| Pcdh7 | 15 | 37 | 21 | 8 | 22 | 50.05823 | 74.37871 | 23.90614 | 12.22792 | 0.29038 |
| Arap2 | 18.0828 | 6.05446 | 74.3427 | 39.8281 | 86.3856 | 60.3462 | 12.17089 | 84.63081 | 60.87684 | 2.006529 |
| 0610040J01Rik | 12 | 13 | 374 | 212 | 352.997 | 40.04659 | 26.13306 | 425.757 | 324.0398 | 11.32972 |
| Fam114a1 | 69.436 | 27.1799 | 68 | 37.0493 | 84.7873 | 231.7229 | 54.63799 | 77.41036 | 56.62947 | 0.46808 |
| Wdr19 | 12 | 9 | 54 | 41 | 80.6867 | 40.04659 | 18.09212 | 61.47293 | 62.66808 | 2.135256 |
| Ugdh | 450 | 565 | 286 | 159 | 295 | 1501.747 | 1135.783 | 325.5789 | 243.0299 | 0.215584 |
| uc008xoj.1 | 2.53964 | 134.795 | 1.12895 | 0 | 1.13999 | 8.475326 | 270.9697 | 1.285183 | 0 | 0.004599 |
| Uchl1 | 39 | 48.9583 | 24 | 17 | 31 | 130.1514 | 98.4177 | 27.3213 | 25.98432 | 0.233214 |
| Atp8a1 | 21.9748 | 5.08396 | 172.917 | 83.4858 | 163.403 | 73.33465 | 10.21996 | 196.8466 | 127.6072 | 3.883135 |
| Atp10d | 11.3407 | 4.40291 | 155.199 | 80.303 | 180.109 | 37.84636 | 8.850885 | 176.6766 | 122.7423 | 6.411918 |
| Ociad2 | 167.933 | 498.075 | 131.873 | 125.06 | 149.073 | 560.4286 | 1001.248 | 150.1226 | 191.1529 | 0.218531 |
| Sgcb | 9 | 10 | 86 | 75 | 89 | 30.03494 | 20.10235 | 97.90134 | 114.6367 | 4.239121 |
| Rasl11b | 26 | 19 | 0 | 3.00262 | 1 | 86.76761 | 38.19447 | 0 | 4.589474 | 0.036727 |
| Srd5a3 | 14 | 32.4346 | 107.183 | 90.3286 | 119.164 | 46.72102 | 65.20118 | 122.0158 | 138.0663 | 2.323776 |
| Igfbp7 | 1001.39 | 1309.05 | 12885.8 | 13266.1 | 15561.4 | 3341.854 | 2631.499 | 14669.04 | 20277.1 | 5.850338 |
| Slc4a4 | 83.1281 | 49 | 744.082 | 409.993 | 733.669 | 277.4164 | 98.50153 | 847.0538 | 626.6701 | 3.920334 |
| Gc | 26 | 65 | 337 | 253 | 391 | 86.76761 | 130.6653 | 383.6366 | 386.7079 | 3.542907 |
| Cxcl5 | 31 | 11 | 20161.1 | 16961.6 | 19881.1 | 103.4537 | 22.11259 | 22951.15 | 25925.63 | 389.2508 |
| Pf4 | 1 | 3.07086 | 60.0827 | 66.3845 | 58.2015 | 3.337216 | 6.173151 | 68.3974 | 101.468 | 17.86108 |
| Cxcl3 | 4 | 1.33333 | 791.895 | 591.681 | 818.645 | 13.34886 | 2.680307 | 901.4835 | 904.3783 | 112.661 |
| Cxcl1 | 19 | 21 | 1570.99 | 1539 | 1532 | 63.4071 | 42.21494 | 1788.396 | 2352.346 | 39.20338 |
| Cxcl2 | 0 | 2.66667 | 2146.11 | 2021.28 | 2325.34 | 0 | 5.360634 | 2443.105 | 3089.506 | 1032.081 |
| Mthfd2l | 26 | 43 | 190 | 137 | 191 | 86.76761 | 86.44012 | 216.2937 | 209.4031 | 2.457724 |
| Areg | 331 | 584 | 235 | 207 | 253 | 1104.618 | 1173.977 | 267.5211 | 316.3974 | 0.256262 |
| Btc | 49.5926 | 59 | 32.0124 | 21.0689 | 30.1247 | 165.5012 | 118.6039 | 36.44252 | 32.2036 | 0.241622 |
| Cxcl10 | 14 | 12.1097 | 84 | 86 | 82 | 46.72102 | 24.34335 | 95.62456 | 131.4501 | 3.195338 |
| Shroom3 | 15 | 16 | 346.994 | 214.592 | 387.374 | 50.05823 | 32.16377 | 395.0137 | 328.0017 | 8.793454 |
| Antxr2 | 216 | 126 | 165 | 56 | 199 | 720.8386 | 253.2896 | 187.834 | 85.59542 | 0.280691 |
| Prkg2 | 24 | 39.3071 | 4.01101 | 3.00427 | 2.00909 | 80.09318 | 79.01652 | 4.566084 | 4.591996 | 0.057558 |
| uc008yhl.1 | 106 | 193.358 | 74.3663 | 90.9998 | 82.8729 | 353.7449 | 388.6951 | 84.65768 | 139.0923 | 0.301371 |
| Hpse | 10 | 6 | 109 | 66.6984 | 119 | 33.37216 | 12.06141 | 124.0843 | 101.9478 | 4.975001 |
| Pkd2 | 94 | 44.0781 | 445.15 | 201.756 | 468.295 | 313.6983 | 88.60735 | 506.7533 | 308.382 | 2.026159 |
| BC057170 | 2 | 1 | 107.063 | 53.4643 | 141.412 | 6.674431 | 2.010235 | 121.8792 | 81.71963 | 23.44348 |
| Mpa2l | 0.5 | 0 | 70.5698 | 28.4317 | 80.5312 | 1.668608 | 0 | 80.33579 | 43.45756 | 74.1896 |
| Cdc7 | 18 | 35 | 27 | 21 | 21 | 60.06988 | 70.35824 | 30.73647 | 32.09828 | 0.481758 |
| Fam69a | 66 | 68 | 465 | 322 | 473.916 | 220.2562 | 136.696 | 529.3503 | 492.1737 | 2.861794 |
| Pcgf3 | 17 | 13 | 93 | 49 | 112 | 56.73267 | 26.13306 | 105.8701 | 74.89599 | 2.181433 |
| uc008ypl.1 | 18.4546 | 25.0847 | 14.7326 | 13.9623 | 17.1432 | 61.58698 | 50.42615 | 16.77141 | 21.34123 | 0.340252 |
| Chfr | 9.29747 | 15 | 117.967 | 42.9767 | 150.987 | 31.02766 | 30.15353 | 134.2922 | 65.68944 | 3.268678 |
| Pole | 36 | 44 | 53.0596 | 25.6829 | 81.2128 | 120.1398 | 88.45035 | 60.40239 | 39.25605 | 0.477772 |
| 2410025L10Rik | 17 | 7 | 244.146 | 137 | 259.851 | 56.73267 | 14.07165 | 277.9328 | 209.4031 | 6.882856 |
| Pus1 | 102 | 133 | 142.746 | 80.6015 | 174 | 340.396 | 267.3613 | 162.5003 | 123.1986 | 0.470087 |
| Iscu | 368.05 | 2978.98 | 845.823 | 1148.06 | 980.225 | 1228.262 | 5988.451 | 962.8744 | 1754.798 | 0.37658 |
| Trpv4 | 9 | 4.01788 | 61 | 27.2347 | 99 | 30.03494 | 8.076884 | 69.44165 | 41.62796 | 2.914308 |
| Gltp | 22 | 53.0233 | 179 | 120 | 166 | 73.41874 | 106.5893 | 203.7714 | 183.4188 | 2.15096 |
| 1500011B03Rik | 7 | 11.4726 | 59.778 | 39.0852 | 51.5398 | 23.36051 | 23.06263 | 68.05054 | 59.74132 | 2.752762 |
| uc008zai.1 | 3.11416 | 21.5652 | 85.0631 | 104.342 | 90.5079 | 10.39262 | 43.35113 | 96.83478 | 159.4857 | 4.769307 |
| Oasl2 | 1 | 1.00127 | 189.053 | 91.9462 | 224.685 | 3.337216 | 2.012788 | 215.2156 | 140.5388 | 66.4961 |
| Sirt4 | 15 | 7 | 72 | 37 | 65 | 50.05823 | 14.07165 | 81.96391 | 56.55412 | 2.159961 |
| uc008zfm.1 | 4.4349 | 45.2517 | 2.57597 | 5.78432 | 7.06978 | 14.80022 | 90.96657 | 2.932452 | 8.841273 | 0.111318 |
| Plbd2 | 51 | 32 | 268.618 | 112 | 354 | 170.198 | 64.32753 | 305.7914 | 171.1908 | 2.033818 |
| Slc24a6 | 10.0103 | 12.0722 | 62.1421 | 39.2796 | 42.5674 | 33.40653 | 24.26796 | 70.7418 | 60.03846 | 2.267558 |
| Tpcn1 | 104 | 57 | 112.197 | 61 | 160 | 347.0704 | 114.5834 | 127.7237 | 93.23787 | 0.47863 |
| Rasal1 | 1 | 0 | 243 | 78 | 206 | 3.337216 | 0 | 276.6282 | 119.2222 | 118.617 |
| Oas1a | 0 | 2 | 179.866 | 66.7612 | 221.847 | 0 | 4.020471 | 204.7572 | 102.0438 | 76.30973 |
| uc008zni.1 | 16.0195 | 14.7285 | 136.096 | 81.9852 | 195.868 | 53.46053 | 29.60775 | 154.93 | 125.3135 | 3.373653 |
| Hip1r | 30 | 13 | 203 | 63 | 256 | 100.1165 | 26.13306 | 231.0927 | 96.29485 | 2.593178 |
| Vps37b | 128 | 91 | 160 | 62 | 166 | 427.1636 | 182.9314 | 182.142 | 94.76636 | 0.453877 |
| Rilpl2 | 232 | 361 | 169 | 143 | 195 | 774.234 | 725.6949 | 192.3875 | 218.574 | 0.273987 |
| Tctn2 | 17.0182 | 15.2135 | 102.884 | 67.16 | 108.591 | 56.7934 | 30.58271 | 117.1219 | 102.6534 | 2.515278 |
| Cldn4 | 16 | 15.1974 | 799.736 | 612.783 | 949.232 | 53.39545 | 30.55035 | 910.4096 | 936.6325 | 22.00279 |
| Rhbdd2 | 24.664 | 33.3061 | 193.415 | 68.5472 | 206.817 | 82.30909 | 66.9531 | 220.1812 | 104.7737 | 2.177075 |
| Orai2 | 16 | 19 | 272 | 115.852 | 276 | 53.39545 | 38.19447 | 309.6414 | 177.0786 | 5.314122 |
| Rabl5 | 8 | 32.8421 | 75.506 | 74 | 107.63 | 26.69773 | 66.02035 | 85.9551 | 113.1082 | 2.146974 |
| Trfr2 | 64 | 18 | 8 | 5 | 8 | 213.5818 | 36.18424 | 9.107101 | 7.642448 | 0.067061 |
| Pcolce | 314 | 358.629 | 174.63 | 116 | 187 | 1047.886 | 720.9287 | 198.7966 | 177.3048 | 0.212629 |
| Pilra | 12.4 | 18.4556 | 146.147 | 157.555 | 181.323 | 41.38147 | 37.1001 | 166.3719 | 240.8212 | 5.188392 |
| Pilrb1 | 6.2 | 22.6309 | 147.948 | 107.969 | 151.044 | 20.69074 | 45.49343 | 168.4222 | 165.0295 | 5.038239 |
| Pilrb2 | 12.4 | 24.8892 | 274.444 | 155.469 | 264.414 | 41.38147 | 50.03315 | 312.4237 | 237.6328 | 6.01716 |
| Fam20c | 30 | 36 | 192 | 109 | 215 | 100.1165 | 72.36847 | 218.5704 | 166.6054 | 2.233098 |
| Micall2 | 6 | 5 | 90 | 46 | 115 | 20.02329 | 10.05118 | 102.4549 | 70.31052 | 5.744587 |
| uc009ajd.1 | 27 | 227 | 9 | 9.375 | 7.35 | 90.10482 | 456.3234 | 10.24549 | 14.32959 | 0.044974 |
| Zfp12 | 56.4905 | 525.068 | 83.1814 | 81.0029 | 98.5071 | 188.521 | 1055.51 | 94.69268 | 123.8121 | 0.175643 |
| Cyth3 | 74 | 60 | 70.0376 | 52 | 93 | 246.954 | 120.6141 | 79.72994 | 79.48146 | 0.433148 |
| uc009ats.1 | 16.9554 | 53.8916 | 8.72649 | 24.2077 | 8.00367 | 56.58383 | 108.3348 | 9.934128 | 37.00122 | 0.284597 |
| Samd9l | 13 | 7 | 235.682 | 111.664 | 247.093 | 43.3838 | 14.07165 | 268.2975 | 170.6773 | 7.640263 |
| Ica1 | 15 | 9 | 94 | 46 | 78.1315 | 50.05823 | 18.09212 | 107.0084 | 70.31052 | 2.601879 |
| Met | 99 | 109 | 601 | 272 | 667.776 | 330.3843 | 219.1156 | 684.171 | 415.7492 | 2.001675 |
| Ptprz1 | 41 | 50 | 2 | 3 | 5.01497 | 136.8258 | 100.5118 | 2.276775 | 4.585469 | 0.028913 |
| Rbm28 | 39 | 84 | 72 | 40 | 82 | 130.1514 | 168.8598 | 81.96391 | 61.13959 | 0.478589 |
| 2310016C08Rik | 87 | 221 | 108 | 85 | 104 | 290.3378 | 444.262 | 122.9459 | 129.9216 | 0.344225 |
| Flnc | 194 | 147 | 192 | 51 | 202 | 647.4198 | 295.5046 | 218.5704 | 77.95297 | 0.314472 |
| Tspan33 | 2 | 2 | 51 | 39 | 56 | 6.674431 | 4.020471 | 58.05777 | 59.6111 | 11.00233 |
| Zc3hc1 | 93 | 86 | 83 | 41 | 104.34 | 310.3611 | 172.8802 | 94.48617 | 62.66808 | 0.325209 |
| Tmem209 | 117.381 | 206.995 | 140.024 | 103 | 176.769 | 391.7257 | 416.1087 | 159.4016 | 157.4344 | 0.392204 |
| Tsga14 | 18 | 23 | 22 | 16 | 26 | 60.06988 | 46.23541 | 25.04453 | 24.45583 | 0.465643 |
| Mest | 35.6191 | 39.1304 | 56.6747 | 20.46 | 100.606 | 118.8686 | 78.66131 | 64.51778 | 31.2729 | 0.484943 |
| 2310005E10Rik | 32.1616 | 26.4194 | 147.138 | 109.801 | 163.898 | 107.3302 | 53.10921 | 167.5001 | 167.8297 | 2.090071 |
| D630045J12Rik | 12.9335 | 38.4752 | 14.3989 | 6.21573 | 22.7272 | 43.16188 | 77.34421 | 16.39153 | 9.500679 | 0.214862 |
| Zc3hav1 | 8 | 15 | 57 | 35 | 68.1125 | 26.69773 | 30.15353 | 64.8881 | 53.49714 | 2.082368 |
| uc009bli.1 | 21 | 4 | 96 | 56 | 93.1435 | 70.08153 | 8.040941 | 109.2852 | 85.59542 | 2.494553 |
| Ezh2 | 122 | 248 | 157 | 103 | 157 | 407.1403 | 498.5384 | 178.7269 | 157.4344 | 0.371171 |
| Atp6v0e2 | 21 | 31 | 210 | 83 | 285 | 70.08153 | 62.31729 | 239.0614 | 126.8646 | 2.763816 |
| Tmem176b | 564 | 671 | 4887 | 3631.97 | 5502 | 1882.19 | 1348.868 | 5563.3 | 5551.429 | 3.439966 |
| Tmem176a | 245 | 236 | 2280.97 | 1566.99 | 2472.97 | 817.6178 | 474.4155 | 2596.628 | 2395.128 | 3.863489 |
| Igf2bp3 | 75.0982 | 94 | 4.00118 | 7.02932 | 10.0468 | 250.6189 | 188.9621 | 4.554894 | 10.74424 | 0.034804 |
| Ccdc126 | 8 | 13 | 41 | 45 | 49 | 26.69773 | 26.13306 | 46.67389 | 68.78204 | 2.185391 |
| Mpp6 | 122 | 112 | 106.268 | 59 | 144.206 | 407.1403 | 225.1464 | 120.9742 | 90.18089 | 0.333955 |
| Cycs | 499.596 | 3577.62 | 1382.51 | 1520.83 | 1359.73 | 1667.26 | 7191.858 | 1573.832 | 2324.573 | 0.440044 |
| Chn2 | 52 | 71 | 27 | 16 | 27.0594 | 173.5352 | 142.7267 | 30.73647 | 24.45583 | 0.174515 |
| Aqp1 | 44 | 69 | 420 | 211 | 544 | 146.8375 | 138.7062 | 478.1228 | 322.5113 | 2.803893 |
| Lsm5 | 109 | 527 | 198 | 192 | 224 | 363.7565 | 1059.394 | 225.4008 | 293.47 | 0.364593 |
| Ppm1k | 117.912 | 490.164 | 148.019 | 97.8636 | 145.597 | 393.4978 | 985.345 | 168.503 | 149.5835 | 0.230691 |
| Abcg2 | 46 | 90 | 48 | 34 | 55 | 153.5119 | 180.9212 | 54.64261 | 51.96865 | 0.318782 |
| Herc3 | 20 | 16 | 101 | 65 | 111 | 66.74431 | 32.16377 | 114.9772 | 99.35183 | 2.166951 |
| Tnip3 | 14 | 8 | 520 | 459.138 | 613 | 46.72102 | 16.08188 | 591.9616 | 701.7877 | 20.60015 |
| Mad2l1 | 80 | 334 | 198 | 142 | 244 | 266.9773 | 671.4186 | 225.4008 | 217.0455 | 0.471492 |
| Eif2ak3 | 35 | 28.1014 | 195.037 | 87.2793 | 174.59 | 116.8025 | 56.49043 | 222.0277 | 133.4055 | 2.051054 |
| Mrpl35 | 169.38 | 237.216 | 183.243 | 157.107 | 218.486 | 565.2576 | 476.86 | 208.6016 | 240.1364 | 0.430602 |
| Ptcd3 | 157 | 214 | 226.7 | 118.184 | 294.26 | 523.9429 | 430.1904 | 258.0725 | 180.643 | 0.459805 |
| Polr1a | 144 | 102.046 | 182.145 | 52 | 175 | 480.5591 | 205.1365 | 207.3516 | 79.48146 | 0.41831 |
| St3gal5 | 10.0113 | 27 | 61 | 72 | 93.3396 | 33.40987 | 54.27635 | 69.44165 | 110.0513 | 2.046991 |
| Ggcx | 34.9646 | 11.2039 | 157.527 | 70.1021 | 145.688 | 116.6844 | 22.52248 | 179.3268 | 107.1503 | 2.057924 |
| Capg | 374 | 528 | 175 | 90 | 213 | 1248.119 | 1061.404 | 199.2178 | 137.5641 | 0.145823 |
| Retsat | 36 | 46 | 46 | 35 | 73 | 120.1398 | 92.47082 | 52.36583 | 53.49714 | 0.49792 |
| Tmsb10 | 4372.98 | 17111 | 6235.92 | 6786 | 7867 | 14593.58 | 34397.14 | 7098.894 | 10372.33 | 0.356623 |
| Mrpl19 | 88.3814 | 135.929 | 125.882 | 86.3834 | 156.59 | 294.9478 | 273.2493 | 143.3025 | 132.0361 | 0.484583 |
| Hk2 | 138 | 89 | 157 | 35.9722 | 164 | 460.5358 | 178.9109 | 178.7269 | 54.98314 | 0.365488 |
| Loxl3 | 119.444 | 85.441 | 137.857 | 57.2124 | 212.931 | 398.6104 | 171.7565 | 156.9347 | 87.44856 | 0.428467 |
| Htra2 | 43.9631 | 181.563 | 70.746 | 71.5572 | 53.855 | 146.7143 | 364.9844 | 80.53637 | 109.3744 | 0.371138 |
| Dqx1 | 18.6768 | 27.9555 | 16.7129 | 13.622 | 15.9688 | 62.32851 | 56.19713 | 19.02576 | 20.82109 | 0.336188 |
| Bola3 | 141 | 447 | 226 | 272 | 257 | 470.5474 | 898.5752 | 257.2756 | 415.7492 | 0.491574 |
| Paip2b | 81 | 106 | 126 | 58 | 162 | 270.3145 | 213.0849 | 143.4368 | 88.6524 | 0.480119 |
| Smyd5 | 118 | 99 | 115 | 42 | 115 | 393.7914 | 199.0133 | 130.9146 | 64.19657 | 0.329132 |
| Add2 | 17 | 22 | 3 | 1 | 2 | 56.73267 | 44.22518 | 3.415163 | 1.52849 | 0.048967 |
| 2010309E21Rik | 226.966 | 287.65 | 229 | 147 | 214 | 757.4345 | 578.2442 | 260.6908 | 224.688 | 0.363395 |
| Snrpg | 1566 | 8001 | 2033 | 2562 | 1915 | 5226.08 | 16083.89 | 2314.342 | 3915.991 | 0.292367 |
| Gfpt1 | 30 | 21 | 22 | 14 | 26.127 | 100.1165 | 42.21494 | 25.04453 | 21.39886 | 0.326305 |
| Isy1 | 104 | 172 | 138 | 84 | 163.676 | 347.0704 | 345.7605 | 157.0975 | 128.3931 | 0.412064 |
| H1fx | 8 | 25 | 80 | 42 | 81 | 26.69773 | 50.25588 | 91.07101 | 64.19657 | 2.017678 |
| Ruvbl1 | 199.995 | 302 | 254 | 132 | 271.922 | 667.4264 | 607.0911 | 289.1505 | 201.7606 | 0.385174 |
| Mcm2 | 135 | 272 | 210 | 99 | 167 | 450.5241 | 546.784 | 239.0614 | 151.3205 | 0.391436 |
| Txnrd3 | 28 | 25 | 37.0629 | 15 | 30 | 93.44204 | 50.25588 | 42.19195 | 22.92735 | 0.453168 |
| Wnt7a | 142 | 91 | 131 | 46 | 138 | 473.8846 | 182.9314 | 149.1288 | 70.31052 | 0.334096 |
| Chchd4 | 69 | 187 | 123 | 105 | 150 | 230.2679 | 375.914 | 140.0217 | 160.4914 | 0.495747 |
| Lsm3 | 125 | 640 | 206 | 178 | 174 | 417.152 | 1286.551 | 234.5079 | 272.0712 | 0.29734 |
| Slc6a6 | 184.797 | 212.797 | 1324.07 | 839.117 | 1153.59 | 616.7074 | 427.772 | 1507.305 | 1282.582 | 2.671078 |
| Lrig1 | 74.25 | 62.4462 | 471.907 | 259.872 | 617.14 | 247.7883 | 125.5316 | 537.2131 | 397.2117 | 2.503014 |
| Tmf1 | 23 | 13 | 18 | 9 | 29 | 76.75596 | 26.13306 | 20.49098 | 13.75641 | 0.332858 |
| Shq1 | 44 | 42 | 45 | 22 | 55 | 146.8375 | 84.42988 | 51.22744 | 33.62677 | 0.36691 |
| Glt8d4 | 2 | 4 | 74 | 51 | 82 | 6.674431 | 8.040941 | 84.24069 | 77.95297 | 11.02206 |
| Pdzrn3 | 13 | 7 | 586 | 402.468 | 651.918 | 43.3838 | 14.07165 | 667.0952 | 615.1682 | 22.31752 |
| Bhlhe40 | 118 | 45 | 623 | 407.612 | 713 | 393.7914 | 90.46059 | 709.2155 | 623.0307 | 2.751142 |
| Lmcd1 | 11 | 12 | 77 | 70 | 88 | 36.70937 | 24.12282 | 87.65585 | 106.9943 | 3.199788 |
| Ogg1 | 26.3786 | 67.0684 | 21.28 | 21.5978 | 26.1714 | 88.03108 | 134.8233 | 24.22489 | 33.01201 | 0.256835 |
| Camk1 | 116.621 | 65.9316 | 111.72 | 70.5466 | 141.017 | 389.1894 | 132.538 | 127.1807 | 107.8297 | 0.450447 |
| Il17re | 3 | 0 | 61.0443 | 39.9265 | 60.0495 | 10.01165 | 0 | 69.49208 | 61.02724 | 13.03675 |
| 1500001M20Rik | 66 | 129 | 85 | 68 | 120 | 220.2562 | 259.3204 | 96.76295 | 103.9373 | 0.418495 |
| Pparg | 56 | 68 | 9 | 8 | 16 | 186.8841 | 136.696 | 10.24549 | 12.22792 | 0.069452 |
| D6Wsu116e | 134 | 86.1994 | 175 | 69 | 201 | 447.1869 | 173.2811 | 199.2178 | 105.4658 | 0.491055 |
| 8430408G22Rik | 16 | 2 | 161.187 | 145.868 | 174.922 | 53.39545 | 4.020471 | 183.4933 | 222.9577 | 7.079065 |
| Bms1 | 104 | 90 | 134 | 58 | 144 | 347.0704 | 180.9212 | 152.5439 | 88.6524 | 0.456819 |
| Zfp9 | 42.8925 | 11.1575 | 23.2802 | 13.2834 | 27.878 | 143.1415 | 22.4292 | 26.50189 | 20.30354 | 0.282691 |
| Bcl2l13 | 193.625 | 154.661 | 265.523 | 105.61 | 269.538 | 646.1684 | 310.905 | 302.2681 | 161.4238 | 0.484489 |
| Nanog | 7.85688 | 101.764 | 6.36873 | 12.14 | 4.79161 | 26.2201 | 204.5696 | 7.250084 | 18.55586 | 0.111816 |
| C1r | 3 | 2 | 179.453 | 116 | 217 | 10.01165 | 4.020471 | 204.2871 | 177.3048 | 27.19418 |
| C1s | 8 | 2 | 716 | 411.628 | 791 | 26.69773 | 4.020471 | 815.0855 | 629.1691 | 47.01626 |
| Phb2 | 568 | 1188.94 | 1008 | 636 | 1279 | 1895.538 | 2390.049 | 1147.495 | 972.1194 | 0.494591 |
| Ptpn6 | 19 | 24 | 25 | 10 | 14 | 63.4071 | 48.24565 | 28.45969 | 15.2849 | 0.391791 |
| Cdca3 | 48 | 478 | 107 | 76 | 114 | 160.1864 | 960.8925 | 121.8075 | 116.1652 | 0.212271 |
| Nop2 | 210 | 225 | 195 | 90 | 219 | 700.8153 | 452.3029 | 221.9856 | 137.5641 | 0.311806 |
| Gapdh | 396.103 | 6043.22 | 1461.68 | 2297.33 | 295.591 | 1321.881 | 12148.29 | 1663.958 | 3511.445 | 0.384212 |
| Ncapd2 | 60 | 180 | 107 | 36 | 127 | 200.2329 | 361.8424 | 121.8075 | 55.02563 | 0.314608 |
| Mrpl51 | 126.301 | 528.371 | 276.912 | 268.941 | 294.691 | 421.4937 | 1062.15 | 315.2332 | 411.0735 | 0.489543 |
| Ndufa9 | 344 | 398 | 394 | 288 | 387 | 1148.002 | 800.0737 | 448.5247 | 440.205 | 0.456209 |
| Rad51ap1 | 22.1222 | 74.1021 | 24.0259 | 16.0509 | 23.0717 | 73.82655 | 148.9627 | 27.35079 | 24.53363 | 0.232886 |
| Tulp3 | 206.845 | 1536.07 | 309.269 | 295.954 | 411.574 | 690.2864 | 3087.862 | 352.068 | 452.3626 | 0.212917 |
| Foxm1 | 27 | 106 | 74 | 23 | 68 | 90.10482 | 213.0849 | 84.24069 | 35.15526 | 0.393799 |
| Fkbp4 | 455 | 650 | 650 | 328 | 722 | 1518.433 | 1306.653 | 739.952 | 501.3446 | 0.439384 |
| Clec2d | 8.07286 | 21.0662 | 84.1019 | 285.285 | 62.0845 | 26.94087 | 42.34802 | 95.74056 | 436.0552 | 7.67505 |
| uc009ehg.1 | 305.73 | 590.125 | 41.6846 | 46.1282 | 48.5416 | 1020.287 | 1186.29 | 47.45323 | 70.50648 | 0.053458 |
| Magohb | 65 | 166 | 85 | 86 | 94 | 216.919 | 333.6991 | 96.76295 | 131.4501 | 0.414467 |
| Styk1 | 87 | 41.2696 | 33.0283 | 25 | 30 | 290.3378 | 82.96161 | 37.59901 | 38.21224 | 0.203084 |
| Csda | 1174 | 1179.47 | 1472 | 748.999 | 1648 | 3917.891 | 2371.012 | 1675.707 | 1144.837 | 0.448495 |
| Etv6 | 45.1411 | 29.1451 | 340.183 | 132.256 | 367.036 | 150.6456 | 58.58851 | 387.2601 | 202.1519 | 2.816998 |
| Mansc1 | 3 | 4 | 83 | 54.123 | 77.5037 | 10.01165 | 8.040941 | 94.48617 | 82.72645 | 9.816466 |
| Dusp16 | 70.4336 | 47 | 504.255 | 214.853 | 631.007 | 235.0521 | 94.48106 | 574.0377 | 328.4006 | 2.738535 |
| uc009elc.1 | 7 | 94 | 18 | 24 | 28 | 23.36051 | 188.9621 | 20.49098 | 36.68375 | 0.269282 |
| Gprc5a | 311 | 315 | 219 | 103 | 289 | 1037.874 | 633.2241 | 249.3069 | 157.4344 | 0.243398 |
| Hebp1 | 51 | 55 | 48 | 30 | 42 | 170.198 | 110.5629 | 54.64261 | 45.85469 | 0.357946 |
| Emp1 | 1836 | 3908.99 | 554 | 323.173 | 596.782 | 6127.128 | 7857.99 | 630.6668 | 493.9666 | 0.080416 |
| Mgst1 | 122 | 50 | 2609 | 2255 | 2763 | 407.1403 | 100.5118 | 2970.053 | 3446.744 | 12.64015 |
| Ldhb | 0 | 1 | 269 | 236 | 302 | 0 | 2.010235 | 306.2263 | 360.7236 | 331.777 |
| Bcat1 | 45 | 12.0226 | 5 | 1.00544 | 4.00232 | 150.1747 | 24.16826 | 5.691938 | 1.536805 | 0.041463 |
| Lrmp | 34.4747 | 46.9998 | 0 | 0 | 0 | 115.0495 | 94.48066 | 0 | 0 | 0 |
| uc009ers.1 | 73 | 56 | 55 | 28 | 72 | 243.6167 | 112.5732 | 62.61132 | 42.79771 | 0.295935 |
| Dennd5b | 166.451 | 1210.71 | 136.609 | 226.068 | 142.108 | 555.4829 | 2433.812 | 155.514 | 345.5426 | 0.167617 |
| 4833442J19Rik | 41 | 17 | 174 | 119.992 | 169.628 | 136.8258 | 34.174 | 198.0794 | 183.4065 | 2.230914 |
| Tmc4 | 8.64055 | 12.8967 | 101.438 | 50.4186 | 124.473 | 28.83538 | 25.9254 | 115.4758 | 77.06431 | 3.516021 |
| Leng8 | 58 | 28.1233 | 380.5 | 101.971 | 456.174 | 193.5585 | 56.53445 | 433.1565 | 155.8616 | 2.355197 |
| Cox6b2 | 13 | 11 | 94 | 98 | 122 | 43.3838 | 22.11259 | 107.0084 | 149.792 | 3.920833 |
| Clcn4-2 | 24 | 26 | 212 | 131 | 322 | 80.09318 | 52.26612 | 241.3382 | 200.2321 | 3.336149 |
| 2310014L17Rik | 38.8975 | 181.505 | 23.9961 | 96.4063 | 4.05992 | 129.8093 | 364.8678 | 27.31686 | 147.356 | 0.353105 |
| Bbc3 | 55 | 18 | 251 | 148 | 330 | 183.5469 | 36.18424 | 285.7353 | 226.2165 | 2.329901 |
| Fkrp | 24 | 37 | 164 | 131 | 217 | 80.09318 | 74.37871 | 186.6956 | 200.2321 | 2.504842 |
| Pglyrp1 | 3 | 4 | 1576 | 1682 | 1498.77 | 10.01165 | 8.040941 | 1794.099 | 2570.92 | 241.7946 |
| Sympk | 165.993 | 1381.66 | 286.374 | 280.241 | 361.541 | 553.9544 | 2777.462 | 326.0046 | 428.3455 | 0.226435 |
| Fosb | 88 | 14 | 902 | 299.709 | 836.687 | 293.675 | 28.14329 | 1026.826 | 458.1021 | 4.614181 |
| Ercc1 | 99 | 136 | 127 | 89 | 180 | 330.3843 | 273.392 | 144.5752 | 136.0356 | 0.46476 |
| Apoc2 | 20 | 448.987 | 0 | 0 | 0 | 66.74431 | 902.5695 | 0 | 0 | 0 |
| Bcam | 77 | 67 | 831 | 410 | 872 | 256.9656 | 134.6858 | 946.0001 | 626.6808 | 4.015512 |
| 2210010C17Rik | 83 | 55 | 30 | 25 | 35 | 276.9889 | 110.5629 | 34.15163 | 38.21224 | 0.18672 |
| Zfp428 | 21 | 102 | 29 | 46 | 43 | 70.08153 | 205.044 | 33.01324 | 70.31052 | 0.375551 |
| Cxcl17 | 8 | 2 | 139 | 144 | 132 | 26.69773 | 4.020471 | 158.2359 | 220.1025 | 12.31643 |
| Ceacam1 | 13.994 | 1 | 102.997 | 42.9988 | 84.9594 | 46.701 | 2.010235 | 117.2505 | 65.72322 | 3.756295 |
| B9d2 | 5 | 26 | 73 | 59 | 96 | 16.68608 | 52.26612 | 83.1023 | 90.18089 | 2.513092 |
| Axl | 703 | 516 | 780 | 289 | 889 | 2346.063 | 1037.281 | 887.9424 | 441.7335 | 0.393006 |
| Cyp2s1 | 73 | 50 | 36 | 10 | 27.0426 | 243.6167 | 100.5118 | 40.98195 | 15.2849 | 0.163505 |
| Numbl | 29 | 11 | 19 | 13 | 27 | 96.77925 | 22.11259 | 21.62937 | 19.87037 | 0.349054 |
| Ltbp4 | 134 | 32 | 59 | 31 | 82 | 447.1869 | 64.32753 | 67.16487 | 47.38318 | 0.223939 |
| Blvrb | 55 | 45 | 42 | 35 | 56 | 183.5469 | 90.46059 | 47.81228 | 53.49714 | 0.369732 |
| Dyrk1b | 7 | 7 | 58 | 29 | 86 | 23.36051 | 14.07165 | 66.02648 | 44.3262 | 2.948072 |
| Zfp36 | 46 | 40 | 552 | 276 | 691 | 153.5119 | 80.40941 | 628.39 | 421.8631 | 4.489771 |
| Spint2 | 509 | 728.997 | 3676.97 | 2559.91 | 3420.95 | 1698.643 | 1465.456 | 4185.817 | 3912.796 | 2.559533 |
| Zfp566 | 26 | 27.6923 | 32.5818 | 17.2097 | 32.5079 | 86.76761 | 55.66804 | 37.09072 | 26.30485 | 0.445082 |
| Aplp1 | 5 | 3 | 85 | 36.0399 | 108 | 16.68608 | 6.030706 | 96.76295 | 55.08661 | 6.684466 |
| Fxyd5 | 35.3745 | 96 | 36 | 24 | 41 | 118.0523 | 192.9826 | 40.98195 | 36.68375 | 0.249701 |
| Fxyd3 | 11.9988 | 11.1429 | 186.283 | 164.457 | 213.963 | 40.04258 | 22.39985 | 212.0623 | 251.3708 | 7.421765 |
| Hpn | 1 | 0 | 66 | 25 | 57 | 3.337216 | 0 | 75.13358 | 38.21224 | 33.96419 |
| Scn1b | 2 | 1 | 48 | 31 | 56 | 6.674431 | 2.010235 | 54.64261 | 47.38318 | 11.74781 |
| Ccne1 | 27 | 89 | 60 | 24 | 72 | 90.10482 | 178.9109 | 68.30326 | 36.68375 | 0.390263 |
| 1600014C10Rik | 20 | 10.09 | 96.5706 | 47.2474 | 99.224 | 66.74431 | 20.28327 | 109.9348 | 72.21716 | 2.093037 |
| Plekhf1 | 12 | 8 | 96 | 73 | 126 | 40.04659 | 16.08188 | 109.2852 | 111.5797 | 3.93499 |
| Zfp715 | 17.0175 | 8 | 80 | 37 | 81.3506 | 56.79107 | 16.08188 | 91.07101 | 56.55412 | 2.025788 |
| Myh14 | 10 | 2 | 68 | 18 | 69 | 33.37216 | 4.020471 | 77.41036 | 27.51281 | 2.805986 |
| Cpt1c | 21 | 7.00305 | 186.885 | 117.111 | 229.654 | 70.08153 | 14.07778 | 212.7476 | 179.003 | 4.654869 |
| Tead2 | 234 | 139 | 263 | 137 | 269 | 780.9085 | 279.4227 | 299.3959 | 209.4031 | 0.479849 |
| Bax | 68 | 335 | 762 | 764 | 931 | 226.9307 | 673.4288 | 867.4514 | 1167.766 | 2.26045 |
| Dbp | 26 | 10 | 14 | 16 | 24 | 86.76761 | 20.10235 | 15.93743 | 24.45583 | 0.377966 |
| Grwd1 | 52 | 112 | 92 | 46 | 124 | 173.5352 | 225.1464 | 104.7317 | 70.31052 | 0.439053 |
| Emp3 | 283 | 1146 | 132 | 101 | 183 | 944.432 | 2303.73 | 150.2672 | 154.3775 | 0.09379 |
| Htatip2 | 39 | 26 | 195 | 131 | 215 | 130.1514 | 52.26612 | 221.9856 | 200.2321 | 2.314568 |
| uc009hcn.1 | 4 | 18.5411 | 38.5523 | 44.2526 | 28.6191 | 13.34886 | 37.27197 | 43.88746 | 67.63964 | 2.203186 |
| Snrpn | 10 | 7.00684 | 93.159 | 50 | 80 | 33.37216 | 14.0854 | 106.0511 | 76.42448 | 3.845026 |
| Chsy1 | 51 | 102.594 | 80.2554 | 39.0316 | 84.5585 | 170.198 | 206.2381 | 91.36176 | 59.6594 | 0.401187 |
| Lrrc28 | 13.2781 | 18.4427 | 87.0408 | 55.4231 | 100.793 | 44.31188 | 37.07417 | 99.08617 | 84.71364 | 2.25837 |
| Arrdc4 | 22 | 14 | 280 | 195.994 | 306 | 73.41874 | 28.14329 | 318.7485 | 299.5748 | 6.088134 |
| Slco3a1 | 99.6818 | 47 | 490 | 277.624 | 557.862 | 332.6597 | 94.48106 | 557.8099 | 424.3454 | 2.299372 |
| Acan | 151 | 172 | 0 | 0 | 2 | 503.9196 | 345.7605 | 0 | 0 | 0 |
| Abhd2 | 14 | 65 | 11 | 8 | 13 | 46.72102 | 130.6653 | 12.52226 | 12.22792 | 0.139527 |
| Prc1 | 88.7017 | 262.266 | 200.559 | 100.823 | 317.994 | 296.0167 | 527.2164 | 228.3139 | 154.1069 | 0.464535 |
| Furin | 116.755 | 57 | 621.988 | 232.07 | 667.571 | 389.6366 | 114.5834 | 708.0635 | 354.7166 | 2.10777 |
| Whamm | 8 | 8 | 88 | 39 | 75.2755 | 26.69773 | 16.08188 | 100.1781 | 59.6111 | 3.735172 |
| Mex3b | 24 | 25 | 31 | 15 | 29 | 80.09318 | 50.25588 | 35.29002 | 22.92735 | 0.446627 |
| Stard5 | 11 | 9.2551 | 158.079 | 98.628 | 186.076 | 36.70937 | 18.60493 | 179.9552 | 150.7519 | 5.97869 |
| Ctsc | 171.175 | 231 | 1015 | 844 | 1180.7 | 571.2479 | 464.3644 | 1155.463 | 1290.045 | 2.361414 |
| Rab38 | 28 | 31 | 161 | 154 | 199.437 | 93.44204 | 62.31729 | 183.2804 | 235.3874 | 2.687915 |
| Tmem135 | 30 | 17 | 176 | 99 | 186 | 100.1165 | 34.174 | 200.3562 | 151.3205 | 2.618776 |
| Sytl2 | 31 | 52 | 0 | 0 | 3 | 103.4537 | 104.5322 | 0 | 0 | 0 |
| Ccdc90b | 20.4615 | 32.3077 | 140 | 77.617 | 120.579 | 68.28444 | 64.94608 | 159.3743 | 118.6368 | 2.086692 |
| Prcp | 10.2146 | 5.32878 | 69.9503 | 38.2316 | 68.0003 | 34.08832 | 10.7121 | 79.63056 | 58.43661 | 3.081827 |
| Ints4 | 39 | 35.1129 | 205.109 | 126 | 188.229 | 130.1514 | 70.58519 | 233.4936 | 192.5897 | 2.122599 |
| 1810020D17Rik | 7 | 17 | 41 | 64 | 59 | 23.36051 | 34.174 | 46.67389 | 97.82334 | 2.511488 |
| Acer3 | 47 | 62 | 460.088 | 317.264 | 453.162 | 156.8491 | 124.6346 | 523.7585 | 484.9347 | 3.583487 |
| Uvrag | 18 | 18 | 136 | 69 | 119 | 60.06988 | 36.18424 | 154.8207 | 105.4658 | 2.70416 |
| Dgat2 | 24 | 19 | 25 | 16 | 32 | 80.09318 | 38.19447 | 28.45969 | 24.45583 | 0.447346 |
| Mtap6 | 70 | 15 | 66 | 23.4668 | 97.0637 | 233.6051 | 30.15353 | 75.13358 | 35.86876 | 0.420848 |
| Gdpd5 | 16 | 9 | 174 | 64 | 176 | 53.39545 | 18.09212 | 198.0794 | 97.82334 | 4.13922 |
| uc009img.1 | 17 | 12.0812 | 161 | 73 | 197 | 56.73267 | 24.28605 | 183.2804 | 111.5797 | 3.639408 |
| Kcne3 | 1 | 4 | 372.997 | 320 | 470 | 3.337216 | 8.040941 | 424.6152 | 489.1167 | 80.30579 |
| Ucp2 | 168.866 | 139 | 1905.55 | 661.142 | 2618.27 | 563.5423 | 279.4227 | 2169.255 | 1010.549 | 3.772165 |
| Arhgef17 | 47 | 45.6862 | 411 | 170 | 528.778 | 156.8491 | 91.84001 | 467.8773 | 259.8432 | 2.926226 |
| P2ry6 | 1 | 1 | 266 | 143 | 304.437 | 3.337216 | 2.010235 | 302.8111 | 218.574 | 97.50162 |
| P2ry2 | 4 | 9 | 375.548 | 212 | 462.446 | 13.34886 | 18.09212 | 427.5192 | 324.0398 | 23.9038 |
| 3200002M19Rik | 22 | 89 | 47 | 39.9749 | 64.0934 | 73.41874 | 178.9109 | 53.50422 | 61.10122 | 0.454189 |
| Trim21 | 7 | 3 | 92.2335 | 37 | 92 | 23.36051 | 6.030706 | 104.9975 | 56.55412 | 5.496595 |
| Trim6 | 13.6557 | 64.2839 | 27.2467 | 20.5222 | 36.0067 | 45.57202 | 129.2258 | 31.01731 | 31.36797 | 0.3569 |
| Smpd1 | 19.0202 | 20.0199 | 148.152 | 88.7247 | 195.234 | 63.47451 | 40.24471 | 168.6544 | 135.6148 | 2.933585 |
| Hpx | 6 | 5 | 233 | 141 | 307 | 20.02329 | 10.05118 | 265.2443 | 215.517 | 15.9857 |
| Trim3 | 15 | 16 | 126 | 50 | 108 | 50.05823 | 32.16377 | 143.4368 | 76.42448 | 2.673996 |
| Rrp8 | 48.1177 | 89.9098 | 84.6302 | 45.7168 | 97.4735 | 160.5791 | 180.7399 | 96.34197 | 69.87766 | 0.486992 |
| Tpp1 | 99 | 112 | 559.232 | 335.685 | 654.628 | 330.3843 | 225.1464 | 636.6228 | 513.0911 | 2.069578 |
| St5 | 22 | 5 | 279.058 | 82 | 303 | 73.41874 | 10.05118 | 317.6762 | 125.3362 | 5.307449 |
| Ampd3 | 1 | 1 | 91.2222 | 37 | 82.1783 | 3.337216 | 2.010235 | 103.8462 | 56.55412 | 29.99566 |
| Pde3b | 31 | 6 | 405 | 183 | 462 | 103.4537 | 12.06141 | 461.047 | 279.7136 | 6.412674 |
| Nucb2 | 29 | 16 | 224 | 120 | 283 | 96.77925 | 32.16377 | 254.9988 | 183.4188 | 3.400088 |
| Itpripl2 | 228.823 | 113.32 | 218.892 | 113 | 260.614 | 763.6317 | 227.7999 | 249.1839 | 172.7193 | 0.42555 |
| uc009jkh.1 | 16.5 | 8.9415 | 71.2866 | 42.8771 | 102.926 | 55.06406 | 17.97452 | 81.15178 | 65.5372 | 2.008377 |
| Gde1 | 16.5 | 31.0585 | 181.713 | 156.123 | 264.074 | 55.06406 | 62.43489 | 206.8598 | 238.6324 | 3.791457 |
| 6330503K22Rik | 8 | 8 | 59 | 24 | 60 | 26.69773 | 16.08188 | 67.16487 | 36.68375 | 2.427526 |
| Gprc5b | 38 | 15 | 223.121 | 117.249 | 250.038 | 126.8142 | 30.15353 | 253.9982 | 179.2139 | 2.75988 |
| Tmem159 | 25 | 9 | 102.371 | 83 | 141 | 83.43039 | 18.09212 | 116.5379 | 126.8646 | 2.397523 |
| Cdr2 | 26 | 45 | 35.0151 | 26 | 63 | 86.76761 | 90.46059 | 39.86076 | 39.74073 | 0.449147 |
| Plk1 | 39.3023 | 202.558 | 108.395 | 60.8391 | 132.343 | 131.1602 | 407.1892 | 123.3955 | 92.99194 | 0.401946 |
| Rbbp6 | 14 | 15 | 113 | 70 | 139 | 46.72102 | 30.15353 | 128.6378 | 106.9943 | 3.065151 |
| Aqp8 | 15 | 39 | 0 | 0 | 1 | 50.05823 | 78.39918 | 0 | 0 | 0 |
| Sbk1 | 24 | 1 | 97 | 51 | 117 | 80.09318 | 2.010235 | 110.4236 | 77.95297 | 2.294382 |
| Cln3 | 8 | 21 | 78 | 46 | 74.9324 | 26.69773 | 42.21494 | 88.79424 | 70.31052 | 2.308788 |
| Nupr1 | 174 | 82 | 919 | 992 | 1185 | 580.6755 | 164.8393 | 1046.178 | 1516.262 | 3.437142 |
| Sult1a1 | 2 | 0 | 249.556 | 216 | 296 | 6.674431 | 0 | 284.0915 | 330.1538 | 92.0296 |
| Ypel3 | 124 | 26 | 472 | 354 | 569 | 413.8147 | 52.26612 | 537.319 | 541.0853 | 2.313771 |
| Kif22 | 34 | 164 | 98 | 44 | 88 | 113.4653 | 329.6786 | 111.562 | 67.25355 | 0.403516 |
| Dctpp1 | 112 | 663 | 212 | 248 | 237 | 373.7682 | 1332.786 | 241.3382 | 379.0654 | 0.363542 |
| Gm166 | 6.78058 | 6.45832 | 51.4436 | 27.6057 | 42.3019 | 22.62826 | 12.98274 | 58.56276 | 42.19503 | 2.829401 |
| Orai3 | 18.0716 | 22 | 150.116 | 78 | 133.212 | 60.30883 | 44.22518 | 170.8902 | 119.2222 | 2.775292 |
| BC017158 | 7 | 17 | 72 | 29 | 80.9487 | 23.36051 | 34.174 | 81.96391 | 44.3262 | 2.195032 |
| Rgs10 | 5 | 13 | 62 | 45 | 58 | 16.68608 | 26.13306 | 70.58003 | 68.78204 | 3.254668 |
| Bag3 | 24 | 45 | 236 | 77 | 318 | 80.09318 | 90.46059 | 268.6595 | 117.6937 | 2.265287 |
| Htra1 | 126 | 70 | 122 | 55.5145 | 125 | 420.4892 | 140.7165 | 138.8833 | 84.85334 | 0.398671 |
| Dmbt1 | 27 | 8 | 2405 | 1037 | 2788 | 90.10482 | 16.08188 | 2737.822 | 1585.044 | 40.71005 |
| uc009kbr.1 | 17.2805 | 123.268 | 0 | 0 | 0.54271 | 57.66875 | 247.7977 | 0 | 0 | 0 |
| Fam53b | 11 | 5 | 59.1326 | 45 | 82. | 36.70937 | 10.05118 | 67.31582 | 68.78204 | 2.910527 |
| Adam12 | 3 | 5 | 76 | 26 | 71 | 10.01165 | 10.05118 | 86.51746 | 39.74073 | 6.293142 |
| Ptpre | 48 | 42.3238 | 37.3607 | 26.3757 | 46.4379 | 160.1864 | 85.0808 | 42.53096 | 40.31499 | 0.337778 |
| Paox | 117 | 137 | 65 | 31 | 68 | 390.4542 | 275.4022 | 73.9952 | 47.38318 | 0.182289 |
| 1190003J15Rik | 0 | 2 | 66 | 54 | 67 | 0 | 4.020471 | 75.13358 | 82.53844 | 39.21731 |
| Athl1 | 15 | 9 | 94 | 39 | 80 | 50.05823 | 18.09212 | 107.0084 | 59.6111 | 2.444881 |
| Ifitm2 | 334.906 | 854.931 | 2314.75 | 2079.83 | 2577.86 | 1117.654 | 1718.612 | 2635.083 | 3178.999 | 2.049907 |
| Ifitm3 | 10.0877 | 8.05599 | 6772.83 | 5824.74 | 9335.79 | 33.66483 | 16.19444 | 7710.106 | 8903.055 | 333.2011 |
| Sigirr | 62 | 49 | 53 | 22 | 64 | 206.9074 | 98.50153 | 60.33454 | 33.62677 | 0.307657 |
| Irf7 | 2 | 1 | 107 | 60 | 133 | 6.674431 | 2.010235 | 121.8075 | 91.70938 | 24.5855 |
| Taldo1 | 113 | 344 | 979 | 692 | 1166 | 377.1054 | 691.5209 | 1114.481 | 1057.715 | 2.0327 |
| Ctsd | 429 | 338 | 2457 | 1120 | 3033.98 | 1431.666 | 679.4595 | 2797.018 | 1711.908 | 2.135793 |
| Syt8 | 12 | 19 | 85 | 49 | 70 | 40.04659 | 38.19447 | 96.76295 | 74.89599 | 2.193975 |
| Cd81 | 390.274 | 282.203 | 3397.91 | 2000.32 | 3489.9 | 1302.428 | 567.2944 | 3868.139 | 3057.468 | 3.704082 |
| Kcnq1 | 0 | 1 | 145 | 69 | 147 | 0 | 2.010235 | 165.0662 | 105.4658 | 134.5773 |
| Phlda2 | 49 | 106 | 11 | 14 | 21 | 163.5236 | 213.0849 | 12.52226 | 21.39886 | 0.09007 |
| Tnfrsf23 | 97.7053 | 79.7231 | 150.687 | 41.4299 | 179.919 | 326.0637 | 160.2622 | 171.5402 | 63.32517 | 0.482938 |
| Pnpla6 | 21 | 18 | 172 | 62 | 160.359 | 70.08153 | 36.18424 | 195.8027 | 94.76636 | 2.734362 |
| uc009krz.1 | 23 | 93 | 179.954 | 241.983 | 152 | 76.75596 | 186.9519 | 204.8574 | 369.8685 | 2.179404 |
| uc009ksj.1 | 4 | 34 | 56 | 79 | 50 | 13.34886 | 68.348 | 63.74971 | 120.7507 | 2.258353 |
| Cars2 | 6.5 | 11 | 43.5 | 36.2938 | 59.5136 | 21.6919 | 22.11259 | 49.51986 | 55.4747 | 2.39689 |
| uc009kwb.1 | 1.16667 | 15.2727 | 42.6545 | 44.88 | 49.6154 | 3.893429 | 30.70172 | 48.55736 | 68.59862 | 3.386485 |
| Gas6 | 7 | 4 | 2034.97 | 1244 | 1908.99 | 23.36051 | 8.040941 | 2316.585 | 1901.441 | 134.3258 |
| Rasa3 | 79 | 69 | 94 | 44 | 113 | 263.64 | 138.7062 | 107.0084 | 67.25355 | 0.433114 |
| Upf3a | 2.0045 | 15.0562 | 55.0402 | 41.0722 | 65.0504 | 6.689449 | 30.2665 | 62.65708 | 62.77843 | 3.39419 |
| uc009laz.1 | 10.2466 | 2 | 64.3146 | 22.156 | 67.4963 | 34.19511 | 4.020471 | 73.21495 | 33.86522 | 2.802003 |
| Nek3 | 12 | 6 | 87 | 36 | 73 | 40.04659 | 12.06141 | 99.03972 | 55.02563 | 2.956655 |
| Al316807 | 23 | 20.119 | 158.861 | 94 | 167 | 76.75596 | 40.44392 | 180.8454 | 143.678 | 2.768974 |
| Tacc1 | 6.46154 | 2 | 128 | 64.6491 | 169.809 | 21.56355 | 4.020471 | 145.7136 | 98.81548 | 9.557883 |
| Fgfr1 | 1 | 2 | 114 | 82.7735 | 105.123 | 3.337216 | 4.020471 | 129.7762 | 126.5184 | 34.83359 |
| Gpr124 | 55 | 21 | 4 | 9 | 7 | 183.5469 | 42.21494 | 4.553551 | 13.75641 | 0.081103 |
| Brf2 | 16 | 24 | 17 | 11 | 23 | 53.39545 | 48.24565 | 19.35259 | 16.81339 | 0.35582 |
| Tmem66 | 92 | 58 | 419 | 266 | 534 | 307.0238 | 116.5936 | 476.9844 | 406.5783 | 2.085756 |
| Slc7a2 | 2.10052 | 3.17411 | 241.22 | 136.799 | 252.729 | 7.009888 | 6.380708 | 274.6019 | 209.0959 | 36.12219 |
| Mtus1 | 2 | 0 | 141 | 59 | 175 | 6.674431 | 0 | 160.5127 | 90.18089 | 37.56029 |
| Tlr3 | 3 | 3 | 87 | 50 | 134 | 10.01165 | 6.030706 | 99.03972 | 76.42448 | 10.93756 |
| Snx25 | 17 | 9 | 79 | 50 | 99 | 56.73267 | 18.09212 | 89.93262 | 76.42448 | 2.223289 |
| Dctd | 36 | 66 | 44 | 30.0262 | 55 | 120.1398 | 132.6755 | 50.08906 | 45.89474 | 0.37966 |
| Aga | 31 | 37 | 150 | 137 | 186 | 103.4537 | 74.37871 | 170.7581 | 209.4031 | 2.13775 |
| Vegfc | 26 | 19 | 7 | 7 | 7 | 86.76761 | 38.19447 | 7.968713 | 10.69943 | 0.14939 |
| Clcn3 | 53.4629 | 50.7804 | 389.407 | 209 | 444.703 | 178.4172 | 102.0806 | 443.2961 | 319.4543 | 2.719274 |
| Sh3rf1 | 77.8373 | 40.9474 | 419.789 | 164.535 | 425.093 | 259.7599 | 82.31391 | 477.8826 | 251.49 | 2.132209 |
| Cbr4 | 21 | 18 | 115.991 | 63.5149 | 118.88 | 70.08153 | 36.18424 | 132.0427 | 97.08187 | 2.156147 |
| Anxa10 | 34 | 220 | 12 | 6 | 17 | 113.4653 | 442.2518 | 13.66065 | 9.170938 | 0.041085 |
| Spock3 | 3 | 0 | 82 | 64 | 90 | 10.01165 | 0 | 93.34779 | 97.82334 | 19.09487 |
| Cpe | 175 | 268 | 1815 | 1076 | 2318 | 584.0127 | 538.7431 | 2066.174 | 1644.655 | 3.305107 |
| Atp6v1b2 | 124.397 | 78.0938 | 912.448 | 447.238 | 1381.17 | 415.1396 | 156.9869 | 1038.72 | 683.5987 | 3.01038 |
| Zfp868 | 26.4906 | 28 | 156.017 | 96.7385 | 164.584 | 88.40484 | 56.28659 | 177.6078 | 147.8638 | 2.249419 |
| Armc6 | 28 | 38 | 54 | 14.3163 | 71.4282 | 93.44204 | 76.38894 | 61.47293 | 21.88232 | 0.490813 |
| Crlf1 | 93 | 36 | 48 | 21 | 48 | 310.3611 | 72.36847 | 54.64261 | 32.09828 | 0.226638 |
| Isyna1 | 106 | 177 | 863 | 473 | 1111 | 353.7449 | 355.8117 | 982.4285 | 722.9756 | 2.403479 |
| Gdf15 | 22 | 3 | 636 | 483 | 699.986 | 73.41874 | 6.030706 | 724.0145 | 738.2605 | 18.4051 |
| Jund | 63 | 125 | 661 | 517 | 668.051 | 210.2446 | 251.2794 | 752.4742 | 790.2292 | 3.342629 |
| Haus8 | 11 | 42 | 20 | 16 | 25 | 36.70937 | 84.42988 | 22.76775 | 24.45583 | 0.389829 |
| Bst2 | 71 | 123 | 2272 | 2943 | 3045 | 236.9423 | 247.2589 | 2586.417 | 4498.345 | 14.63185 |
| Glt25d1 | 227.317 | 151.048 | 1629.4 | 810.026 | 1562.59 | 758.6058 | 303.642 | 1854.889 | 1238.116 | 2.911755 |
| uc009meg.1 | 48.6137 | 5.68545 | 328.163 | 65.5898 | 427.261 | 162.2344 | 11.42909 | 373.5767 | 100.2533 | 2.728438 |
| Arhgap10 | 27.84 | 24.6388 | 30.0274 | 15.3549 | 27.8453 | 92.90808 | 49.52979 | 34.18282 | 23.46981 | 0.404756 |
| Lsm6 | 69 | 475.607 | 198.241 | 208 | 283 | 230.2679 | 956.082 | 225.6751 | 317.9259 | 0.458213 |
| Asf1b | 19 | 99 | 56 | 44 | 80 | 63.4071 | 199.0133 | 63.74971 | 67.25355 | 0.499211 |
| Samd1 | 80 | 60 | 124 | 31 | 111 | 266.9773 | 120.6141 | 141.1601 | 47.38318 | 0.486449 |
| Rad23a | 9.28351 | 23.3415 | 95.5359 | 66.8731 | 73.501 | 30.98107 | 46.92191 | 108.7569 | 102.2148 | 2.708134 |
| Hook2 | 64.8047 | 690.402 | 163.129 | 215.773 | 231.145 | 216.2673 | 1387.87 | 185.704 | 329.8068 | 0.321363 |
| Fbxw9 | 17 | 19 | 280.569 | 190.384 | 345 | 56.73267 | 38.19447 | 319.3963 | 291 | 6.430156 |
| uc009mqo.1 | 23 | 28 | 128 | 80 | 159 | 76.75596 | 56.28659 | 145.7136 | 122.2792 | 2.014339 |
| Chd9 | 9 | 13 | 87.9886 | 53 | 92 | 30.03494 | 26.13306 | 100.1651 | 81.00995 | 3.225593 |
| Bbs2 | 3 | 2 | 58 | 24 | 72 | 10.01165 | 4.020471 | 66.02648 | 36.68375 | 7.319653 |
| Mt2 | 108.343 | 205 | 3502.92 | 2444.44 | 3936.3 | 361.564 | 412.0982 | 3987.681 | 3736.301 | 9.983662 |
| Mt1 | 304 | 615 | 4543.65 | 3717 | 4629.06 | 1014.514 | 1236.295 | 5172.435 | 5681.396 | 4.822193 |
| Herpud1 | 180 | 79 | 1616 | 1275 | 1737 | 600.6988 | 158.8086 | 1839.634 | 1948.824 | 4.988047 |
| Gpr56 | 44 | 19 | 36 | 30 | 45 | 146.8375 | 38.19447 | 40.98195 | 45.85469 | 0.469306 |
| Kifc3 | 86.3299 | 59.1772 | 840.733 | 397.181 | 930.754 | 288.1015 | 118.9601 | 957.0801 | 607.0871 | 3.842581 |
| 2210023G05Rik | 9.18568 | 17.3352 | 110.223 | 75.7619 | 122.214 | 30.65459 | 34.84783 | 125.4765 | 115.8013 | 3.683494 |
| Elmo3 | 16 | 7 | 106 | 53 | 84 | 53.39545 | 14.07165 | 120.6691 | 81.00995 | 2.989295 |
| Tppp3 | 1 | 23 | 55 | 36 | 62 | 3.337216 | 46.23541 | 62.61132 | 55.02563 | 2.373022 |
| Zdhhc1 | 6 | 5 | 100 | 29 | 118 | 20.02329 | 10.05118 | 113.8388 | 44.3262 | 5.259111 |
| Atp6v0d1 | 60 | 43 | 398 | 274 | 413 | 200.2329 | 86.44012 | 453.0783 | 418.8062 | 3.04139 |
| Smpd3 | 32 | 41.0223 | 397.918 | 225.181 | 515.802 | 106.7909 | 82.46448 | 452.9849 | 344.1868 | 4.212149 |
| Cdh1 | 273.786 | 308.773 | 2373.09 | 1062.67 | 3247.98 | 913.6829 | 620.7064 | 2701.496 | 1624.28 | 2.819217 |
| Cirh1a | 111 | 198.632 | 177 | 119 | 190 | 370.4309 | 399.2971 | 201.4946 | 181.8903 | 0.498078 |
| Marveld3 | 2 | 0 | 49 | 30 | 67 | 6.674431 | 0 | 55.78099 | 45.85469 | 15.22762 |
| Exosc6 | 63 | 191.368 | 104.697 | 102.65 | 135.415 | 210.2446 | 384.6947 | 119.1858 | 156.8995 | 0.464056 |
| uc009nlz.1 | 14 | 6 | 129 | 49.1794 | 154.825 | 46.72102 | 12.06141 | 146.852 | 75.1702 | 3.777016 |
| Znrf1 | 92.9368 | 61.7632 | 494.18 | 258.052 | 535.991 | 310.1501 | 124.1586 | 562.5684 | 394.4298 | 2.203498 |
| Ldhd | 16.0632 | 12.1652 | 102.715 | 59.4916 | 124.043 | 53.60636 | 24.45491 | 116.9295 | 90.9323 | 2.662803 |
| 4932417I16Rik | 6 | 7.02532 | 61 | 33.0354 | 108 | 20.02329 | 14.12255 | 69.44165 | 50.49427 | 3.51246 |
| Mon1b | 11 | 7 | 82 | 42.1499 | 117 | 36.70937 | 14.07165 | 93.34779 | 64.42569 | 3.106938 |
| Nudt7 | 10 | 16 | 52 | 63 | 65 | 33.37216 | 32.16377 | 59.19616 | 96.29485 | 2.372607 |
| Wwox | 7.08129 | 5 | 76 | 32.1551 | 68.1662 | 23.63179 | 10.05118 | 86.51746 | 49.14874 | 4.027739 |
| Maf | 6 | 8 | 64 | 46 | 80.091 | 20.02329 | 16.08188 | 72.85681 | 70.31052 | 3.965286 |
| uc009npo.1 | 23.7392 | 0.547095 | 120.324 | 36.5793 | 121.657 | 79.22283 | 1.09979 | 136.9754 | 55.91108 | 2.401396 |
| Hsdl1 | 16 | 11.1024 | 95.4331 | 40.3063 | 116.432 | 53.39545 | 22.31844 | 108.6399 | 61.60776 | 2.248565 |
| Wfdc1 | 4 | 4 | 46 | 37 | 44 | 13.34886 | 8.040941 | 52.36583 | 56.55412 | 5.092143 |
| Cotl1 | 350.221 | 365.532 | 371.528 | 229.429 | 495.837 | 1168.763 | 734.8053 | 422.9429 | 350.6799 | 0.406407 |
| Gins2 | 46.4559 | 144.632 | 53.1729 | 43.2399 | 69.9399 | 155.0334 | 290.7444 | 60.53137 | 66.09174 | 0.28405 |
| Zfpm1 | 77 | 52 | 35 | 13 | 36 | 256.9656 | 104.5322 | 39.84357 | 19.87037 | 0.165185 |
| Cdt1 | 112.118 | 203.805 | 104.449 | 67.495 | 120.954 | 374.1619 | 409.696 | 118.9035 | 103.1654 | 0.283302 |
| Aprt | 511 | 1282 | 678 | 582 | 671 | 1705.317 | 2577.122 | 771.8268 | 889.581 | 0.387958 |
| Spire2 | 21 | 24 | 19 | 13 | 38 | 70.08153 | 48.24565 | 21.62937 | 19.87037 | 0.35072 |
| Agt | 2 | 0 | 66 | 35 | 65 | 6.674431 | 0 | 75.13358 | 53.49714 | 19.27216 |
| 2810004N23Rik | 63.9233 | 105.506 | 102.701 | 59.915 | 107.02 | 213.3258 | 212.0919 | 116.9135 | 91.57946 | 0.49009 |
| Sipa1l2 | 7 | 7 | 68 | 27 | 80.1457 | 23.36051 | 14.07165 | 77.41036 | 41.26922 | 3.170525 |
| Kcnk1 | 0 | 5 | 286 | 199.171 | 362.396 | 0 | 10.05118 | 325.5789 | 304.4308 | 62.68019 |
| Casp4 | 1.01089 | 2 | 132.192 | 91 | 191.332 | 3.373558 | 4.020471 | 150.4857 | 139.0926 | 39.16381 |
| Casp12 | 5 | 1 | 141 | 99 | 154 | 16.68608 | 2.010235 | 160.5127 | 151.3205 | 16.67886 |
| Mmp13 | 50 | 15 | 32 | 21 | 20 | 166.8608 | 30.15353 | 36.4284 | 32.09828 | 0.347826 |
| Tmem123 | 71 | 90 | 871 | 567 | 884 | 236.9423 | 180.9212 | 991.5356 | 866.6536 | 4.446881 |
| Birc2 | 30 | 25.6149 | 209.371 | 105.329 | 210.387 | 100.1165 | 51.49198 | 238.3454 | 160.9943 | 2.63402 |
| Birc3 | 19.733 | 16.0133 | 354.631 | 214.728 | 333.567 | 65.85328 | 32.1905 | 403.7075 | 328.2095 | 7.465207 |
| uc009oek.1 | 38 | 22 | 244.114 | 156 | 304 | 126.8142 | 44.22518 | 277.8964 | 238.4444 | 3.018841 |
| Endod1 | 15 | 16 | 472.915 | 224 | 525 | 50.05823 | 32.16377 | 538.3606 | 342.3817 | 10.71176 |
| 1700012B09Rik | 10 | 34 | 0 | 0 | 0 | 33.37216 | 68.348 | 0 | 0 | 0 |
| Mre11a | 30 | 41 | 47 | 23 | 38 | 100.1165 | 82.41965 | 53.50422 | 35.15526 | 0.485709 |
| 2200002K05Rik | 61 | 74 | 18 | 15 | 35 | 203.5702 | 148.7574 | 20.49098 | 22.92735 | 0.123233 |
| A230050P20Rik | 4.62575 | 2.66667 | 71.7778 | 52.5405 | 101.583 | 15.43713 | 5.360634 | 81.71096 | 80.30761 | 7.790194 |
| Angptl6 | 32.3742 | 5.33333 | 4.22222 | 1.45946 | 12.5245 | 108.0397 | 10.72125 | 4.806523 | 2.23077 | 0.059256 |
| Icam1 | 31.0953 | 36.7529 | 1328.1 | 726.337 | 1523.76 | 103.7717 | 73.88198 | 1511.893 | 1110.199 | 14.75956 |
| Qtrt1 | 64 | 58 | 82 | 42 | 91 | 213.5818 | 116.5936 | 93.34779 | 64.19657 | 0.477153 |
| Spc24 | 2.01921 | 126.337 | 50.3542 | 45.5765 | 61.2611 | 6.738539 | 253.9671 | 57.3226 | 69.66321 | 0.487085 |
| Rab3d | 16.0252 | 12.1082 | 150.793 | 75.8901 | 156.306 | 53.47955 | 24.34033 | 171.6609 | 115.9972 | 3.696461 |
| Tmem205 | 54 | 73 | 315 | 240.976 | 370 | 180.2096 | 146.7472 | 358.5921 | 368.3293 | 2.223295 |
| Rgl3 | 20 | 2 | 119 | 48 | 122 | 66.74431 | 4.020471 | 135.4681 | 73.3675 | 2.951124 |
| Acp5 | 28 | 47 | 29 | 23 | 39 | 93.44204 | 94.48106 | 33.01324 | 35.15526 | 0.362747 |
| Zfp809 | 5 | 6 | 54 | 30.0038 | 75 | 16.68608 | 12.06141 | 61.47293 | 45.8605 | 3.733663 |
| Zfp810 | 5 | 6 | 78 | 30 | 76 | 16.68608 | 12.06141 | 88.79424 | 45.85469 | 4.68385 |
| Eepd1 | 14 | 34 | 15 | 10 | 17 | 46.72102 | 68.348 | 17.07581 | 15.2849 | 0.281229 |
| St14 | 1 | 1 | 130 | 53 | 201 | 3.337216 | 2.010235 | 147.9904 | 81.00995 | 42.82421 |
| Aplp2 | 108 | 101 | 1688 | 825 | 1650 | 360.4193 | 203.0338 | 1921.598 | 1261.004 | 5.648389 |
| St3gal4 | 81 | 88.8667 | 1179.34 | 900.894 | 1608.86 | 270.3145 | 178.643 | 1342.546 | 1377.007 | 6.057486 |
| Ei24 | 48 | 49 | 452.998 | 209 | 580 | 160.1864 | 98.50153 | 515.6873 | 319.4543 | 3.228376 |
| Tmem218 | 11 | 13 | 102 | 79 | 145 | 36.70937 | 26.13306 | 116.1155 | 120.7507 | 3.769208 |
| Vwa5a | 42 | 18 | 199 | 139 | 236 | 140.1631 | 36.18424 | 226.5391 | 212.4601 | 2.489401 |
| uc009pak.1 | 30.4119 | 28.1435 | 17.4184 | 12.0175 | 18.6571 | 101.4911 | 56.57506 | 19.82889 | 18.36862 | 0.241655 |
| C2cd2l | 7.01886 | 10.0285 | 78.048 | 29.0117 | 88.0622 | 23.42345 | 20.15964 | 88.84888 | 44.34408 | 3.056069 |
| Dpagt1 | 24 | 28.2027 | 170.239 | 100.418 | 227.235 | 80.09318 | 56.69406 | 193.798 | 153.4879 | 2.538876 |
| Slc37a4 | 11 | 12 | 78 | 38 | 95 | 36.70937 | 24.12282 | 88.79424 | 58.08261 | 2.414459 |
| Cxcr5 | 13 | 4 | 56 | 28 | 77 | 43.3838 | 8.040941 | 63.74971 | 42.79771 | 2.07191 |
| Phldb1 | 103.873 | 64 | 112 | 42 | 158.01 | 346.6466 | 128.6551 | 127.4994 | 64.19657 | 0.403314 |
| Mpzl2 | 16 | 46 | 583 | 338.92 | 586.877 | 53.39545 | 92.47082 | 663.68 | 518.0357 | 8.101364 |
| Bace1 | 9.16291 | 18.021 | 133 | 80.54 | 188.108 | 30.57861 | 36.22645 | 151.4056 | 123.1046 | 4.109122 |
| Tagln | 1.16667 | 4.38095 | 70.5263 | 47.5732 | 83.3581 | 3.893429 | 8.80674 | 80.28627 | 72.71514 | 12.04719 |
| Sidt2 | 78 | 55 | 405 | 186 | 508 | 260.3028 | 110.5629 | 461.047 | 284.2991 | 2.009746 |
| Ncam1 | 142.248 | 45.0104 | 171.903 | 54.0403 | 228.048 | 474.7122 | 90.4815 | 195.6923 | 82.60004 | 0.492384 |
| 1600029D21Rik | 1 | 3 | 133.54 | 119 | 204.442 | 3.337216 | 6.030706 | 152.0203 | 181.8903 | 35.64404 |
| Pts | 11.9718 | 19.116 | 166.322 | 126.232 | 164.511 | 39.95248 | 38.42766 | 189.3389 | 192.9443 | 4.877297 |
| 2310030G06Rik | 11 | 10 | 143.154 | 95 | 152 | 36.70937 | 20.10235 | 162.9647 | 145.2065 | 5.424431 |
| Cryab | 9.9 | 9 | 535.863 | 465.58 | 648.84 | 33.03843 | 18.09212 | 610.0198 | 711.6342 | 25.84862 |
| 1110032A03Rik | 20 | 15 | 97.1372 | 76 | 117 | 66.74431 | 30.15353 | 110.5798 | 116.1652 | 2.340042 |
| Acsbg1 | 111 | 128 | 25 | 15 | 27 | 370.4309 | 257.3101 | 28.45969 | 22.92735 | 0.08186 |
| Dnaja4 | 7 | 6 | 56 | 45 | 74 | 23.36051 | 12.06141 | 63.74971 | 68.78204 | 3.741518 |
| Nrg4 | 5.43565 | 2.47784 | 73.7099 | 37.1253 | 72.872 | 18.13994 | 4.981041 | 83.91044 | 56.74564 | 6.083483 |
| 1700017B05Rik | 37 | 34 | 277 | 85 | 271.37 | 123.477 | 68.348 | 315.3334 | 129.9216 | 2.321152 |
| Scamp2 | 45 | 58 | 341 | 165.988 | 373 | 150.1747 | 116.5936 | 388.1902 | 253.7109 | 2.406212 |
| Sema7a | 26 | 14 | 7 | 1 | 7 | 86.76761 | 28.14329 | 7.968713 | 1.52849 | 0.082648 |
| uc009pwy.1 | 85 | 109 | 32 | 22 | 31 | 283.6633 | 219.1156 | 36.4284 | 33.62677 | 0.139336 |
| Neo1 | 203 | 121 | 199 | 92 | 230 | 677.4548 | 243.2385 | 226.5391 | 140.621 | 0.398787 |
| Kif23 | 15.4958 | 129.836 | 83.7389 | 33.1923 | 65.1498 | 51.71283 | 261.0009 | 95.32733 | 50.73409 | 0.467077 |
| Paqr5 | 27 | 20 | 23.011 | 13 | 34.0094 | 90.10482 | 40.20471 | 26.19544 | 19.87037 | 0.353511 |
| Cln6 | 7 | 3 | 94 | 22 | 124 | 23.36051 | 6.030706 | 107.0084 | 33.62677 | 4.78494 |
| Calml4 | 94 | 98 | 81.0658 | 79 | 92 | 313.6983 | 197.0031 | 92.2843 | 120.7507 | 0.417142 |
| Zwilch | 37 | 102 | 63.8393 | 41 | 72.0939 | 123.477 | 205.044 | 72.67387 | 62.66808 | 0.411974 |
| Snapc5 | 15 | 47 | 113 | 153 | 140 | 50.05823 | 94.48106 | 128.6378 | 233.8589 | 2.507946 |
| uc009qci.1 | 43.7554 | 1263.15 | 23.8763 | 203.776 | 108.224 | 146.0212 | 2539.229 | 27.18048 | 311.4695 | 0.126115 |
| Ptplad1 | 47 | 64 | 332 | 144.286 | 395.792 | 156.8491 | 128.6551 | 377.9447 | 220.5397 | 2.096237 |
| Oaz2 | 57 | 73 | 323 | 208 | 570 | 190.2213 | 146.7472 | 367.6992 | 317.9259 | 2.034686 |
| Rab8b | 12.2311 | 16.2633 | 98.0012 | 61.7596 | 119.207 | 40.81782 | 32.69306 | 111.5634 | 94.39891 | 2.801793 |
| Lactb | 6 | 10 | 52 | 30 | 40 | 20.02329 | 20.10235 | 59.19616 | 45.85469 | 2.618047 |
| Gcnt3 | 215.3 | 194.605 | 149.703 | 68.0477 | 147.253 | 718.5025 | 391.2018 | 170.42 | 104.0102 | 0.2473 |
| B230380D07Rik | 21.4375 | 10.0588 | 92.6588 | 57.95 | 128.365 | 71.54156 | 20.22055 | 105.4816 | 88.57598 | 2.11479 |
| uc009qqk.1 | 16 | 9 | 75 | 60 | 77 | 53.39545 | 18.09212 | 85.37907 | 91.70938 | 2.477192 |
| Ccpg1 | 28.184 | 10.597 | 241.117 | 152.3 | 272.395 | 94.05609 | 21.30246 | 274.4846 | 232.789 | 4.397365 |
| Lysmd2 | 42 | 74 | 289 | 219.773 | 256 | 140.1631 | 148.7574 | 328.994 | 335.9208 | 2.301377 |
| Gclc | 4 | 20 | 82 | 122 | 31 | 13.34886 | 40.20471 | 93.34779 | 186.4757 | 5.225114 |
| Fbxo9 | 23 | 38 | 178.145 | 139 | 137 | 76.75596 | 76.38894 | 202.7981 | 212.4601 | 2.711537 |
| Gsta4 | 272 | 276 | 3106 | 2426 | 3719 | 907.7227 | 554.8249 | 3535.832 | 3708.116 | 4.952966 |
| Gsta1 | 36.8375 | 60.753 | 14.0422 | 19.0908 | 28.7674 | 122.9347 | 122.1278 | 15.98547 | 29.18009 | 0.184302 |
| uc009qub.1 | 0.902009 | 53.237 | 0 | 10.9092 | 0.291951 | 3.010199 | 107.0189 | 0 | 16.6746 | 0.151547 |
| Mto1 | 40.5168 | 42.1357 | 50.5054 | 26.7894 | 65.7814 | 135.2133 | 84.70267 | 57.49472 | 40.94732 | 0.447635 |
| Slc17a5 | 34.471 | 12.8364 | 129.258 | 126.347 | 165.317 | 115.0372 | 25.80418 | 147.1457 | 193.1201 | 2.415951 |
| Col12a1 | 4 | 0 | 127.746 | 81 | 119.738 | 13.34886 | 0 | 145.4245 | 123.8077 | 20.16892 |
| Myo6 | 82 | 43 | 484.515 | 185.456 | 585 | 273.6517 | 86.44012 | 551.5659 | 283.4676 | 2.318946 |
| Irak1bp1 | 7 | 20 | 49 | 52 | 58.8056 | 23.36051 | 40.20471 | 55.78099 | 79.48146 | 2.127932 |
| Hmgn3 | 4 | 28 | 69.8182 | 54.7639 | 69.9559 | 13.34886 | 56.28659 | 79.48018 | 83.70606 | 2.343436 |
| Sh3bgrl2 | 12 | 22.9163 | 105.718 | 43 | 88.0655 | 40.04659 | 46.06716 | 120.3481 | 65.72506 | 2.160783 |
| Nt5e | 209.631 | 66 | 4 | 3.00416 | 12 | 699.5838 | 132.6755 | 4.553551 | 4.591828 | 0.010989 |
| 2810026P18Rik | 143.921 | 457.937 | 231.454 | 182.909 | 214.935 | 480.2954 | 920.5611 | 263.4844 | 279.5745 | 0.387662 |
| Tmed3 | 62 | 54 | 288 | 200 | 372 | 206.9074 | 108.5527 | 327.8556 | 305.6979 | 2.008348 |
| Ctsh | 7 | 13 | 449 | 320 | 617 | 23.36051 | 26.13306 | 511.136 | 489.1167 | 20.20975 |
| Adamts7 | 4 | 1 | 85 | 33 | 90 | 13.34886 | 2.010235 | 96.76295 | 50.44016 | 9.584099 |
| Tbc1d2b | 43.063 | 11 | 206.703 | 90 | 256 | 143.7105 | 22.11259 | 235.3081 | 137.5641 | 2.248614 |
| Plscr1 | 372.863 | 510.789 | 2784.36 | 1954.05 | 2815.54 | 1244.324 | 1026.806 | 3169.681 | 2986.745 | 2.710732 |
| Pcolce2 | 51 | 96 | 5 | 1 | 1.00058 | 170.198 | 192.9826 | 5.691938 | 1.52849 | 0.019881 |
| Tfdp2 | 37 | 45 | 47 | 24.0423 | 48 | 123.477 | 90.46059 | 53.50422 | 36.74841 | 0.421864 |
| Slc25a36 | 64 | 22 | 372 | 160 | 347.873 | 213.5818 | 44.22518 | 423.4802 | 244.5583 | 2.591235 |
| Nmnat3 | 9 | 13 | 87.4041 | 42 | 85.4998 | 30.03494 | 26.13306 | 99.49975 | 64.19657 | 2.914405 |
| Il20rb | 4 | 2 | 108 | 69 | 149 | 13.34886 | 4.020471 | 122.9459 | 105.4658 | 13.15028 |
| Nck1 | 52 | 73 | 337 | 259 | 321 | 173.5352 | 146.7472 | 383.6366 | 395.8788 | 2.433838 |
| Pccb | 7 | 12 | 146.196 | 102.165 | 168.789 | 23.36051 | 24.12282 | 166.4277 | 156.1581 | 6.793665 |
| Amotl2 | 189 | 120 | 1228 | 576.837 | 1421 | 630.7338 | 241.2282 | 1397.94 | 881.6894 | 2.614368 |
| Slco2a1 | 52 | 62.0054 | 33 | 15.0584 | 41.9526 | 173.5352 | 124.6454 | 37.56679 | 23.01661 | 0.203177 |
| Trf | 299.861 | 73.0724 | 2712.42 | 1299.03 | 3302.5 | 1000.701 | 146.8927 | 3087.785 | 1985.554 | 4.42085 |
| Acad11 | 15 | 10.6364 | 78.4468 | 62.3274 | 104.374 | 50.05823 | 21.38167 | 89.30287 | 95.26679 | 2.583565 |
| Nudt16 | 10.0429 | 4 | 47.3091 | 37 | 63.1266 | 33.51532 | 8.040941 | 53.85609 | 56.55412 | 2.656885 |
| Acy1 | 21 | 41 | 33 | 18 | 47 | 70.08153 | 82.41965 | 37.56679 | 27.51281 | 0.426748 |
| Rrp9 | 30 | 93 | 67 | 32 | 80 | 100.1165 | 186.9519 | 76.27197 | 48.91167 | 0.436076 |
| Mapkapk3 | 9 | 1 | 54.7834 | 31 | 57 | 30.03494 | 2.010235 | 62.36475 | 47.38318 | 3.424788 |
| Cish | 13 | 5 | 68 | 56 | 78 | 43.3838 | 10.05118 | 77.41036 | 85.59542 | 3.050544 |
| Ifrd2 | 100 | 198 | 170.299 | 112 | 199 | 333.7216 | 398.0266 | 193.8663 | 171.1908 | 0.498884 |
| Sema3b | 26.9916 | 33.989 | 4 | 5.9994 | 10.9255 | 90.07679 | 68.32589 | 4.553551 | 9.170021 | 0.086637 |
| Sema3f | 53 | 9 | 816 | 276 | 878 | 176.8724 | 18.09212 | 928.9243 | 421.8631 | 6.928375 |
| Ube1l | 6 | 3 | 146 | 88 | 153.63 | 20.02329 | 6.030706 | 166.2046 | 134.5071 | 11.54186 |
| Tcta | 7 | 9 | 120 | 79 | 108 | 23.36051 | 18.09212 | 136.6065 | 120.7507 | 6.208465 |
| Ccdc36 | 14.5488 | 655.075 | 17.6578 | 74.2925 | 59.3236 | 48.55248 | 1316.855 | 20.10142 | 113.5553 | 0.097888 |
| Klhdc8b | 16 | 7 | 120.139 | 40 | 135 | 53.39545 | 14.07165 | 136.7648 | 61.13959 | 2.933346 |
| Lamb2 | 78 | 29 | 421 | 201 | 512 | 260.3028 | 58.29682 | 479.2612 | 307.2264 | 2.468577 |
| Dalrd3 | 8.38468 | 63.871 | 180.747 | 111.195 | 184.638 | 27.98149 | 128.3957 | 205.7602 | 169.9604 | 2.402655 |
| Slc25a20 | 27 | 30 | 143 | 103 | 191 | 90.10482 | 60.30706 | 162.7894 | 157.4344 | 2.12898 |
| Col7a1 | 37 | 32.305 | 39 | 13 | 35 | 123.477 | 64.94065 | 44.39712 | 19.87037 | 0.341091 |
| Shisa5 | 131 | 100 | 859 | 405 | 1022 | 437.1752 | 201.0235 | 977.875 | 619.0383 | 2.502219 |
| Plxnb1 | 37 | 17 | 370.115 | 127 | 422.078 | 123.477 | 34.174 | 421.3343 | 194.1182 | 3.903893 |
| Nradd | 166 | 146 | 201 | 123 | 205 | 553.9778 | 293.4944 | 228.8159 | 188.0042 | 0.491839 |
| Epm2aip1 | 26 | 14.0251 | 183 | 105 | 210.648 | 86.76761 | 28.19375 | 208.3249 | 160.4914 | 3.208177 |
| Stac | 21 | 17 | 0 | 0 | 1 | 70.08153 | 34.174 | 0 | 0 | 0 |
| uc009rwm.1 | 31.2592 | 39 | 279.977 | 139 | 304 | 104.3187 | 78.39918 | 318.7224 | 212.4601 | 2.907118 |
| Cmtm7 | 74 | 86 | 65 | 81.0107 | 61.0394 | 246.954 | 172.8802 | 73.9952 | 123.824 | 0.471184 |
| Cmtm8 | 39.1319 | 40.5961 | 27.1836 | 24.1081 | 27.1078 | 130.5916 | 81.60771 | 30.94547 | 36.84898 | 0.319485 |
| Oxsr1 | 23 | 20 | 16 | 3.00525 | 15 | 76.75596 | 40.20471 | 18.2142 | 4.593494 | 0.195003 |
| Myd88 | 18 | 26 | 131 | 74 | 163 | 60.06988 | 52.26612 | 149.1288 | 113.1082 | 2.334399 |
| Myrip | 3 | 0 | 57 | 29 | 88 | 10.01165 | 0 | 64.8881 | 44.3262 | 10.90872 |
| Trak1 | 46.0095 | 15.0326 | 230.211 | 105.782 | 288.686 | 153.5436 | 30.21906 | 262.0694 | 161.6867 | 2.305996 |
| Snrk | 31 | 12 | 254.977 | 158.47 | 313 | 103.4537 | 24.12282 | 290.2627 | 242.2198 | 4.173828 |
| Abhd5 | 29.8529 | 41.9318 | 169.709 | 129.527 | 196.401 | 99.62556 | 84.29279 | 193.1946 | 197.9807 | 2.126897 |
| Tmem42 | 5 | 12 | 52.1918 | 42 | 53 | 16.68608 | 24.12282 | 59.4145 | 64.19657 | 3.029022 |
| Cdcp1 | 14 | 12.0035 | 104 | 53.6603 | 107.967 | 46.72102 | 24.12986 | 118.3923 | 82.01921 | 2.828639 |
| Tmem158 | 91 | 76 | 28 | 22.0031 | 44 | 303.6866 | 152.7779 | 31.87485 | 33.63151 | 0.143508 |
| Fyco1 | 17 | 7 | 89.4043 | 33 | 96.5312 | 56.73267 | 14.07165 | 101.7767 | 50.44016 | 2.149825 |
| 2010204K13Rik | 37 | 132.8 | 25.0456 | 22 | 21 | 123.477 | 266.9592 | 28.5116 | 33.62677 | 0.159151 |
| Prickle3 | 423.08 | 692.309 | 569.543 | 356.512 | 759.351 | 1411.909 | 1391.704 | 648.3607 | 544.9249 | 0.425624 |
| Plp2 | 197.409 | 635.744 | 351.987 | 334.875 | 512.677 | 658.7964 | 1277.995 | 400.6976 | 511.853 | 0.471166 |
| Glod5 | 16 | 24 | 1 | 1 | 0 | 53.39545 | 48.24565 | 1.138388 | 1.52849 | 0.026238 |
| Suv39h1 | 116 | 129 | 92 | 41 | 101 | 387.117 | 259.3204 | 104.7317 | 62.66808 | 0.258957 |
| Wdr13 | 14 | 15.0043 | 117 | 45 | 158 | 46.72102 | 30.16217 | 133.1914 | 68.78204 | 2.627016 |
| Cybb | 2.06785 | 1 | 124.507 | 76.3269 | 109.898 | 6.900861 | 2.010235 | 141.7372 | 116.6649 | 28.99779 |
| Klhl13 | 30 | 23 | 248 | 119 | 262 | 100.1165 | 46.23541 | 282.3201 | 181.8903 | 3.171879 |
| Wdr44 | 1.06449 | 8.14314 | 42.4383 | 34.563 | 39.0534 | 3.552433 | 16.36963 | 48.31124 | 52.82919 | 5.076805 |
| Il13ra1 | 14 | 9 | 232.186 | 138 | 254.348 | 46.72102 | 18.09212 | 264.3177 | 210.9316 | 7.332607 |
| 6-Sep | 30 | 29 | 30.1097 | 14.0046 | 33 | 100.1165 | 58.29682 | 34.27651 | 21.40589 | 0.351501 |
| Gria3 | 0 | 4.1153 | 62.4596 | 40 | 76.3931 | 0 | 8.272721 | 71.10324 | 61.13959 | 15.98541 |
| Utp14a | 133.692 | 557.241 | 280.624 | 258.23 | 290.624 | 446.159 | 1120.186 | 319.4589 | 394.7019 | 0.455941 |
| Rbmx2 | 16.1019 | 57 | 31.2153 | 31 | 37 | 53.73551 | 114.5834 | 35.53511 | 47.38318 | 0.492626 |
| uc009tee.1 | 4 | 1 | 86 | 33 | 95 | 13.34886 | 2.010235 | 97.90134 | 50.44016 | 9.658217 |
| Gpc4 | 7 | 1 | 81 | 38 | 76 | 23.36051 | 2.010235 | 92.2094 | 58.08261 | 5.923831 |
| Cxx1a | 39.1109 | 30.9816 | 179.979 | 140.294 | 246.401 | 130.5215 | 62.28031 | 204.8859 | 214.4379 | 2.174896 |
| Cxx1b | 2.35525 | 8.01842 | 73.3039 | 42.1022 | 82.5522 | 7.859977 | 16.11891 | 83.44825 | 64.35278 | 6.163798 |
| Fgf13 | 17 | 36.0752 | 156 | 113.911 | 163.116 | 56.73267 | 72.51964 | 177.5885 | 174.1118 | 2.721037 |
| Ids | 45 | 39 | 273.462 | 148.959 | 289 | 150.1747 | 78.39918 | 311.3058 | 227.6823 | 2.358047 |
| BC023829 | 31 | 14 | 133 | 75 | 124 | 103.4537 | 28.14329 | 151.4056 | 114.6367 | 2.021644 |
| Mtm1 | 8.40102 | 17.2178 | 70.5094 | 49.5942 | 99.1313 | 28.03602 | 34.61183 | 80.26703 | 75.80422 | 2.491247 |
| Mtmr1 | 10 | 17 | 115 | 69 | 143 | 33.37216 | 34.174 | 130.9146 | 105.4658 | 3.499538 |
| Cetn2 | 36 | 73 | 288 | 207 | 337.86 | 120.1398 | 146.7472 | 327.8556 | 316.3974 | 2.413955 |
| Xlr3a | 79.4993 | 83.3092 | 1.1821 | 0 | 30.2308 | 265.3063 | 167.4711 | 1.345688 | 0 | 0.003109 |
| F8a | 14 | 10 | 105 | 61 | 92 | 46.72102 | 20.10235 | 119.5307 | 93.23787 | 3.184044 |
| Dusp9 | 41 | 43 | 17 | 3.00426 | 21 | 136.8258 | 86.44012 | 19.35259 | 4.59198 | 0.107247 |
| Slc6a8 | 46 | 32 | 362.589 | 163 | 444 | 153.5119 | 64.32753 | 412.7668 | 249.1438 | 3.038525 |
| Taz | 34 | 20 | 175 | 77 | 199 | 113.4653 | 40.20471 | 199.2178 | 117.6937 | 2.062286 |
| Plxna3 | 31 | 26 | 34 | 13 | 33 | 103.4537 | 52.26612 | 38.70518 | 19.87037 | 0.37616 |
| Ubl4 | 36 | 214 | 92 | 84 | 82 | 120.1398 | 430.1904 | 104.7317 | 128.3931 | 0.423609 |
| Fam3a | 10 | 15 | 74 | 33 | 72 | 33.37216 | 30.15353 | 84.24069 | 50.44016 | 2.120101 |
| G6pdx | 65 | 37 | 417 | 205 | 513 | 216.919 | 74.37871 | 474.7076 | 313.3404 | 2.705301 |
| uc009trd.1 | 48.1443 | 1.18666 | 29.7984 | 7.34643 | 35.5445 | 160.6679 | 2.385466 | 33.92213 | 11.22894 | 0.27691 |
| Pola1 | 280.993 | 4277.64 | 458.048 | 1360.38 | 600.452 | 937.7342 | 8599.063 | 521.4362 | 2079.327 | 0.272708 |
| uc009ttr.1 | 13.2996 | 17.5596 | 89.0146 | 49.6646 | 87.8142 | 44.38363 | 35.29893 | 101.3331 | 75.91183 | 2.224388 |
| Yipf6 | 41.1665 | 31 | 214.084 | 120.154 | 232 | 137.3815 | 62.31729 | 243.7106 | 183.6541 | 2.140047 |
| Slc16a2 | 7 | 1 | 453 | 119.012 | 530.039 | 23.36051 | 2.010235 | 515.6896 | 181.9086 | 27.49617 |
| 2610002M06Rik | 21 | 12 | 84 | 77 | 77 | 70.08153 | 24.12282 | 95.62456 | 117.6937 | 2.264421 |
| uc009ucr.1 | 3.95212 | 124.546 | 4.18206 | 31.9118 | 4.34968 | 13.18908 | 250.3668 | 4.760805 | 48.77686 | 0.203136 |
| Sh3bgrl | 166.267 | 156.238 | 1302.07 | 947.661 | 1477.44 | 554.8688 | 314.0751 | 1482.26 | 1448.49 | 3.372773 |
| Tspan6 | 3 | 2 | 85 | 72 | 96 | 10.01165 | 4.020471 | 96.76295 | 110.0513 | 14.73863 |
| Trmt2b | 11 | 9 | 73.0373 | 51.1846 | 94.0079 | 36.70937 | 18.09212 | 83.14476 | 78.23513 | 2.944808 |
| Hnrnph2 | 89.2639 | 92.1335 | 565.192 | 329.841 | 547.465 | 297.8929 | 185.21 | 643.4076 | 504.1586 | 2.375407 |
| Armcx1 | 4 | 3 | 62 | 53 | 72 | 13.34886 | 6.030706 | 70.58003 | 81.00995 | 7.822155 |
| Armcx3 | 9 | 2 | 205 | 123 | 259 | 30.03494 | 4.020471 | 233.3695 | 188.0042 | 12.37318 |
| Armcx2 | 10.0375 | 4 | 108.126 | 50.2655 | 124.732 | 33.4973 | 8.040941 | 123.0893 | 76.8303 | 4.812905 |
| Bhlhb9 | 0 | 1 | 74 | 54 | 82 | 0 | 2.010235 | 84.24069 | 82.53844 | 82.96498 |
| Tceal8 | 35 | 50 | 456 | 428 | 499 | 116.8025 | 100.5118 | 519.1048 | 654.1936 | 5.399085 |
| Bex1 | 6 | 11 | 114.29 | 121.379 | 125.552 | 20.02329 | 22.11259 | 130.1063 | 185.5265 | 7.490833 |
| Tceal1 | 9 | 5 | 36 | 44 | 33 | 30.03494 | 10.05118 | 40.98195 | 67.25355 | 2.700074 |
| Rnf128 | 107 | 161.109 | 148 | 96 | 167 | 357.0821 | 323.867 | 168.4814 | 146.735 | 0.462907 |
| Cldn2 | 298 | 225 | 3718 | 2173 | 4007.71 | 994.4903 | 452.3029 | 4232.525 | 3321.408 | 5.221156 |
| Tsc22d3 | 34.2486 | 19.4334 | 220.069 | 143.753 | 229.781 | 114.295 | 39.06571 | 250.5238 | 219.725 | 3.066293 |
| Mid2 | 6 | 1 | 82 | 39 | 98 | 20.02329 | 2.010235 | 93.34779 | 59.6111 | 6.942096 |
| Vsig1 | 41 | 108 | 16 | 14 | 17 | 136.8258 | 217.1054 | 18.2142 | 21.39886 | 0.111923 |
| Psmd10 | 48 | 52 | 306 | 278 | 356 | 160.1864 | 104.5322 | 348.3466 | 424.9201 | 2.92109 |
| Atg4a | 14 | 6.75 | 137 | 100.96 | 188 | 46.72102 | 13.56909 | 155.9591 | 154.3163 | 5.146374 |
| Nxt2 | 17 | 21 | 161 | 74 | 118.599 | 56.73267 | 42.21494 | 183.2804 | 113.1082 | 2.99541 |
| Acsl4 | 149 | 108.2 | 1008.34 | 547.227 | 1102.11 | 497.2451 | 217.5075 | 1147.882 | 836.4308 | 2.776223 |
| Amot | 11 | 15 | 78 | 43.211 | 109.026 | 36.70937 | 30.15353 | 88.79424 | 66.04757 | 2.31581 |
| Fam120c | 8.13426 | 17 | 92.1937 | 40.0011 | 85.5598 | 27.14578 | 34.174 | 104.9522 | 61.14127 | 2.708644 |
| Iqsec2 | 13 | 5 | 75 | 25 | 96 | 43.3838 | 10.05118 | 85.37907 | 38.21224 | 2.312929 |
| Mageh1 | 13 | 18 | 80 | 69 | 76 | 43.3838 | 36.18424 | 91.07101 | 105.4658 | 2.470047 |
| uc009ura.1 | 34 | 39 | 171 | 135 | 188 | 113.4653 | 78.39918 | 194.6643 | 206.3461 | 2.090071 |
| Sat1 | 245 | 129 | 3163.99 | 2411.99 | 2348 | 817.6178 | 259.3204 | 3601.847 | 3686.702 | 6.767843 |
| Sms | 97.9417 | 132 | 90 | 65 | 91 | 326.8526 | 265.3511 | 102.4549 | 99.35183 | 0.340773 |
| Gpr64 | 19 | 25 | 119 | 80.5574 | 121.359 | 63.4071 | 50.25588 | 135.4681 | 123.1312 | 2.275141 |
| S100g | 2 | 24 | 448 | 743 | 402 | 6.674431 | 48.24565 | 509.9977 | 1135.668 | 29.96473 |
| Pir | 44 | 52 | 38 | 24 | 44.1965 | 146.8375 | 104.5322 | 43.25873 | 36.68375 | 0.318027 |
| Gpm6b | 185 | 98 | 174.745 | 128 | 192 | 617.3849 | 197.0031 | 198.9275 | 195.6467 | 0.484504 |
| Tmsb4x | 2605.94 | 11538 | 4758.98 | 4864 | 5368 | 8696.584 | 23194.1 | 5417.564 | 7434.574 | 0.403006 |
| Prps2 | 36 | 58 | 391 | 234 | 428 | 120.1398 | 116.5936 | 445.1096 | 357.6666 | 3.391056 |
| Vamp7 | 65.0142 | 45 | 334.41 | 232.683 | 329.228 | 216.9664 | 90.46059 | 380.6882 | 355.6536 | 2.395176 |
| Eif2s3y | 55 | 39 | 296 | 168.411 | 381 | 183.5469 | 78.39918 | 336.9627 | 257.4145 | 2.269083 |
| uc009vch.1 | 28.4407 | 33.861 | 159.98 | 119.322 | 142.58 | 94.91275 | 68.06858 | 182.1193 | 182.3824 | 2.236463 |
| uc009vfd.1 | 252.107 | 1796.81 | 95.727 | 616.604 | 233.76 | 841.3354 | 3612.011 | 108.9744 | 942.4728 | 0.236103 |

What is claimed is:

1. A method for analyzing cellular nucleic acid, the method comprising the steps of:
capturing RNA from a lysed cell onto a substrate;
producing a cDNA/RNA duplex;
removing the RNA from the cDNA/RNA duplex;
priming the cDNA to produce a primer/cDNA duplex;
exposing the primer/cDNA duplex to at least one detectably labeled nucleotide in the presence of a polymerase capable of catalyzing addition of the nucleotide to the primer/cDNA duplex;
detecting incorporation of the nucleotide into the primer portion; and
repeating the exposing and detecting steps at least once.

2. The method according to claim 1, wherein prior to the capturing step, the method further comprises lysing a cell to release RNA from that cell.

3. The method according to claim 1, wherein capturing comprises:
adding a poly(A) tail to the RNA; and
hybridizing the poly(A) tailed RNA to poly(T) primers that are attached to the substrate.

4. The method according to claim 3, wherein producing the cDNA/RNA duplex comprises: performing a sequencing reaction on the poly(A) tailed RNA that is hybridized to the poly(T) primer, thereby producing the cDNA/RNA duplex.

5. The method according to claim 4, wherein the sequencing reaction is a sequencing-by-synthesis reaction.

6. The method according to claim 1, wherein priming comprises:
determining the sequence of the cDNA;
synthesizing a primer that corresponds to a portion of the cDNA; and
hybridizing the primer to the corresponding portion of the cDNA.

7. The method according to claim 1, wherein priming comprises:
adding a poly(G) tail to a 3′ end of the cDNA; and
hybridizing a poly(C) primer to the poly(G) tail.

8. The method according to claim 7, wherein prior to the exposing step, the method further comprises: adding dCTP to the primer/cDNA duplex to fill remaining unpaired poly(G) nucleotides on the poly(G) tail of the cDNA, thereby ensuring alignment of the poly(C) primer with the poly(G) tail.

9. The method according to claim 8, further comprising: exposing the primer/cDNA duplex to locked nucleic acids.

10. The method according to claim 3, wherein prior to the exposing step, the method further comprises: adding dATP in order to fill poly(T) primers on the surface of the substrate that did not pair with an RNA.

11. The method according to claim 1, further comprising: determining a sequence of the cDNA based upon the order of incorporation of the labeled nucleotides.

12. The method according to claim 1, further comprising: removing unincorporated nucleotide and polymerase in all or some repetitions of the exposing and detecting steps.

13. The method according to claim 1, further comprising: neutralizing the label on the labeled nucleotide after the detecting step.

14. The method according to claim 1, further comprising:
removing a complementary strand of the cDNA; and
resequencing the cDNA at least once.

15. The method according to claim 1, wherein at least a portion of the primer/cDNA duplexes are individually optically resolvable.

16. The method according to claim 1, wherein the nucleotide is a non-native nucleotide.

17. The method according to claim 1, wherein the detectably labeled nucleotide is an optically labeled nucleotide.

18. The method according to claim 17, wherein the optical label is a fluorescent label.

19. The method according to claim 18, wherein the fluorescent label is selected from the group consisting of cyanine, rhodamine, fluorescien, coumarin, BODIPY, alexa, and conjugated multi-dyes.

20. The method according to claim 1, wherein the lysed cells are selected from the group consisting of bacterial cells, fungi cells, and human cells.

21. The method according to claim 20, wherein the human cells are cancer cells.
